(19) United States
(12) Patent Application Publication
(10) Pub. No.: US 2026/0273100 A1
(43) Pub. Date: Sep. 17, 2026

(54) USE OF PRIME EDITING FOR TREATING SICKLE CELL DISEASE

(71) Applicants: The Broad Institute, Inc., Cambridge, MA (US); President and Fellows of Harvard College, Cambridge, MA (US); St. Jude Children's Research Hospital, Inc., Memphis, TN (US)

(72) Inventors: David R. Liu, Cambridge, MA (US); Kelcee Everette, Cambridge, MA (US); Gregory Newby, Cambridge, MA (US); Jonathan S. Yen, Memphis, TN (US); Mitchell J. Weiss, Memphis, TN (US)

(73) Assignees: The Broad Institute, Inc., Cambridge, MA (US); President and Fellows of Harvard College, Cambridge, MA (US); St. Jude Children's Research Hospital, Inc., Memphis, TN (US)

(21) Appl. No.: 19/168,198

(22) PCT Filed: Mar. 22, 2024
(86) PCT No.: PCT/US2024/021103

§ 371 Date: Sep. 23, 2025

Related U.S. Application Data
(60) Provisional application No. 63/454,583, filed on Mar. 24, 2023.

(51) Int. Cl. A61K 48/00; A61K 31/712; A61K 31/7125; A61K 35/00; A61P 7/06; C12N 5/0789; C12N 15/10; C12N 15/113; C12N 15/63; C12N 15/87

(52) U.S. Cl. CPC A61K 48/005; A61K 31/712; A61K 31/7125; A61K 35/00; A61P 7/06; C12N 5/0647; C12N 15/102; C12N 15/113; C12N 15/63; C12N 15/87; C12N 2310/315; C12N 2310/321; C12N 2510/00

(57) ABSTRACT

The present disclosure provides methods for treating sickle cell disease using prime editing. The present disclosure also provides epegRNAs targeting the β-globin (HBB) gene, which may be useful for treating sickle cell disease. Also provided herein are prime editor complexes, polynucleotides, vectors, pharmaceutical compositions, kits, and cells useful for performing the methods described herein.

### RELATED APPLICATIONS

This application claims priority under 35 U.S.C. § 119 (e) to U.S. Provisional application, U.S. Ser. No. 63/454,583, filed Mar. 24, 2023, which is incorporated herein by reference.

### GOVERNMENT SUPPORT

This invention was made with government support under Grant Nos. U01 AI142756, RM1 HG009490, R35 GM118062, R01 HL156647, and R01 HL136135, awarded by the National Institutes of Health. The government has certain rights in the invention.

### REFERENCE TO AN ELECTRONIC SEQUENCE LISTING

The contents of the electronic sequence listing (B119570181WO00-SEQ-TNG.xml; Size: 499,442 bytes; and Date of Creation: Mar. 21, 2024) is herein incorporated by reference in its entirety.

### BACKGROUND OF THE INVENTION

Sickle cell disease (SCD) is an autosomal recessive disorder caused by an A·T-to-T·A mutation at nucleotide position 20 in the hemoglobin subunit beta (HBB) gene, resulting in the pathogenic HBB^{S} allele encoding a Glu→Val substitution. This mutation changes normal adult β-globin (β^{A}) to sickle β-globin (β^{S}) and results in replacement of normal adult hemoglobin (HbA, α₂β₂) with sickle hemoglobin (HbS, α₂β^{S}₂). At low oxygen tension, HbS forms rigid polymers that cause characteristic red blood cell (RBC) shape changes and initiate a complex pathophysiology that includes hemolysis, microvascular occlusions, and inflammation. Clinical manifestations include anemia, immunodeficiency, multi-organ damage, severe acute and chronic pain, and premature death¹.

The only FDA-approved cure for SCD is allogeneic hematopoietic stem cell transplantation (HSCT). However, most patients lack ideal donors, and the procedure is associated with serious toxicities, including graft-vs-host disease and graft rejection⁷. Correction of the patient's own hematopoietic stem cells (HSCs) bypasses immune complications and eliminates the need for a tissue-compatible donor. Current strategies for therapeutic manipulation of SCD HSPCs being examined in clinical trials include lentiviral expression of an anti-sickling β-like globin⁸, the use of genome editing nucleases or base editors to activate-globin gene transcription for induction of fetal hemoglobin (HbF, α₂ψ₂)⁹⁻¹⁴, and Cas9 nuclease-initiated homology-directed repair (HDR) using an AAV6 DNA template to correct the SCD mutation¹⁵. A base editing strategy using an adenine base editor to convert the pathogenic HBB^{S} allele into the non-pathogenic, naturally occurring Makassar allele (HBB^{G}) has also been developed¹⁶. While each of these strategies has distinct advantages and disadvantages, reverting the SCD Glu→Val substitution, which requires a T·A-to-A·T transversion, represents the most physiological approach for disease correction. However, base editors developed thus far cannot convert T·A to A·T, and nuclease-mediated homology directed repair (HDR) requires double-stranded DNA breaks (DSB) that can cause on-target loss-of-function insertion and deletion (indel) mutations¹⁸, p53 activation, and chromosomal abnormalities^{19-23,25}. Moreover, co-delivery of the HDR template by AAV transduction^{15,26} has the potential to impair HSC engraftment^{18,27}. A treatment for SCD that would permanently revert HBB^{S} to wild-type HBB^{A} with minimal or no deleterious genomic alterations or cell state changes is therefore needed.

### SUMMARY OF THE INVENTION

Prime editing replaces a target segment of DNA with a specified new sequence up to hundreds of base pairs in length, thus enabling the installation of targeted insertions, deletions, and any base-to-base substitutions directly into the genome of living cells and animals, without requiring DSBs^{2-4,28-32}. Described herein is the development of a prime editing strategy that reverts the SCD allele back to wild-type HBB^{A} with high on-target efficiency, low frequencies of indel byproducts, and minimal off-target editing. Edited HSPCs of SCD human patients maintained prime editing levels at 17 weeks after transplantation in mice, with engrafted cells showing an allele correction frequency of up to 41%, and up to 42% of blood cells containing at least one wild-type HBB^{A} allele, indicating robust editing of hematopoietic stem cells at levels that exceed the estimated therapeutic threshold (a correction frequency of approximately 20% is estimated to be the therapeutic threshold for improvement of at least some disease parameters). Treated cells also showed a significant reduction in sickling when cultured in hypoxic conditions. Minimal off-target editing was detected following the analysis of over 100 experimentally identified CIRCLE-seq-nominated candidate off-target sites engaged by the prime editing system, suggesting a high degree of target DNA specificity. Taken together, these results are among the first examples of therapeutic prime editing in human HSCs and demonstrate a potential strategy for a one-time autologous SCD treatment that corrects the sickle globin allele back to wild-type HBB without requiring DSBs or donor DNA templates.

Thus, in one aspect, the present disclosure provides methods of treating sickle cell disease comprising contacting a target nucleotide sequence with a prime editor and an engineered prime editing guide RNA (epegRNA), wherein the epegRNA comprises the structure 5′-[spacer sequence]-[single guide RNA (sgRNA) scaffold sequence]-[extension arm sequence]-[optional engineered linker sequence]-[3′ structured motif]-3′, wherein each instance of]-[comprises an optional linker sequence. In some embodiments, the epegRNA comprises the spacer sequence 5′-CATGGTGCACCTGACTCCTG-3′ (SEQ ID NO: 127), or a sequence at least 90%, at least 95%, at least 96%, at least 97%, at least 98%, or at least 99% identical to the sequence 5′-CATGGTGCACCTGACTCCTG-3′ (SEQ ID NO: 127) or a fragment thereof. In certain embodiments, the epegRNA comprises the sequence 5′-CATGGTGCACCTGACTCCTGGTTTTAGAGCTAGAAATAGCAAGTTAAAATAAGGC TAGTCCGTTATCAACTTGAAAAAGTGGCACCGAGTCGGTGCAGACTTCTCTTCAGG AGTCAGGTGCACAGAATAAACGCGGTTCTATCTAGTTACGCGTTAAACCAACTAG AA-3′ (SEQ ID NO: 115), or a sequence at least 90%, at least 95%, at least 96%, at least 97%, at least 98%, or at least 99% identical to the sequence of SEQ ID NO: 115. In certain embodiments, the epegRNA comprises the sequence 5′-CATGGTGCACCTGACTCCTGGTTTTAGAGCTAGAAATAGCAAGTTAAAATAAGGC TAGTCCGTTATCAACTTGAAAAAGTGGCACCGAGTCGGTGCAGACTTCTCTTCAGG AGTCAGGTGCACAGAATAAACGCGGTTCTATCTAGTTACGCGTTAAACCAACTAG AATTT-3′ (SEQ ID NO: 116), or a sequence at least 90%, at least 95%, at least 96%, at least 97%, at least 98%, or at least 99% identical to the sequence of SEQ ID NO: 116.

The methods described herein may result in the correction of an A·T:T·A transversion mutation at nucleotide position 20 in the β-globin gene (HBB). In some embodiments, correction of the A·T:T·A transversion mutation in HBB reverts the sickle cell disease allele to the wild type allele. In some embodiments, correction of the A·T:T·A transversion mutation in HBB results in the correction of a valine mutation in the β-globin protein to a wild type glutamic acid residue. In certain embodiments, the method further results in the introduction of a silent PAM-disrupting edit in the HBB gene, e.g., a G→A silent PAM-disrupting edit at the nucleotide position following the A·T:T·A transversion mutation. Introduction of such a PAM-disrupting edit in the HBB gene may help the prime editing intermediate that is produced prior to incorporation of the edit to avoid reversion to the unedited sequence by the cellular DNA mismatch repair pathway.

In some embodiments, the methods provided herein further comprise nicking the non-PAM-containing strand of the target nucleotide sequence using a nicking sgRNA. The use of such a nicking gRNA may facilitate incorporation of the edit by cellular DNA repair mechanisms. In certain embodiments, the spacer sequence of the nicking sgRNA comprises the nucleotide sequence 5′-CCTTGATACCAACCTGCCCA-3′ (SEQ ID NO: 128), 5′-GTAACGGCAGACTTCTCTTC-3′ (SEQ ID NO: 129), 5′-CACGTTCACCTTGCCCCACA-3′ (SEQ ID NO: 130), or 5′-TCCACATGCCCAGTTTCTAT-3′ (SEQ ID NO: 131), or a sequence at least 90%, at least 95%, at least 96%, at least 97%, at least 98%, or at least 99% identical to the sequence 5′-CCTTGATACCAACCTGCCCA-3′ (SEQ ID NO: 128), 5′-GTAACGGCAGACTTCTCTTC-3′ (SEQ ID NO: 129), 5′-CACGTTCACCTTGCCCCACA-3′ (SEQ ID NO: 130), or 5′-TCCACATGCCCAGTTTCTAT-3′ (SEQ ID NO: 131) or a fragment thereof.

In some embodiments, the prime editor comprises PEmax architecture (e.g., PE3max, PE3bmax, PE4max, PE5max, or PE5bmax). In certain embodiments, the prime editor is PE3max. In certain embodiments, the prime editor is PE3bmax.

In some embodiments, the contacting is performed in a cell (e.g., a eukaryotic cell, such as a human cell). In certain embodiments, the cell is a hematopoietic stem or progenitor cell (HSPC), for example, an HSPC from a patient who is being treated for SCD. In some embodiments, the cell is a hemateopoetic stem cell (HSC). In some embodiments, an HSC edited using the methods described herein is able to retain the edits introduced into its sub-lineages (e.g., CD34⁺ cells, CD235a⁺ cells, CD33⁺ cells, CD19⁺ cells, etc.).

As described herein, delivery of DNA, such as plasmids or other vectors, to HSPCs is generally difficult and inefficient, but HSPCs are significantly more amenable to delivery of RNA, e.g., specifically by electroporation. Thus, in some embodiments, the prime editor is delivered to the cell as mRNA, and the epegRNA is delivered to the cell as RNA. In certain embodiments, a nicking sgRNA is also delivered to the cell as RNA. In some embodiments, the prime editor mRNA, epegRNA, and/or nicking sgRNA are delivered to a cell by electroporation (e.g., facilitating the editing of HSPCs ex vivo that can then be delivered to a sickle cell disease patient). In certain embodiments, the prime editor mRNA, epegRNA, and nicking sgRNA comprise approximately 20% of the total electroporation volume. In certain embodiments, the molar ratio of the amount of epegRNA to the amount of nicking sgRNA delivered to the cell is approximately 1.5:1.

Any of the methods described herein may be performed in vitro. In some embodiments, the methods described herein are performed ex vivo. In certain embodiments, the methods described herein are performed in a cell (e.g., an HSPC, HSC, or other human cell type) ex vivo, and the edited cell is subsequently transplanted into a subject to be treated for sickle cell disease. In some embodiments, an HSC edited using the methods described herein is able to retain the edits introduced into its sub-lineages (e.g., CD34⁺ cells, CD235a⁺ cells, CD33⁺ cells, CD19⁺ cells, etc.). In some embodiments, the methods described herein are performed in vivo (e.g., in a subject). In some embodiments, bone marrow cells in a subject are edited.

The methods provided herein provide several advantages over previously disclosed gene editing methods for treating sickle cell disease, including increased editing efficiency and lower rates of off-target effects. In some embodiments, the methods provided herein may result in a greater than 20%, greater than 25%, greater than 30%, greater than 35%, or greater than 40% efficiency of conversion of an A·T:T·A transversion mutation at nucleotide position 20 in the β-globin gene to the wild type sequence. In certain embodiments, a greater than 40% efficiency of conversion of an A·T:T·A transversion mutation at nucleotide position 20 in the β-globin gene to the wild type sequence is observed. In some embodiments, the methods provided herein result in an edit-to-indel ratio of greater than 5, greater than 5.5, greater than 6, greater than 6.5, greater than 7, or greater than 7.5. In certain embodiments, a greater than 7.5 edit-to-indel ratio is observed. In some embodiments, the methods provided herein result in at least 30%, at least 35%, or at least 40% of cells edited using the method retaining the edit following transplantation into a subject. In certain embodiments, the methods provided herein result in at least 40% of cells edited using the method retaining the edit following transplantation into a subject. In some embodiments, the methods provided herein are performed with a prime editing efficiency of at least 60%, at least 65%, at least 70%, at least 75%, or at least 80%. In certain embodiments, the methods provided herein are performed with a prime editing efficiency of at least 80%. In some embodiments, the methods provided herein result in an indel frequency of less than 10%, less than 9%, less than 8%, less than 7%, less than 6%, less than 5%, or less than 4%. In certain embodiments, the methods provided herein result in an indel frequency of less than 4%.

In another aspect, the present disclosure provides methods of treating sickle cell disease comprising contacting a target nucleotide sequence with a prime editor and a prime editing guide RNA (pegRNA), wherein the prime editor is PE3max or PE3bmax, and wherein the pegRNA comprises the structure 5′-[spacer sequence]-[single guide RNA (sgRNA) scaffold sequence]-[extension arm sequence]-3′, wherein each instance of]-[comprises an optional linker sequence.

In another aspect, the present disclosure provides methods of treating sickle cell disease comprising contacting a target nucleotide sequence with a prime editor, a prime editing guide RNA (pegRNA), and a nicking single guide RNA (sgRNA), wherein the pegRNA comprises the structure 5′-[spacer sequence]-[sgRNA scaffold sequence]-[extension arm sequence]-3′, wherein each instance of]-[comprises an optional linker sequence, and wherein the spacer sequence of the nicking sgRNA comprises the nucleotide sequence 5′-GTAACGGCAGACTTCTCTTC-3′ (SEQ ID NO: 129), 5′-CACGTTCACCTTGCCCCACA-3′ (SEQ ID NO: 130), or 5′-TCCACATGCCCAGTTTCTAT-3′ (SEQ ID NO: 131), or a sequence at least 90%, at least 95%, at least 96%, at least 97%, at least 98%, or at least 99% identical to the sequence 5′-GTAACGGCAGACTTCTCTTC-3′ (SEQ ID NO: 129), 5′-CACGTTCACCTTGCCCCACA-3′ (SEQ ID NO: 130), or 5′-TCCACATGCCCAGTTTCTAT-3′ (SEQ ID NO: 131), or a fragment thereof.

In another aspect, the present disclosure provides engineered prime editing guide RNAs (epegRNAs) targeting the β-globin gene (HBB), wherein the epegRNA comprises the structure 5′-[spacer sequence]-[single guide RNA (sgRNA) scaffold sequence]-[extension arm sequence]-[optional engineered linker sequence]-[3′ structured motif]-3′, wherein each instance of]-[comprises an optional linker sequence.

In another aspect, the present disclosure provides complexes comprising a prime editor (e.g., PE3max or PE3bmax) and any of the epegRNAs disclosed herein.

In another aspect, the present disclosure provides polynucleotides encoding any of the epegRNAs provided herein, or multiple polynucleotides encoding an epegRNA, a prime editor, and/or a nicking sgRNA as provided herein. In another aspect, the present disclosure provides vectors comprising any of the polynucleotides provided herein.

In another aspect, the present disclosure provides pharmaceutical compositions comprising any of the epegRNAs, nicking sgRNAs, complexes, polynucleotides, or vectors provided herein. In some embodiments, a pharmaceutical composition comprises any of the epegRNAs provided herein and an mRNA encoding a prime editor.

In another aspect, the present disclosure provides cells comprising any of the epegRNAs, nicking sgRNAs, complexes, polynucleotides, vectors, or combinations thereof provided herein. In certain embodiments, the cell is an HSPC. In certain embodiments, the cell is an HSC. In certain embodiments, the cell is a CD34⁺ cell. In certain embodiments, the cell is a CD235a⁺ cell. In certain embodiments, the cell is a CD33⁺ cell. In certain embodiments, the cell is a CD19⁺ cell. In certain embodiments, the cell is a human cell.

In another aspect, the present disclosure provides kits comprising any of the epegRNAs, nicking sgRNAs, complexes, polynucleotides, vectors, or combinations thereof provided herein.

In another aspect, the present disclosure provides for the use of any of the epegRNAs, nicking sgRNAs, complexes, polynucleotides, vectors, or pharmaceutical compositions provided herein for the treatment of sickle cell disease.

In another aspect, the present disclosure provides for the use of any of the epegRNAs, nicking sgRNAs, complexes, polynucleotides, vectors, or pharmaceutical compositions provided herein for the manufacture of a medicament for the treatment of sickle cell disease, for example, in a patient whose genome contains the A·T:T·A transversion mutation at nucleotide position 20 in the β-globin gene.

In another aspect, the present disclosure provides cells comprising DNA comprising the sequence 5′-ATGGTGCACCTGACTCCTGAAGAGAAG-3′ (SEQ ID NO: 78) (e.g., in the chromosomal DNA of the cell). In some embodiments, a cell comprises DNA comprising a sequence at least 90%, at least 95%, at least 96%, at least 97%, at least 98%, or at least 99% identical to the sequence 5′-ATGGTGCACCTGACTCCTGAAGAGAAG-3′ (SEQ ID NO: 78), wherein the underlined nucleotide is an A (i.e., the PAM-disrupting mutation introduced into the DNA as discussed above). In certain embodiments, the cell is an HSPC. In certain embodiments, the cell is an HSC. In certain embodiments, the cell is a CD34⁺ cell. In certain embodiments, the cell is a CD235a⁺ cell. In certain embodiments, the cell is a CD33⁺ cell. In certain embodiments, the cell is a CD19⁺ cell. In certain embodiments, the cell is a human cell.

It should be appreciated that the foregoing concepts, and additional concepts discussed below, may be arranged in any suitable combination, as the present disclosure is not limited in this respect. Further, other advantages and novel features of the present disclosure will become apparent from the following detailed description of various non-limiting embodiments when considered in conjunction with the accompanying Figures.

### BRIEF DESCRIPTION OF THE DRAWINGS

The following Figures form part of the present specification and are included to further demonstrate certain aspects of the present disclosure, which can be better understood by reference to one or more of these drawings in combination with the detailed description of specific embodiments presented herein.

FIGS. 1A-1D: Assessment of prime editing strategies in healthy human CD34⁺ hematopoietic stem and progenitor cells (HSPCs). Cells were thawed and allowed to recover 24 h before electroporation. Bars reflect mean±SD of n=3 independent biological replicates, with replicate values shown as individual dots. All intended edit values include only the desired prime editing product with no indels or other changes at the target site. Indels are shown as separate bars in each plot. FIG. 1A shows that PE can revert the HBB^{S} allele back to wild-type HBB^{A} by correcting the pathogenic T at position +4 (bold). Including a +5 G>A PAM-disrupting edit (underline) improves editing by eliminating the NGG PAM after the edit has been made and may also help the prime editing intermediate evade mismatch repair³. Sequences shown correspond (top-bottom) to SEQ ID NOs: 372-375. FIG. 1B shows prime editing efficiencies 3 days post-electroporation at various endogenous genomic loci in 5×10⁵ human CD34⁺ hematopoietic stem and progenitor cells (HSPCs) when epegRNAs were used with the canonical PE3 system or with the PE3 system using the improved PEmax prime editor architecture (PE3max). FIG. 1C shows editing efficiencies for 5×10⁵ cells per condition for PE2max and PE4max 3 days post-electroporation. FIG. 1D shows editing efficiencies for 5×10⁵ cells per condition for PE3max and PE5max 3 days post-electroporation.

FIGS. 2A-2F: Optimization of prime editing HBB in human CD34⁺ HSPCs from healthy donors and from SCD patients. To assess on-target editing, 5×10⁵ healthy donor HSPCs and 4-5×10⁶ sickle cell disease (SCD) patient HPSCs were electroporated. All healthy donor HSPCs and 1×10⁵ SCD patient HSPCs were cultured for 72 hours following electroporation. The remaining SCD patient HSPCs were cryopreserved for later mouse engraftment experiments. Editing efficiencies for healthy donor cells are for the +5 G>A PAM-disrupting edit, and editing efficiencies reported for SCD patient HSPCs reference correction of the pathogenic+4 T>A HBB^{S} mutation. Bars reflect mean±SD with replicate values shown as individual dots for both healthy and patient cells. For healthy donor HSPC editing n=3 independent biological replicates. For SCD patient donor editing n=4 independent biological replicates. All editing values include only the desired prime editing product with no indels or other changes at the target site. Indels are shown as separate bars in each plot. FIG. 2A shows quantification of editing efficiencies for different nicking sgRNAs targeting HBB. FIG. 2B shows the distance between the epegRNA-induced nick and the nicking sgRNA-induced nick on the opposite strand for four nicking sgRNA candidates at HBB. In the PE3b strategy, nicking of the unedited strand cannot take place until after editing and ligation of the other strand is complete. FIG. 2C shows electroporation components and total combined volume of PEmax mRNA, epegRNA, and nicking sgRNAs for various PE3max electroporation strategies. FIG. 2D shows editing efficiency quantification for each condition listed in FIG. 2C. FIG. 2E shows quantification of editing efficiencies using the 2×PE3max strategy with the top-performing nicking sgRNAs from FIG. 2A. FIG. 2F shows editing efficiency of reversion of the pathogenic HBB^{S} allele back to HBB^{A} in SCD patient CD34⁺ HSPCs with PE3max NG1. 4-5×10⁶ cells total were edited per donor in parallel electroporations of 5×10⁵ cells per replicate and pooled together for subsequent in vitro culture or cryopreserved for later injection into mice.

FIGS. 3A-3F: Engraftment of prime edited SCD patient CD34⁺ HSPCs after transplantation into immunodeficient mice. 2×10⁵ 2× PE3max edited HSPCs were transplanted from SCD patients into the tail vein of two to five busulfan-treated NBSGW mice per donor. Mouse bone marrow was harvested and analyzed 17 weeks post-transplantation. FIG. 3A shows an experimental overview of engraftment experiments. FIG. 3B shows human cell engraftment in recipient bone marrow measured by percentage of human CD45⁺ cells (hCD45⁺). Lines represent mean±SD and each individual shape represents a single mouse. Each donor is coded with a unique shape: healthy donor=grey diamonds; SCD donor 1=circles; SCD donor 2=squares; SCD donor 3=triangles; SCD donor 4=upside-down triangle. FIG. 3C shows percentages of human T cells (hCD3⁺), myeloid cells (hCD33⁺), and B cells (hCD19⁺) in the hCD45⁺ cell population in recipient bone marrow. Bar height represents cumulative average of each lineage with error bars for +SD. FIG. 3D shows the percentage of human erythroid precursor cells (hCD235⁺) as a percentage of human and mouse CD45⁻ cells in recipient bone marrow. Lines shown at mean±SD. FIG. 3E shows HBB^{S}-to-HBB^{A} editing efficiency for desired editing with no indels or other undesired products at the target site across human CD34⁺ cell-derived lineages in recipient bone marrow. Each lineage was isolated using antibodies against appropriate surface markers: hCD235 for erythroid lineages, hCD34 for HSPCs, hCD33 for myeloid cells, and CD19 for B cells. Lines shown at mean±SD. Each individual dot represents a single mouse. FIG. 3F shows average allelic editing for each of the four patient donors across 454 BFU-E colonies derived from PE treated CD34⁺ HSPCs. Lin cells were isolated and plated to achieve 95-120 individual colonies per donor. After 12 days in culture, colonies were picked into cell lysis buffer, and desired prime editing at the HBB locus was measured by high-throughput sequencing (HTS). Colonies were categorized by whether they had a biallelic edit without indels, a monoallelic edit without indels, no desired editing, or indels. Bar height represents cumulative average of each outcome with error bars for +SD.

FIGS. 4A-4C: Phenotypic rescue of sickle-cell disease characteristics in ex vivo-differentiated PE3max-treated human reticulocytes from HSPCs transplanted into immunodeficient mice. FIG. 4A shows the percentage of β-like globins measured by ion exchange HPLC in CD235a⁺ cells from human SCD patient cells. Bars represent cumulative averages of each protein with error bars for +SD across 2-5 mice per donor. FIG. 4B shows representative phase-contrast images of reticulocytes derived from PE3max edited or untreated SCD HSPCs incubated for 8 h with 2% oxygen. Scale bars, 100 μM. FIG. 4C shows quantification of sickle reticulocytes from images as in FIG. 4B from over 400 randomly selected cells per image. Cells were counted by a blinded observer for all conditions. Lines with error bars represent mean±SD, with each dot representing the percentage of sickled cells in one image from the specified donor. Significance was determined with one-sided multiple-paired t-tests correcting for multiple comparisons using the Holm-Šídák correction method. **p<0.01; ***p<0.001.

FIGS. 5A-5B: Off-target editing and DNA damage response in prime-edited CD34⁺ HSPCs. Bars reflect mean±SD of n=3 independent biological replicates, with replicate values shown as individual dots. Significance for both epegRNA and nicking sgRNA off-target editing was determined with one-sided multiple-paired t-tests correcting for multiple comparisons using the Holm-Šídák correction method. FIG. 5A shows rhAmpSeq quantification of the first epegRNA-encoded mismatch at CIRCLE-seq nominated off-target loci in SCD patient HSPCs. The graph also includes epegRNA OT49, which had to be analyzed separately with HTS since the primers for the locus were not amenable to pooled rhAmpSeq analysis. FIG. 5B shows rhAmpSeq quantification of indels at nicking sgRNA off-target loci nominated by CIRCLE-seq in SCD patient HSPCs. Nick OT32 was not amenable to rhAmpSeq analysis or PCR amplification and therefore could not be analyzed. Nick OT22 was the on-target NG1 site.

FIGS. 6A-6C: Optimization of epegRNA modification, viability and recovery of healthy donor CD34⁺ HSPCs following editing, and PE3max editing outcomes in SCD patient and healthy donor CD34⁺ HSPCs. FIG. 6A shows that 5×10⁵ cells per condition were electroporated with 2×PE3max with an epegRNA with or without a modified (phosphorothioate-linked) UUU trinucleotide at the 3′ end (Table 2). Bars reflect mean±SD of n=2 independent biological replicates, with replicate values shown as individual dots. All intended edit values include only desired prime editing products with no indels or other changes at the target site. Editing efficiency was measured 6 days after electroporation by high-throughput DNA sequencing. FIG. 6B shows that 48 hours-post electroporation, cell viability and recovery were measured using a Chemometec Nucleocounter-300. Acridine orange and DAPI were used to stain the total cell number and dead, permeabilized cells, respectively. The percent viability was calculated as the DAPI stained cells divided by the acridine orange stained cells within each sample. The percent recovery was normalized to the cell count of the unedited sample, which was not electroporated. Bar values represent mean±SD for n=2 to 5 independent biological replicate values, with replicate values shown as individual dots. All replates with near 0% cell recovery originate from the same vial of HSPCs in which there was massive cell death upon initial thaw, before editing occurred.

FIG. 6C shows quantification of editing efficiencies for both SCD patient and healthy donor HSPCs. Both the reversion of the pathogenic+4 T>A edit and the +5 A>G edit could be measured directly in SCD patient cells, while only the latter edit can be made in healthy donor cells. Bar values represent mean±SD for n=5 independent biological replicates for SCD patient HSPCs or n=3 independent biological replicates for healthy donor HSPCs, with individual dots representing replicate values. All intended edit values include only desired prime editing products with no indels or other changes at the target site.

FIGS. 7A-7B: Flow cytometry analysis of human HSC lineage populations and indel analysis of BFU-E colonies. FIG. 7A shows representative immune-flow cytometry for T cells (hCD3+), B cells (hCD19+), myeloid cells (hCD33+), and erythroid cells (hCD235a+). SSC-A, side scatter area; SSC-W, side scatter width; FSC-A, forward scatter area; DAPI, live-dead stain. FIG. 7B shows average allelic editing across each of the four patient donors for the 4% of colonies (16 colonies out of 454 total BFU-E colonies) derived from PE-treated CD34⁺ HSPCs with indels as in FIG. 3F. Colonies were categorized by whether they had a biallelic edit with indels, a monoallelic edit with indels, or indels without any desired editing. Bar heights represent cumulative averages of each outcome.

FIGS. 8A-8E: In vitro erythroid differentiation of SCD CD34⁺ HSPCs. FIG. 8A shows representative immuno-flow cytometry for erythroid maturation markers at days 8 and 13 in culture for in vitro differentiation. Gating strategy to identify single hCD235a⁺ cells.

FIG. 8B shows a gating strategy to track progression of erythroid differentiation based on hCD49d and B and3 expression in hCD235a⁺ cells^{13,61}. SSC-A, side scatter area; SSC-W, side scatter width; FSC-A, forward scatter area. FIG. 8C shows percentage of β-like globins measured by ion exchange HPLC in differentiated reticulocytes from human SCD patient cells. 5×10⁴ cells were differentiated per donor, per condition. Bars represent cumulative averages of each protein. FIG. 8D shows quantification of terminally differentiating HSPCs in vitro based on B and3 and CD49d at differentiation days 8, 13, 18, and 21^{13,61} as in FIG. 8B. Bars represent cumulative averages with error bars reflecting standard deviation (SD).

FIG. 8E shows the percentage of enucleated differentiated erythrocytes at day 21. Bars represent mean±SD for n=1-4 biological replicates, with individual dots representing values for each replicate.

FIG. 9: Identification of off-target prime editing. FIG. 9 shows an example of how epegRNA-encoded off-target prime editing was identified, as described herein. Since DNA sequencing errors and cellular genomic heterogeneity (observed in both edited cells and untreated cells) were the sources of the vast majority of DNA sequence differences between samples and the reference sequence, potential off-target prime editing was identified by counting sequences that contain the first mismatch encoded by the epegRNA reverse transcriptase template (RTT) as putative epegRNA-encoded off-target prime edits. In addition to epegRNA-encoded off-target editing, nick-induced indels near sites targeted by epegRNAs or nicking sgRNAs were also separately identified (see FIGS. 5A-5B). Sequences shown correspond (top-bottom) to SEQ ID NOs: 376-397.

### DEFINITIONS

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

### Aptamer

An “aptamer” refers to an oligonucleotide or peptide molecule that binds to a specific target molecule. In some embodiments, an epegRNA comprises an aptamer as a structured motif attached to its 3′ end. Aptamers include DNA or RNA aptamers that are short single-stranded DNA- or RNA-based oligonucleotides that can selectively bind to small molecular ligands or protein targets with high affinity and specificity, when folded into their unique three-dimensional structures. On the molecular level, aptamers bind to its cognate target through various non-covalent interactions, electrostatic interactions, hydrophobic interactions, and induced fitting. Further reference can be made to Ku et al., Nucleic Acid Aptamers: An Emerging Tool for Biotechnology and Biomedical Sensing. Sensors 2015, 15 (7): 16281-16313. The present disclosure contemplates the use of any aptamer, including those obtained from commercial sources. For example, numerous aptamers may be obtained from APTAGEN (aptagen.com) and include, but are not limited to, thrombin (15mer), HIV-1 TAR RNA hairpin loop (β₂₂-19), human immunoglobulin G (IgG) (Apt 8), reactive green 19 (GR-30), abrin toxin (TA6), malachite green (MG-4), PSMA aptamer (A10-3), tenascin-C (GBI-10), and methylenedianiline (M1). Another example is prequeosine₁-1 riboswitch aptamer—one of the smallest natural tertiary RNA structures (also known as evopreQ₁-1).

### Cas9

The term “Cas9” or “Cas9 nuclease” refers to an RNA-guided nuclease comprising a Cas9 domain, or a fragment thereof (e.g., a protein comprising an active or inactive DNA cleavage domain of Cas9, and/or the gRNA binding domain of Cas9). A “Cas9 domain,” as used herein, is a protein fragment comprising an active or fully or partly inactive cleavage domain of Cas9 and/or the gRNA binding domain of Cas9. A “Cas9 protein” is a full length Cas9 protein. A Cas9 nuclease is also referred to sometimes as a casn1 nuclease or a CRISPR (Clustered Regularly Interspaced Short Palindromic Repeat)-associated nuclease. CRISPR is an adaptive immune system that provides protection against mobile genetic elements (viruses, transposable elements, and conjugative plasmids). CRISPR clusters contain spacers, sequences complementary to antecedent mobile elements, and target invading nucleic acids. CRISPR clusters are transcribed and processed into CRISPR RNA (crRNA). In type II CRISPR systems, correct processing of pre-crRNA requires a trans-encoded small RNA (tracrRNA), endogenous ribonuclease 3 (rnc), and a Cas9 domain. The tracrRNA serves as a guide for ribonuclease 3-aided processing of pre-crRNA. Subsequently, Cas9/crRNA/tracrRNA endonucleolytically cleaves a linear or circular dsDNA target complementary to the spacer. The strand in the target DNA not complementary to crRNA is first cut endonucleolytically, then trimmed 3′-5′ exonucleolytically. In nature, DNA-binding and cleavage typically requires protein and both RNAs. However, single guide RNAs (“sgRNA”, or simply “gRNA”) can be engineered so as to incorporate aspects of both the crRNA and tracrRNA into a single RNA species. See, e.g., Jinek M., Chylinski K., Fonfara I., Hauer M., Doudna J. A., Charpentier E. Science 337:816-821 (2012), the contents of which are incorporated herein by reference. Cas9 recognizes a short motif in the CRISPR repeat sequences (the PAM or protospacer adjacent motif) to help distinguish self versus non-self. Cas9 nuclease sequences and structures are well known to those of skill in the art (see, e.g., “Complete genome sequence of an M1 strain of Streptococcus pyogenes.” Ferretti et al., J. J., McShan W. M., Ajdic D. J., Savic D. J., Savic G., Lyon K., Primeaux C., Sezate S., Suvorov A. N., Kenton S., Lai H. S., Lin S. P., Qian Y., Jia H. G., Najar F. Z., Ren Q., Zhu H., Song L., White J., Yuan X., Clifton S. W., Roe B. A., Mclaughlin R. E., Proc. Natl. Acad. Sci. U.S.A. 98:4658-4663 (2001); “CRISPR RNA maturation by trans-encoded small RNA and host factor RNase III.” Deltcheva E., Chylinski K., Sharma C. M., Gonzales K., Chao Y., Pirzada Z. A., Eckert M. R., Vogel J., Charpentier E., Nature 471:602-607 (2011); and “A programmable dual-RNA-guided DNA endonuclease in adaptive bacterial immunity.” Jinek M., Chylinski K., Fonfara I., Hauer M., Doudna J. A., Charpentier E. Science 337:816-821 (2012), the entire contents of each of which are incorporated herein by reference). Cas9 orthologs have been described in various species, including, but not limited to, S. pyogenes and S. thermophilus. Additional suitable Cas9 nucleases and sequences will be apparent to those of skill in the art based on this disclosure, and such Cas9 nucleases and sequences include Cas9 sequences from the organisms and loci disclosed in Chylinski, Rhun, and Charpentier, “The tracrRNA and Cas9 families of type II CRISPR-Cas immunity systems” (2013) RNA Biology 10:5, 726-737; the entire contents of which are incorporated herein by reference. In some embodiments, a Cas9 nuclease comprises one or more mutations that partially impair or inactivate the DNA cleavage domain.

A nuclease-inactivated Cas9 domain may interchangeably be referred to as a “dCas9” protein (for nuclease-“dead” Cas9). Methods for generating a Cas9 domain (or a fragment thereof) having an inactive DNA cleavage domain are known (see, e.g., Jinek et al., Science. 337:816-821 (2012); Qi et al., “Repurposing CRISPR as an RNA-Guided Platform for Sequence-Specific Control of Gene Expression” (2013) Cell. 28; 152 (5): 1173-83, the entire contents of each of which are incorporated herein by reference). For example, the DNA cleavage domain of Cas9 is known to include two subdomains, the HNH nuclease subdomain and the RuvC1 subdomain. The HNH subdomain cleaves the strand complementary to the gRNA, whereas the RuvC1 subdomain cleaves the non-complementary strand. Mutations within these subdomains can silence the nuclease activity of Cas9. For example, the mutations D10A and H840A (or H839A in SEQ ID NO: 8) completely inactivate the nuclease activity of S. pyogenes Cas9 (Jinek et al., Science. 337:816-821 (2012); Qi et al., Cell. 28; 152 (5): 1173-83 (2013)). In some embodiments, proteins comprising fragments of a Cas9 protein are provided. For example, in some embodiments, a protein comprises one of two Cas9 domains: (1) the gRNA binding domain of Cas9; or (2) the DNA cleavage domain of Cas9. In some embodiments, proteins comprising Cas9, or fragments thereof, are referred to as “Cas9 variants.” A Cas9 variant shares homology to Cas9, or a fragment thereof. For example, a Cas9 variant is at least about 70% identical, at least about 80% identical, at least about 90% identical, at least about 95% identical, at least about 96% identical, at least about 97% identical, at least about 98% identical, at least about 99% identical, at least about 99.5% identical, at least about 99.8% identical, or at least about 99.9% identical to wild type Cas9 (e.g., SpCas9 of SEQ ID NO: 6). In some embodiments, the Cas9 variant may have 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50, or more amino acid changes compared to wild type Cas9 (e.g., SpCas9 of SEQ ID NO: 6). In some embodiments, the Cas9 variant comprises a fragment of SEQ ID NO: 6 Cas9 (e.g., a gRNA binding domain or a DNA-cleavage domain), such that the fragment is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to the corresponding fragment of wild type Cas9 (e.g., SpCas9 of SEQ ID NO: 6). In some embodiments, the fragment is at least 30%, at least 35%, at least 40%, at least 45%, at least 50%, at least 55%, at least 60%, at least 65%, at least 70%, at least 75%, at least 80%, at least 85%, at least 90%, at least 95% identical, at least 96%, at least 97%, at least 98%, at least 99%, or at least 99.5% of the amino acid length of a corresponding wild type Cas9 (e.g., SpCas9 of SEQ ID NO: 6).

### CRISPR

CRISPR is a family of DNA sequences (i.e., CRISPR clusters) in bacteria and archaea that represent snippets of prior infections by a virus that have invaded the prokaryote. The snippets of DNA are used by the prokaryotic cell to detect and destroy DNA from subsequent attacks by similar viruses and effectively compose, along with an array of CRISPR-associated proteins (including Cas9 and homologs thereof) and CRISPR-associated RNA, a prokaryotic immune defense system. In nature, CRISPR clusters are transcribed and processed into CRISPR RNA (crRNA). In certain types of CRISPR systems (e.g., type II CRISPR systems), correct processing of pre-crRNA requires a trans-encoded small RNA (tracrRNA), endogenous ribonuclease 3 (rnc), and a Cas9 protein. The tracrRNA serves as a guide for ribonuclease 3-aided processing of pre-crRNA. Subsequently, Cas9/crRNA/tracrRNA endonucleolytically cleaves a linear or circular dsDNA target complementary to the RNA. Specifically, the DNA strand in the target that is not complementary to crRNA is first cut endonucleolytically, then trimmed 3′-5′ exonucleolytically. In nature, DNA-binding and cleavage typically requires protein and both RNAs. However, single guide RNAs (“sgRNA”, or simply “gRNA”) can be engineered so as to incorporate aspects of both the crRNA and tracrRNA into a single RNA species—the guide RNA. See, e.g., Jinek M., Chylinski K., Fonfara I., Hauer M., Doudna J. A., Charpentier E. Science 337:816-821 (2012), the entire contents of which is hereby incorporated by reference. Cas9 recognizes a short motif in the CRISPR repeat sequences (the PAM or protospacer adjacent motif) to help distinguish self versus non-self. CRISPR biology, as well as Cas9 nuclease sequences and structures are well known to those of skill in the art (see, e.g., “Complete genome sequence of an M1 strain of Streptococcus pyogenes.” Ferretti et al., J. J., McShan W. M., Ajdic D. J., Savic D. J., Savic G., Lyon K., Primeaux C., Sezate S., Suvorov A. N., Kenton S., Lai H. S., Lin S. P., Qian Y., Jia H. G., Najar F. Z., Ren Q., Zhu H., Song L., White J., Yuan X., Clifton S. W., Roe B. A., McLaughlin R. E., Proc. Natl. Acad. Sci. U.S.A. 98:4658-4663 (2001); “CRISPR RNA maturation by trans-encoded small RNA and host factor RNase III.” Deltcheva E., Chylinski K., Sharma C. M., Gonzales K., Chao Y., Pirzada Z. A., Eckert M. R., Vogel J., Charpentier E., Nature 471:602-607 (2011); and “A programmable dual-RNA-guided DNA endonuclease in adaptive bacterial immunity.” Jinek M., Chylinski K., Fonfara I., Hauer M., Doudna J. A., Charpentier E. Science 337:816-821 (2012), the entire contents of each of which are incorporated herein by reference). Cas9 orthologs have been described in various species, including, but not limited to, S. pyogenes and S. thermophilus. Additional suitable Cas9 nucleases and sequences will be apparent to those of skill in the art based on this disclosure, and such Cas9 nucleases and sequences include Cas9 sequences from the organisms and loci disclosed in Chylinski, Rhun, and Charpentier, “The tracrRNA and Cas9 families of type II CRISPR-Cas immunity systems” (2013) RNA Biology 10:5, 726-737; the entire contents of which are incorporated herein by reference.

In general, a “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g., tracrRNA or an active partial tracrRNA), a tracr mate sequence (encompassing a “direct repeat” and a tracrRNA-processed partial direct repeat in the context of an endogenous CRISPR system), a guide sequence (also referred to as a “spacer”), or other sequences and transcripts from a CRISPR locus. The tracrRNA of the system is complementary (fully or partially) to the tracr mate sequence present on the guide RNA.

### DNA Synthesis Template (or Reverse Transcription Template (RTT))

As used herein, the terms “DNA synthesis template” and “reverse transcription template (RTT)” refer to the region or portion of the extension arm of a PEgRNA or epegRNA that is utilized as a template by a polymerase of a prime editor to encode a 3′ single-strand DNA flap that contains the desired edit and which then, through the mechanism of prime editing, replaces the corresponding endogenous strand of DNA at the target site. The extension arm, including the DNA synthesis template, may be comprised of DNA or RNA. In the case of RNA, the polymerase of the prime editor can be an RNA-dependent DNA polymerase (e.g., a reverse transcriptase). In the case of DNA, the polymerase of the prime editor can be a DNA-dependent DNA polymerase. In various embodiments, the DNA synthesis template may comprise the “edit template” and the “homology arm”, and all or a portion of an optional 5′ end modifier region and/or an optional 3′ end modifier region. Said another way, in the case of a 3′ extension arm, the DNA synthesis template can include the portion of the extension arm that spans from the 5′ end of the primer binding site (PBS) to 3′ end of the gRNA core that may operate as a template for the synthesis of a single-strand of DNA by a polymerase (e.g., a reverse transcriptase). In the case of a 5′ extension arm, the DNA synthesis template can include the portion of the extension arm that spans from the 5′ end of the PERNA molecule to the 5′ end of the PBS. Certain embodiments described here refer to a “DNA synthesis template,” an “RT template,” or an “RTT,” which is also inclusive of the edit template and the homology arm, but wherein the RT edit template reflects the use of a prime editor having a polymerase that is a reverse transcriptase, and wherein the DNA synthesis template reflects more broadly the use of a prime editor having any polymerase. In certain embodiments, an RT template may be used to refer to a template polynucleotide for reverse transcription, e.g., in a prime editing system, complex, or method using a prime editor having a polymerase that is a reverse transcriptase. In some embodiments, a DNA synthesis template may be used to refer to a template polynucleotide for DNA polymerization, e.g., RNA-dependent DNA polymerization or DNA-dependent polymerization, e.g., in a prime editing system, complex, or method using a prime editor having a polymerase that is an RNA-dependent DNA polymerase or a DNA-dependent DNA polymerase. The term “edit template” refers to a portion of the extension arm that encodes the desired edit in the single strand 3′ DNA flap that is synthesized by the polymerase, e.g., a DNA-dependent DNA polymerase or an RNA-dependent DNA polymerase (e.g., a reverse transcriptase).

As used herein, the term “DNA synthesis template” refers to the region or portion of the extension arm of a pegRNA that is utilized as a template strand by a polymerase of a prime editor to encode a 3′ single-strand DNA flap that contains the desired edit and which then, through the mechanism of prime editing, replaces the corresponding endogenous strand of DNA at the target site. The extension arm, including the DNA synthesis template, may be comprised of DNA or RNA. In the case of RNA, the polymerase of the prime editor can be an RNA-dependent DNA polymerase (e.g., a reverse transcriptase). In the case of DNA, the polymerase of the prime editor can be a DNA-dependent DNA polymerase. In various embodiments, the DNA synthesis template comprises an the “edit template” and a “homology arm.” In various embodiments, the DNA synthesis template may comprise the “edit template” and a “homology arm”, and all or a portion of the optional 5′ end modifier region, e2. That is, depending on the nature of the e2 region (e.g., whether it includes a hairpin, toeloop, or stem/loop secondary structure), the polymerase may encode none, some, or all of the e2 region, as well. Said another way, in the case of a 3′ extension arm, the DNA synthesis template can include the portion of the extension arm that spans from the 5′ end of the primer binding site (PBS) to 3′ end of the gRNA core that may operate as a template for the synthesis of a single-strand of DNA by a polymerase (e.g., a reverse transcriptase). In the case of a 5′ extension arm, the DNA synthesis template can include the portion of the extension arm that spans from the 5′ end of the pegRNA molecule to the 3′ end of the edit template. In some embodiments, the DNA synthesis template excludes the primer binding site (PBS) of pegRNAs either having a 3′ extension arm or a 5′ extension arm. Certain embodiments refer to an “RT template,” which is inclusive of the edit template and the homology arm, i.e., the sequence of the pegRNA extension arm which is actually used as a template during DNA synthesis. The term “RT template” is equivalent to the term “DNA synthesis template.” In certain embodiments, an RT template may be used to refer to a template polynucleotide for reverse transcription, e.g., in a prime editing system, complex or method using a prime editor having a polymerase that is a reverse transcriptase. In some embodiments, a DNA synthesis template may be used to refer to a template polynucleotide for DNA polymerization, e.g., RNA-dependent DNA polymerization or DNA-dependent polymerization, e.g., in a prime editing system, complex, or method using a prime editor having a polymerase that is an RNA-dependent DNA polymerase or a DNA-dependent DNA polymerase.

In some embodiments, the DNA synthesis template is a single-stranded portion of the PEgRNA that is 5′ of the PBS and comprises a region of complementarity to the PAM strand (i.e., the non-target strand or the edit strand), and comprises one or more nucleotide edits compared to the endogenous sequence of the double stranded target DNA. In some embodiments, the DNA synthesis template is complementary or substantially complementary to a sequence on the non-target strand that is downstream of a nick site, except for one or more non-complementary nucleotides at the intended nucleotide edit positions. In some embodiments, the DNA synthesis template is complementary or substantially complementary to a sequence on the non-target strand that is immediately downstream (i.e., directly downstream) of a nick site, except for one or more non-complementary nucleotides at the intended nucleotide edit positions. In some embodiments, one or more of the non-complementary nucleotides at the intended nucleotide edit positions are immediately downstream of a nick site. In some embodiments, the DNA synthesis template comprises one or more nucleotide edits relative to the double-stranded target DNA sequence. In some embodiments, the DNA synthesis template comprises one or more nucleotide edits relative to the non-target strand of the double-stranded target DNA sequence. For each PEgRNA described herein, a nick site is characteristic of the particular napDNAbp to which the gRNA core of the PEgRNA associates and is also characteristic of the particular PAM required for recognition and function of the napDNAbp. For example, for a PERNA that comprises a gRNA core that associates with a SpCas9, the nick site in the phosphodiester bond between bases three (“−3” position relative to the position 1 of the PAM sequence) and four (“−4” position relative to position 1 of the PAM sequence). In some embodiments, the DNA synthesis template and the primer binding site are immediately adjacent to each other. The terms “nucleotide edit”, “nucleotide change”, “desired nucleotide change”, and “desired nucleotide edit” are used interchangeably to refer to a specific nucleotide edit, e.g., a specific deletion of one or more nucleotides, a specific insertion of one or more nucleotides, a specific substitution(s) of one or more nucleotides, or a combination thereof, at a specific position in a DNA synthesis template of a PEgRNA to be incorporated in a target DNA sequence. In some embodiments, the DNA synthesis template comprises more than one nucleotide edit relative to the double-stranded target DNA sequence. In such embodiments, each nucleotide edit is a specific nucleotide edit at a specific position in the DNA synthesis template, each nucleotide edit is at a different specific position relative to any of the other nucleotide edits in the DNA synthesis template, and each nucleotide edit is independently selected from a specific deletion of one or more nucleotides, a specific insertion of one or more nucleotides, a specific substitution(s) of one or more nucleotides, or a combination thereof. A nucleotide edit may refer to the edit on the DNA synthesis template as compared to the sequence on the target strand of the double stranded target DNA, or may refer to the edit encoded by the DNA synthesis template on the newly synthesized single stranded DNA that replaces the endogenous target DNA sequence on the non-target strand. In some embodiments, the DNA synthesis template/reverse transcriptase template encodes a sequence comprising the correction of an A·T:T·A transversion mutation at nucleotide position 20 in the β-globin gene (HBB). In some embodiments, the DNA synthesis template/reverse transcriptase template further encodes a G→A silent PAM-disrupting edit at the nucleotide position following the A·T:T·A transversion mutation.

### Edit Strand and Non-Edit Strand

The terms “edit strand” and “non-edit strand” are terms that may be used when describing the mechanism of a prime editing system on a double-stranded DNA substrate. The “edit strand” refers to the strand of DNA that is nicked by the prime editor complex to form a 3′ end, which is then extended as a newly synthesized single stranded DNA (also referred herein as the newly synthesized 3′ DNA flap), which comprises a desired edit and ultimately displaces and replaces the single strand region of DNA just downstream of the nick, thereby installing the 3′ DNA flap containing the desired edit downstream of the nick on the “edit strand.” In some embodiments, the newly synthesized 3′ DNA flap comprising the nucleotide edit is paired in a heteroduplex with the non-edit strand that does not comprise the nucleotide edit, thereby creating a mismatch. In some embodiments, the mismatch is recognized by DNA repair machinery, and/or replication machinery, e.g., an endogenous DNA repair machinery. In some embodiments, through DNA repair, the intended nucleotide edit is incorporated into both strands of the target double-stranded DNA substrate. The application may also refer to the “edit strand” as the “protospacer strand” or the “PAM strand” since these elements are present in that strand. The “edit strand” may also be called the “non-target strand” since the edit strand is not the strand that becomes annealed to the spacer of the PERNA molecule, but rather is the complement of the strand that is annealed by the spacer of the PERNA. The “non-edit” strand is not directly edited by the PE system. Rather, the desired edit created by the PE system in the 3′ DNA flap is incorporated into the “non-edited strand” through DNA replication and/or repair. In some embodiments, the “non-edit strand” is the strand that anneals to the spacer of the PEgRNA, and thus is also called the “target strand.”

### Extension Arm

The term “extension arm” refers to a nucleotide sequence component of a PEgRNA which comprises a primer binding site (PBS) and a DNA synthesis template for a polymerase (e.g., an RT template for reverse transcriptase). In some embodiments, the extension arm is located at the 3′ end of the guide RNA. In other embodiments, the extension arm is located at the 5′ end of the guide RNA. In some embodiments, the extension arm comprises a DNA synthesis template and a primer binding site. In some embodiments, the extension arm comprises the following components in a 5′ to 3′ direction: the DNA synthesis template, and the primer binding site. In some embodiments, the extension arm also includes a homology arm. In various embodiments, the extension arm comprises the following components in a 5′ to 3′ direction: the homology arm, the edit template, and the primer binding site. Since polymerization activity of the reverse transcriptase is in the 5′ to 3′ direction, the preferred arrangement of the homology arm, edit template, and primer binding site is in the 5′ to 3′ direction such that the reverse transcriptase, once primed by an annealed primer sequence, polymerizes a single strand of DNA using the edit template as a complementary template strand.

The extension arm may be described as comprising generally two regions: a primer binding site (PBS) and a DNA synthesis template, for instance. The primer binding site binds to a primer sequence, for example, a single stranded primer sequence containing a free 3′ end at the nick site that is formed from the endogenous DNA strand of the target site when it becomes nicked by the prime editor complex, thereby exposing a 3′ end on the endogenous nicked strand. As explained herein, the binding of the primer sequence to the primer binding site on the extension arm of the PERNA creates a duplex region with an exposed 3′ end (i.e., the 3′ of the primer sequence), which then provides a substrate for a polymerase to begin polymerizing a single strand of DNA from the exposed 3′ end along the length of the DNA synthesis template. The sequence of the single strand DNA product is the complement of the DNA synthesis template. Polymerization continues towards the 5′ of the DNA synthesis template (or extension arm) until polymerization terminates. Thus, the DNA synthesis template represents the portion of the extension arm that is encoded into a single strand DNA product (i.e., the 3′ single strand DNA flap containing the desired nucleotide edit) by the polymerase of the prime editor complex and that ultimately replaces the corresponding endogenous DNA strand of the target site that sits immediately downstream of the PE-induced nick site. Without being bound by theory, polymerization of the DNA synthesis template continues towards the 5′ end of the extension arm until a termination event. Polymerization may terminate in a variety of ways, including, but not limited to (a) reaching a 5′ terminus of the PEgRNA (e.g., in the case of the 5′ extension arm wherein the DNA polymerase simply runs out of template), (b) reaching an impassable RNA secondary structure (e.g., hairpin or stem/loop), or (c) reaching a replication termination signal, e.g., a specific nucleotide sequence that blocks or inhibits the polymerase, or a nucleic acid topological signal, such as supercoiled DNA or RNA.

### Fusion Protein

The term “fusion protein” as used herein refers to a hybrid polypeptide which comprises protein domains from at least two different proteins. One protein may be located at the amino-terminal (N-terminal) portion of the fusion protein or at the carboxy-terminal (C-terminal) protein thus forming an “amino-terminal fusion protein” or a “carboxy-terminal fusion protein,” respectively. A protein may comprise different domains, for example, a nucleic acid-programmable DNA-binding domain (e.g., the gRNA binding domain of Cas9 that directs the binding of the protein to a target site) and a reverse transcriptase (i.e., a prime editor). Any of the fusion proteins provided herein may be produced by any method known in the art. For example, the prime editor fusion proteins provided herein may be produced via recombinant protein expression and purification, which is especially suited for fusion proteins comprising a peptide linker. Methods for recombinant protein expression and purification are well known, and include those described by Green and Sambrook, Molecular Cloning: A Laboratory Manual (4th ed., Cold Spring Harbor Laboratory Press, Cold Spring Harbor, N.Y. (2012)), which is incorporated herein by reference.

Guide RNA (“gRNA”)

As used herein, the term “guide RNA” is a particular type of guide nucleic acid that is mostly commonly associated with a Cas protein of a CRISPR-Cas9 and that associates with Cas9, directing the Cas9 protein to a specific sequence in a DNA molecule that includes complementarity to the spacer sequence of the guide RNA. However, this term also embraces the equivalent guide nucleic acid molecules that associate with Cas9 equivalents, homologs, orthologs, or paralogs, whether naturally occurring or non-naturally occurring (e.g., engineered or recombinant), and which otherwise program the Cas9 equivalent to localize to a specific target nucleotide sequence. The Cas9 equivalents may include other napDNAbp from any type of CRISPR system (e.g., type II, V, VI), including Cpf1 (a type-V CRISPR-Cas systems), C2c1 (a type V CRISPR-Cas system), C2c2 (a type VI CRISPR-Cas system), and C2c3 (a type V CRISPR-Cas system). Further Cas-equivalents are described in Makarova et al., “C2c2 is a single-component programmable RNA-guided RNA-targeting CRISPR effector,” Science 2016; 353 (6299), the contents of which are incorporated herein by reference. Exemplary sequences and structures of guide RNAs are provided herein. In addition, methods for designing appropriate guide RNA sequences are provided herein. As used herein, the “guide RNA” may also be referred to as a “traditional guide RNA” to contrast it with the modified forms of guide RNA termed “prime editing guide RNAs” (or “PEgRNAs”) and “engineered PEgRNAs” (or epegRNAs”). The term “single guide RNA (sgRNA)” refers to a single guide RNA molecule that contains both a spacer sequence (designed to target a particular nucleotide sequence) and a guide RNA scaffold sequence.

Guide RNAs, sgRNAs, or PEgRNAs/epegRNAs may comprise various structural elements that include, but are not limited to:

Spacer sequence—the sequence in the guide RNA or pegRNA/epegRNA (having about 20 nts in length) that has the same sequence as the protospacer in the target DNA, except that the guide RNA or PEgRNA/epegRNA comprises uracil and the target protospacer contains thymine.

gRNA core (or gRNA scaffold or backbone sequence)—the sequence within the gRNA that is responsible for binding with a nucleic acid programmable DNA binding protein, e.g., a Cas9. It does not include the spacer sequence that is used to guide Cas9 to target DNA.

Transcription terminator—the guide RNA or PEgRNA may comprise a transcriptional termination sequence at the 3′ of the molecule.

In some embodiments, a pegRNA or epegRNA may also comprise an extension arm—a single strand extension at the 3′ end or the 5′ end of the PEgRNA which comprises a primer binding site and a DNA synthesis template sequence that encodes via a polymerase (e.g., a reverse transcriptase) a single stranded DNA flap containing the desired nucleotide change, which then integrates into the endogenous DNA by replacing the corresponding endogenous strand, thereby installing the desired nucleotide change.

In some embodiments, a guide RNA is a “nicking guide RNA.” Nicking guide RNAs may be used to nick the non-edited strand of a target nucleic acid molecule, which may facilitate incorporation of the edit by cellular DNA repair mechanisms.

### G-Quadruplex

A “G-quadruplex” is a complex three-dimensional nucleic acid moiety formed in nucleic acid sequences that are rich in guanine (G). They are helical in shape and formed from interconnected stacks of guanine tetrads (or “G-tetrads”), which individually are flat, ring-shaped structures formed from four guanines, and which can be stabilized by the presence of a cation (e.g., potassium) that sits in a central channel between pairs of G-tetrads. G-quadruplexes are a diverse collection of structures and not a single structure. Further reference to G-quadruplexes can be found in (1) Kwok et al., G-Quadruplexes: Prediction, Characterization, and Biological Application, Trends in Biotechnology, 2017, Vol. 35 (10; pp. 997-1013; (2) Hansel-Hertsch R. et al., DNA G-quadruplexes in the human genome: detection, functions and therapeutic potential, Nat. Rev. Mol. Cell Biol., 2017; 18:279-284; and (3) Millevoi S. et al., “G-quadruplexes in RNA biology, “Wiley Interdiscip. Rev. RNA, 2012; 3:495-507, each of which are incorporated herein by reference. In some embodiments, an epegRNA comprises a G-quadruplex as a structured motif on its 3′ end, which may facilitate the stabilization of the epegRNA or otherwise prevent its degradation.

### Linker

The term “linker,” as used herein, refers to a molecule linking two other molecules or moieties. The linker can be an amino acid sequence in the case of a peptide linker joining two domains of a fusion protein. For example, a napDNAbp (e.g., Cas9) can be fused to a reverse transcriptase by an amino acid linker sequence. The linker can also be a nucleotide sequence in the case of joining two nucleotide sequences together (e.g., in a gRNA). For example, in the instant case, the traditional guide RNA is linked via a spacer or linker nucleotide sequence to the RNA extension of a prime editing guide RNA which may comprise an RT template sequence and an RT primer binding site. In other embodiments, the linker is an organic molecule, group, polymer, or chemical moiety. In some embodiments, the linker is 5-200 amino acids in length, for example, 5, 6, 7, 8, 9, 10, 11, 12, 13, 14, 15, 16, 17, 18, 19, 20, 21, 22, 23, 24, 25, 26, 27, 28, 29, 30, 30-35, 35-40, 40-45, 45-50, 50-60, 60-70, 70-80, 80-90, 90-100, 100-150, or 150-200 amino acids in length. Longer or shorter linkers are also contemplated.

In some embodiments, the epegRNAs provided herein comprise a linker that has been engineered to allow the attachment of a 3′ structured motif to the pegRNA without disrupting the structure or function of the pegRNA. In some embodiments, such an engineered linker comprises the sequence 5′-AGAATAAA-3′ and is placed between the extension arm sequence and the 3′ structured motif. In some embodiments, the engineered linker is at least 90%, at least 95%, at least 96%, at least 97%, at least 98%, or at least 99% identical to the sequence 5′-AGAATAAA-3′ or a fragment thereof.

napDNAbp

As used herein, the term “nucleic acid programmable DNA binding protein” or “napDNAbp,” of which Cas9 is an example, refers to a protein that uses RNA:DNA hybridization to target and bind to specific sequences in a DNA molecule. Each napDNAbp is associated with at least one guide nucleic acid (e.g., guide RNA), which localizes the napDNAbp to a DNA sequence that comprises a DNA strand (i.e., a target strand) that is complementary to the guide nucleic acid, or a portion thereof (e.g., the protospacer of a guide RNA). In other words, the guide nucleic-acid “programs” the napDNAbp (e.g., Cas9 or equivalent) to localize and bind to a complementary sequence.

Without being bound by theory, the binding mechanism of a napDNAbp-guide RNA complex, in general, includes the step of forming an R-loop whereby the napDNAbp induces the unwinding of a double-strand DNA target, thereby separating the strands in the region bound by the napDNAbp. The guide RNA protospacer then hybridizes to the “target strand.” This displaces a “non-target strand” that is complementary to the target strand, which forms the single strand region of the R-loop. In some embodiments, the napDNAbp includes one or more nuclease activities, which then cut the DNA, leaving various types of lesions. For example, the napDNAbp may comprise a nuclease activity that cuts the non-target strand at a first location, and/or cuts the target strand at a second location. Depending on the nuclease activity, the target DNA can be cut to form a “double-stranded break” whereby both strands are cut. In other embodiments, the target DNA can be cut at only a single site, i.e., the DNA is “nicked” on one strand. Exemplary napDNAbp with different nuclease activities include “Cas9 nickase” (“nCas9”) and a deactivated Cas9 having no nuclease activities (“dead Cas9” or “dCas9”). Exemplary sequences for these and other napDNAbp are provided herein.

### Nickase

As used herein, a “nickase” refers to a napDNAbp (e.g., a Cas protein) which is capable of cleaving only one of the two complementary strands of a double-stranded target DNA sequence, thereby generating a nick in that strand. In some embodiments, the nickase cleaves a non-target strand of a double stranded target DNA sequence. In some embodiments, the nickase comprises an amino acid sequence with one or more mutations in a catalytic domain of a canonical napDNAbp (e.g., a Cas protein), wherein the one or more mutations reduces or abolishes nuclease activity of the catalytic domain. In some embodiments, the nickase is a Cas9 that comprises one or more mutations in a RuvC-like domain relative to a wild type Cas9 sequence or to an equivalent amino acid position in other Cas9 variants or Cas9 equivalents. In some embodiments, the nickase is a Cas9 that comprises one or more mutations in an HNH-like domain relative to a wild type Cas9 sequence or to an equivalent amino acid position in other Cas9 variants or Cas9 equivalents. In some embodiments, the nickase is a Cas9 that comprises an aspartate-to-alanine substitution (D10A) in the RuvC I catalytic domain of Cas9 relative to a canonical SpCas9 sequence or to an equivalent amino acid position in other Cas9 variants or Cas9 equivalents. In some embodiments, the nickase is a Cas9 that comprises an H840A (or H839A in SEQ ID NO: 8), N854A, and/or N863A mutation relative to a canonical SpCas9 sequence, or to an equivalent amino acid position in other Cas9 variants or Cas9 equivalents. In some embodiments, the term “Cas9 nickase” refers to a Cas9 with one of the two nuclease domains inactivated. This enzyme is capable of cleaving only one strand of a target DNA. In some embodiments, the nickase is a Cas protein that is not a Cas9 nickase.

In some embodiments, the napDNAbp of the prime editing complex comprises an endonuclease having nucleic acid programmable DNA binding ability. In some embodiments, the napDNAbp comprises an active endonuclease capable of cleaving both strands of a double stranded target DNA. In some embodiments, the napDNAbp is a nuclease active endonuclease, e.g., a nuclease active Cas protein, that can cleave both strands of a double stranded target DNA by generating a nick on each strand. For example, a nuclease active Cas protein can generate a cleavage (a nick) on each strand of a double stranded target DNA. In some embodiments, the two nicks on both strands are staggered nicks, for example, generated by a napDNAbp comprising a Cas12a or Cas12b1. In some embodiments, the two nicks on both strands are at the same genomic position, for example, generated by a napDNAbp comprising a nuclease active Cas9. In some embodiments, the napDNAbp comprises an endonuclease that is a nickase. For example, in some embodiments, the napDNAbp comprises an endonuclease comprising one or more mutations that reduce nuclease activity of the endonuclease, rendering it a nickase. In some embodiments, the napDNAbp comprises an inactive endonuclease, for example, in some embodiments, the napDNAbp comprises an endonuclease comprising one or more mutations that abolish the nuclease activity. In various embodiments, the napDNAbp is a Cas9 protein or variant thereof. The napDNAbp can also be a nuclease active Cas9, a nuclease inactive Cas9 (dCas9), or a Cas9 nickase (nCas9). In a preferred embodiment, the napDNAbp is Cas9 nickase (nCas9) that nicks only a single strand. In other embodiments, the napDNAbp can be selected from the group consisting of: Cas9, Cas12e, Cas12d, Cas12a, Cas12b1, Cas12b2, Cas13a, Cas12c, Cas12d, Cas12e, Cas12h, Cas12i, Cas12g, Cas12f (Cas14), Cas12f1, Cas12j (CasΦ), and Argonaute and optionally has a nickase activity such that only one strand is cut. In some embodiments, the napDNAbp is selected from Cas9, Cas12e, Cas12d, Cas12a, Cas12b1, Cas12b2, Cas13a, Cas12c, Cas12d, Cas12e, Cas12h, Cas12i, Cas12g, Cas12f (Cas14), Cas12f1, Cas12j (CasΦ), and Argonaute and optionally has a nickase activity such that one DNA strand is cut preferentially to the other DNA strand.

### Nuclear Localization Sequence (NLS)

The term “nuclear localization sequence” or “NLS” refers to an amino acid sequence that promotes import of a protein into the cell nucleus, for example, by nuclear transport. Nuclear localization sequences are known in the art and would be apparent to the skilled artisan. For example, NLS sequences are described in Plank et al., international PCT application, PCT/EP2000/011690, filed Nov. 23, 2000, published as WO 2001/038547 on May 31, 2001, the contents of which are incorporated herein by reference for its disclosure of exemplary nuclear localization sequences. In some embodiments, an NLS comprises the amino acid sequence PKKKRKV (SEQ ID NO: 94), MDSLLMNRRKFLYQFKNVRWAKGRRETYLC (SEQ ID NO: 99), KRTADGSEFESPKKKRKV (SEQ ID NO: 97), KRTADGSEFEPKKKRKV (SEQ ID NO: 106), NLSKRPAAIKKAGQAKKKK (SEQ ID NO: 107), PAAKRVKLD (SEQ ID NO: 98), RQRRNELKRSF (SEQ ID NO: 108), or NQSSNFGPMKGGNFGGRSSGPYGGGGQYFAKPRNQGGY (SEQ ID NO: 109).

### Nucleic Acid

The term “nucleic acid,” as used herein, refers to a polymer of nucleotides. The polymer may include natural nucleosides (i.e., adenosine, thymidine, guanosine, cytidine, uridine, deoxyadenosine, deoxythymidine, deoxyguanosine, and deoxycytidine), nucleoside analogs (e.g., 2-aminoadenosine, 2-thiothymidine, inosine, pyrrolo-pyrimidine, 3-methyl adenosine, 5-methylcytidine, C5 bromouridine, C5 fluorouridine, C5 iodouridine, C5 propynyl uridine, C5 propynyl cytidine, C5 methylcytidine, 7-deazaadenosine, 7-deazaguanosine, 8-oxoadenosine, 8-oxoguanosine, O (6)-methylguanine, 4-acetylcytidine, 5-(carboxyhydroxymethyl) uridine, dihydrouridine, methylpseudouridine, 1-methyl adenosine, 1-methyl guanosine, N6-methyl adenosine, and 2-thiocytidine), chemically modified bases, biologically modified bases (e.g., methylated bases), intercalated bases, modified sugars (e.g., 2′-fluororibose, ribose, 2′-deoxyribose, 2′-O-methylcytidine, arabinose, and hexose), or modified phosphate groups (e.g., phosphorothioates and 5′ N phosphoramidite linkages). In some embodiments, a nucleic acid is a pegRNA or an epegRNA. In certain embodiments, a nucleic acid comprises one or more 2′-O-methyl modified thymidines. In certain embodiments, a nucleic acid comprises one or more phosphorothioate linkages, e.g., between one or more thymidines.

### PEgRNA

As used herein, the terms “prime editing guide RNA” or “PEgRNA” or “pegRNA” or “extended guide RNA” refer to a specialized form of a guide RNA that has been modified to include one or more additional sequences for implementing the prime editing methods and compositions described herein. As described herein, the prime editing guide RNAs comprise one or more “extended regions,” also referred to herein as “extension arms,” of nucleic acid sequence. The extended regions may comprise, but are not limited to, single-stranded RNA or DNA. Further, the extended regions may occur at the 3′ end of a traditional guide RNA. In other arrangements, the extended regions may occur at the 5′ end of a traditional guide RNA. In still other arrangements, the extended region may occur at an intramolecular region of the traditional guide RNA, for example, in the gRNA core region which associates and/or binds to the napDNAbp. The extended region comprises a “DNA synthesis template” or “reverse transcriptase template” that encodes (by the polymerase/reverse transcriptase of the prime editor) a single-stranded DNA which, in turn, has been designed to be (a) homologous with the endogenous target DNA to be edited, and (b) which comprises at least one desired nucleotide change (e.g., a transition, a transversion, a deletion, or an insertion) to be introduced or integrated into the endogenous target DNA. The extended region may also comprise other functional sequence elements, such as, but not limited to, a “primer binding site” and a “linker” sequence, or other structural elements, such as, but not limited to, aptamers, stem loops, hairpins, toe-loops (e.g., a 3′ toeloop), or an RNA-protein recruitment domain (e.g., MS2 hairpin). As used herein, the “primer binding site” comprises a sequence that hybridizes to a single-strand DNA sequence having a 3′ end generated from the nicked DNA of the R-loop.

In certain embodiments, the PEgRNAs have a 3′ extension arm, a spacer, and a gRNA core. The 3′ extension arm further comprises in the 5′ to 3′ direction a DNA synthesis template, a primer binding site, and a linker. The DNA synthesis template may also be referred to more broadly as the “DNA synthesis template” where the polymerase of a prime editor described herein is not an RT, but another type of polymerase.

In certain other embodiments, the PEgRNAs have a 5′ extension arm, a spacer, and a gRNA core. The 5′ extension further comprises in the 5′ to 3′ direction a DNA synthesis template, a primer binding site, and a linker. The DNA synthesis template may also be referred to more broadly as the “DNA synthesis template” where the polymerase of a prime editor described herein is not an RT, but another type of polymerase.

In still other embodiments, the PEgRNAs have in the 5′ to 3′ direction a spacer, a gRNA core, and an extension arm. The extension arm is at the 3′ end of the PEgRNA. The extension arm further comprises in the 5′ to 3′ direction a homology arm, an edit template, and a primer binding site. The extension arm may also comprise an optional modifier region at the 3′ and 5′ ends, which may be the same sequences or different sequences. In addition, the 3′ end of the PEgRNA may comprise a transcriptional terminator sequence. These sequence elements of the PEgRNAs are further described and defined herein.

In still other embodiments, the PEgRNAs have in the 5′ to 3′ direction an extension arm, a spacer, and a gRNA core. The extension arm is at the 5′ end of the PEgRNA. The extension arm further comprises in the 3′ to 5′ direction a primer binding site, an edit template, and a homology arm. The extension arm may also comprise an optional modifier region at the 3′ and 5′ ends, which may be the same sequences or different sequences. The PEgRNAs may also comprise a transcriptional terminator sequence at the 3′ end. These sequence elements of the PEgRNAs are further described and defined herein.

In some embodiments, epegRNAs provided in the present disclosure comprise a spacer sequence 5′-CATGGTGCACCTGACTCCTG-3′ (SEQ ID NO: 127), or a sequence at least 90%, at least 95%, at least 96%, at least 97%, at least 98%, or at least 99% identical to the sequence 5′-CATGGTGCACCTGACTCCTG-3′ (SEQ ID NO: 127) or a fragment thereof (e.g., at least 5, 6, 7, 8, 9, 10, 11, 12, 13, 14, 15, or more nucleotides of the sequence). In some embodiments, epegRNAs provided in the present disclosure comprise the sgRNA scaffold sequence 5′-GTTTTAGAGCTAGAAATAGCAAGTTAAAATAAGGCTAGTCCGTTATCAACTTGAA AAAGTGGCACCGAGTCGGTGC-3′ (SEQ ID NO: 126), or a sequence at least 90%, at least 95%, at least 96%, at least 97%, at least 98%, or at least 99% identical to the sequence 5′-GTTTTAGAGCTAGAAATAGCAAGTTAAAATAAGGCTAGTCCGTTATCAACTTGAA AAAGTGGCACCGAGTCGGTGC-3′ (SEQ ID NO: 126) or a fragment thereof (e.g., at least 5, 6, 7, 8, 9, 10, 11, 12, 13, 14, 15, or more nucleotides of the sequence). In some embodiments, epegRNAs provided in the present disclosure comprise the extension arm sequence 5′-AGACTTCTCTTCAGGAGTCAGGTGCAC-3′ (SEQ ID NO: 111), or a sequence at least 90%, at least 95%, at least 96%, at least 97%, at least 98%, or at least 99% identical to the sequence 5′-AGACTTCTCTTCAGGAGTCAGGTGCAC-3′ (SEQ ID NO: 111) or a fragment thereof (e.g., at least 5, 6, 7, 8, 9, 10, 11, 12, 13, 14, 15, or more nucleotides of the sequence). In certain embodiments, epegRNAs provided in the present disclosure comprise the sequence 5′-CATGGTGCACCTGACTCCTGGTTTTAGAGCTAGAAATAGCAAGTTAAAATAAGG CTAGTCCGTTATCAACTTGAAAAAGTGGCACCGAGTCGGTGCAGACTTCTCTTCA GGAGTCAGGTGCACAGAATAAACGCGGTTCTATCTAGTTACGCGTTAAACCAAC TAGAA-3′ (SEQ ID NO: 115), or a sequence at least 90%, at least 95%, at least 96%, at least 97%, at least 98%, or at least 99% identical to the sequence of SEQ ID NO: 115. In certain embodiments, epegRNAs provided herein comprise the sequence 5′-CATGGTGCACCTGACTCCTGGTTTTAGAGCTAGAAATAGCAAGTTAAAATAAGG CTAGTCCGTTATCAACTTGAAAAAGTGGCACCGAGTCGGTGCAGACTTCTCTTCA GGAGTCAGGTGCACAGAATAAACGCGGTTCTATCTAGTTACGCGTTAAACCAAC TAGAATTT-3′ (SEQ ID NO: 116), or a sequence at least 90%, at least 95%, at least 96%, at least 97%, at least 98%, or at least 99% identical to the sequence of SEQ ID NO: 116.

In some embodiments, a pegRNA is an “engineered pegRNA” (“epegRNA”). Relative to a pegRNA, an epegRNA comprises an additional structured motif, for example, attached to its 3′ end. Such additional structured motifs may stabilize the pegRNA or otherwise prevent it from being degraded. Suitable structured motifs include, but are not limited to, toe-loops, hairpins, stem-loops, pseudoknots, aptamers, G-quadruplexes, tRNAs, riboswitches, and ribozymes. In some embodiments, a 3′ structured motif comprises evopreq1. In some embodiments, a 3′ structured motif comprises a nucleotide sequence of any one of SEQ ID NOs: 48-77, or a sequence at least 90%, at least 95%, at least 96%, at least 97%, at least 98%, or at least 99% identical to the sequence of any one of SEQ ID NOs: 48-77 or a fragment thereof (e.g., at least 5, 6, 7, 8, 9, 10, 11, 12, 13, 14, 15, or more nucleotides of the sequence). In certain embodiments, a 3′ structured motif comprises the sequence 5′-CGCGGTTCTATCTAGTTACGCGTTAAACCAACTAGAA-3′ (SEQ ID NO: 72), or a sequence at least 90%, at least 95%, at least 96%, at least 97%, at least 98%, or at least 99% identical to the sequence 5′-CGCGGTTCTATCTAGTTACGCGTTAAACCAACTAGAA-3′ (SEQ ID NO: 72), or a fragment thereof (e.g., at least 5, 6, 7, 8, 9, 10, 11, 12, 13, 14, 15, or more nucleotides of the sequence). In some embodiments, an epegRNA comprises a 3′ structured motif connected to the rest of the pegRNA by an engineered linker (e.g., an engineered linker comprising the sequence 5′-AGAATAAA-3′ between the extension arm sequence and the 3′ structured motif, or a sequence at least 90%, at least 95%, at least 96%, at least 97%, at least 98%, or at least 99% identical to the sequence 5′-AGAATAAA-3′ or a fragment thereof (e.g., at least 4, 5, 6, or more nucleotides of the sequence).

### PE1

As used herein, “PE1” refers to a prime editing composition comprising 1) a fusion protein comprising a Cas9 protein variant Cas9 (H840A) and a wild type MMLV RT having the following structure: [NLS]-[Cas9 (H840A)]-[linker]-[MMLV_RT (wt)]-NLS and 2) a desired PEgRNA, wherein the fusion protein (referred to as the PE1 protein) has the amino acid sequence of SEQ ID NO: 3, which is shown as follows.

(SEQ ID NO: 3)
MKRTADGSEFESPKKKRKVDKKYSIGLDIGTNSVGWAVITDEYKVPSKKFK
VLGNTDRHSIKKNLIGALLFDSGETAEATRLKRTARRRYTRRKNRICYLQ
EIFSNEMAKVDDSFFHRLEESFLVEEDKKHERHPIFGNIVDEVAYHEKYP
TIYHLRKKLVDSTDKADLRLIYLALAHMIKFRGHFLIEGDLNPDNSDVDK
LFIQLVQTYNQLFEENPINASGVDAKAILSARLSKSRRLENLIAQLPGEK
KNGLFGNLIALSLGLTPNFKSNFDLAEDAKLQLSKDTYDDDLDNLLAQIG
DQYADLFLAAKNLSDAILLSDILRVNTEITKAPLSASMIKRYDEHHQDLT
LLKALVRQQLPEKYKEIFFDQSKNGYAGYIDGGASQEEFYKFIKPILEKM
DGTEELLVKLNREDLLRKQRTFDNGSIPHQIHLGELHAILRRQEDFYPFL
KDNREKIEKILTFRIPYYVGPLARGNSRFAWMTRKSEETITPWNFEEVVD
KGASAQSFIERMTNFDKNLPNEKVLPKHSLLYEYFTVYNELTKVKYVTEG
MRKPAFLSGEQKKAIVDLLFKTNRKVTVKQLKEDYFKKIECFDSVEISGV
EDRFNASLGTYHDLLKIIKDKDFLDNEENEDILEDIVLTLTLFEDREMIE
ERLKTYAHLFDDKVMKQLKRRRYTGWGRLSRKLINGIRDKQSGKTILDFL
KSDGFANRNFMQLIHDDSLTFKEDIQKAQVSGQGDSLHEHIANLAGSPAI
KKGILQTVKVVDELVKVMGRHKPENIVIEMARENQTTQKGQKNSRERMKR
IEEGIKELGSQILKEHPVENTQLQNEKLYLYYLQNGRDMYVDQELDINRL
SDYDVDAIVPQSFLKDDSIDNKVLTRSDKNRGKSDNVPSEEVVKKMKNYW
RQLLNAKLITQRKFDNLTKAERGGLSELDKAGFIKRQLVETRQITKHVAQ
ILDSRMNTKYDENDKLIREVKVITLKSKLVSDFRKDFQFYKVREINNYHH
AHDAYLNAVVGTALIKKYPKLESEFVYGDYKVYDVRKMIAKSEQEIGKAT
AKYFFYSNIMNFFKTEITLANGEIRKRPLIETNGETGEIVWDKGRDFATV
RKVLSMPQVNIVKKTEVQTGGFSKESILPKRNSDKLIARKKDWDPKKYGG
FDSPTVAYSVLVVAKVEKGKSKKLKSVKELLGITIMERSSFEKNPIDFLE
AKGYKEVKKDLIIKLPKYSLFELENGRKRMLASAGELQKGNELALPSKYV
NFLYLASHYEKLKGSPEDNEQKQLFVEQHKHYLDEIIEQISEFSKRVILA
DANLDKVLSAYNKHRDKPIREQAENIIHLFTLTNLGAPAAFKYFDTTIDR
KRYTSTKEVLDATLIHQSITGLYETRIDLSQLGGDSGGSSGGSSGSETPG
TSESATPESSGGSSGGSSTLNIEDEYRLHETSKEPDVSLGSTWLSDFPQA
WAETGGMGLAVRQAPLIIPLKATSTPVSIKQYPMSQEARLGIKPHIQRLL
DQGILVPCQSPWNTPLLPVKKPGTNDYRPVQDLREVNKRVEDIHPTVPNP
YNLLSGLPPSHQWYTVLDLKDAFFCLRLHPTSQPLFAFEWRDPEMGISGQ
LTWTRLPQGFKNSPTLFDEALHRDLADFRIQHPDLILLQYVDDLLLAATS
ELDCQQGTRALLQTLGNLGYRASAKKAQICQKQVKYLGYLLKEGQRWLTE
ARKETVMGQPTPKTPRQLREFLGTAGFCRLWIPGFAEMAAPLYPLTKTGT
LFNWGPDQQKAYQEIKQALLTAPALGLPDLTKPFELFVDEKQGYAKGVLT
QKLGPWRRPVAYLSKKLDPVAAGWPPCLRMVAAIAVLTKDAGKLTMGQPL
VILAPHAVEALVKQPPDRWLSNARMTHYQALLLDTDRVQFGPVVALNPAT
LLPLPEEGLQHNCLDILAEAHGTRPDLTDQPLPDADHTWYTDGSSLLQEG
QRKAGAAVTTETEVIWAKALPAGTSAQRAELIALTQALKMAEGKKLNVYT
DSRYAFATAHIHGEIYRRRGLLTSEGKEIKNKDEILALLKALFLPKRLSI
IHCPGHQKGHSAEARGNRMADQAARKAAITETPDTSTLLIENSSPSGGSK
RTADGSEFEPKKKRKV
KEY:
NUCLEAR LOCALIZATION SEQUENCE (NLS) TOP:
(SEQ ID NO: 95),
BOTTOM: (SEQ ID NO: 96)
CAS9(H840A) (SEQ ID NO: 10)
33-AMINO ACID LINKER (SEQ ID NO: 80)
M-MLV reverse transcriptase (SEQ ID NO: 30).

### PE2

As used herein, “PE2” refers to a prime editing composition comprising 1) a fusion protein comprising a Cas9 protein variant Cas9 (H840A) and a variant MMLV RT having the following structure: [NLS]-[Cas9 (H840A)]-[linker]-[MMLV_RT (D200N)(T330P)(L603W)(T306K)(W313F)]-NLS and 2) a desired PERNA, wherein the fusion protein (referred to as the PE2 protein) has the amino acid sequence of SEQ ID NO: 4, which is shown as follows:

(SEQ ID NO: 4)
MKRTADGSEFESPKKKRKVDKKYSIGLDIGTNSVGWAVITDEYKVPSKKF
KVLGNTDRHSIKKNLIGALLFDSGETAEATRLKRTARRRYTRRKNRICYL
QEIFSNEMAKVDDSFFHRLEESFLVEEDKKHERHPIFGNIVDEVAYHEKY
PTIYHLRKKLVDSTDKADLRLIYLALAHMIKFRGHFLIEGDLNPDNSDVD
KLFIQLVQTYNQLFEENPINASGVDAKAILSARLSKSRRLENLIAQLPGE
KKNGLFGNLIALSLGLTPNFKSNFDLAEDAKLQLSKDTYDDDLDNLLAQI
GDQYADLFLAAKNLSDAILLSDILRVNTEITKAPLSASMIKRYDEHHQDL
TLLKALVRQQLPEKYKEIFFDQSKNGYAGYIDGGASQEEFYKFIKPILEK
MDGTEELLVKLNREDLLRKQRTFDNGSIPHQIHLGELHAILRRQEDFYPF
LKDNREKIEKILTFRIPYYVGPLARGNSRFAWMTRKSEETITPWNFEEVV
DKGASAQSFIERMTNFDKNLPNEKVLPKHSLLYEYFTVYNELTKVKYVTE
GMRKPAFLSGEQKKAIVDLLFKTNRKVTVKQLKEDYFKKIECFDSVEISG
VEDRFNASLGTYHDLLKIIKDKDFLDNEENEDILEDIVLTLTLFEDREMI
EERLKTYAHLFDDKVMKQLKRRRYTGWGRLSRKLINGIRDKQSGKTILDF
LKSDGFANRNFMQLIHDDSLTFKEDIQKAQVSGQGDSLHEHIANLAGSPA
IKKGILQTVKVVDELVKVMGRHKPENIVIEMARENQTTQKGQKNSRERMK
RIEEGIKELGSQILKEHPVENTQLQNEKLYLYYLQNGRDMYVDQELDINR
LSDYDVDAIVPQSFLKDDSIDNKVLTRSDKNRGKSDNVPSEEVVKKMKNY
WRQLLNAKLITQRKFDNLTKAERGGLSELDKAGFIKRQLVETRQITKHVA
QILDSRMNTKYDENDKLIREVKVITLKSKLVSDFRKDFQFYKVREINNYH
HAHDAYLNAVVGTALIKKYPKLESEFVYGDYKVYDVRKMIAKSEQEIGKA
TAKYFFYSNIMNFFKTEITLANGEIRKRPLIETNGETGEIVWDKGRDFAT
VRKVLSMPQVNIVKKTEVQTGGFSKESILPKRNSDKLIARKKDWDPKKYG
GFDSPTVAYSVLVVAKVEKGKSKKLKSVKELLGITIMERSSFEKNPIDFL
EAKGYKEVKKDLIIKLPKYSLFELENGRKRMLASAGELQKGNELALPSKY
VNFLYLASHYEKLKGSPEDNEQKQLFVEQHKHYLDEIIEQISEFSKRVIL
ADANLDKVLSAYNKHRDKPIREQAENIIHLFTLTNLGAPAAFKYFDTTID
RKRYTSTKEVLDATLIHQSITGLYETRIDLSQLGGDSGGSSGGSSGSETP
GTSESATPESSGGSSGGSSTLNIEDEYRLHETSKEPDVSLGSTWLSDFPQ
AWAETGGMGLAVRQAPLIIPLKATSTPVSIKQYPMSQEARLGIKPHIQRL
LDQGILVPCQSPWNTPLLPVKKPGTNDYRPVQDLREVNKRVEDIHPTVPN
PYNLLSGLPPSHQWYTVLDLKDAFFCLRLHPTSQPLFAFEWRDPEMGISG
QLTWTRLPQGFKNSPTLFNEALHRDLADFRIQHPDLILLQYVDDLLLAAT
SELDCQQGTRALLQTLGNLGYRASAKKAQICQKQVKYLGYLLKEGQRWLT
EARKETVMGQPTPKTPRQLREFLGKAGFCRLFIPGFAEMAAPLYPLTKPG
TLFNWGPDQQKAYQEIKQALLTAPALGLPDLTKPFELFVDEKQGYAKGVL
TQKLGPWRRPVAYLSKKLDPVAAGWPPCLRMVAAIAVLTKDAGKLTMGQP
LVILAPHAVEALVKQPPDRWLSNARMTHYQALLLDTDRVQFGPVVALNPA
TLLPLPEEGLOHNCLDILAEAHGTRPDLTDQPLPDADHTWYTDGSSLLQE
GQRKAGAAVTTETEVIWAKALPAGTSAQRAELIALTQALKMAEGKKLNVY
TDSRYAFATAHIHGEIYRRRGWLTSEGKEIKNKDEILALLKALFLPKRLS
IIHCPGHQKGHSAEARGNRMADQAARKAAITETPDTSTLLIENSSPSGGS
KRTADGSEFEPKKKRKV
KEY:
NUCLEAR LOCALIZATION SEQUENCE (NLS) TOP: (SEQ ID NO: 95), BOTTOM: (SEQ ID NO: 96)
CAS9(H840A) (SEQ ID NO: 10)
33-AMINO ACID LINKER (SEQ ID NO: 80)
M-MLV reverse transcriptase (SEQ ID NO: 31)

### PE3

As used herein, “PE3” refers to a prime editing composition comprising a PE2 prime editor and further comprising a second-strand nicking guide RNA that complexes with PE2 and introduces a nick in the non-edit DNA strand in order to induce preferential replacement of the edit strand.

### PE3b

As used herein, “PE3b” refers to a prime editing composition comprising PE2 and further comprising a second-strand nicking guide RNA that complexes with PE2 and introduces a nick in the non-edit DNA strand, wherein the second-strand nicking guide RNA is designed for temporal control such that the second strand nick is not introduced until after the installation of the desired edit. This is achieved by designing the second strand nicking guide RNA with a spacer sequence that comprises complementarity to, and only hybridizes with, the edited strand after installation of the desired nucleotide edit(s), but not the endogenous target DNA sequence. Using this strategy, mismatches between the nicking guide RNA spacer and the unedited target DNA should disfavor nicking by the sgRNA until after the editing event on the PAM strand takes place.

### PE4

As used herein, “PE4” refers to a prime editing composition comprising a PE2 and further comprising an MLH1 dominant negative protein variant (i.e., wild-type MLH1 with amino acids 754-756 truncated, which may be referred to herein as “MLH1 4754-756” or “MLH1dn”). The MLH1 dominant negative protein variant may be expressed in trans in some embodiments. In some embodiments, a PE4 system comprises a fusion protein comprising a PE2 protein and an MLH1 dominant negative protein joined via an optional linker.

### PE5 and PE5b

As used herein, “PE5” refers to a prime editing composition comprising a PE3 prime editor and further comprising an MLH1 dominant negative protein variant (i.e., wild-type MLH1 with amino acids 754-756 truncated, which may be referred to as “MLH1 Δ754-756” or “MLH1dn”). The MLH1 dominant negative variant may be expressed in trans in some embodiments. In some embodiments, a PE5 system comprises a fusion protein comprising a PE2 protein and an MLH1 dominant negative protein joined via an optional linker. “PE5b” refers to a prime editing composition comprising a PE3 and an MLH1 dominant negative protein, wherein the second-strand nicking guide RNA is designed for temporal control such that the second strand nick is not introduced until after the installation of the desired edit. This is achieved by designing the second strand nicking guide RNA with a spacer sequence that comprise complementarity to, and hybridize with, only the edited strand after installation of the desired nucleotide edit(s), but not the endogenous target DNA sequence.

### PEmax

As used herein, “PEmax” refers to a prime editing composition comprising 1) a fusion protein comprising a Cas9 protein variant Cas9(R221K N39K H840A) and a variant MMLV RT having the following structure: [bipartite NLS]-[Cas9(R221K)(N394K)(H840A)]-[linker]-[MMLV_RT (D200N)(T330P)(L603W)]-[bipartite NLS]-[NLS] and 2) a desired PEgRNA. A prime editor may be said to have “PEmax architecture” as long as it comprises such mutations and structure (e.g., PE3 may comprise PEmax architecture to form “PE3max”). In some embodiments, a PEmax fusion protein has the amino acid sequence of SEQ ID NO: 5, which is shown as follows:

(SEQ ID NO: 5)
MKRTADGSEFESPKKKRKVDKKYSIGLDIGTNSVGWAVITDEYKVPSKKF
KVLGNTDRHSIKKNLIGALLFDSGETAEATRLKRTARRRYTRRKNRICYL
QEIFSNEMAKVDDSFFHRLEESFLVEEDKKHERHPIFGNIVDEVAYHEKY
PTIYHLRKKLVDSTDKADLRLIYLALAHMIKFRGHFLIEGDLNPDNSDVD
KLFIQLVQTYNQLFEENPINASGVDAKAILSARLSKSRKLENLIAQLPGE
KKNGLFGNLIALSLGLTPNFKSNFDLAEDAKLQLSKDTYDDDLDNLLAQI
GDQYADLFLAAKNLSDAILLSDILRVNTEITKAPLSASMIKRYDEHHQDL
TLLKALVRQQLPEKYKEIFFDQSKNGYAGYIDGGASQEEFYKFIKPILEK
MDGTEELLVKLKREDLLRKQRTFDNGSIPHQIHLGELHAILRRQEDFYPF
LKDNREKIEKILTFRIPYYVGPLARGNSRFAWMTRKSEETITPWNFEEVV
DKGASAQSFIERMTNFDKNLPNEKVLPKHSLLYEYFTVYNELTKVKYVTE
GMRKPAFLSGEQKKAIVDLLFKTNRKVTVKQLKEDYFKKIECFDSVEISG
VEDRFNASLGTYHDLLKIIKDKDFLDNEENEDILEDIVLTLTLFEDREMI
EERLKTYAHLFDDKVMKQLKRRRYTGWGRLSRKLINGIRDKQSGKTILDF
LKSDGFANRNFMQLIHDDSLTFKEDIQKAQVSGQGDSLHEHIANLAGSPA
IKKGILQTVKVVDELVKVMGRHKPENIVIEMARENQTTQKGQKNSRERMK
RIEEGIKELGSQILKEHPVENTQLQNEKLYLYYLQNGRDMYVDQELDINR
LSDYDVDAIVPQSFLKDDSIDNKVLTRSDKNRGKSDNVPSEEVVKKMKNY
WRQLLNAKLITQRKFDNLTKAERGGLSELDKAGFIKRQLVETRQITKHVA
QILDSRMNTKYDENDKLIREVKVITLKSKLVSDFRKDFQFYKVREINNYH
HAHDAYLNAVVGTALIKKYPKLESEFVYGDYKVYDVRKMIAKSEQEIGKA
TAKYFFYSNIMNFFKTEITLANGEIRKRPLIETNGETGEIVWDKGRDFAT
VRKVLSMPQVNIVKKTEVQTGGFSKESILPKRNSDKLIARKKDWDPKKYG
GFDSPTVAYSVLVVAKVEKGKSKKLKSVKELLGITIMERSSFEKNPIDFL
EAKGYKEVKKDLIIKLPKYSLFELENGRKRMLASAGELQKGNELALPSKY
VNFLYLASHYEKLKGSPEDNEQKQLFVEQHKHYLDEIIEQISEFSKRVIL
ADANLDKVLSAYNKHRDKPIREQAENIIHLFTLTNLGAPAAFKYFDTTID
RKRYTSTKEVLDATLIHQSITGLYETRIDLSQLGGDSGGSSGGSKRTADG
SEFESPKKKRKVSGGSSGGSTLNIEDEYRLHETSKEPDVSLGSTWLSDFP
QAWAETGGMGLAVRQAPLIIPLKATSTPVSIKQYPMSQEARLGIKPHIQR
LLDQGILVPCQSPWNTPLLPVKKPGTNDYRPVQDLREVNKRVEDIHPTVP
NPYNLLSGLPPSHQWYTVLDLKDAFFCLRLHPTSQPLFAFEWRDPEMGIS
GQLTWTRLPQGFKNSPTLFNEALHRDLADFRIQHPDLILLQYVDDLLLAA
TSELDCQQGTRALLQTLGNLGYRASAKKAQICQKQVKYLGYLLKEGQRWL
TEARKETVMGQPTPKTPRQLREFLGKAGFCRLFIPGFAEMAAPLYPLTKP
GTLFNWGPDQQKAYQEIKQALLTAPALGLPDLTKPFELFVDEKQGYAKGV
LTQKLGPWRRPVAYLSKKLDPVAAGWPPCLRMVAAIAVLTKDAGKLTMGQ
PLVILAPHAVEALVKQPPDRWLSNARMTHYQALLLDTDRVQFGPVVALNP
ATLLPLPEEGLOHNCLDILAEAHGTRPDLTDQPLPDADHTWYTDGSSLLQ
EGQRKAGAAVTTETEVIWAKALPAGTSAQRAELIALTQALKMAEGKKLNV
YTDSRYAFATAHIHGEIYRRRGWLTSEGKEIKNKDEILALLKALFLPKRL
SIIHCPGHQKGHSAEARGNRMADQAARKAAITETPDTSTLLIENSSPSGG
SKRTADGSEFESPKKKRKVGSGPAAKRVKLD
KEY:
BIPARTITE SV40 NUCLEAR LOCALIZATION SEQUENCE (NLS) TOP: (SEQ ID NO: 95),
CAS9(R221K N39K H840A) (SEQ ID NO: 11)
SGGSx2-BIPARTITE SV40NLS-SGGSx2 LINKER (SEQ ID NO: 79)
M-MLV reverse transcriptase(D200N T306K W313F T330P L603W) (SEQ ID NO: 31)
Other linker sequence (SEQ ID NO: 82)
BIPARTITE SV40NLS (SEQ ID NO: 97)
Other linker sequence c-Myc NLS (SEQ ID NO: 98)

### PE3max and PE3bmax

As used herein, “PE3max” refers to a prime editing composition comprising a PEmax protein, a desired pegRNA, and a second strand nicking guide RNA. In some embodiments, PE3max can be considered as PE3 except wherein the PE2 component is substituted with PEmax. “PE3bmax” refers to a prime editing composition comprising a PEmax protein, a desired pegRNA, and a second strand nicking guide RNA, wherein the second-strand nicking guide RNA is designed for temporal control such that the second strand nick is not introduced until after the installation of the desired edit. This is achieved by designing the second strand nicking guide RNA with a spacer sequence that comprise complementarity to, and hybridizes with, only the edited strand after installation of the desired nucleotide edit(s), but not the endogenous target DNA sequence.

### PE4max

As used herein, “PE4max” refers to PE4 but wherein the PE2 component is substituted with PEmax.

### PE5max and PE5bmax

As used herein, “PE5max” refers to PE5, but wherein the PE2 component of PE3 is substituted with PEmax. “PE5bmax” refers to PE5b wherein the PE2 component of PE3 is substituted with PEmax.

### Polymerase

As used herein, the term “polymerase” refers to an enzyme that synthesizes a nucleotide strand and that may be used in connection with the prime editor delivery systems described herein. The polymerase can be a “template-dependent” polymerase (i.e., a polymerase that synthesizes a nucleotide strand based on the order of nucleotide bases of a template strand). The polymerase can also be a “template-independent” polymerase (i.e., a polymerase that synthesizes a nucleotide strand without the requirement of a template strand). A polymerase may also be further categorized as a “DNA polymerase” or an “RNA polymerase.” In various embodiments, the prime editor system comprises a DNA polymerase. In various embodiments, the DNA polymerase can be a “DNA-dependent DNA polymerase” (i.e., whereby the template molecule is a strand of DNA). In such cases, the DNA template molecule can be a PEgRNA, wherein the extension arm comprises a strand of DNA. In such cases, the PERNA may be referred to as a chimeric or hybrid PEgRNA that comprises an RNA portion (i.e., the guide RNA components, including the spacer and the gRNA core) and a DNA portion (i.e., the extension arm). In various other embodiments, the DNA polymerase can be an “RNA-dependent DNA polymerase” (i.e., whereby the template molecule is a strand of RNA). In such cases, the PEgRNA is RNA, i.e., including an RNA extension. The term “polymerase” may also refer to an enzyme that catalyzes the polymerization of nucleotides (i.e., the polymerase activity). Generally, the enzyme will initiate synthesis at the 3′-end of a primer annealed to a polynucleotide template sequence (e.g., such as a primer sequence annealed to the primer binding site of a PEgRNA) and will proceed toward the 5′ end of the template strand. A “DNA polymerase” catalyzes the polymerization of deoxynucleotides. As used herein in reference to a DNA polymerase, the term DNA polymerase includes a “functional fragment thereof.” A “functional fragment thereof” refers to any portion of a wild-type or mutant DNA polymerase that encompasses less than the entire amino acid sequence of the polymerase and which retains the ability, under at least one set of conditions, to catalyze the polymerization of a polynucleotide. Such a functional fragment may exist as a separate entity, or it may be a constituent of a larger polypeptide, such as a fusion protein.

### Prime Editing

As used herein, the term “prime editing” refers to an approach for gene editing using napDNAbps, a polymerase (e.g., a reverse transcriptase), and specialized guide RNAs that include a primer binding site and a DNA synthesis template for encoding desired new genetic information (or deleting genetic information) that is then incorporated into a target DNA sequence. Prime editing is described in Anzalone, A. V. et al., Search-and-replace genome editing without double-strand breaks or donor DNA. Nature 576, 149-157 (2019), which is incorporated herein by reference. See also International PCT application, PCT/US2020/023721, filed Mar. 19, 2020, and published as WO 2020/191239, which is incorporated herein by reference.

Prime editing represents a platform for genome editing that is a versatile and precise method to directly write new genetic information into a specified DNA site using a nucleic acid programmable DNA binding protein (“napDNAbp”) working in association with a polymerase (i.e., in the form of a fusion protein or otherwise provided in trans with the napDNAbp), wherein the prime editing system is programmed with a prime editing (PE) guide RNA (“PEgRNA”) that both specifies the target site and templates the synthesis of the desired edit in the form of a replacement DNA strand by way of an extension (either DNA or RNA) engineered onto a guide RNA (e.g., at the 5′ or 3′ end, or at an internal portion of a guide RNA). The replacement strand containing the desired edit (e.g., a single nucleobase substitution) shares the same sequence as the endogenous strand (or is homologous to it) immediately downstream of the nick site of the target site to be edited (with the exception that it includes the desired edit). Through DNA repair and/or replication machinery, the endogenous strand downstream of the nick site is replaced by the newly synthesized replacement strand containing the desired edit. In some cases, prime editing may be thought of as a “search-and-replace” genome editing technology since the prime editors, as described herein, not only search and locate the desired target site to be edited, but at the same time, encode a replacement strand containing a desired edit that is installed in place of the corresponding target site endogenous DNA strand. The prime editors of the present disclosure relate, in part, to the discovery that the mechanism of target-primed reverse transcription (TPRT) or “prime editing” can be leveraged or adapted for conducting precision CRISPR/Cas-based genome editing with high efficiency and genetic flexibility. TPRT is naturally used by mobile DNA elements, such as mammalian non-LTR retrotransposons and bacterial Group II introns. Cas protein-reverse transcriptase fusions or related systems are used to target a specific DNA sequence with a guide RNA, generate a single strand nick at the target site, and use the nicked DNA as a primer for reverse transcription of an engineered DNA synthesis template that is integrated with the guide RNA. However, while the concept begins with prime editors that use reverse transcriptase as the DNA polymerase component, the prime editors described herein are not limited to reverse transcriptases but may include the use of virtually any DNA polymerase. Indeed, while the application throughout may refer to prime editors with “reverse transcriptases,” it is set forth here that reverse transcriptases are only one type of DNA polymerase that may work with prime editing. Thus, wherever the specification mentions a “reverse transcriptase,” the person having ordinary skill in the art should appreciate that any suitable DNA polymerase may be used in place of the reverse transcriptase. Thus, in one aspect, the prime editors may comprise Cas9 (or an equivalent napDNAbp), which is programmed to target a DNA sequence by associating it with a specialized guide RNA (i.e., PERNA) containing a spacer sequence that anneals to a complementary sequence (the complementary sequence to an endogenous protospacer sequence) in the target DNA. The PEgRNA also contains new genetic information in the form of an extension that encodes a replacement strand of DNA containing a desired nucleotide change which is used to replace a corresponding endogenous DNA strand at the target site. To transfer information from the PEgRNA to the target DNA, the mechanism of prime editing involves nicking the target site in one strand of the DNA to expose a 3′-hydroxyl group. The exposed 3′-hydroxyl group can then be used to prime the DNA polymerization of the edit-encoding extension on PERNA directly into the target site. In various embodiments, the extension-which provides the template for polymerization of the replacement strand containing the edit—can be formed from RNA or DNA. In the case of an RNA extension, the polymerase of the prime editor can be an RNA-dependent DNA polymerase (such as a reverse transcriptase). In the case of a DNA extension, the polymerase of the prime editor may be a DNA-dependent DNA polymerase. The newly synthesized strand (i.e., the replacement DNA strand containing the desired nucleotide edit) that is formed by the prime editor would be homologous to the genomic target sequence (i.e., have the same sequence as), except for the inclusion of one or more desired nucleotide changes (e.g., a single nucleotide substitution, a deletion, or an insertion, or a combination thereof). The newly synthesized (or replacement) strand of DNA may also be referred to as a single strand DNA flap, which would compete for hybridization with the complementary homologous endogenous DNA strand, thereby displacing the corresponding endogenous strand. Resolution of the hybridized intermediate (also referred to as a heteroduplex, comprising the single strand DNA flap synthesized by the reverse transcriptase hybridized to the endogenous DNA strand with the exception of mismatches at positions where desired nucleotide edits are installed in the edit strand) can include removal of the resulting displaced flap of endogenous DNA (e.g., with a 5′ end DNA flap endonuclease, FEN1), ligation of the synthesized single strand DNA flap to the target DNA, and assimilation of the desired nucleotide changes as a result of cellular DNA repair and/or replication processes.

In various embodiments, prime editing operates by contacting a target DNA molecule (for which a change in the nucleotide sequence is desired to be introduced) with a nucleic acid programmable DNA binding protein (napDNAbp) complexed with a prime editing guide RNA (PEgRNA). In various embodiments, the prime editing guide RNA (PEgRNA) comprises an extension at the 3′ or 5′ end of the guide RNA, or at an intramolecular location in the guide RNA, and encodes the desired nucleotide change (e.g., single nucleotide substitution, insertion, or deletion). First, the napDNAbp/extended gRNA complex contacts the DNA molecule, and the extended gRNA guides the napDNAbp to bind to a target locus. Next, a nick in one of the strands of DNA of the target locus is introduced (e.g., by a nuclease or chemical agent), thereby creating an available 3′ end in one of the strands of the target locus. In certain embodiments, the nick is created in the strand of DNA that corresponds to the R-loop strand, i.e., the strand that is not hybridized to the guide RNA sequence, i.e., the “non-target strand.” The nick, however, could be introduced in either of the strands. That is, the nick could be introduced into the R-loop “target strand” (i.e., the strand hybridized to the protospacer of the extended gRNA) or the “non-target strand” (i.e., the strand forming the single-stranded portion of the R-loop and which is complementary to the target strand). In the next step, the 3′ end of the DNA strand (formed by the nick) interacts with the extended portion of the guide RNA in order to prime reverse transcription (i.e., “target-primed RT”). In certain embodiments, the 3′ end DNA strand hybridizes to a specific RT priming sequence on the extended portion of the guide RNA, i.e., the “reverse transcriptase priming sequence” or “primer binding site” on the PEgRNA. In the next step, a reverse transcriptase (or other suitable DNA polymerase) is introduced that synthesizes a single strand of DNA from the 3′ end of the primed site towards the 5′ end of the prime editing guide RNA. The DNA polymerase (e.g., reverse transcriptase) can be fused to the napDNAbp or alternatively can be provided in trans to the napDNAbp. This forms a single-strand DNA flap comprising the desired nucleotide change (e.g., the single base change, insertion, or deletion, or a combination thereof) and that is otherwise homologous to the endogenous DNA at or adjacent to the nick site. In the next step, the napDNAbp and guide RNA are released. The final two steps relate to the resolution of the single strand DNA flap such that the desired nucleotide change becomes incorporated into the target locus. This process can be driven towards the desired product formation by removing the corresponding 5′ endogenous DNA flap that forms once the 3′ single strand DNA flap invades and hybridizes to the endogenous DNA sequence. Without being bound by theory, the cell's endogenous DNA repair and replication processes resolve the mismatched DNA to incorporate the nucleotide change(s) to form the desired altered product. The process can also be driven towards product formation with “second strand nicking.” This process may introduce at least one or more of the following genetic changes: transversions, transitions, deletions, and insertions.

### Prime Editor

The term “prime editor” refers to the polypeptide or polypeptide components involved in prime editing as described herein. In some embodiments, a prime editor comprises a fusion construct comprising a napDNAbp (e.g., Cas9 nickase) and a reverse transcriptase. In some embodiments, a prime editor is capable of carrying out prime editing on a target nucleotide sequence in the presence of a PERNA (or “extended guide RNA”). In some embodiments, a prime editor comprises a napDNAbp (e.g., Cas9 nickase) and a reverse transcriptase provided in trans, i.e., the napDNAbp and the reverse transcriptase are not fused. The in trans napDNAbp and the reverse transcriptase may be tethered via a non-peptide linkage, e.g., an MS2 RNA-protein binding RNA sequence and a MS2 coat protein fused to either the napDNAbp or the reverse transcriptase, or may be unlinked to each other and simply recruited by the pegRNA. In some embodiments, a prime editor composition, system, or complex provided herein comprises a fusion protein or a fusion protein complexed with a PEgRNA, and/or further complexed with a second-strand nicking sgRNA. In some embodiments, the prime editor system may also refer to the complex comprising a fusion protein (reverse transcriptase fused to a napDNAbp), a PEgRNA, and a regular guide RNA capable of directing the second-site nicking step of the non-edited strand as described herein.

### Primer Binding Site

The term “primer binding site” or “PBS” refers to the portion of a PEgRNA as a component of the extension arm (e.g., at the 3′ end of the extension arm), and is a single-stranded portion of the PEgRNA as a component of the extension arm that comprises a region of complementarity to a sequence on the non-target strand of a double stranded target DNA. In some embodiments, the primer binding site is complementary to a region upstream of a nick site in a non-target strand. In some embodiments, the primer binding site is complementary to a region immediately upstream of a nick site in the non-target strand. In some embodiments, the primer binding site is capable of binding to the primer sequence that is formed after nicking of the edit strand (the non-target strand) of the target DNA sequence by the prime editor. When the prime editor (e.g., by a Cas9 nickase component of a prime editor) nicks the edit strand of the target DNA sequence, a free 3′ end is formed in the edit strand, which serves as a primer sequence that anneals to the primer binding site on the PEgRNA to prime reverse transcription. In some embodiments, the PBS is complementary to or substantially complementary to and can anneal to a free 3′ end on the non-target strand of the double stranded target DNA at the nick site. In some embodiments, the PBS anneals to the free 3′ end on the non-target strand can initiate target-primed DNA synthesis.

### Protein, Peptide, and Polypeptide

The terms “protein,” “peptide,” and “polypeptide” are used interchangeably herein and refer to a polymer of amino acid residues linked together by peptide (amide) bonds. The terms refer to a protein, peptide, or polypeptide of any size, structure, or function. Typically, a protein, peptide, or polypeptide will be at least three amino acids long. A protein, peptide, or polypeptide may refer to an individual protein or a collection of proteins. One or more of the amino acids in a protein, peptide, or polypeptide may be modified, for example, by the addition of a chemical entity such as a carbohydrate group, a hydroxyl group, a phosphate group, a farnesyl group, an isofarnesyl group, a fatty acid group, a linker for conjugation, functionalization, or other modification, etc. A protein, peptide, or polypeptide may also be a single molecule or may be a multi-molecular complex. A protein, peptide, or polypeptide may be just a fragment of a naturally occurring protein or peptide. A protein, peptide, or polypeptide may be naturally occurring, recombinant, or synthetic, or any combination thereof. Any of the proteins provided herein may be produced by any method known in the art. For example, the proteins provided herein may be produced via recombinant protein expression and purification, which is especially suited for fusion proteins comprising a peptide linker. Methods for recombinant protein expression and purification are well known, and include those described by Green and Sambrook, Molecular Cloning: A Laboratory Manual (4th ed., Cold Spring Harbor Laboratory Press, Cold Spring Harbor, N.Y. (2012)), the contents of which are incorporated herein by reference.

### Protospacer

As used herein, the term “protospacer” refers to the sequence (e.g., of ~20 bp) in DNA adjacent to the PAM (protospacer adjacent motif) sequence. The protospacer shares the same sequence as the spacer sequence of the guide RNA (except that a protospacer contains Thymine and the spacer sequence contains Uracil). The guide RNA anneals to the complement of the protospacer sequence on the target DNA (specifically, one strand thereof, i.e., the “target strand” versus the “non-target strand” of the target DNA sequence). In some embodiments, in order for a Cas nickase component of a prime editor to function, it also requires a specific protospacer adjacent motif (PAM) that varies depending on the Cas protein component itself, e.g., the type of Cas protein and the bacterial species from which it is derived. The most commonly used Cas9 nuclease, derived from S. pyogenes, recognizes a PAM sequence of NGG that is directly downstream of the protospacer sequence in the genomic DNA, on the non-target strand.

### Protospacer Adjacent Motif (PAM)

As used herein, the term “protospacer adjacent motif” or “PAM” refers to a DNA sequence (e.g., an approximately 2-6 nucleotide sequence) that is an important targeting component of a Cas nuclease, e.g., a Cas9. For example, in some embodiments for a Cas9 nuclease, the PAM sequence is on either strand and is downstream in the 5′ to 3′ direction of the Cas9 cut site. The canonical PAM sequence (i.e., the PAM sequence that is associated with the Cas9 nuclease of Streptococcus pyogenes or SpCas9) is 5′-NGG-3′, wherein “N” is any nucleobase followed by two guanine (“G”) nucleobases. In some embodiments, SpCas9 can also recognize additional non-canonical PAMs (e.g., NAG and NGA).

Different PAM sequences can be associated with different Cas9 nucleases or equivalent proteins from different organisms. In addition, any given Cas9 nuclease, e.g., SpCas9, may be modified to alter the PAM specificity of the nuclease such that the nuclease recognizes an alternative PAM sequence.

### Reverse Transcriptase

The term “reverse transcriptase” describes a class of polymerases characterized as RNA-dependent DNA polymerases. All known reverse transcriptases require a primer to synthesize a DNA transcript from an RNA template. Historically, reverse transcriptase has been used primarily to transcribe mRNA into cDNA, which can then be cloned into a vector for further manipulation. Avian myoblastosis virus (AMV) reverse transcriptase was the first widely used RNA-dependent DNA polymerase (Verma, Biochim. Biophys. Acta 473:1 (1977)). The enzyme has 5′-3′ RNA-directed DNA polymerase activity, 5′-3′ DNA-directed DNA polymerase activity, and RNase H activity. RNase H is a processive 5′ and 3′ ribonuclease specific for the RNA strand for RNA-DNA hybrids (Perbal, A Practical Guide to Molecular Cloning, New York: Wiley & Sons (1984)). Errors in transcription cannot be corrected by reverse transcriptase because known viral reverse transcriptases lack the 3′-5′ exonuclease activity necessary for proofreading (Saunders and Saunders, Microbial Genetics Applied to Biotechnology, London: Croom Helm (1987)). A detailed study of the activity of AMV reverse transcriptase and its associated RNaseH activity has been presented by Berger et al., Biochemistry 22:2365-2372 (1983). Another reverse transcriptase that is used extensively in molecular biology is reverse transcriptase originating from Moloney murine leukemia virus (M-MLV or “MMLV”). See, e.g., Gerard, G. R., DNA 5:271-279 (1986) and Kotewicz, M. L., et al., Gene 35:249-258 (1985). M-MLV reverse transcriptase substantially lacking in RNase H activity has also been described. See, e.g., U.S. Pat. No. 5,244,797. The invention contemplates the use of any such reverse transcriptases, or variants or mutants thereof. In some embodiments, the prime editors used in the systems and methods provided herein comprise MMLV RT, or a variant or fragment of MMLV RT.

### Reverse Transcription

As used herein, the term “reverse transcription” indicates the capability of an enzyme to synthesize a DNA strand (that is, complementary DNA or cDNA) using RNA as a template. In some embodiments, the reverse transcription can be “error-prone reverse transcription,” which refers to the properties of certain reverse transcriptase enzymes that are error-prone in their DNA polymerization activity.

### Second-Strand Nicking

In some embodiments, prime editing involves the resolution of heteroduplex DNA (i.e., containing one edited and one non-edited strand) formed as a result of installation of one or more desired nucleotide changes in the edit strand but not (yet) in the non-edit strand of the target DNA sequence. Resolution of the heteroduplex DNA (the edited strand paired with the endogenous non-edited strand) and installation of nucleotide changes corresponding to the desired nucleotide edits in the non-edit strand permanently integrates the desired edits in the target DNA sequence. The approach of “second-strand nicking” can be used herein to help drive the resolution of heteroduplex DNA in favor of permanent integration of the edited strand into the DNA molecule. As used herein, the concept of “second-strand nicking” refers to the introduction of a second nick on the unedited strand. In some embodiments, a second nick is introduced at a location on the non-edit strand corresponding to a position downstream of the first nick (i.e., the initial nick site that provides the free 3′ end for use in priming of the reverse transcriptase on the extended portion of the guide RNA) on the edit strand. Thus, the first nick (introduced by the prime editor in combination with the PEgRNA) and the second nick (introduced by the prime editor and a second-strand nicking guide RNA) are on opposite strands. Said another way, the first nick is on the non-target strand (i.e., the strand that forms the single strand portion of the R-loop), and the second nick is on the target strand. Said still another way, the first nick (introduced by the prime editor in combination with the PEgRNA) is on the edit strand, and the second nick (introduced by the prime editor and second strand nicking guide RNA) is on the non-edit strand. The second nick can be introduced in the non-edit strand at a position that is opposite at least 1, 2, 3, 4, or 5 nucleotides downstream or upstream of the first nick of the edit strand, or that is opposite at least 6, 7, 8, 9, 10, 11, 12, 13, 14, 15, 16, 17, 18, 19, 20, 21, 22, 23, 24, 25, 26, 27, 28, 29, 30, 40, 50, 60, 70, 80, 90, 100, 110, 120, 130, 140, or 150 or more nucleotides downstream or upstream of the first nick of the edit strand. The second nick can also be introduced in the non-edit strand at a position that is opposite at least 1, 2, 3, 4, or 5 nucleotides downstream or upstream of the edit site of the edit strand, or that is opposite at least 6, 7, 8, 9, 10, 11, 12, 13, 14, 15, 16, 17, 18, 19, 20, 21, 22, 23, 24, 25, 26, 27, 28, 29, 30, 40, 50, 60, 70, 80, 90, 100, 110, 120, 130, 140, or 150 or more nucleotides downstream or upstream of the edit site of the edit strand. The second nick, in certain embodiments, can be introduced in the non-edit strand at a position that is opposite about 1-150 nucleotides downstream or upstream of the first nick of the edit strand, or that is opposite about 1-140, or about 1-130, or about 1-120, or about 1-110, or about 1-100, or about 1-90, or about 1-80, or about 1-70, or about 1-60, or about 1-50, or about 1-40, or about 1-30, or about 1-20, or about 1-10 nucleotides downstream or upstream of the first nick of the edit strand. Without being bound by theory, the second nick induces the cell's endogenous DNA repair and replication processes towards replacement of the non-edit strand, thereby permanently installing the edited sequence on both strands of the target DNA and resolving the heteroduplex that is formed as a result of PE.

In certain embodiments, the second strand nicking guide RNA (also referred to herein as the nicking guide RNA, ngRNA, secondary nicking RNA, or second strand nicking sgRNA) may include a spacer sequence that preferentially and/or selectively only anneals to the edit strand after the desired nucleotide edit(s) are installed but not to the original strand of DNA the becomes replaced by the edited strand (i.e., the 5′ single-strand DNA flap that is displaced and ultimately removed during heteroduplex resolution). This can operate by designing the second strand nicking guide RNA to comprise a spacer sequence that anneals only to the edited region of the edited strand (and thus, wherein the spacer of the second strand nicking guide RNA comprises a nucleotide sequence that is the complement of the edited sequence or region thereof and includes the complement of the edit) and thus, can discriminate between the edited strand and the original strand of the displaced 5′ single-strand DNA flap that is immediately downstream of the cut site of the edited strand. This can be referred to as “temporal second-strand nicking” because the second strand nicking occurs only after prime editing has generated the new 3′ DNA flap containing the desired edit. This avoids the introduction of a double strand cut during prime editing that would otherwise result from the simultaneous or approximately simultaneous cutting of opposite strands by the PE complex comprising the PEgRNA and the PE complex comprising the second-strand cutting guide RNA.

### Sickle Cell Disease (SCD)

Sickle cell disease (SCD) is an autosomal recessive disorder caused by an A·T-to-T·A mutation in the hemoglobin subunit beta (HBB) gene, resulting in the pathogenic HBB^{S} allele encoding a Glu→Val substitution. This mutation changes normal adult β-globin (β^{A}) to sickle β-globin (β^{S}) and results in replacement of normal adult hemoglobin (HbA, α₂β₂) with sickle hemoglobin (HbS, α₂β^{S}₂). At low oxygen tension, HbS forms rigid polymers that cause characteristic red blood cell shape changes and initiate a complex pathophysiology that includes hemolysis, microvascular occlusions, and inflammation. The result is sickled red blood cells that are poor oxygen transporters and prone to aggregation. Clinical manifestations include anemia, immunodeficiency, multi-organ damage, severe acute and chronic pain, and premature death. Symptoms can be life-threatening.

Sickle cell disease occurs when a person inherits two abnormal copies of the β-globin gene (HBB) that makes hemoglobin, one from each parent. This gene occurs in chromosome 11. Several subtypes exist, depending on the exact mutation in each hemoglobin gene. An attack can be set off by temperature changes, stress, dehydration, and high altitude. A person with a single abnormal copy of HBB does not usually have symptoms and is said to have sickle cell trait. Such people are also referred to as carriers. Diagnosis is by a blood test, and some countries test all babies at birth for the disease. Diagnosis is also possible during pregnancy.

The amino acid sequence of the wild type HBB protein is provided below, with the position at which the pathogenic Glu (E)→Val (V) substitution occurs in those with sickle cell disease shown in bold:

(SEQ ID NO: 1)
MVHLTPEEKSAVTALWGKVNVDEVGGEALGRLLVVYPWTQRFFESFGDLS
TPDAVMGNPKVKAHGKKVLGAFSDGLAHLDNLKGTFATLSELHCDKLHVD
PENFRLLGNVLVCVLAHHFGKEFTPPVQAAYQKVVAGVANALAHKYH.

The nucleotide sequence of the HBB gene cDNA is provided below (NCBI Gene ID #3043), with the position at which the pathogenic A·T-to-T·A substitution occurs shown in bold:

(SEQ ID NO: 2)
ATGGTGCACCTGACTCCTGAGGAGAAGTCTGCCGTTACTGCCCTGTGGGG
CAAGGTGAACGTGGATGAAGTTGGTGGTGAGGCCCTGGGCAGGCTGCTGG
TGGTCTACCCTTGGACCCAGAGGTTCTTTGAGTCCTTTGGGGATCTGTCC
ACTCCTGATGCTGTTATGGGCAACCCTAAGGTGAAGGCTCATGGCAAGAA
AGTGCTCGGTGCCTTTAGTGATGGCCTGGCTCACCTGGACAACCTCAAGG
GCACCTTTGCCACACTGAGTGAGCTGCACTGTGACAAGCTGCACGTGGAT
CCTGAGAACTTCAGGCTCCTGGGCAACGTGCTGGTCTGTGTGCTGGCCCA
TCACTTTGGCAAAGAATTCACCCCACCAGTGCAGGCTGCCTATCAGAAAG
TGGTGGCTGGTGTGGCTAATGCCCTGGCCCACAAGTATCACTAA

The nucleotide sequence of the HBB gene in the genome (including introns and other non-coding sequences) is provided below (NCBI Gene ID #3043), with the position at which the pathogenic A·T-to-T·A substitution occurs shown in bold:

(SEQ ID NO: 133)
ACATTTGCTTCTGACACAACTGTGTTCACTAGCAACCTCAAACAGACACC
ATGGTGCACCTGACTCCTGAGGAGAAGTCTGCCGTTACTGCCCTGTGGGG
CAAGGTGAACGTGGATGAAGTTGGTGGTGAGGCCCTGGGCAGGTTGGTAT
CAAGGTTACAAGACAGGTTTAAGGAGACCAATAGAAACTGGGCATGTGGA
GACAGAGAAGACTCTTGGGTTTCTGATAGGCACTGACTCTCTCTGCCTAT
TGGTCTATTTTCCCACCCTTAGGCTGCTGGTGGTCTACCCTTGGACCCAG
AGGTTCTTTGAGTCCTTTGGGGATCTGTCCACTCCTGATGCTGTTATGGG
CAACCCTAAGGTGAAGGCTCATGGCAAGAAAGTGCTCGGTGCCTTTAGTG
ATGGCCTGGCTCACCTGGACAACCTCAAGGGCACCTTTGCCACACTGAGT
GAGCTGCACTGTGACAAGCTGCACGTGGATCCTGAGAACTTCAGGGTGAG
TCTATGGGACGCTTGATGTTTTCTTTCCCCTTCTTTTCTATGGTTAAGTT
CATGTCATAGGAAGGGGATAAGTAACAGGGTACAGTTTAGAATGGGAAAC
AGACGAATGATTGCATCAGTGTGGAAGTCTCAGGATCGTTTTAGTTTCTT
TTATTTGCTGTTCATAACAATTGTTTTCTTTTGTTTAATTCTTGCTTTCT
TTTTTTTTCTTCTCCGCAATTTTTACTATTATACTTAATGCCTTAACATT
GTGTATAACAAAAGGAAATATCTCTGAGATACATTAAGTAACTTAAAAAA
AAACTTTACACAGTCTGCCTAGTACATTACTATTTGGAATATATGTGTGC
TTATTTGCATATTCATAATCTCCCTACTTTATTTTCTTTTATTTTTAATT
GATACATAATCATTATACATATTTATGGGTTAAAGTGTAATGTTTTAATA
TGTGTACACATATTGACCAAATCAGGGTAATTTTGCATTTGTAATTTTAA
AAAATGCTTTCTTCTTTTAATATACTTTTTTGTTTATCTTATTTCTAATA
CTTTCCCTAATCTCTTTCTTTCAGGGCAATAATGATACAATGTATCATGC
CTCTTTGCACCATTCTAAAGAATAACAGTGATAATTTCTGGGTTAAGGCA
ATAGCAATATCTCTGCATATAAATATTTCTGCATATAAATTGTAACTGAT
GTAAGAGGTTTCATATTGCTAATAGCAGCTACAATCCAGCTACCATTCTG
CTTTTATTTTATGGTTGGGATAAGGCTGGATTATTCTGAGTCCAAGCTAG
GCCCTTTTGCTAATCATGTTCATACCTCTTATCTTCCTCCCACAGCTCCT
GGGCAACGTGCTGGTCTGTGTGCTGGCCCATCACTTTGGCAAAGAATTCA
CCCCACCAGTGCAGGCTGCCTATCAGAAAGTGGTGGCTGGTGTGGCTAAT
GCCCTGGCCCACAAGTATCACTAAGCTCGCTTTCTTGCTGTCCAATTTCT
ATTAAAGGTTCCTTTGTTCCCTAAGTCCAACTACTAAACTGGGGGATATT
ATGAAGGGCCTTGAGCATCTGGATTCTGCCTAATAAAAAACATTTATTTT
CATTGC

### Spacer Sequence

As used herein, the term “spacer sequence” in connection with a guide RNA or a PEgRNA refers to the portion of the guide RNA or PEgRNA of about 20 nucleotides that contains a nucleotide sequence that shares the same sequence as the protospacer sequence in the target DNA sequence. The spacer sequence anneals to the complement of the protospacer sequence to form a ssRNA/ssDNA hybrid structure at the target site and a corresponding R loop ssDNA structure of the endogenous DNA strand.

### Subject

The term “subject,” as used herein, refers to an individual organism, for example, an individual mammal. In some embodiments, the subject is a human. In some embodiments, the subject is a non-human mammal. In some embodiments, the subject is a non-human primate. In some embodiments, the subject is a rodent. In some embodiments, the subject is a sheep, a goat, a cow, a cat, or a dog. In some embodiments, the subject is a vertebrate, an amphibian, a reptile, a fish, an insect, a fly, or a nematode. In some embodiments, the subject is a research animal. In some embodiments, the subject is genetically engineered, e.g., a genetically engineered non-human subject. The subject may be of either sex and may be at any stage of development. In some embodiments, the subject has sickle cell disease, or is suspected of having sickle cell disease. In some embodiments, the genome of the subject encodes the pathogenic Glu (E)→Val (V) substitution in the HBB protein.

### Target Site

The term “target site” refers to a sequence within a nucleic acid molecule (i.e., within the HBB gene sequence) that is edited by a prime editor (PE) disclosed herein. The target site further refers to the sequence within a nucleic acid molecule to which a complex of the prime editor (PE) and gRNA binds.

### Treatment

The terms “treatment,” “treat,” and “treating” refer to a clinical intervention aimed to reverse, alleviate, delay the onset of, or inhibit the progress of a disease or disorder (e.g., sickle cell disease), or one or more symptoms thereof, as described herein. As used herein, the terms “treatment,” “treat,” and “treating” refer to a clinical intervention aimed to reverse, alleviate, delay the onset of, or inhibit the progress of sickle cell disease, or one or more symptoms thereof, as described herein. In some embodiments, treatment may be administered after one or more symptoms have developed and/or after sickle cell disease has been diagnosed. In other embodiments, treatment may be administered in the absence of symptoms, e.g., to prevent or delay onset of a symptom or inhibit onset or progression of sickle cell disease. For example, treatment may be administered to a susceptible individual prior to the onset of symptoms (e.g., in light of a history of symptoms and/or in light of genetic or other susceptibility factors). Treatment may also be continued after symptoms have resolved, for example, to prevent or delay their recurrence.

### Variant

As used herein, the term “variant” should be taken to mean the exhibition of qualities that have a pattern that deviates from what occurs in nature. The term “variant” encompasses homologous proteins having at least 70%, at least 75%, at least 80%, at least 85%, at least 90%, at least 95%, at least 96%, at least 975, at least 98%, or at least 99% identity with a reference sequence and having the same or substantially the same functional activity or activities as the reference sequence. The term also encompasses mutants, truncations, or domains of a reference sequence that display the same or substantially the same functional activity or activities as the reference sequence.

### Vector

The term “vector,” as used herein, refers to a nucleic acid that can be modified to encode a gene of interest and that is able to enter a host cell, mutate, and replicate within the host cell, and then transfer a replicated form of the vector into another host cell. Exemplary suitable vectors include viral vectors, such as retroviral vectors or bacteriophages and filamentous phage, and conjugative plasmids. Additional suitable vectors will be apparent to those of skill in the art based on the instant disclosure.

### Wild Type

As used herein the term “wild type” is a term of the art understood by skilled persons and means the typical form of an organism, strain, gene, or characteristic as it occurs in nature as distinguished from mutant or variant forms.

### DETAILED DESCRIPTION OF CERTAIN EMBODIMENTS

Sickle cell disease (SCD) is a monogenic disorder that affects millions of individuals and is caused by an A·T-to-T·A transversion mutation at nucleotide position 20 in the β-globin gene (HBB). The present disclosure provides prime editing systems developed and optimized to directly revert the SCD allele to the wild-type allele with high ratios of desired edit to indel by-products. Sickle-cell patient hematopoietic stem and progenitor cells (HSPCs) electroporated with PEmax mRNA, an engineered pegRNA (epegRNA), and a nicking single-guide RNA (sgRNA) yielded up to 41% conversion of the disease allele to the wild type allele. Extensive analysis of candidate off-target sites detected minimal off-target editing from all prime editing components used for the corrective edit. These results represent one of the first prime editing-based therapeutic strategies for HSPCs, demonstrating the feasibility of a one-time treatment for sickle cell disease that directly corrects the pathogenic allele to wild type, does not require delivery of any viral or non-viral DNA template, and minimizes undesired consequences associated with DNA double-strand breaks. HSCs were also editing in vivo, and were able to retain the edits introduced into its sub-lineages (e.g., CD34⁺ cells, CD235a⁺ cells, CD33⁺ cells, CD19⁺ cells, etc.).

Thus, the present disclosure provides methods, compositions, and systems for treating sickle cell disease using prime editing. The present disclosure also provides epegRNAs targeting the HBB gene, which may be useful for treating sickle cell disease. Also provided herein are prime editor complexes, polynucleotides, vectors, pharmaceutical compositions, kits, and cells useful for performing the methods described herein.

Methods and epegRNAs for Treating Sickle Cell Disease

In some aspects, the present disclosure provides methods for editing the β-globin (HBB) gene. In some aspects, the present disclosure provides methods for treating sickle cell disease. In some embodiments, the methods provided herein comprise contacting a target nucleotide sequence with a prime editor and an engineered prime editing guide RNA (epegRNA), wherein the epegRNA comprises the structure 5′-[spacer sequence]-[single guide RNA (sgRNA) scaffold sequence]-[extension arm sequence]-[optional engineered linker sequence]-[3′ structured motif]-3′. Each instance of]-[may independently comprise an optional linker sequence (e.g., an optional nucleic acid linker sequence).

In some embodiments, the epegRNAs used in the methods provided herein comprise a spacer sequence that targets a prime editor to a portion of the HBB gene. For example, the epegRNAs used in the methods provided herein may comprise the spacer sequence 5′-CATGGTGCACCTGACTCCTG-3′ (SEQ ID NO: 127). In some embodiments, the epegRNAs used in the methods provided herein comprise a sequence at least 90%, at least 95%, at least 96%, at least 97%, at least 98%, or at least 99% identical to the sequence 5′-CATGGTGCACCTGACTCCTG-3′ (SEQ ID NO: 127), or to a fragment thereof (e.g., at least 5, 6, 7, 8, 9, 10, 11, 12, 13, 14, 15, or more nucleotides of the sequence). The spacer sequence may also be shifted in the 5′ or 3′ direction on the HBB gene sequence. For example, the spacer sequence may be shifted 1, 2, 3, 4, 5, or more nucleotides in the 5′ or 3′ direction on the HBB gene sequence.

In some embodiments, the sgRNA scaffold sequence is a sequence that can be recognized and bound by a Cas9. In certain embodiments, the sgRNA scaffold sequence is a sequence that can be recognized and bound by SpCas9. In some embodiments, the sgRNA scaffold sequence of the epegRNAs used in the methods described herein comprises the sequence 5′-GTTTTAGAGCTAGAAATAGCAAGTTAAAATAAGGCTAGTCCGTTATCAACTTGAA AAAGTGGCACCGAGTCGGTGC-3′ (SEQ ID NO: 126), or a sequence at least 90%, at least 95%, at least 96%, at least 97%, at least 98%, or at least 99% identical to the sequence 5′-GTTTTAGAGCTAGAAATAGCAAGTTAAAATAAGGCTAGTCCGTTATCAACTTGAA AAAGTGGCACCGAGTCGGTGC-3′ (SEQ ID NO: 126), or a fragment thereof (e.g., at least 5, 6, 7, 8, 9, 10, 11, 12, 13, 14, 15, or more nucleotides of the sequence). The use of any sgRNA scaffold sequence known in the art in the epegRNAs provided herein is contemplated by the present disclosure. In particular, the use of any sgRNA scaffold sequences capable of being recognized and bound by the Cas9 protein of a prime editor is contemplated by the present disclosure.

In some embodiments, the epegRNAs used in the methods described herein comprise an extension arm sequence encoding the correction of a mutation in the HBB gene, e.g., a mutation known to cause, thought to cause, or otherwise associated with sickle cell disease. For example, the epegRNAs used in the methods provided herein may comprise the extension arm sequence 5′-AGACTTCTCTTCAGGAGTCAGGTGCAC-3′ (SEQ ID NO: 111). In some embodiments, the epegRNAs used in the methods provided herein comprise a sequence at least 90%, at least 95%, at least 96%, at least 97%, at least 98%, or at least 99% identical to the sequence 5′-AGACTTCTCTTCAGGAGTCAGGTGCAC-3′ (SEQ ID NO: 111), or a fragment thereof (e.g., at least 5, 6, 7, 8, 9, 10, 11, 12, 13, 14, 15, or more nucleotides of the sequence). In some embodiments, an epegRNA comprises the extension arm sequence 5′-AGACTCCTCTTCAGGAGTCAGGTGCAC-3′ (SEQ ID NO: 112), or a sequence at least 90%, at least 95%, at least 96%, at least 97%, at least 98%, or at least 99% identical to the sequence 5′-AGACTCCTCTTCAGGAGTCAGGTGCAC-3′ (SEQ ID NO: 112), or a fragment thereof (e.g., at least 5, 6, 7, 8, 9, 10, 11, 12, 13, 14, 15, or more nucleotides of the sequence)

In some embodiments, the epegRNAs used in the methods described herein comprise an additional structured motif that may, for example, be appended to the 3′ end of a pegRNA sequence, optionally via a linker. The inclusion of such a 3′ structured motif may facilitate the structural stabilization of the pegRNA, prevent or reduce its degradation, or otherwise increase the efficiency of prime editing by increasing the stability or lifespan of the pegRNA. Examples of such 3′ structured motifs that may be used in the epegRNAs provided herein include, but are not limited to, toe-loops, hairpins, stem-loops, pseudoknots, aptamers, G-quadruplexes, tRNAs, riboswitches, and ribozymes. In certain embodiments, the structured motif in the epegRNAs described herein is a pseudoknot. In certain embodiments, the structured motif in the epegRNAs described herein comprise evopreq1. Exemplary sequences of 3′ structured motifs that may be used in the epegRNAs provided herein include, but are not limited to, those of SEQ ID NOs: 48-77, or those of any sequence at least 90%, at least 95%, at least 96%, at least 97%, at least 98%, or at least 99% identical to the sequence of any one of SEQ ID NOs: 48-77 or a fragment thereof:

(SEQ ID NO: 48)
GGGTCAGGAGCCCCCCCCCTGAACCCAGGATAACCCTCAAAGTCGGGGG
GCAACCC;
(SEQ ID NO: 49)
GGGTCAGGAGCCCCCCCCCTGAACCCAGGATAACCCTCAAAGTCGGGGG
GC;
(SEQ ID NO: 50)
GTCAGGGTCAGGAGCCCCCCCCCTGAACCCAGGATAACCCTCAAAGTCG
GGGGGCAACCC;
(SEQ ID NO: 51)
GGGTCAGGAGCCCCCCCCCTGAACCCAGGAAAACCCTCAAAGTCGGGGG
GCAACCC;
(SEQ ID NO: 52)
GGGTCAGGAGCCCCCCCCCTGCACCCAGGAAAACCCTCAAAGTCGGGGG
GCAACCC;
(SEQ ID NO: 53)
GGGTCAGGAGCCCCCCCCCTGCACCCAGGATAACCCTCAAAGTCGGGGG
GCAACCC;
(SEQ ID NO: 54)
GTCAGGGTCAGGAGCCCCCCCCCTGAACCCAGGAAAACCCTCAAAGTCG
GGGGGCAACCC;
(SEQ ID NO: 55)
GTCAGGGTCAGGAGCCCCCCCCCTGCACCCAGGAAAACCCTCAAAGTCG
GGGGGCAACCC;
(SEQ ID NO: 56)
GTCAGGGTCAGGAGCCCCCCCCCTGCACCCAGGATAACCCTCAAAGTCG
GGGGGCAACCC;
(SEQ ID NO: 57)
GGGCTGGGATGGGAAAGGG;
(SEQ ID NO: 58)
GGGACAGGGCAGGGACAGGG;
(SEQ ID NO: 59)
GGGTCCGGGTCTGGGTCTGGG;
(SEQ ID NO: 60)
GGGCTCTGGGTGGGCCGGG;
(SEQ ID NO: 61)
GGGTGGGCTGGGAAGGG;
(SEQ ID NO: 62)
GGGAGGGAGGGCTAGGG;
(SEQ ID NO: 63)
GGGCTGGGCTGGGCAGGG;
(SEQ ID NO: 64)
GGGCAGGGCTGGGAGGG;
(SEQ ID NO: 65)
GGGTGGGAGGGCTGGG;
(SEQ ID NO: 66)
GGGCAGGGTCTGGGCTGGG;
(SEQ ID NO: 67)
TGGTGGTGGTGG;
(SEQ ID NO: 68)
TTGACGCGGTTCTATCTAGTTACGCGTTAAACCAACTAGAAA;
(SEQ ID NO: 69)
CGCGAGTCTAGGGGATAACGCGTTAAACTTCCTAGAAGGCGGTT;
(SEQ ID NO: 70)
CGCGGATCTAGATTGTAACGCGTTAAACCATCTAGAAGGCGGTT;
(SEQ ID NO: 71)
CGCGTCGCTACCGCCCGGCGCGTTAAACACACTAGAAGGCGGTT;
(SEQ ID NO: 72)
CGCGGTTCTATCTAGTTACGCGTTAAACCAACTAGAA;
(SEQ ID NO: 73)
TTGACGCGCTTCTATCTAGTTACGCGTTAAACCAACTAGAAA;
(SEQ ID NO: 74)
TTGACGCGGTTCTATCTACTTACGCGTTAAACCAACTAGAAA;
(SEQ ID NO: 75)
GGCGGGGCTCGTTGGTCTAGGGGTATGATTCTCGCTTCGGGTGCGAGAG
GTCCCGGGTTCAAATCCCGGACGAGCCCCGCC;
(SEQ ID NO: 76)
GCGTAACCTCCATCCGAGTTGCAAGAGAGGGAAACGCAGTCTC;
and
(SEQ ID NO: 77)
GGAATTGCGGGAAAGGGGTCAACAGCCGTTCAGTACCAAGTCTCAGGGG
AAACTTTGAGATGGCCTTGCAAAGGGTATGGTAATAAGCTGACGGACAT
GGTCCTAACCACGCAGCCAAGTCCTAAGTCAACAGATCTTCTGTTGATA
TGGATGCAGTTCA.

In certain embodiments, the epegRNAs provided herein comprise a 3′ structured motif comprising the sequence 5′-CGCGGTTCTATCTAGTTACGCGTTAAACCAACTAGAA-3′ (SEQ ID NO: 72), or a sequence at least 90%, at least 95%, at least 96%, at least 97%, at least 98%, or at least 99% identical to the sequence 5′-CGCGGTTCTATCTAGTTACGCGTTAAACCAACTAGAA-3′ (SEQ ID NO: 72), or a fragment thereof.

In some embodiments, a structured motif is appended directly to the 3′ end of an epegRNA. In some embodiments, a structured motif is joined to the 3′ end of an epegRNA via a linker. Any of the linkers described herein may be used to connect a structured motif to an epegRNA. In some embodiments, the epegRNAs provided herein comprise a linker that has been engineered to optimize the efficiency of prime editing at a particular target site in the HBB gene. For example, the epegRNAs provided herein may comprise the engineered linker sequence 5′-AGAATAAA-3′ between the extension arm sequence and the 3′ structured motif. In some embodiments, the epegRNAs provided herein comprise an engineered linker sequence at least 90%, at least 95%, at least 96%, at least 97%, at least 98%, or at least 99% identical to the sequence 5′-AGAATAAA-3′, or a fragment thereof (e.g., at least 4, 5, 6, or more nucleotides of the sequence), between the extension arm sequence and the 3′ structured motif.

In certain embodiments, an epegRNA for treating sickle cell disease using the methods described herein comprises the sequence 5′-CATGGTGCACCTGACTCCTGGTTTTAGAGCTAGAAATAGCAAGTTAAAATAAGG CTAGTCCGTTATCAACTTGAAAAAGTGGCACCGAGTCGGTGCAGACTTCTCTTCA GGAGTCAGGTGCACAGAATAAACGCGGTTCTATCTAGTTACGCGTTAAACCAAC TAGAA-3′ (SEQ ID NO: 115), or a sequence at least 90%, at least 95%, at least 96%, at least 97%, at least 98%, or at least 99% identical to the sequence of SEQ ID NO: 115. In certain embodiments, an epegRNA for treating sickle cell disease using the methods described herein comprises the sequence 5′-CATGGTGCACCTGACTCCTGGTTTTAGAGCTAGAAATAGCAAGTTAAAATAAGG CTAGTCCGTTATCAACTTGAAAAAGTGGCACCGAGTCGGTGCAGACTCCTCTTCA GGAGTCAGGTGCACAGAATAAACGCGGTTCTATCTAGTTACGCGTTAAACCAAC TAGAA-3′ (SEQ ID NO: 113), or a sequence at least 90%, at least 95%, at least 96%, at least 97%, at least 98%, or at least 99% identical to the sequence of SEQ ID NO: 113.

In some embodiments, the epegRNAs used in the methods provided herein further comprise additional nucleotides on their 3′ ends. In some embodiments, the epegRNAs further comprise one or more thymine nucleotides on their 3′ ends (e.g., 1, 2, 3, 4, 5, 6, 7, 8, 9, 10, or more than 10 thymine nucleotides). In certain embodiments, an epegRNA further comprises the sequence 5′-TTT-3′ on its 3′ end.

In certain embodiments, an epegRNA for treating sickle cell disease using the methods described herein comprises the sequence 5′-CATGGTGCACCTGACTCCTGGTTTTAGAGCTAGAAATAGCAAGTTAAAATAAGG CTAGTCCGTTATCAACTTGAAAAAGTGGCACCGAGTCGGTGCAGACTTCTCTTCA GGAGTCAGGTGCACAGAATAAACGCGGTTCTATCTAGTTACGCGTTAAACCAAC TAGAATTT-3′ (SEQ ID NO: 116), or a sequence at least 90%, at least 95%, at least 96%, at least 97%, at least 98%, or at least 99% identical to the sequence of SEQ ID NO: 116. In certain embodiments, an epegRNA for treating sickle cell disease using the methods described herein comprises the sequence 5′-CATGGTGCACCTGACTCCTGGTTTTAGAGCTAGAAATAGCAAGTTAAAATAAGG CTAGTCCGTTATCAACTTGAAAAAGTGGCACCGAGTCGGTGCAGACTCCTCTTCA GGAGTCAGGTGCACAGAATAAACGCGGTTCTATCTAGTTACGCGTTAAACCAAC TAGAATTT-3′ (SEQ ID NO: 114), or a sequence at least 90%, at least 95%, at least 96%, at least 97%, at least 98%, or at least 99% identical to the sequence of SEQ ID NO: 114.

In some embodiments, at least one nucleotide of the 5′-TTT-3′ sequence comprises a chemical modification. In certain embodiments, the chemical modification is a 2′-O-methyl modification. In some embodiments, all three nucleotides of the 5′-TTT-3′ sequence comprise a chemical modification. In certain embodiments, all three nucleotides of the 5′-TTT-3′ sequence comprise a 2′-O-methyl modification. In some embodiments, at least one nucleotide of the 5′-TTT-3′ sequence is connected by a phosphorothioate linkage. In certain embodiments, all three nucleotides of the 5′-TTT-3′ sequence are connected by phosphorothioate linkages.

In various embodiments, the methods provided herein may result in the correction of a mutation in the β-globin gene (HBB). In some embodiments, the methods result in the correction of an A·T:T·A transversion mutation in HBB. In certain embodiments, the A·T:T·A transversion mutation that is corrected is at nucleotide position 20 in HBB. Correcting the A·T:T·A transversion mutation at nucleotide position 20 in HBB using the methods provided herein results in the reversion the sickle cell disease allele to the wild type allele, thus treating sickle cell disease. In some embodiments, correction of the A·T:T·A transversion mutation in HBB results in the correction of a valine mutation in the β-globin protein to a glutamic acid residue, as in wild type HBB.

For example, in some embodiments, the methods provided herein result in the conversion of the bolded and underlined amino acid in the mutant β-globin protein sequence:

(SEQ ID NO: 132)
MVHLTPVEKSAVTALWGKVNVDEVGGEALGRLLVVYPWTQRFFESFGDLS
TPDAVMGNPKVKAHGKKVLGAFSDGLAHLDNLKGTFATLSELHCDKLHVD
PENFRLLGNVLVCVLAHHFGKEFTPPVQAAYQKVVAGVANALAHKYH

to the bolded and underlined amino acid in the wild type β-globin protein sequence:

(SEQ ID NO: 1)
MVHLTPEEKSAVTALWGKVNVDEVGGEALGRLLVVYPWTQRFFESFGDLS
TPDAVMGNPKVKAHGKKVLGAFSDGLAHLDNLKGTFATLSELHCDKLHVD
PENFRLLGNVLVCVLAHHFGKEFTPPVQAAYQKVVAGVANALAHKYH

In some embodiments, the methods provided herein result in the conversion of the bolded and underlined nucleotide in the mutant β-globin gene sequence:

(SEQ ID NO: 135)
ATGGTGCACCTGACTCCTGTGGAGAAGTCTGCCGTTACTGCCCTGTGGGG
CAAGGTGAACGTGGATGAAGTTGGTGGTGAGGCCCTGGGCAGGCTGCTGG
TGGTCTACCCTTGGACCCAGAGGTTCTTTGAGTCCTTTGGGGATCTGTCC
ACTCCTGATGCTGTTATGGGCAACCCTAAGGTGAAGGCTCATGGCAAGAA
AGTGCTCGGTGCCTTTAGTGATGGCCTGGCTCACCTGGACAACCTCAAGG
GCACCTTTGCCACACTGAGTGAGCTGCACTGTGACAAGCTGCACGTGGAT
CCTGAGAACTTCAGGCTCCTGGGCAACGTGCTGGTCTGTGTGCTGGCCCA
TCACTTTGGCAAAGAATTCACCCCACCAGTGCAGGCTGCCTATCAGAAAG
TGGTGGCTGGTGTGGCTAATGCCCTGGCCCACAAGTATCACTAA

to the bolded and underlined nucleotide in the wild-type β-globin gene sequence:

(SEQ ID NO: 2)
ATGGTGCACCTGACTCCTGAGGAGAAGTCTGCCGTTACTGCCCTGTGGGG
CAAGGTGAACGTGGATGAAGTTGGTGGTGAGGCCCTGGGCAGGCTGCTGG
TGGTCTACCCTTGGACCCAGAGGTTCTTTGAGTCCTTTGGGGATCTGTCC
ACTCCTGATGCTGTTATGGGCAACCCTAAGGTGAAGGCTCATGGCAAGAA
AGTGCTCGGTGCCTTTAGTGATGGCCTGGCTCACCTGGACAACCTCAAGG
GCACCTTTGCCACACTGAGTGAGCTGCACTGTGACAAGCTGCACGTGGAT
CCTGAGAACTTCAGGCTCCTGGGCAACGTGCTGGTCTGTGTGCTGGCCCA
TCACTTTGGCAAAGAATTCACCCCACCAGTGCAGGCTGCCTATCAGAAAG
TGGTGGCTGGTGTGGCTAATGCCCTGGCCCACAAGTATCACTAA

In some embodiments, the methods provided herein result in the conversion of the bolded and underlined nucleotide in the mutant β-globin gene sequence:

(SEQ ID NO: 134)
ACATTTGCTTCTGACACAACTGTGTTCACTAGCAACCTCAAACAGACACC
ATGGTGCACCTGACTCCTGTGGAGAAGTCTGCCGTTACTGCCCTGTGGGG
CAAGGTGAACGTGGATGAAGTTGGTGGTGAGGCCCTGGGCAGGTTGGTAT
CAAGGTTACAAGACAGGTTTAAGGAGACCAATAGAAACTGGGCATGTGGA
GACAGAGAAGACTCTTGGGTTTCTGATAGGCACTGACTCTCTCTGCCTAT
TGGTCTATTTTCCCACCCTTAGGCTGCTGGTGGTCTACCCTTGGACCCAG
AGGTTCTTTGAGTCCTTTGGGGATCTGTCCACTCCTGATGCTGTTATGGG
CAACCCTAAGGTGAAGGCTCATGGCAAGAAAGTGCTCGGTGCCTTTAGTG
ATGGCCTGGCTCACCTGGACAACCTCAAGGGCACCTTTGCCACACTGAGT
GAGCTGCACTGTGACAAGCTGCACGTGGATCCTGAGAACTTCAGGGTGAG
TCTATGGGACGCTTGATGTTTTCTTTCCCCTTCTTTTCTATGGTTAAGTT
CATGTCATAGGAAGGGGATAAGTAACAGGGTACAGTTTAGAATGGGAAAC
AGACGAATGATTGCATCAGTGTGGAAGTCTCAGGATCGTTTTAGTTTCTT
TTATTTGCTGTTCATAACAATTGTTTTCTTTTGTTTAATTCTTGCTTTCT
TTTTTTTTCTTCTCCGCAATTTTTACTATTATACTTAATGCCTTAACATT
GTGTATAACAAAAGGAAATATCTCTGAGATACATTAAGTAACTTAAAAAA
AAACTTTACACAGTCTGCCTAGTACATTACTATTTGGAATATATGTGTGC
TTATTTGCATATTCATAATCTCCCTACTTTATTTTCTTTTATTTTTAATT
GATACATAATCATTATACATATTTATGGGTTAAAGTGTAATGTTTTAATA
TGTGTACACATATTGACCAAATCAGGGTAATTTTGCATTTGTAATTTTAA
AAAATGCTTTCTTCTTTTAATATACTTTTTTGTTTATCTTATTTCTAATA
CTTTCCCTAATCTCTTTCTTTCAGGGCAATAATGATACAATGTATCATGC
CTCTTTGCACCATTCTAAAGAATAACAGTGATAATTTCTGGGTTAAGGCA
ATAGCAATATCTCTGCATATAAATATTTCTGCATATAAATTGTAACTGAT
GTAAGAGGTTTCATATTGCTAATAGCAGCTACAATCCAGCTACCATTCTG
CTTTTATTTTATGGTTGGGATAAGGCTGGATTATTCTGAGTCCAAGCTAG
GCCCTTTTGCTAATCATGTTCATACCTCTTATCTTCCTCCCACAGCTCCT
GGGCAACGTGCTGGTCTGTGTGCTGGCCCATCACTTTGGCAAAGAATTCA
CCCCACCAGTGCAGGCTGCCTATCAGAAAGTGGTGGCTGGTGTGGCTAAT
GCCCTGGCCCACAAGTATCACTAAGCTCGCTTTCTTGCTGTCCAATTTCT
ATTAAAGGTTCCTTTGTTCCCTAAGTCCAACTACTAAACTGGGGGATATT
ATGAAGGGCCTTGAGCATCTGGATTCTGCCTAATAAAAAACATTTATTTT
CATTGC

to the bolded and underlined nucleotide in the wild-type β-globin gene sequence:

(SEQ ID NO: 133)
ACATTTGCTTCTGACACAACTGTGTTCACTAGCAACCTCAAACAGACACC
ATGGTGCACCTGACTCCTGAGGAGAAGTCTGCCGTTACTGCCCTGTGGGG
CAAGGTGAACGTGGATGAAGTTGGTGGTGAGGCCCTGGGCAGGTTGGTAT
CAAGGTTACAAGACAGGTTTAAGGAGACCAATAGAAACTGGGCATGTGGA
GACAGAGAAGACTCTTGGGTTTCTGATAGGCACTGACTCTCTCTGCCTAT
TGGTCTATTTTCCCACCCTTAGGCTGCTGGTGGTCTACCCTTGGACCCAG
AGGTTCTTTGAGTCCTTTGGGGATCTGTCCACTCCTGATGCTGTTATGGG
CAACCCTAAGGTGAAGGCTCATGGCAAGAAAGTGCTCGGTGCCTTTAGTG
ATGGCCTGGCTCACCTGGACAACCTCAAGGGCACCTTTGCCACACTGAGT
GAGCTGCACTGTGACAAGCTGCACGTGGATCCTGAGAACTTCAGGGTGAG
TCTATGGGACGCTTGATGTTTTCTTTCCCCTTCTTTTCTATGGTTAAGTT
CATGTCATAGGAAGGGGATAAGTAACAGGGTACAGTTTAGAATGGGAAAC
AGACGAATGATTGCATCAGTGTGGAAGTCTCAGGATCGTTTTAGTTTCTT
TTATTTGCTGTTCATAACAATTGTTTTCTTTTGTTTAATTCTTGCTTTCT
TTTTTTTTCTTCTCCGCAATTTTTACTATTATACTTAATGCCTTAACATT
GTGTATAACAAAAGGAAATATCTCTGAGATACATTAAGTAACTTAAAAAA
AAACTTTACACAGTCTGCCTAGTACATTACTATTTGGAATATATGTGTGC
TTATTTGCATATTCATAATCTCCCTACTTTATTTTCTTTTATTTTTAATT
GATACATAATCATTATACATATTTATGGGTTAAAGTGTAATGTTTTAATA
TGTGTACACATATTGACCAAATCAGGGTAATTTTGCATTTGTAATTTTAA
AAAATGCTTTCTTCTTTTAATATACTTTTTTGTTTATCTTATTTCTAATA
CTTTCCCTAATCTCTTTCTTTCAGGGCAATAATGATACAATGTATCATGC
CTCTTTGCACCATTCTAAAGAATAACAGTGATAATTTCTGGGTTAAGGCA
ATAGCAATATCTCTGCATATAAATATTTCTGCATATAAATTGTAACTGAT
GTAAGAGGTTTCATATTGCTAATAGCAGCTACAATCCAGCTACCATTCTG
CTTTTATTTTATGGTTGGGATAAGGCTGGATTATTCTGAGTCCAAGCTAG
GCCCTTTTGCTAATCATGTTCATACCTCTTATCTTCCTCCCACAGCTCCT
GGGCAACGTGCTGGTCTGTGTGCTGGCCCATCACTTTGGCAAAGAATTCA
CCCCACCAGTGCAGGCTGCCTATCAGAAAGTGGTGGCTGGTGTGGCTAAT
GCCCTGGCCCACAAGTATCACTAAGCTCGCTTTCTTGCTGTCCAATTTCT
ATTAAAGGTTCCTTTGTTCCCTAAGTCCAACTACTAAACTGGGGGATATT
ATGAAGGGCCTTGAGCATCTGGATTCTGCCTAATAAAAAACATTTATTTT
CATTGC

In some embodiments, any of the methods provided herein may further result in the introduction of a silent PAM-disrupting edit in the HBB gene. Introduction of such a PAM-disrupting edit in the HBB gene may help the prime editing intermediate that is produced prior to incorporation of the edit to avoid reversion to the unedited sequence by the cellular DNA mismatch repair pathway. In certain embodiments, the methods provided herein further result in the introduction of a G→A silent PAM-disrupting edit at the nucleotide position following the A·T:T·A transversion mutation in HBB that is corrected using the methods described herein.

In some embodiments, any of the methods provided herein may further comprise nicking the non-PAM-containing strand of the target nucleotide sequence using a nicking sgRNA. Use of a nicking sgRNA in the methods described herein allows the Cas9 protein of the prime editor to nick the unedited strand at a position determined by the nicking guide. Nicking the unedited strand may bias the cellular DNA repair process to incorporate the edit introduced by the prime editor into both DNA strands rather than reverting the edit to the unedited sequence. In some embodiments, a nicking sgRNA used in the methods provided herein comprises the nucleotide sequence 5′-CCTTGATACCAACCTGCCCA-3′ (SEQ ID NO: 128), 5′-GTAACGGCAGACTTCTCTTC-3′ (SEQ ID NO: 129), 5′-CACGTTCACCTTGCCCCACA-3′ (SEQ ID NO: 130), or 5′-TCCACATGCCCAGTTTCTAT-3′ (SEQ ID NO: 131), or a sequence at least 90%, at least 95%, at least 96%, at least 97%, at least 98%, or at least 99% identical to the sequence 5′-CCTTGATACCAACCTGCCCA-3′ (SEQ ID NO: 128), 5′-GTAACGGCAGACTTCTCTTC-3′ (SEQ ID NO: 129), 5′-CACGTTCACCTTGCCCCACA-3′ (SEQ ID NO: 130), or 5′-TCCACATGCCCAGTTTCTAT-3′ (SEQ ID NO: 131) or a fragment thereof. In certain embodiments, the spacer sequence of the nicking sgRNA comprises the nucleotide sequence 5′-CCTTGATACCAACCTGCCCA-3′ (SEQ ID NO: 128), or a sequence at least 90%, at least 95%, at least 96%, at least 97%, at least 98%, or at least 99% identical to the sequence 5′-CCTTGATACCAACCTGCCCA-3′ (SEQ ID NO: 128) or a fragment thereof. In certain embodiments, the spacer sequence of the nicking sgRNA comprises the nucleotide sequence 5′-GTAACGGCAGACTTCTCTTC-3′ (SEQ ID NO: 129), 5′-CACGTTCACCTTGCCCCACA-3′ (SEQ ID NO: 130), or 5′-TCCACATGCCCAGTTTCTAT-3′ (SEQ ID NO: 131), or a sequence at least 90%, at least 95%, at least 96%, at least 97%, at least 98%, or at least 99% identical to the sequence 5′-GTAACGGCAGACTTCTCTTC-3′ (SEQ ID NO: 129), 5′-CACGTTCACCTTGCCCCACA-3′ (SEQ ID NO: 130), or 5′-TCCACATGCCCAGTTTCTAT-3′ (SEQ ID NO: 131) or a fragment thereof.

Use of any prime editor described herein or known in the art in the methods provided herein is contemplated by the present disclosure. The prime editors used in the methods described herein may comprise any nucleic acid-programmable DNA-binding protein (napDNAbp) provided herein, and any reverse transcriptase provided herein. In some embodiments, the napDNAbp is a Cas9 protein. In certain embodiments, the napDNAbp is a Cas9 nickase (e.g., an SpCas9 nickase). In some embodiments, the prime editors used in the methods provided herein comprise PEmax architecture, i.e., as described elsewhere herein, they comprise the Cas9 protein variant Cas9(R221K N39K H840A), the MMLV RT variant MMLV_RT (D200N)(T330P)(L603W), and the following structure: [bipartite NLS]-[Cas9(R221K)(N394K)(H840A)]-[linker]-[MMLV_RT (D200N)(T330P)(L603W)]-[bipartite NLS]-[NLS]. In some embodiments, the prime editor is PE3max, PE3bmax, PE4max, PE5max, or PE5bmax. In certain embodiments, the prime editor is PE3max. In certain embodiments, the prime editor is PE3bmax.

In some embodiments, the methods described herein are performed in a cell. In some embodiments, the methods described herein are performed in a eukaryotic cell. In some embodiments, the methods described herein are performed in a human cell. In certain embodiments, the cell is a hematopoietic stem or progenitor cell (HSPC).

In various embodiments, the prime editor may be delivered to the cell as a protein, and the epegRNA may be delivered to the cell as RNA. In various embodiments, the prime editor and epegRNA may be delivered to the cell as a ribonucleoprotein (RNP). In various embodiments, the prime editor may be delivered to the cell as mRNA, and the epegRNA may be delivered to the cell as RNA. In some embodiments, a nicking sgRNA is also delivered to the cell as RNA. As described herein, HSPCs are particularly amenable to electroporation of RNA, while electroporation of DNA and other means of transfection or transformation are significantly less efficient. Thus, in some embodiments of the methods provided herein, a prime editor is delivered to a cell (e.g., an HSPC, an HSC, or any other human cell type) as mRNA by electroporation, and an epegRNA is delivered to the cell as RNA by electroporation. In certain embodiments, a nicking sgRNA is also delivered to the cell as RNA by electroporation. In some embodiments, the prime editor mRNA, epegRNA, and nicking sgRNA comprise approximately 15%, approximately 16%, approximately 17%, approximately 18%, approximately 19%, approximately 20%, approximately 21%, approximately 22%, approximately 23%, approximately 24%, or approximately 25% of the total electroporation volume. In certain embodiments, the prime editor mRNA, epegRNA, and nicking sgRNA comprise approximately 20% of the total electroporation volume. In some embodiments, the molar ratio of the amount of epegRNA to the amount of nicking sgRNA delivered to the cell is approximately 2:1, approximately 1.9:1, approximately 1.8:1, approximately 1.7:1, approximately 1.6:1, approximately 1.5:1, approximately 1.4:1, approximately 1.3:1, approximately 1.2:1, approximately 1.1:1, or approximately 1:1. In certain embodiments, the molar ratio of the amount of epegRNA to the amount of nicking sgRNA delivered to the cell is approximately 1.5:1.

In some embodiments, the prime editors, epegRNAs, and optional nicking gRNAs are delivered to the cell using lipid nanoparticles (LNPs). In some embodiments, the prime editors, epegRNAs, and optional nicking gRNAs are delivered to the cell using AAVs. In some embodiments, the prime editors, epegRNAs, and optional nicking gRNAs are delivered to the cell using non-viral vectors. In some embodiments, the prime editors, epegRNAs, and optional nicking gRNAs are delivered to the cell using virus-like particles (VLPs).

Any of the methods described herein may be performed in vitro. Any of the methods described herein may also be performed ex vivo. In some embodiments, the method is performed in a cell ex vivo, and then the edited cell is subsequently transplanted into a subject to be treated for sickle cell disease. Any of the methods described herein may also be performed in vivo. In some embodiments, the method is performed in a subject. In certain embodiments, the subject is a human. In some embodiments, the subject has or is suspected of having sickle cell disease. In some embodiments, the genome of the subject has an A·T:T·A transversion mutation at nucleotide position 20 in HBB.

In some embodiments, the methods provided herein result in a greater than 20%, greater than 25%, greater than 30%, greater than 35%, or greater than 40% efficiency of conversion of an A·T:T·A transversion mutation at nucleotide position 20 in HBB to the wild type sequence. In certain embodiments, the methods provided herein result in a greater than 40% efficiency of conversion of an A·T:T·A transversion mutation at nucleotide position 20 in HBB to the wild type sequence. In some embodiments, the methods provided herein result in an edit-to-indel ratio of greater than 5, greater than 5.5, greater than 6, greater than 6.5, greater than 7, or greater than 7.5. In certain embodiments, the methods provided herein result in an edit-to-indel ratio of greater than 7.5. In some embodiments, the methods provided herein result in at least 30%, at least 35%, or at least 40% of cells edited using the method retaining the edit following transplantation into a subject. In certain embodiments, the methods provided herein result in at least 40% of cells edited using the method retaining the edit following transplantation into a subject. In some embodiments, the methods provided herein are performed with a prime editing efficiency of at least 60%, at least 65%, at least 70%, at least 75%, or at least 80%. In certain embodiments, the methods provided herein are performed with a prime editing efficiency of at least 80%. In some embodiments, the methods provided herein result in an indel frequency of less than 10%, less than 9%, less than 8%, less than 7%, less than 6%, less than 5%, or less than 4%. In certain embodiments, the methods provided herein result in an indel frequency of less than 4%.

In some embodiments, the present disclosure provides methods of treating sickle cell disease comprising contacting a target nucleotide sequence with a prime editor and a prime editing guide RNA (pegRNA), wherein the prime editor is PE3max or PE3bmax, and wherein the pegRNA comprises the structure 5′-[spacer sequence]-[single guide RNA (sgRNA) scaffold sequence]-[extension arm sequence]-3′, wherein each instance of]-[comprises an optional linker sequence.

In some embodiments, the present disclosure provides methods of treating sickle cell disease comprising contacting a target nucleotide sequence with a prime editor, a prime editing guide RNA (pegRNA), and a nicking single guide RNA (sgRNA), wherein the pegRNA comprises the structure 5′-[spacer sequence]-[sgRNA scaffold sequence]-[extension arm sequence]-3′, wherein each instance of]-[comprises an optional linker sequence, and wherein the spacer sequence of the nicking sgRNA comprises the nucleotide sequence 5′-GTAACGGCAGACTTCTCTTC-3′ (SEQ ID NO: 129), 5′-CACGTTCACCTTGCCCCACA-3′ (SEQ ID NO: 130), or 5′-TCCACATGCCCAGTTTCTAT-3′ (SEQ ID NO: 131), or a sequence at least 90%, at least 95%, at least 96%, at least 97%, at least 98%, or at least 99% identical to the sequence 5′-GTAACGGCAGACTTCTCTTC-3′ (SEQ ID NO: 129), 5′-CACGTTCACCTTGCCCCACA-3′ (SEQ ID NO: 130), or 5′-TCCACATGCCCAGTTTCTAT-3′ (SEQ ID NO: 131) or a fragment thereof.

In some embodiments, the present disclosure provides for the use of any of the epegRNAs, complexes, polynucleotides, vectors, pharmaceutical compositions, or combinations thereof provided herein for the treatment of sickle cell disease.

In some embodiments, the present disclosure provides for the use of any of the epegRNAs, complexes, polynucleotides, vectors, pharmaceutical compositions, or combinations thereof provided herein for the manufacture of a medicament for the treatment of sickle cell disease.

napDNAbp

In various embodiments, the prime editors utilized in the methods and complexes described herein comprise a nucleic acid programmable DNA binding protein (napDNAbp).

In various embodiments, prime editors may include a napDNAbp domain having a wild type Cas9 sequence, including, for example, the canonical Streptococcus pyogenes Cas9 sequence of SEQ ID NO: 6, shown as follows.

| Description | Sequence | SEQ ID NO: |
|---|---|---|
| SpCas9 | MDKKYSIGLDIGTNSVGWAVITDEYKVPSKKFKVLGNTDRHSIKK | 6 |
| Streptococcus | NLIGALLFDSGETAEATRLKRTARRRYTRRKNRICYLQEIFSNEMA | |
| pyogenes | KVDDSFFHRLEESFLVEEDKKHERHPIFGNIVDEVAYHEKYPTIYH | |
| M1 | LRKKLVDSTDKADLRLIYLALAHMIKFRGHFLIEGDLNPDNSDVD | |
| SwissProt | KLFIQLVQTYNQLFEENPINASGVDAKAILSARLSKSRRLENLIAQL | |
| Accession | PGEKKNGLFGNLIALSLGLTPNFKSNFDLAEDAKLQLSKDTYDDD | |
| No. | LDNLLAQIGDQYADLFLAAKNLSDAILLSDILRVNTEITKAPLSAS | |
| Q99ZW2 | MIKRYDEHHQDLTLLKALVRQQLPEKYKEIFFDQSKNGYAGYIDG | |
| Wild type | GASQEEFYKFIKPILEKMDGTEELLVKLNREDLLRKQRTFDNGSIP | |
| | HQIHLGELHAILRRQEDFYPFLKDNREKIEKILTFRIPYYVGPLARG | |
| | NSRFAWMTRKSEETITPWNFEEVVDKGASAQSFIERMTNFDKNLP | |
| | NEKVLPKHSLLYEYFTVYNELTKVKYVTEGMRKPAFLSGEQKKAI | |
| | VDLLFKTNRKVTVKQLKEDYFKKIECFDSVEISGVEDRFNASLGTY | |
| | HDLLKIIKDKDFLDNEENEDILEDIVLTLTLFEDREMIEERLKTYAH | |
| | LFDDKVMKQLKRRRYTGWGRLSRKLINGIRDKQSGKTILDFLKSD | |
| | GFANRNFMQLIHDDSLTFKEDIQKAQVSGQGDSLHEHIANLAGSP | |
| | AIKKGILQTVKVVDELVKVMGRHKPENIVIEMARENQTTQKGQK | |
| | NSRERMKRIEEGIKELGSQILKEHPVENTQLQNEKLYLYYLQNGRD | |
| | MYVDQELDINRLSDYDVDHIVPQSFLKDDSIDNKVLTRSDKNRGK | |
| | SDNVPSEEVVKKMKNYWRQLLNAKLITQRKFDNLTKAERGGLSE | |
| | LDKAGFIKRQLVETRQITKHVAQILDSRMNTKYDENDKLIREVKVI | |
| | TLKSKLVSDFRKDFQFYKVREINNYHHAHDAYLNAVVGTALIKK | |
| | YPKLESEFVYGDYKVYDVRKMIAKSEQEIGKATAKYFFYSNIMNF | |
| | FKTEITLANGEIRKRPLIETNGETGEIVWDKGRDFATVRKVLSMPQ | |
| | VNIVKKTEVQTGGFSKESILPKRNSDKLIARKKDWDPKKYGGFDS | |
| | PTVAYSVLVVAKVEKGKSKKLKSVKELLGITIMERSSFEKNPIDFL | |
| | EAKGYKEVKKDLIIKLPKYSLFELENGRKRMLASAGELQKGNELA | |
| | LPSKYVNFLYLASHYEKLKGSPEDNEQKQLFVEQHKHYLDEIIEQI | |
| | SEFSKRVILADANLDKVLSAYNKHRDKPIREQAENIIHLFTLTNLG | |
| | APAAFKYFDTTIDRKRYTSTKEVLDATLIHQSITGLYETRIDLSQLG | |
| | GD | |

In other embodiments, the prime editors may include a napDNAbp domain having a modified Cas9 sequence, including, for example the nickase variant of Streptococcus pyogenes Cas9 of SEQ ID NO: 7 having an H840A substitution relative to the wild type SpCas9 (of SEQ ID NO: 6), shown as follows:

Cas9 nickase Streptococcus pyogenes
Q99ZW2 Cas9 with H840A
SEQ ID NO: 7
MDKKYSIGLDIGTNSVGWAVITDEYKVPSKKFKVLGNTDRHSIKKNLIGA
LLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKAD
LRLIYLALAHMIKFRGHFLIEGDLNPDNSDVDKLFIQLVQTYNQLFEENP
INASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEI
FFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPY
YVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKI
IKDKDFLDNEENEDILEDIVLTLTLFEDREMIEERLKTYAHLFDDKVMKQ
LKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHP
VENTQLQNEKLYLYYLQNGRDMYVDQELDINRLSDYDVDAIVPQSFLKDD
SIDNKVLTRSDKNRGKSDNVPSEEVVKKMKNYWRQLLNAKLITQRKFDNL
TKAERGGLSELDKAGFIKRQLVETRQITKHVAQILDSRMNTKYDENDKLI
REVKVITLKSKLVSDFRKDFQFYKVREINNYHHAHDAYLNAVVGTALIKK
YPKLESEFVYGDYKVYDVRKMIAKSEQEIGKATAKYFFYSNIMNFFKTEI
TLANGEIRKRPLIETNGETGEIVWDKGRDFATVRKVLSMPQVNIVKKTEV
QTGGFSKESILPKRNSDKLIARKKDWDPKKYGGFDSPTVAYSVLVVAKVE
KGKSKKLKSVKELLGITIMERSSFEKNPIDFLEAKGYKEVKKDLIIKLPK
YSLFELENGRKRMLASAGELQKGNELALPSKYVNFLYLASHYEKLKGSPE
DNEQKQLFVEQHKHYLDEIIEQISEFSKRVILADANLDKVLSAYNKHRDK
PIREQAENIIHLFTLTNLGAPAAFKYFDTTIDRKRYTSTKEVLDATLIHQ
SITGLYETRIDLSQLGGD

The prime editors used in the methods and complexes described herein may include any of the modified Cas9 sequences described above, or any variant thereof having at least 80%, at least 85%, at least 90%, at least 95%, or at least 99% sequence identity thereto. In some embodiments, the prime editors used in the methods and complexes described herein include any of the following other wild type SpCas9 sequences, which may be modified with one or more of the mutations described herein at corresponding amino acid positions:

| Description | Sequence |
|---|---|
| SpCas9 | ATGGATAAGAAATACTCAATAGGCTTAGATATCGGCACAAATAGCGTCGGATG |
| Streptococcus | GGCGGTGATCACTGATGATTATAAGGTTCCGTCTAAAAAGTTCAAGGTTCTGGG |
| pyogenes | AAATACAGACCGCCACAGTATCAAAAAAAATCTTATAGGGGCTCTTTTATTTGG |
| MGAS1882 | CAGTGGAGAGACAGCGGAAGCGACTCGTCTCAAACGGACAGCTCGTAGAAGGT |
| wild type | ATACACGTCGGAAGAATCGTATTTGTTATCTACAGGAGATTTTTTCAAATGAGA |
| NC_017053.1 | TGGCGAAAGTAGATGATAGTTTCTTTCATCGACTTGAAGAGTCTTTTTTGGTGGA |
| | AGAAGACAAGAAGCATGAACGTCATCCTATTTTTGGAAATATAGTAGATGAAGT |
| | TGCTTATCATGAGAAATATCCAACTATCTATCATCTGCGAAAAAAATTGGCAGA |
| | TTCTACTGATAAAGCGGATTTGCGCTTAATCTATTTGGCCTTAGCGCATATGATT |
| | AAGTTTCGTGGTCATTTTTTGATTGAGGGAGATTTAAATCCTGATAATAGTGATG |
| | TGGACAAACTATTTATCCAGTTGGTACAAATCTACAATCAATTATTTGAAGAAA |
| | ACCCTATTAACGCAAGTAGAGTAGATGCTAAAGCGATTCTTTCTGCACGATTGA |
| | GTAAATCAAGACGATTAGAAAATCTCATTGCTCAGCTCCCCGGTGAGAAGAGAA |
| | ATGGCTTGTTTGGGAATCTCATTGCTTTGTCATTGGGATTGACCCCTAATTTTAA |
| | ATCAAATTTTGATTTGGCAGAAGATGCTAAATTACAGCTTTCAAAAGATACTTA |
| | CGATGATGATTTAGATAATTTATTGGCGCAAATTGGAGATCAATATGCTGATTT |
| | GTTTTTGGCAGCTAAGAATTTATCAGATGCTATTTTACTTTCAGATATCCTAAGA |
| | GTAAATAGTGAAATAACTAAGGCTCCCCTATCAGCTTCAATGATTAAGCGCTAC |
| | GATGAACATCATCAAGACTTGACTCTTTTAAAAGCTTTAGTTCGACAACAACTTC |
| | CAGAAAAGTATAAAGAAATCTTTTTTGATCAATCAAAAAACGGATATGCAGGTT |
| | ATATTGATGGGGGAGCTAGCCAAGAAGAATTTTATAAATTTATCAAACCAATTT |
| | TAGAAAAAATGGATGGTACTGAGGAATTATTGGTGAAACTAAATCGTGAAGATT |
| | TGCTGCGCAAGCAACGGACCTTTGACAACGGCTCTATTCCCCATCAAATTCACTT |
| | GGGTGAGCTGCATGCTATTTTGAGAAGACAAGAAGACTTTTATCCATTTTTAAA |
| | AGACAATCGTGAGAAGATTGAAAAAATCTTGACTTTTCGAATTCCTTATTATGTT |
| | GGTCCATTGGCGCGTGGCAATAGTCGTTTTGCATGGATGACTCGGAAGTCTGAA |
| | GAAACAATTACCCCATGGAATTTTGAAGAAGTTGTCGATAAAGGTGCTTCAGCT |
| | CAATCATTTATTGAACGCATGACAAACTTTGATAAAAATCTTCCAAATGAAAAA |
| | GTACTACCAAAACATAGTTTGCTTTATGAGTATTTTACGGTTTATAACGAATTGA |
| | CAAAGGTCAAATATGTTACTGAGGGAATGCGAAAACCAGCATTTCTTTCAGGTG |
| | AACAGAAGAAAGCCATTGTTGATTTACTCTTCAAAACAAATCGAAAAGTAACCG |
| | TTAAGCAATTAAAAGAAGATTATTTCAAAAAAATAGAATGTTTTGATAGTGTTG |
| | AAATTTCAGGAGTTGAAGATAGATTTAATGCTTCATTAGGCGCCTACCATGATTT |
| | GCTAAAAATTATTAAAGATAAAGATTTTTTGGATAATGAAGAAAATGAAGATAT |
| | CTTAGAGGATATTGTTTTAACATTGACCTTATTTGAAGATAGGGGGATGATTGA |
| | GGAAAGACTTAAAACATATGCTCACCTCTTTGATGATAAGGTGATGAAACAGCT |
| | TAAACGTCGCCGTTATACTGGTTGGGGACGTTTGTCTCGAAAATTGATTAATGGT |
| | ATTAGGGATAAGCAATCTGGCAAAACAATATTAGATTTTTTGAAATCAGATGGT |
| | TTTGCCAATCGCAATTTTATGCAGCTGATCCATGATGATAGTTTGACATTTAAAG |
| | AAGATATTCAAAAAGCACAGGTGTCTGGACAAGGCCATAGTTTACATGAACAG |
| | ATTGCTAACTTAGCTGGCAGTCCTGCTATTAAAAAAGGTATTTTACAGACTGTA |
| | AAAATTGTTGATGAACTGGTCAAAGTAATGGGGCATAAGCCAGAAAATATCGTT |
| | ATTGAAATGGCACGTGAAAATCAGACAACTCAAAAGGGCCAGAAAAATTCGCG |
| | AGAGCGTATGAAACGAATCGAAGAAGGTATCAAAGAATTAGGAAGTCAGATTC |
| | TTAAAGAGCATCCTGTTGAAAATACTCAATTGCAAAATGAAAAGCTCTATCTCT |
| | ATTATCTACAAAATGGAAGAGACATGTATGTGGACCAAGAATTAGATATTAATC |
| | GTTTAAGTGATTATGATGTCGATCACATTGTTCCACAAAGTTTCATTAAAGACGA |
| | TTCAATAGACAATAAGGTACTAACGCGTTCTGATAAAAATCGTGGTAAATCGGA |
| | TAACGTTCCAAGTGAAGAAGTAGTCAAAAAGATGAAAAACTATTGGAGACAAC |
| | TTCTAAACGCCAAGTTAATCACTCAACGTAAGTTTGATAATTTAACGAAAGCTG |
| | AACGTGGAGGTTTGAGTGAACTTGATAAAGCTGGTTTTATCAAACGCCAATTGG |
| | TTGAAACTCGCCAAATCACTAAGCATGTGGCACAAATTTTGGATAGTCGCATGA |
| | ATACTAAATACGATGAAAATGATAAACTTATTCGAGAGGTTAAAGTGATTACCT |
| | TAAAATCTAAATTAGTTTCTGACTTCCGAAAAGATTTCCAATTCTATAAAGTACG |
| | TGAGATTAACAATTACCATCATGCCCATGATGCGTATCTAAATGCCGTCGTTGG |
| | AACTGCTTTGATTAAGAAATATCCAAAACTTGAATCGGAGTTTGTCTATGGTGA |
| | TTATAAAGTTTATGATGTTCGTAAAATGATTGCTAAGTCTGAGCAAGAAATAGG |
| | CAAAGCAACCGCAAAATATTTCTTTTACTCTAATATCATGAACTTCTTCAAAACA |
| | GAAATTACACTTGCAAATGGAGAGATTCGCAAACGCCCTCTAATCGAAACTAAT |
| | GGGGAAACTGGAGAAATTGTCTGGGATAAAGGGCGAGATTTTGCCACAGTGCG |
| | CAAAGTATTGTCCATGCCCCAAGTCAATATTGTCAAGAAAACAGAAGTACAGAC |
| | AGGCGGATTCTCCAAGGAGTCAATTTTACCAAAAAGAAATTCGGACAAGCTTAT |
| | TGCTCGTAAAAAAGACTGGGATCCAAAAAAATATGGTGGTTTTGATAGTCCAAC |
| | GGTAGCTTATTCAGTCCTAGTGGTTGCTAAGGTGGAAAAAGGGAAATCGAAGA |
| | AGTTAAAATCCGTTAAAGAGTTACTAGGGATCACAATTATGGAAAGAAGTTCCT |
| | TTGAAAAAAATCCGATTGACTTTTTAGAAGCTAAAGGATATAAGGAAGTTAAAA |
| | AAGACTTAATCATTAAACTACCTAAATATAGTCTTTTTGAGTTAGAAAACGGTC |
| | GTAAACGGATGCTGGCTAGTGCCGGAGAATTACAAAAAGGAAATGAGCTGGCT |
| | CTGCCAAGCAAATATGTGAATTTTTTATATTTAGCTAGTCATTATGAAAAGTTGA |
| | AGGGTAGTCCAGAAGATAACGAACAAAAACAATTGTTTGTGGAGCAGCATAAG |
| | CATTATTTAGATGAGATTATTGAGCAAATCAGTGAATTTTCTAAGCGTGTTATTT |
| | TAGCAGATGCCAATTTAGATAAAGTTCTTAGTGCATATAACAAACATAGAGACA |
| | AACCAATACGTGAACAAGCAGAAAATATTATTCATTTATTTACGTTGACGAATC |
| | TTGGAGCTCCCGCTGCTTTTAAATATTTTGATACAACAATTGATCGTAAACGATA |
| | TACGTCTACAAAAGAAGTTTTAGATGCCACTCTTATCCATCAATCCATCACTGGT |
| | CTTTATGAAACACGCATTGATTTGAGTCAGCTAGGAGGTGACTGA |
| | (SEQ ID NO: 25) |
| SpCas9 | MDKKYSIGLDIGTNSVGWAVITDDYKVPSKKFKVLGNTDRHSIKKNLIGALLFGSG |
| Streptococcus | ETAEATRLKRTARRRYTRRKNRICYLQEIFSNEMAKVDDSFFHRLEESFL VEEDKKH |
| pyogenes | ERHPIFGNIVDEVAYHEKYPTIYHLRKKLADSTDKADLRLIYLALAHMIKFRGHFLIE |
| MGAS1882 | GDLNPDNSDVDKLFIQLVQIYNQLFEENPINASRVDAKAILSARLSKSRRLENLIAQL |
| wild type | PGEKRNGLFGNLIALSLGLTPNFKSNFDLAEDAKLQLSKDTYDDDLDNLLAQIGDQ |
| NC_017053.1 | YADLFLAAKNLSDAILLSDILRVNSEITKAPLSASMIKRYDEHHQDLTLLKALVRQQ |
| | LPEKYKEIFFDQSKNGYAGYIDGGASQEEFYKFIKPILEKMDGTEELLVKLNREDLL |
| | RKQRTFDNGSIPHQIHLGELHAILRRQEDFYPFLKDNREKIEKILTFRIPYYVGPLARG |
| | NSRFAWMTRKSEETITPWNFEEVVDKGASAQSFIERMTNFDKNLPNEKVLPKHSLL |
| | YEYFTVYNELTKVKYVTEGMRKPAFLSGEQKKAIVDLLFKTNRKVTVKQLKEDYF |
| | KKIECFDSVEISGVEDRFNASLGAYHDLLKIIKDKDFLDNEENEDILEDIVLTLTLFED |
| | RGMIEERLKTYAHLFDDKVMKQLKRRRYTGWGRLSRKLINGIRDKQSGKTILDFLK |
| | SDGFANRNFMQLIHDDSLTFKEDIQKAQVSGQGHSLHEQIANLAGSPAIKKGILQTV |
| | KIVDELVKVMGHKPENIVIEMARENQTTQKGQKNSRERMKRIEEGIKELGSQILKEH |
| | PVENTQLQNEKLYLYYLQNGRDMYVDQELDINRLSDYDVDHIVPQSFIKDDSIDNK |
| | VLTRSDKNRGKSDNVPSEEVVKKMKNYWRQLLNAKLITQRKFDNLTKAERGGLSE |
| | LDKAGFIKRQLVETRQITKHVAQILDSRMNTKYDENDKLIREVKVITLKSKLVSDFR |
| | KDFQFYKVREINNYHHAHDAYLNAVVGTALIKKYPKLESEFVYGDYKVYDVRKMI |
| | AKSEQEIGKATAKYFFYSNIMNFFKTEITLANGEIRKRPLIETNGETGEIVWDKGRDF |
| | ATVRKVLSMPQVNIVKKTEVQTGGFSKESILPKRNSDKLIARKKDWDPKKYGGFDS |
| | PTVAYSVLVVAKVEKGKSKKLKSVKELLGITIMERSSFEKNPIDFLEAKGYKEVKKD |
| | LIIKLPKYSLFELENGRKRMLASAGELQKGNELALPSKYVNFLYLASHYEKLKGSPE |
| | DNEQKQLFVEQHKHYLDEIIEQISEFSKRVILADANLDKVLSAYNKHRDKPIREQAE |
| | NIIHLFTLTNLGAPAAFKYFDTTIDRKRYTSTKEVLDATLIHQSITGLYETRIDLSQLG |
| | GD (SEQ ID NO: 8) |
| SpCas9 | ATGGATAAAAAGTATTCTATTGGTTTAGACATCGGCACTAATTCCGTTGGATGG |
| Streptococcus | GCTGTCATAACCGATGAATACAAAGTACCTTCAAAGAAATTTAAGGTGTTGGGG |
| pyogenes wild | AACACAGACCGTCATTCGATTAAAAAGAATCTTATCGGTGCCCTCCTATTCGAT |
| type | AGTGGCGAAACGGCAGAGGCGACTCGCCTGAAACGAACCGCTCGGAGAAGGTA |
| SWBC2D7W014 | TACACGTCGCAAGAACCGAATATGTTACTTACAAGAAATTTTTAGCAATGAGAT |
| | GGCCAAAGTTGACGATTCTTTCTTTCACCGTTTGGAAGAGTCCTTCCTTGTCGAA |
| | GAGGACAAGAAACATGAACGGCACCCCATCTTTGGAAACATAGTAGATGAGGT |
| | GGCATATCATGAAAAGTACCCAACGATTTATCACCTCAGAAAAAAGCTAGTTGA |
| | CTCAACTGATAAAGCGGACCTGAGGTTAATCTACTTGGCTCTTGCCCATATGAT |
| | AAAGTTCCGTGGGCACTTTCTCATTGAGGGTGATCTAAATCCGGACAACTCGGA |
| | TGTCGACAAACTGTTCATCCAGTTAGTACAAACCTATAATCAGTTGTTTGAAGA |
| | GAACCCTATAAATGCAAGTGGCGTGGATGCGAAGGCTATTCTTAGCGCCCGCCT |
| | CTCTAAATCCCGACGGCTAGAAAACCTGATCGCACAATTACCCGGAGAGAAGA |
| | AAAATGGGTTGTTCGGTAACCTTATAGCGCTCTCACTAGGCCTGACACCAAATT |
| | TTAAGTCGAACTTCGACTTAGCTGAAGATGCCAAATTGCAGCTTAGTAAGGACA |
| | CGTACGATGACGATCTCGACAATCTACTGGCACAAATTGGAGATCAGTATGCGG |
| | ACTTATTTTTGGCTGCCAAAAACCTTAGCGATGCAATCCTCCTATCTGACATACT |
| | GAGAGTTAATACTGAGATTACCAAGGCGCCGTTATCCGCTTCAATGATCAAAAG |
| | GTACGATGAACATCACCAAGACTTGACACTTCTCAAGGCCCTAGTCCGTCAGCA |
| | ACTGCCTGAGAAATATAAGGAAATATTCTTTGATCAGTCGAAAAACGGGTACGC |
| | AGGTTATATTGACGGCGGAGCGAGTCAAGAGGAATTCTACAAGTTTATCAAACC |
| | CATATTAGAGAAGATGGATGGGACGGAAGAGTTGCTTGTAAAACTCAATCGCG |
| | AAGATCTACTGCGAAAGCAGCGGACTTTCGACAACGGTAGCATTCCACATCAAA |
| | TCCACTTAGGCGAATTGCATGCTATACTTAGAAGGCAGGAGGATTTTTATCCGTT |
| | CCTCAAAGACAATCGTGAAAAGATTGAGAAAATCCTAACCTTTCGCATACCTTA |
| | CTATGTGGGACCCCTGGCCCGAGGGAACTCTCGGTTCGCATGGATGACAAGAAA |
| | GTCCGAAGAAACGATTACTCCATGGAATTTTGAGGAAGTTGTCGATAAAGGTGC |
| | GTCAGCTCAATCGTTCATCGAGAGGATGACCAACTTTGACAAGAATTTACCGAA |
| | CGAAAAAGTATTGCCTAAGCACAGTTTACTTTACGAGTATTTCACAGTGTACAA |
| | TGAACTCACGAAAGTTAAGTATGTCACTGAGGGCATGCGTAAACCCGCCTTTCT |
| | AAGCGGAGAACAGAAGAAAGCAATAGTAGATCTGTTATTCAAGACCAACCGCA |
| | AAGTGACAGTTAAGCAATTGAAAGAGGACTACTTTAAGAAAATTGAATGCTTCG |
| | ATTCTGTCGAGATCTCCGGGGTAGAAGATCGATTTAATGCGTCACTTGGTACGT |
| | ATCATGACCTCCTAAAGATAATTAAAGATAAGGACTTCCTGGATAACGAAGAGA |
| | ATGAAGATATCTTAGAAGATATAGTGTTGACTCTTACCCTCTTTGAAGATCGGG |
| | AAATGATTGAGGAAAGACTAAAAACATACGCTCACCTGTTCGACGATAAGGTTA |
| | TGAAACAGTTAAAGAGGCGTCGCTATACGGGCTGGGGACGATTGTCGCGGAAA |
| | CTTATCAACGGGATAAGAGACAAGCAAAGTGGTAAAACTATTCTCGATTTTCTA |
| | AAGAGCGACGGCTTCGCCAATAGGAACTTTATGCAGCTGATCCATGATGACTCT |
| | TTAACCTTCAAAGAGGATATACAAAAGGCACAGGTTTCCGGACAAGGGGACTC |
| | ATTGCACGAACATATTGCGAATCTTGCTGGTTCGCCAGCCATCAAAAAGGGCAT |
| | ACTCCAGACAGTCAAAGTAGTGGATGAGCTAGTTAAGGTCATGGGACGTCACA |
| | AACCGGAAAACATTGTAATCGAGATGGCACGCGAAAATCAAACGACTCAGAAG |
| | GGGCAAAAAAACAGTCGAGAGCGGATGAAGAGAATAGAAGAGGGTATTAAAG |
| | AACTGGGCAGCCAGATCTTAAAGGAGCATCCTGTGGAAAATACCCAATTGCAG |
| | AACGAGAAACTTTACCTCTATTACCTACAAAATGGAAGGGACATGTATGTTGAT |
| | CAGGAACTGGACATAAACCGTTTATCTGATTACGACGTCGATCACATTGTACCC |
| | CAATCCTTTTTGAAGGACGATTCAATCGACAATAAAGTGCTTACACGCTCGGAT |
| | AAGAACCGAGGGAAAAGTGACAATGTTCCAAGCGAGGAAGTCGTAAAGAAAAT |
| | GAAGAACTATTGGCGGCAGCTCCTAAATGCGAAACTGATAACGCAAAGAAAGT |
| | TCGATAACTTAACTAAAGCTGAGAGGGGTGGCTTGTCTGAACTTGACAAGGCCG |
| | GATTTATTAAACGTCAGCTCGTGGAAACCCGCCAAATCACAAAGCATGTTGCAC |
| | AGATACTAGATTCCCGAATGAATACGAAATACGACGAGAACGATAAGCTGATT |
| | CGGGAAGTCAAAGTAATCACTTTAAAGTCAAAATTGGTGTCGGACTTCAGAAAG |
| | GATTTTCAATTCTATAAAGTTAGGGAGATAAATAACTACCACCATGCGCACGAC |
| | GCTTATCTTAATGCCGTCGTAGGGACCGCACTCATTAAGAAATACCCGAAGCTA |
| | GAAAGTGAGTTTGTGTATGGTGATTACAAAGTTTATGACGTCCGTAAGATGATC |
| | GCGAAAAGCGAACAGGAGATAGGCAAGGCTACAGCCAAATACTTCTTTTATTCT |
| | AACATTATGAATTTCTTTAAGACGGAAATCACTCTGGCAAACGGAGAGATACGC |
| | AAACGACCTTTAATTGAAACCAATGGGGAGACAGGTGAAATCGTATGGGATAA |
| | GGGCCGGGACTTCGCGACGGTGAGAAAAGTTTTGTCCATGCCCCAAGTCAACAT |
| | AGTAAAGAAAACTGAGGTGCAGACCGGAGGGTTTTCAAAGGAATCGATTCTTCC |
| | AAAAAGGAATAGTGATAAGCTCATCGCTCGTAAAAAGGACTGGGACCCGAAAA |
| | AGTACGGTGGCTTCGATAGCCCTACAGTTGCCTATTCTGTCCTAGTAGTGGCAA |
| | AAGTTGAGAAGGGAAAATCCAAGAAACTGAAGTCAGTCAAAGAATTATTGGGG |
| | ATAACGATTATGGAGCGCTCGTCTTTTGAAAAGAACCCCATCGACTTCCTTGAG |
| | GCGAAAGGTTACAAGGAAGTAAAAAAGGATCTCATAATTAAACTACCAAAGTA |
| | TAGTCTGTTTGAGTTAGAAAATGGCCGAAAACGGATGTTGGCTAGCGCCGGAGA |
| | GCTTCAAAAGGGGAACGAACTCGCACTACCGTCTAAATACGTGAATTTCCTGTA |
| | TTTAGCGTCCCATTACGAGAAGTTGAAAGGTTCACCTGAAGATAACGAACAGAA |
| | GCAACTTTTTGTTGAGCAGCACAAACATTATCTCGACGAAATCATAGAGCAAAT |
| | TTCGGAATTCAGTAAGAGAGTCATCCTAGCTGATGCCAATCTGGACAAAGTATT |
| | AAGCGCATACAACAAGCACAGGGATAAACCCATACGTGAGCAGGCGGAAAATA |
| | TTATCCATTTGTTTACTCTTACCAACCTCGGCGCTCCAGCCGCATTCAAGTATTTT |
| | GACACAACGATAGATCGCAAACGATACACTTCTACCAAGGAGGTGCTAGACGC |
| | GACACTGATTCACCAATCCATCACGGGATTATATGAAACTCGGATAGATTTGTC |
| | ACAGCTTGGGGGTGACGGATCCCCCAAGAAGAAGAGGAAAGTCTCGAGCGACT |
| | ACAAAGACCATGACGGTGATTATAAAGATCATGACATCGATTACAAGGATGAC |
| | GATGACAAGGCTGCAGGA (SEQ ID NO: 26) |
| SpCas9 | MDKKYSIGLDIGTNSVGWAVITDEYKVPSKKFKVLGNTDRHSIKKNLIGALLFDSGE |
| Streptococcus | TAEATRLKRTARRRYTRRKNRICYLQEIFSNEMAKVDDSFFHRLEESFLVEEDKKHE |
| pyogenes wild | RHPIFGNIVDEVAYHEKYPTIYHLRKKLVDSTDKADLRLIYLALAHMIKFRGHFLIEG |
| type | DLNPDNSDVDKLFIQLVQTYNQLFEENPINASGVDAKAILSARLSKSRRLENLIAQLP |
| Encoded | GEKKNGLFGNLIALSLGLTPNFKSNFDLAEDAKLQLSKDTYDDDLDNLLAQIGDQY |
| product of | ADLFLAAKNLSDAILLSDILRVNTEITKAPLSASMIKRYDEHHQDLTLLKALVRQQL |
| SWBC2D7W014 | PEKYKEIFFDQSKNGYAGYIDGGASQEEFYKFIKPILEKMDGTEELLVKLNREDLLR |
| | KQRTFDNGSIPHQIHLGELHAILRRQEDFYPFLKDNREKIEKILTFRIPYYVGPLARGN |
| | SRFAWMTRKSEETITPWNFEEVVDKGASAQSFIERMTNFDKNLPNEKVLPKHSLLY |
| | EYFTVYNELTKVKYVTEGMRKPAFLSGEQKKAIVDLLFKTNRKVTVKQLKEDYFK |
| | KIECFDSVEISGVEDRFNASLGTYHDLLKIIKDKDFLDNEENEDILEDIVLTLTLFEDR |
| | EMIEERLKTYAHLFDDKVMKQLKRRRYTGWGRLSRKLINGIRDKQSGKTILDFLKS |
| | DGFANRNFMQLIHDDSLTFKEDIQKAQVSGQGDSLHEHIANLAGSPAIKKGILQTVK |
| | VVDELVKVMGRHKPENIVIEMARENQTTQKGQKNSRERMKRIEEGIKELGSQILKE |
| | HPVENTQLQNEKLYLYYLQNGRDMYVDQELDINRLSDYDVDHIVPQSFLKDDSIDN |
| | KVLTRSDKNRGKSDNVPSEEVVKKMKNYWRQLLNAKLITQRKFDNLTKAERGGLS |
| | ELDKAGFIKRQLVETRQITKHVAQILDSRMNTKYDENDKLIREVKVITLKSKLVSDF |
| | RKDFQFYKVREINNYHHAHDAYLNAVVGTALIKKYPKLESEFVYGDYKVYDVRK |
| | MIAKSEQEIGKATAKYFFYSNIMNFFKTEITLANGEIRKRPLIETNGETGEIVWDKGR |
| | DFATVRKVLSMPQVNIVKKTEVQTGGFSKESILPKRNSDKLIARKKDWDPKKYGGF |
| | DSPTVAYSVLVVAKVEKGKSKKLKSVKELLGITIMERSSFEKNPIDFLEAKGYKEVK |
| | KDLIIKLPKYSLFELENGRKRMLASAGELQKGNELALPSKYVNFLYLASHYEKLKGS |
| | PEDNEQKQLFVEQHKHYLDEIIEQISEFSKRVILADANLDKVLSAYNKHRDKPIREQ |
| | AENIIHLFTLTNLGAPAAFKYFDTTIDRKRYTSTKEVLDATLIHQSITGLYETRIDLSQ |
| | LGGDGSPKKKRKVSSDYKDHDGDYKDHDIDYKDDDDKAAG (SEQ ID NO: 9) |
| SpCas9 | ATGGATAAGAAATACTCAATAGGCTTAGATATCGGCACAAATAGCGTCGGATG |
| Streptococcus | GGCGGTGATCACTGATGAATATAAGGTTCCGTCTAAAAAGTTCAAGGTTCTGGG |
| pyogenes | AAATACAGACCGCCACAGTATCAAAAAAAATCTTATAGGGGCTCTTTTATTTGA |
| MIGAS wild | CAGTGGAGAGACAGCGGAAGCGACTCGTCTCAAACGGACAGCTCGTAGAAGGT |
| type | ATACACGTCGGAAGAATCGTATTTGTTATCTACAGGAGATTTTTTCAAATGAGA |
| NC_002737.2 | TGGCGAAAGTAGATGATAGTTTCTTTCATCGACTTGAAGAGTCTTTTTTGGTGGA |
| | AGAAGACAAGAAGCATGAACGTCATCCTATTTTTGGAAATATAGTAGATGAAGT |
| | TGCTTATCATGAGAAATATCCAACTATCTATCATCTGCGAAAAAAATTGGTAGA |
| | TTCTACTGATAAAGCGGATTTGCGCTTAATCTATTTGGCCTTAGCGCATATGATT |
| | AAGTTTCGTGGTCATTTTTTGATTGAGGGAGATTTAAATCCTGATAATAGTGATG |
| | TGGACAAACTATTTATCCAGTTGGTACAAACCTACAATCAATTATTTGAAGAAA |
| | ACCCTATTAACGCAAGTGGAGTAGATGCTAAAGCGATTCTTTCTGCACGATTGA |
| | GTAAATCAAGACGATTAGAAAATCTCATTGCTCAGCTCCCCGGTGAGAAGAAAA |
| | ATGGCTTATTTGGGAATCTCATTGCTTTGTCATTGGGTTTGACCCCTAATTTTAA |
| | ATCAAATTTTGATTTGGCAGAAGATGCTAAATTACAGCTTTCAAAAGATACTTA |
| | CGATGATGATTTAGATAATTTATTGGCGCAAATTGGAGATCAATATGCTGATTT |
| | GTTTTTGGCAGCTAAGAATTTATCAGATGCTATTTTACTTTCAGATATCCTAAGA |
| | GTAAATACTGAAATAACTAAGGCTCCCCTATCAGCTTCAATGATTAAACGCTAC |
| | GATGAACATCATCAAGACTTGACTCTTTTAAAAGCTTTAGTTCGACAACAACTTC |
| | CAGAAAAGTATAAAGAAATCTTTTTTGATCAATCAAAAAACGGATATGCAGGTT |
| | ATATTGATGGGGGAGCTAGCCAAGAAGAATTTTATAAATTTATCAAACCAATTT |
| | TAGAAAAAATGGATGGTACTGAGGAATTATTGGTGAAACTAAATCGTGAAGATT |
| | TGCTGCGCAAGCAACGGACCTTTGACAACGGCTCTATTCCCCATCAAATTCACTT |
| | GGGTGAGCTGCATGCTATTTTGAGAAGACAAGAAGACTTTTATCCATTTTTAAA |
| | AGACAATCGTGAGAAGATTGAAAAAATCTTGACTTTTCGAATTCCTTATTATGTT |
| | GGTCCATTGGCGCGTGGCAATAGTCGTTTTGCATGGATGACTCGGAAGTCTGAA |
| | GAAACAATTACCCCATGGAATTTTGAAGAAGTTGTCGATAAAGGTGCTTCAGCT |
| | CAATCATTTATTGAACGCATGACAAACTTTGATAAAAATCTTCCAAATGAAAAA |
| | GTACTACCAAAACATAGTTTGCTTTATGAGTATTTTACGGTTTATAACGAATTGA |
| | CAAAGGTCAAATATGTTACTGAAGGAATGCGAAAACCAGCATTTCTTTCAGGTG |
| | AACAGAAGAAAGCCATTGTTGATTTACTCTTCAAAACAAATCGAAAAGTAACCG |
| | TTAAGCAATTAAAAGAAGATTATTTCAAAAAAATAGAATGTTTTGATAGTGTTG |
| | AAATTTCAGGAGTTGAAGATAGATTTAATGCTTCATTAGGTACCTACCATGATTT |
| | GCTAAAAATTATTAAAGATAAAGATTTTTTGGATAATGAAGAAAATGAAGATAT |
| | CTTAGAGGATATTGTTTTAACATTGACCTTATTTGAAGATAGGGAGATGATTGA |
| | GGAAAGACTTAAAACATATGCTCACCTCTTTGATGATAAGGTGATGAAACAGCT |
| | TAAACGTCGCCGTTATACTGGTTGGGGACGTTTGTCTCGAAAATTGATTAATGGT |
| | ATTAGGGATAAGCAATCTGGCAAAACAATATTAGATTTTTTGAAATCAGATGGT |
| | TTTGCCAATCGCAATTTTATGCAGCTGATCCATGATGATAGTTTGACATTTAAAG |
| | AAGACATTCAAAAAGCACAAGTGTCTGGACAAGGCGATAGTTTACATGAACAT |
| | ATTGCAAATTTAGCTGGTAGCCCTGCTATTAAAAAAGGTATTTTACAGACTGTA |
| | AAAGTTGTTGATGAATTGGTCAAAGTAATGGGGCGGCATAAGCCAGAAAATAT |
| | CGTTATTGAAATGGCACGTGAAAATCAGACAACTCAAAAGGGCCAGAAAAATT |
| | CGCGAGAGCGTATGAAACGAATCGAAGAAGGTATCAAAGAATTAGGAAGTCAG |
| | ATTCTTAAAGAGCATCCTGTTGAAAATACTCAATTGCAAAATGAAAAGCTCTAT |
| | CTCTATTATCTCCAAAATGGAAGAGACATGTATGTGGACCAAGAATTAGATATT |
| | AATCGTTTAAGTGATTATGATGTCGATCACATTGTTCCACAAAGTTTCCTTAAAG |
| | ACGATTCAATAGACAATAAGGTCTTAACGCGTTCTGATAAAAATCGTGGTAAAT |
| | CGGATAACGTTCCAAGTGAAGAAGTAGTCAAAAAGATGAAAAACTATTGGAGA |
| | CAACTTCTAAACGCCAAGTTAATCACTCAACGTAAGTTTGATAATTTAACGAAA |
| | GCTGAACGTGGAGGTTTGAGTGAACTTGATAAAGCTGGTTTTATCAAACGCCAA |
| | TTGGTTGAAACTCGCCAAATCACTAAGCATGTGGCACAAATTTTGGATAGTCGC |
| | ATGAATACTAAATACGATGAAAATGATAAACTTATTCGAGAGGTTAAAGTGATT |
| | ACCTTAAAATCTAAATTAGTTTCTGACTTCCGAAAAGATTTCCAATTCTATAAAG |
| | TACGTGAGATTAACAATTACCATCATGCCCATGATGCGTATCTAAATGCCGTCG |
| | TTGGAACTGCTTTGATTAAGAAATATCCAAAACTTGAATCGGAGTTTGTCTATG |
| | GTGATTATAAAGTTTATGATGTTCGTAAAATGATTGCTAAGTCTGAGCAAGAAA |
| | TAGGCAAAGCAACCGCAAAATATTTCTTTTACTCTAATATCATGAACTTCTTCAA |
| | AACAGAAATTACACTTGCAAATGGAGAGATTCGCAAACGCCCTCTAATCGAAAC |
| | TAATGGGGAAACTGGAGAAATTGTCTGGGATAAAGGGCGAGATTTTGCCACAG |
| | TGCGCAAAGTATTGTCCATGCCCCAAGTCAATATTGTCAAGAAAACAGAAGTAC |
| | AGACAGGCGGATTCTCCAAGGAGTCAATTTTACCAAAAAGAAATTCGGACAAG |
| | CTTATTGCTCGTAAAAAAGACTGGGATCCAAAAAAATATGGTGGTTTTGATAGT |
| | CCAACGGTAGCTTATTCAGTCCTAGTGGTTGCTAAGGTGGAAAAAGGGAAATCG |
| | AAGAAGTTAAAATCCGTTAAAGAGTTACTAGGGATCACAATTATGGAAAGAAG |
| | TTCCTTTGAAAAAAATCCGATTGACTTTTTAGAAGCTAAAGGATATAAGGAAGT |
| | TAAAAAAGACTTAATCATTAAACTACCTAAATATAGTCTTTTTGAGTTAGAAAA |
| | CGGTCGTAAACGGATGCTGGCTAGTGCCGGAGAATTACAAAAAGGAAATGAGC |
| | TGGCTCTGCCAAGCAAATATGTGAATTTTTTATATTTAGCTAGTCATTATGAAAA |
| | GTTGAAGGGTAGTCCAGAAGATAACGAACAAAAACAATTGTTTGTGGAGCAGC |
| | ATAAGCATTATTTAGATGAGATTATTGAGCAAATCAGTGAATTTTCTAAGCGTG |
| | TTATTTTAGCAGATGCCAATTTAGATAAAGTTCTTAGTGCATATAACAAACATA |
| | GAGACAAACCAATACGTGAACAAGCAGAAAATATTATTCATTTATTTACGTTGA |
| | CGAATCTTGGAGCTCCCGCTGCTTTTAAATATTTTGATACAACAATTGATCGTAA |
| | ACGATATACGTCTACAAAAGAAGTTTTAGATGCCACTCTTATCCATCAATCCATC |
| | ACTGGTCTTTATGAAACACGCATTGATTTGAGTCAGCTAGGAGGTGACTGA |
| | (SEQ ID NO: 27) |
| SpCas9 | MDKKYSIGLDIGTNSVGWAVITDEYKVPSKKFKVLGNTDRHSIKKNLIGALLFDSGE |
| Streptococcus | TAEATRLKRTARRRYTRRKNRICYLQEIFSNEMAKVDDSFFHRLEESFLVEEDKKHE |
| pyogenes | RHPIFGNIVDEVAYHEKYPTIYHLRKKLVDSTDKADLRLIYLALAHMIKFRGHFLIEG |
| MIGAS wild | DLNPDNSDVDKLFIQLVQTYNQLFEENPINASGVDAKAILSARLSKSRRLENLIAQLP |
| type | GEKKNGLFGNLIALSLGLTPNFKSNFDLAEDAKLQLSKDTYDDDLDNLLAQIGDQY |
| Encoded | ADLFLAAKNLSDAILLSDILRVNTEITKAPLSASMIKRYDEHHQDLTLLKALVRQQL |
| product of | PEKYKEIFFDQSKNGYAGYIDGGASQEEFYKFIKPILEKMDGTEELLVKLNREDLLR |
| NC_002737.2 | KQRTFDNGSIPHQIHLGELHAILRRQEDFYPFLKDNREKIEKILTFRIPYYVGPLARGN |
| (100% identical | SRFAWMTRKSEETITPWNFEEVVDKGASAQSFIERMTNFDKNLPNEKVLPKHSLLY |
| to the canonical | EYFTVYNELTKVKYVTEGMRKPAFLSGEQKKAIVDLLFKTNRKVTVKQLKEDYFK |
| Q99ZW2 | KIECFDSVEISGVEDRFNASLGTYHDLLKIIKDKDFLDNEENEDILEDIVLTLTLFEDR |
| wild type) | EMIEERLKTYAHLFDDKVMKQLKRRRYTGWGRLSRKLINGIRDKQSGKTILDFLKS |
| | DGFANRNFMQLIHDDSLTFKEDIQKAQVSGQGDSLHEHIANLAGSPAIKKGILQTVK |
| | VVDELVKVMGRHKPENIVIEMARENQTTQKGQKNSRERMKRIEEGIKELGSQILKE |
| | HPVENTQLQNEKLYLYYLQNGRDMYVDQELDINRLSDYDVDHIVPQSFLKDDSIDN |
| | KVLTRSDKNRGKSDNVPSEEVVKKMKNYWRQLLNAKLITQRKFDNLTKAERGGLS |
| | ELDKAGFIKRQLVETRQITKHVAQILDSRMNTKYDENDKLIREVKVITLKSKLVSDF |
| | RKDFQFYKVREINNYHHAHDAYLNAVVGTALIKKYPKLESEFVYGDYKVYDVRK |
| | MIAKSEQEIGKATAKYFFYSNIMNFFKTEITLANGEIRKRPLIETNGETGEIVWDKGR |
| | DFATVRKVLSMPQVNIVKKTEVQTGGFSKESILPKRNSDKLIARKKDWDPKKYGGF |
| | DSPTVAYSVLVVAKVEKGKSKKLKSVKELLGITIMERSSFEKNPIDFLEAKGYKEVK |
| | KDLIIKLPKYSLFELENGRKRMLASAGELQKGNELALPSKYVNFLYLASHYEKLKGS |
| | PEDNEQKQLFVEQHKHYLDEIIEQISEFSKRVILADANLDKVLSAYNKHRDKPIREQ |
| | AENIIHLFTLTNLGAPAAFKYFDTTIDRKRYTSTKEVLDATLIHQSITGLYETRIDLSQ |
| | LGGD (SEQ ID NO: 6) |

The prime editors used in the methods and complexes described herein may include any of the above SpCas9 sequences, or any variant thereof having at least 80%, at least 85%, at least 90%, at least 95%, or at least 99% sequence identity thereto. In other embodiments, the Cas9 protein can be a wild type Cas9 ortholog from another bacterial species different from the canonical Cas9 from S. pyogenes. For example, modified versions of the following Cas9 orthologs can be used in connection with the prime editors described in this specification by making mutations at positions corresponding to H840A (or H839A in SEQ ID NO: 8) or any other amino acids of interest in wild type SpCas9. In addition, any variant Cas9 orthologs having at least 80%, at least 85%, at least 90%, at least 95%, or at least 99% sequence identity to any of the below orthologs may also be used with the prime editors.

| Description | Sequence |
|---|---|
| LfCas9 | MKEYHIGLDIGTSSIGWAVTDSQFKLMRIKGKTAIGVRLFEEGKTAAERRTFRTTRRRLK |
| Lactobacillus | RRKWRLHYLDEIFAPHLQEVDENFLRRLKQSNIHPEDPTKNQAFIGKLLFPDLLKKNERG |
| fermentum | YPTLIKMRDELPVEQRAHYPVMNIYKLREAMINEDRQFDLREVYLAVHHIVKYRGHFL |
| wild type | NNASVDKFKVGRIDFDKSFNVLNEAYEELQNGEGSFTIEPSKVEKIGQLLLDTKMRKLD |
| GenBank: | RQKAVAKLLEVKVADKEETKRNKQIATAMSKLVLGYKADFATVAMANGNEWKIDLSS |
| SNX31424.1 1 | ETSEDEIEKFREELSDAQNDILTEITSLFSQIMLNEIVPNGMSISESMMDRYWTHERQLAE |
| | VKEYLATQPASARKEFDQVYNKYIGQAPKERGFDLEKGLKKILSKKENWKEIDELLKAG |
| | DFLPKQRTSANGVIPHQMHQQELDRIIEKQAKYYPWLATENPATGERDRHQAKYELDQ |
| | LVSFRIPYYVGPLVTPEVQKATSGAKFAWAKRKEDGEITPWNLWDKIDRAESAEAFIKR |
| | MTVKDTYLLNEDVLPANSLLYQKYNVLNELNNVRVNGRRLSVGIKQDIYTELFKKKKT |
| | VKASDVASLVMAKTRGVNKPSVEGLSDPKKFNSNLATYLDLKSIVGDKVDDNRYQTDL |
| | ENIIEWRSVFEDGEIFADKLTEVEWLTDEQRSALVKKRYKGWGRLSKKLLTGIVDENGQ |
| | RIIDLMWNTDQNFKEIVDQPVFKEQIDQLNQKAITNDGMTLRERVESVLDDAYTSPQNK |
| | KAIWQVVRVVEDIVKAVGNAPKSISIEFARNEGNKGEITRSRRTQLQKLFEDQAHELVK |
| | DTSLTEELEKAPDLSDRYYFYFTQGGKDMYTGDPINFDEISTKYDIDHILPQSFVKDNSL |
| | DNRVLTSRKENNKKSDQVPAKLYAAKMKPYWNQLLKQGLITQRKFENLTKDVDQNIK |
| | YRSLGFVKRQLVETRQVIKLTANILGSMYQEAGTEIIETRAGLTKQLREEFDLPKVREVN |
| | DYHHAVDAYLTTFAGQYLNRRYPKLRSFFVYGEYMKFKHGSDLKLRNFNFFHELMEG |
| | DKSQGKVVDQQTGELITTRDEVAKSFDRLLNMKYMLVSKEVHDRSDQLYGATIVTAKE |
| | SGKLTSPIEIKKNRLVDLYGAYTNGTSAFMTIIKFTGNKPKYKVIGIPTTSAASLKRAGKP |
| | GSESYNQELHRIIKSNPKVKKGFEIVVPHVSYGQLIVDGDCKFTLASPTVQHPATQLVLS |
| | KKSLETISSGYKILKDKPAIANERLIRVFDEVVGQMNRYFTIFDQRSNRQKVADARDKFL |
| | SLPTESKYEGAKKVQVGKTEVITNLLMGLHANATQGDLKVLGLATFGFFQSTTGLSLSE |
| | DTMIVYQSPTGLFERRICLKDI |
| | (SEQ ID NO: 12) |
| SaCas9 | MDKKYSIGLDIGTNSVGWAVITDEYKVPSKKFKVLGNTDRHSIKKNLIGALLFDSGETA |
| Staphylococcus | EATRLKRTARRRYTRRKNRICYLQEIFSNEMAKVDDSFFHRLEESFLVEEDKKHERHPIF |
| aureus | GNIVDEVAYHEKYPTIYHLRKKLVDSTDKADLRLIYLALAHMIKFRGHFLIEGDLNPDNS |
| wild type | DVDKLFIQLVQTYNQLFEENPINASGVDAKAILSARLSKSRRLENLIAQLPGEKKNGLFG |
| GenBank: | NLIALSLGLTPNFKSNFDLAEDAKLQLSKDTYDDDLDNLLAQIGDQYADLFLAAKNLSD |
| AYD60528.1 | AILLSDILRVNTEITKAPLSASMIKRYDEHHQDLTLLKALVRQQLPEKYKEIFFDQSKNGY |
| | AGYIDGGASQEEFYKFIKPILEKMDGTEELLVKLNREDLLRKQRTFDNGSIPHQIHLGEL |
| | HAILRRQEDFYPFLKDNREKIEKILTFRIPYYVGPLARGNSRFAWMTRKSEETITPWNFEE |
| | VVDKGASAQSFIERMTNFDKNLPNEKVLPKHSLLYEYFTVYNELTKVKYVTEGMRKPA |
| | FLSGEQKKAIVDLLFKTNRKVTVKQLKEDYFKKIECFDSVEISGVEDRFNASLGTYHDLL |
| | KIIKDKDFLDNEENEDILEDIVLTLTLFEDREMIEERLKTYAHLFDDKVMKQLKRRRYTG |
| | WGRLSRKLINGIRDKQSGKTILDFLKSDGFANRNFMQLIHDDSLTFKEDIQKAQVSGQG |
| | DSLHEHIANLAGSPAIKKGILQTVKVVDELVKVMGRHKPENIVIEMARENQTTQKGQKN |
| | SRERMKRIEEGIKELGSQILKEHPVENTQLQNEKLYLYYLQNGRDMYVDQELDINRLSD |
| | YDVDHIVPQSFLKDDSIDNKVLTRSDKNRGKSDNVPSEEVVKKMKNYWRQLLNAKLIT |
| | QRKFDNLTKAERGGLSELDKAGFIKRQLVETRQITKHVAQILDSRMNTKYDENDKLIRE |
| | VKVITLKSKLVSDFRKDFQFYKVREINNYHHAHDAYLNAVVGTALIKKYPKLESEFVYG |
| | DYKVYDVRKMIAKSEQEIGKATAKYFFYSNIMNFFKTEITLANGEIRKRPLIETNGETGEI |
| | VWDKGRDFATVRKVLSMPQVNIVKKTEVQTGGFSKESILPKRNSDKLIARKKDWDPKK |
| | YGGFDSPTVAYSVLVVAKVEKGKSKKLKSVKELLGITIMERSSFEKNPIDFLEAKGYKEV |
| | KKDLIIKLPKYSLFELENGRKRMLASAGELQKGNELALPSKYVNFLYLASHYEKLKGSP |
| | EDNEQKQLFVEQHKHYLDEIIEQISEFSKRVILADANLDKVLSAYNKHRDKPIREQAENII |
| | HLFTLTNLGAPAAFKYFDTTIDRKRYTSTKEVLDATLIHQSITGLYETRIDLSQLGGD |
| | (SEQ ID NO: 28) |
| SaCas9 | MGKRNYILGLDIGITSVGYGIIDYETRDVIDAGVRLFKEANVENNEGRRSKRGARRLKRR |
| Staphylococcus | RRHRIQRVKKLLFDYNLLTDHSELSGINPYEARVKGLSQKLSEEEFSAALLHLAKRRGV |
| aureus | HNVNEVEEDTGNELSTKEQISRNSKALEEKYVAELQLERLKKDGEVRGSINRFKTSDYV |
| | KEAKQLLKVQKAYHQLDQSFIDTYIDLLETRRTYYEGPGEGSPFGWKDIKEWYEMLMG |
| | HCTYFPEELRSVKYAYNADLYNALNDLNNLVITRDENEKLEYYEKFQIIENVFKQKKKP |
| | TLKQIAKEILVNEEDIKGYRVTSTGKPEFTNLKVYHDIKDITARKEIIENAELLDQIAKILTI |
| | YQSSEDIQEELTNLNSELTQEEIEQISNLKGYTGTHNLSLKAINLILDELWHTNDNQIAIFN |
| | RLKLVPKKVDLSQQKEIPTTLVDDFILSPVVKRSFIQSIKVINAIIKKYGLPNDIIIELAREK |
| | NSKDAQKMINEMQKRNRQTNERIEEIIRTTGKENAKYLIEKIKLHDMQEGKCLYSLEAIP |
| | LEDLLNNPFNYEVDHIIPRSVSFDNSFNNKVLVKQEENSKKGNRTPFQYLSSSDSKISYET |
| | FKKHILNLAKGKGRISKTKKEYLLEERDINRFSVQKDFINRNLVDTRYATRGLMNLLRSY |
| | FRVNNLDVKVKSINGGFTSFLRRKWKFKKERNKGYKHHAEDALIIANADFIFKEWKKL |
| | DKAKKVMENQMFEEKQAESMPEIETEQEYKEIFITPHQIKHIKDFKDYKYSHRVDKKPN |
| | RKLINDTLYSTRKDDKGNTLIVNNLNGLYDKDNDKLKKLINKSPEKLLMYHHDPQTYQ |
| | KLKLIMEQYGDEKNPLYKYYEETGNYLTKYSKKDNGPVIKKIKYYGNKLNAHLDITDD |
| | YPNSRNKVVKLSLKPYRFDVYLDNGVYKFVTVKNLDVIKKENYYEVNSKCYEEAKKL |
| | KKISNQAEFIASFYKNDLIKINGELYRVIGVNNDLLNRIEVNMIDITYREYLENMNDKRPP |
| | HIIKTIASKTQSIKKYSTDILGNLYEVKSKKHPQIIKK |
| | (SEQ ID NO: 13) |
| StCas9 | MLFNKCIIISINLDFSNKEKCMTKPYSIGLDIGTNSVGWAVITDNYKVPSKKMKVLGNTS |
| Streptococcus | KKYIKKNLLGVLLFDSGITAEGRRLKRTARRRYTRRRNRILYLQEIFSTEMATLDDAFFQ |
| thermophilus | RLDDSFLVPDDKRDSKYPIFGNLVEEKVYHDEFPTIYHLRKYLADSTKKADLRLVYLAL |
| UniProtKB/S | AHMIKYRGHFLIEGEFNSKNNDIQKNFQDFLDTYNAIFESDLSLENSKQLEEIVKDKISKL |
| wiss-Prot: | EKKDRILKLFPGEKNSGIFSEFLKLIVGNQADFRKCFNLDEKASLHFSKESYDEDLETLLG |
| G3ECR1.2 | YIGDDYSDVFLKAKKLYDAILLSGFLTVTDNETEAPLSSAMIKRYNEHKEDLALLKEYIR |
| Wild type | NISLKTYNEVFKDDTKNGYAGYIDGKTNQEDFYVYLKNLLAEFEGADYFLEKIDREDFL |
| | RKQRTFDNGSIPYQIHLQEMRAILDKQAKFYPFLAKNKERIEKILTFRIPYYVGPLARGNS |
| | DFAWSIRKRNEKITPWNFEDVIDKESSAEAFINRMTSFDLYLPEEKVLPKHSLLYETFNV |
| | YNELTKVRFIAESMRDYQFLDSKQKKDIVRLYFKDKRKVTDKDIIEYLHAIYGYDGIELK |
| | GIEKQFNSSLSTYHDLLNIINDKEFLDDSSNEAIIEEIIHTLTIFEDREMIKQRLSKFENIF |
| | DKSVLKKLSRRHYTGWGKLSAKLINGIRDEKSGNTILDYLIDDGISNRNFMQLIHDDALSFK |
| | KKIQKAQIIGDEDKGNIKEVVKSLPGSPAIKKGILQSIKIVDELVKVMGGRKPESIVVEMA |
| | RENQYTNQGKSNSQQRLKRLEKSLKELGSKILKENIPAKLSKIDNNALQNDRLYLYYLQ |
| | NGKDMYTGDDLDIDRLSNYDIDHIIPQAFLKDNSIDNKVLVSSASNRGKSDDFPSLEVVK |
| | KRKTFWYQLLKSKLISQRKFDNLTKAERGGLLPEDKAGFIQRQLVETRQITKHVARLLD |
| | EKFNNKKDENNRAVRTVKIITLKSTLVSQFRKDFELYKVREINDFHHAHDAYLNAVIAS |
| | ALLKKYPKLEPEFVYGDYPKYNSFRERKSATEKVYFYSNIMNIFKKSISLADGRVIERPLI |
| | EVNEETGESVWNKESDLATVRRVLSYPQVNVVKKVEEQNHGLDRGKPKGLFNANLSS |
| | KPKPNSNENLVGAKEYLDPKKYGGYAGISNSFAVLVKGTIEKGAKKKITNVLEFQGISIL |
| | DRINYRKDKLNFLLEKGYKDIELIIELPKYSLFELSDGSRRMLASILSTNNKRGEIHKGNQI |
| | FLSQKFVKLLYHAKRISNTINENHRKYVENHKKEFEELFYYILEFNENYVGAKKNGKLL |
| | NSAFQSWQNHSIDELCSSFIGPTGSERKGLFELTSRGSAADFEFLGVKIPRYRDYTPSSLL |
| | KDATLIHQSVTGLYETRIDLAKLGEG |
| | (SEQ ID NO: 14) |
| LcCas9 | MKIKNYNLALTPSTSAVGHVEVDDDLNILEPVHHQKAIGVAKFGEGETAEARRLARSAR |
| Lactobacillus | RTTKRRANRINHYFNEIMKPEIDKVDPLMFDRIKQAGLSPLDERKEFRTVIFDRPNIASYY |
| crispatus | HNQFPTIWHLQKYLMITDEKADIRLIYWALHSLLKHRGHFFNTTPMSQFKPGKLNLKDD |
| NCBI | MLALDDYNDLEGLSFAVANSPEIEKVIKDRSMHKKEKIAELKKLIVNDVPDKDLAKRNN |
| Reference | KIITQIVNAIMGNSFHLNFIFDMDLDKLTSKAWSFKLDDPELDTKFDAISGSMTDNQIGIF |
| Sequence: | ETLQKIYSAISLLDILNGSSNVVDAKNALYDKHKRDLNLYFKFLNTLPDEIAKTLKAGYT |
| WP_1334780 | LYIGNRKKDLLAARKLLKVNVAKNFSQDDFYKLINKELKSIDKQGLQTRFSEKVGELVA |
| 44.1 | QNNFLPVQRSSDNVFIPYQLNAITFNKILENQGKYYDFLVKPNPAKKDRKNAPYELSQL |
| Wild type | MQFTIPYYVGPLVTPEEQVKSGIPKTSRFAWMVRKDNGAITPWNFYDKVDIEATADKFI |
| | KRSIAKDSYLLSELVLPKHSLLYEKYEVFNELSNVSLDGKKLSGGVKQILFNEVFKKTNK |
| | VNTSRILKALAKHNIPGSKITGLSNPEEFTSSLQTYNAWKKYFPNQIDNFAYQQDLEKMI |
| | EWSTVFEDHKILAKKLDEIEWLDDDQKKFVANTRLRGWGRLSKRLLTGLKDNYGKSIM |
| | QRLETTKANFQQIVYKPEFREQIDKISQAAAKNQSLEDILANSYTSPSNRKAIRKTMSVV |
| | DEYIKLNHGKEPDKIFLMFQRSEQEKGKQTEARSKQLNRILSQLKADKSANKLFSKQLA |
| | DEFSNAIKKSKYKLNDKQYFYFQQLGRDALTGEVIDYDELYKYTVLHIIPRSKLTDDSQN |
| | NKVLTKYKIVDGSVALKFGNSYSDALGMPIKAFWTELNRLKLIPKGKLLNLTTDFSTLN |
| | KYQRDGYIARQLVETQQIVKLLATIMQSRFKHTKIIEVRNSQVANIRYQFDYFRIKNLNE |
| | YYRGFDAYLAAVVGTYLYKVYPKARRLFVYGQYLKPKKTNQENQDMHLDSEKKSQGF |
| | NFLWNLLYGKQDQIFVNGTDVIAFNRKDLITKMNTVYNYKSQKISLAIDYHNGAMFKA |
| | TLFPRNDRDTAKTRKLIPKKKDYDTDIYGGYTSNVDGYMLLAEIIKRDGNKQYGFYGVP |
| | SRLVSELDTLKKTRYTEYEEKLKEIIKPELGVDLKKIKKIKILKNKVPFNQVIIDKGSKFFI |
| | TSTSYRWNYRQLILSAESQQTLMDLVVDPDFSNHKARKDARKNADERLIKVYEEILYQV |
| | KNYMPMFVELHRCYEKLVDAQKTFKSLKISDKAMVLNQILILLHSNATSPVLEKLGYHT |
| | RFTLGKKHNLISENAVLVTQSITGLKENHVSIKQML |
| | (SEQ ID NO: 15) |
| PdCas9 | MTNEKYSIGLDIGTSSIGFAVVNDNNRVIRVKGKNAIGVRLFDEGKAAADRRSFRTTRRS |
| Pedicoccus | FRTTRRRLSRRRWRLKLLREIFDAYITPVDEAFFIRLKESNLSPKDSKKQYSGDILENDRS |
| damnosus | DKDFYEKYPTIYHLRNALMTEHRKFDVREIYLAIHHIMKFRGHFLNATPANNFKVGRLN |
| NCBI | LEEKFEELNDIYQRVFPDESIEFRTDNLEQIKEVLLDNKRSRADRQRTLVSDIYQSSEDKD |
| Reference | IEKRNKAVATEILKASLGNKAKLNVITNVEVDKEAAKEWSITFDSESIDDDLAKIEGQMT |
| Sequence: | DDGHEIIEVLRSLYSGITLSAIVPENHTLSQSMVAKYDLHKDHLKLFKKLINGMTDTKKA |
| WP_ | KNLRAAYDGYIDGVKGKVLPQEDFYKQVQVNLDDSAEANEIQTYIDQDIFMPKQRTKA |
| 062913273.1 | NGSIPHQLQQQELDQIIENQKAYYPWLAELNPNPDKKRQQLAKYKLDELVTFRVPYYV |
| Wild type | GPMITAKDQKNQSGAEFAWMIRKEPGNITPWNFDQKVDRMATANQFIKRMTTTDTYLL |
| | GEDVLPAQSLLYQKFEVLNELNKIRIDHKPISIEQKQQIFNDLFKQFKNVTIKHLQDYLVS |
| | QGQYSKRPLIEGLADEKRFNSSLSTYSDLCGIFGAKLVEENDRQEDLEKIIEWSTIFEDKKI |
| | YRAKLNDLTWLTDDQKEKLATKRYQGWGRLSRKLLVGLKNSEHRNIMDILWITNENF |
| | MQIQAEPDFAKLVTDANKGMLEKTDSQDVINDLYTSPQNKKAIRQILLVVHDIQNAMH |
| | GQAPAKIHVEFARGEERNPRRSVQRQRQVEAAYEKVSNELVSAKVRQEFKEAINNKRD |
| | FKDRLFLYFMQGGIDIYTGKQLNIDQLSSYQIDHILPQAFVKDDSLTNRVLTNENQVKAD |
| | SVPIDIFGKKMLSVWGRMKDQGLISKGKYRNLTMNPENISAHTENGFINRQLVETRQVI |
| | KLAVNILADEYGDSTQIISVKADLSHQMREDFELLKNRDVNDYHHAFDAYLAAFIGNYL |
| | LKRYPKLESYFVYGDFKKFTQKETKMRRFNFIYDLKHCDQVVNKETGEILWTKDEDIKY |
| | IRHLFAYKKILVSHEVREKRGALYNQTIYKAKDDKGSGQESKKLIRIKDDKETKIYGGYS |
| | GKSLAYMTIVQITKKNKVSYRVIGIPTLALARLNKLENDSTENNGELYKIIKPQFTHYKV |
| | DKKNGEIIETTDDFKIVVSKVRFQQLIDDAGQFFMLASDTYKNNAQQLVISNNALKAINN |
| | TNITDCPRDDLERLDNLRLDSAFDEIVKKMDKYFSAYDANNFREKIRNSNLIFYQLPVED |
| | QWENNKITELGKRTVLTRILQGLHANATTTDMSIFKIKTPFGQLRQRSGISLSENAQLIYQ |
| | SPTGLFERRVQLNKIK |
| | (SEQ ID NO: 16) |
| FnCas9 | MKKQKFSDYYLGFDIGTNSVGWCVTDLDYNVLRFNKKDMWGSRLFEEAKTAAERRVQ |
| Fusobaterium | RNSRRRLKRRKWRLNLLEEIFSNEILKIDSNFFRRLKESSLWLEDKSSKEKFTLENDDNY |
| nucleatum | KDYDFYKQYPTIFHLRNELIKNPEKKDIRLVYLAIHSIFKSRGHFLFEGQNLKEIKNFETL |
| NCBI | YNNLIAFLEDNGINKIIDKNNIEKLEKIVCDSKKGLKDKEKEFKEIFNSDKQLVAIFKLSV |
| Reference | GSSVSLNDLFDTDEYKKGEVEKEKISFREQIYEDDKPIYYSILGEKIELLDIAKTFYDFMV |
| Sequence: | LNNILADSQYISEAKVKLYEEHKKDLKNLKYIIRKYNKGNYDKLFKDKNENNYSAYIGLNK |
| WP_ | EKSKKEVIEKSRLKIDDLIKNIKGYLPKVEEIEEKDKAIFNKILNKIELKTILPKQRISDN |
| 060798984.1 | GTLPYQIHEAELEKILENQSKYYDFLNYEENGIITKDKLLMTFKFRIPYYVGPLNSYHKD |
| | KGGNSWIVRKEEGKILPWNFEQKVDIEKSAEEFIKRMTNKCTYLNGEDVIPKDTFLYSEY |
| | VILNELNKVQVNDEFLNEENKRKIIDELFKENKKVSEKKFKEYLLVKQIVDGTIELKGVK |
| | DSFNSNYISYIRFKDIFGEKLNLDIYKEISEKSILWKCLYGDDKKIFEKKIKNEYGDILTKD |
| | EIKKINTFKFNNWGRLSEKLLTGIEFINLETGECYSSVMDALRRTNYNLMELLSSKFTLQE |
| | SINNENKEMNEASYRDLIEESYVSPSLKRAIFQTLKIYEEIRKITGRVPKKVFIEMARGGD |
| | ESMKNKKIPARQEQLKKLYDSCGNDIANFSIDIKEMKNSLISYDNNSLRQKKLYLYYLQF |
| | GKCMYTGREIDLDRLLQNNDTYDIDHIYPRSKVIKDDSFDNLVLVLKNENAEKSNEYPV |
| | KKEIQEKMKSFWRFLKEKNFISDEKYKRLTGKDDFELRGFMARQLVNVRQTTKEVGKIL |
| | QQIEPEIKIVYSKAEIASSFREMFDFIKVRELNDTHHAKDAYLNIVAGNVYNTKFTEKPYR |
| | YLQEIKENYDVKKIYNYDIKNAWDKENSLEIVKKNMEKNTVNITRFIKEKKGQLFDLNPI |
| | KKGETSNEIISIKPKVYNGKDDKLNEKYGYYKSLNPAYFLYVEHKEKNKRIKSFERVNL |
| | VDVNNIKDEKSLVKYLIENKKLVEPRVIKKVYKRQVILINDYPYSIVTLDSNKLMDFENL |
| | KPLFLENKYEKILKNVIKFLEDNQGKSEENYKFIYLKKKDRYEKNETLESVKDRYNLEFN |
| | EMYDKFLEKLDSKDYKNYMNNKKYQELLDVKEKFIKLNLFDKAFTLKSFLDLFNRKTM |
| | ADFSKVGLTKYLGKIQKISSNVLSKNELYLLEESVTGLFVKKIKL |
| | (SEQ ID NO: 17) |
| EcCas9 | RRKQRIQILQELLGEEVLKTDPGFFHRMKESRYVVEDKRTLDGKQVELPYALFVDKDYT |
| Enterococcus | DKEYYKQFPTINHLIVYLMTTSDTPDIRLVYLALHYYMKNRGNFLHSGDINNVKDINDIL |
| cecorum | EQLDNVLETFLDGWNLKLKSYVEDIKNIYNRDLGRGERKKAFVNTLGAKTKAEKAFCSL |
| NCBI | ISGGSTNLAELFDDSSLKEIETPKIEFASSSLEDKIDGIQEALEDRFAVIEAAKRLYDWKT |
| Reference | LTDILGDSSSLAEARVNSYQMHHEQLLELKSLVKEYLDRKVFQEVFVSLNVANNYPAYI |
| Sequence: | GHTKINGKKKELEVKRTKRNDFYSYVKKQVIEPIKKKVSDEAVLTKLSEIESLIEVDKYL |
| WP_ | PLQVNSDNGVIPYQVKLNELTRIFDNLENRIPVLRENRDKIIKTFKFRIPYYVGSLNGVVK |
| 047338501.1 | NGKCTNWMVRKEEGKIYPWNFEDKVDLEASAEQFIRRMTNKCTYLVNEDVLPKYSLL |
| Wild type | YSKYLVLSELNNLRIDGRPLDVKIKQDIYENVFKKNRKVTLKKIKKYLLKEGIITDDDEL |
| | SGLADDVKSSLTAYRDFKEKLGHLDLSEAQMENIILNITLFGDDKKLLKKRLAALYPFID |
| | DKSLNRIATLNYRDWGRLSERFLSGITSVDQETGELRTIIQCMYETQANLMQLLAEPYHF |
| | VEAIEKENPKVDLESISYRIVNDLYVSPAVKRQIWQTLLVIKDIKQVMKHDPERIFIEMAR |
| | EKQESKKTKSRKQVLSEVYKKAKEYEHLFEKLNSLTEEQLRSKKIYLYFTQLGKCMYSG |
| | EPIDFENLVSANSNYDIDHIYPQSKTIDDSFNNIVLVKKSLNAYKSNHYPIDKNIRDNEKV |
| | KTLWNTLVSKGLITKEKYERLIRSTPFSDEELAGFIARQLVETRQSTKAVAEILSNWFPES |
| | EIVYSKAKNVSNFRQDFEILKVRELNDCHHAHDAYLNIVVGNAYHTKFTNSPYRFIKNK |
| | ANQEYNLRKLLQKVNKIESNGVVAWVGQSENNPGTIATVKKVIRRNTVLISRMVKEVD |
| | GQLFDLTLMKKGKGQVPIKSSDERLTDISKYGGYNKATGAYFTFVKSKKRGKVVRSFE |
| | YVPLHLSKQFENNNELLKEYIEKDRGLTDVEILIPKVLINSLFRYNGSLVRITGRGDTRLL |
| | LVHEQPLYVSNSFVQQLKSVSSYKLKKSENDNAKLTKTATEKLSNIDELYDGLLRKLDL |
| | PIYSYWFSSIKEYLVESRTKYIKLSIEEKALVIFEILHLFQSDAQVPNLKILGLSTKPSRI |
| | RIQKNLKDTDKMSIIHQSPSGIFEHEIELTSL |
| | (SEQ ID NO: 18) |
| AhCas9 | MQNGFLGITVSSEQVGWAVTNPKYELERASRKDLWGVRLFDKAETAEDRRMFRTNRR |
| Anaerostipes | LNQRKKNRIHYLRDIFHEEVNQKDPNFFQQLDESNFCEDDRTVEFNFDTNLYKNQFPTV |
| hadrus | YHLRKYLMETKDKPDIRLVYLAFSKFMKNRGHFLYKGNLGEVMDFENSMKGFCESLEK |
| NCBI | FNIDFPTLSDEQVKEVRDILCDHKIAKTVKKKNIITITKVKSKTAKAWIGLFCGCSVPVKV |
| Reference | LFQDIDEEIVTDPEKISFEDASYDDYIANIEKGVGIYYEAIVSAKMLFDWSILNEILGDHQL |
| Sequence: | LSDAMIAEYNKHHDDLKRLQKIIKGTGSRELYQDIFINDVSGNYVCYVGHAKTMSSADQ |
| WP_ | KQFYTFLKNRLKNVNGISSEDAEWIDTEIKNGTLLPKQTKRDNSVIPHQLQLREFELILDN |
| 044924278.1 | MQEMYPFLKENREKLLKIFNFVIPYYVGPLKGVVRKGESTNWMVPKKDGVIHPWNFDE |
| Wild type | MVDKEASAECFISRMTGNCSYLFNEKVLPKNSLLYETFEVLNELNPLKINGEPISVELKQ |
| | RIYEQLFLTGKKVTKKSLTKYLIKNGYDKDIELSGIDNEFHSNLKSHIDFEDYDNLSDEEV |
| | EQIILRITVFEDKQLLKDYLNREFVKLSEDERKQICSLSYKGWGNLSEMLLNGITVTDSN |
| | GVEVSVMDMLWNTNLNLMQILSKKYGYKAEIEHYNKEHEKTIYNREDLMDYLNIPPAQ |
| | RRKVNQLITIVKSLKKTYGVPNKIFFKISREHQDDPKRTSSRKEQLKYLYKSLKSEDEKH |
| | LMKELDELNDHELSNDKVYLYFLQKGRCIYSGKKLNLSRLRKSNYQNDIDYIYPLSAVN |
| | DRSMNNKVLTGIQENRADKYTYFPVDSEIQKKMKGFWMELVLQGFMTKEKYFRLSREND |
| | FSKSELVSFIEREISDNQQSGRMIASVLQYYFPESKIVFVKEKLISSFKRDFHLISSYGH |
| | NHLQAAKDAYITIVVGNVYHTKFTMDPAIYFKNHKRKDYDLNRLFLENISRDGQIAWES |
| | GPYGSIQTVRKEYAQNHIAVTKRVVEVKGGLFKQMPLKKGHGEYPLKTNDPRFGNIAQ |
| | YGGYTNVTGSYFVLVESMEKGKKRISLEYVPVYLHERLEDDPGHKLLKEYLVDHRKLN |
| | HPKILLAKVRKNSLLKIDGFYYRLNGRSGNALILTNAVELIMDDWQTKTANKISGYMKR |
| | RAIDKKARVYQNEFHIQELEQLYDFYLDKLKNGVYKNRKNNQAELIHNEKEQFMELKT |
| | EDQCVLLTEIKKLFVCSPMQADLTLIGGSKHTGMIAMSSNVTKADFAVIAEDPLGLRNK |
| | VIYSHKGEK |
| | (SEQ ID NO: 19) |
| KvCas9 | MSQNNNKIYNIGLDIGDASVGWAVVDEHYNLLKRHGKHMWGSRLFTQANTAVERRSS |
| Kandleria | RSTRRRYNKRRERIRLLREIMEDMVLDVDPTFFIRLANVSFLDQEDKKDYLKENYHSNY |
| vitulina | NLFIDKDFNDKTYYDKYPTIYHLRKHLCESKEKEDPRLIYLALHHIVKYRGNFLYEGQKF |
| NCBI | SMDVSNIEDKMIDVLRQFNEINLFEYVEDRKKIDEVLNVLKEPLSKKHKAEKAFALFDTT |
| Reference | KDNKAAYKELCAALAGNKFNVTKMLKEAELHDEDEKDISFKFSDATFDDAFVEKQPLL |
| Sequence: | GDCVEFIDLLHDIYSWVELQNILGSAHTSEPSISAAMIQRYEDHKNDLKLLKDVIRKYLP |
| WP_0315899 | KKYFEVFRDEKSKKNNYCNYINHPSKTPVDEFYKYIKKLIEKIDDPDVKTILNKIELESFM |
| 69.1 | LKQNSRTNGAVPYQMQLDELNKILENQSVYYSDLKDNEDKIRSILTFRIPYYFGPLNITK |
| Wild type | DRQFDWIIKKEGKENERILPWNANEIVDVDKTADEFIKRMRNFCTYFPDEPVMAKNSLT |
| | VSKYEVLNEINKLRINDHLIKRDMKDKMLHTLFMDHKSISANAMKKWLVKNQYFSNTDDI |
| | KIEGFQKENACSTSLTPWIDFTKIFGKINESNYDFIEKIIYDVTVFEDKKILRRRLKKEY |
| | DLDEEKIKKILKLKYSGWSRLSKKLLSGIKTKYKDSTRTPETVLEVMERTNMNLMQVIN |
| | DEKLGFKKTIDDANSTSVSGKFSYAEVQELAGSPAIKRGIWQALLIVDEIKKIMKHEPAH |
| | VYIEFARNEDEKERKDSFVNQMLKLYKDYDFEDETEKEANKHLKGEDAKSKIRSERLKL |
| | YYTQMGKCMYTGKSLDIDRLDTYQVDHIVPQSLLKDDSIDNKVLVLSSENQRKLDDLVI |
| | PSSIRNKMYGFWEKLFNNKIISPKKFYSLIKTEFNEKDQERFINRQIVETRQITKHVAQIID |
| | NHYENTKVVTVRADLSHQFRERYHIYKNRDINDFHHAHDAYIATILGTYIGHRFESLDA |
| | KYIYGEYKRIFRNQKNKGKEMKKNNDGFILNSMRNIYADKDTGEIVWDPNYIDRIKKCF |
| | YYKDCFVTKKLEENNGTFFNVTVLPNDTNSDKDNTLATVPVNKYRSNVNKYGGFSGV |
| | NSFIVAIKGKKKKGKKVIEVNKLTGIPLMYKNADEEIKINYLKQAEDLEEVQIGKEILKN |
| | QLIEKDGGLYYIVAPTEIINAKQLILNESQTKLVCEIYKAMKYKNYDNLDSEKIIDLYRLL |
| | INKMELYYPEYRKQLVKKFEDRYEQLKVISIEEKCNIIKQILATLHCNSSIGKIMYSDFKIS |
| | TTIGRLNGRTISLDDISFIAESPTGMYSKKYKL |
| | (SEQ ID NO: 20) |
| EfCas9 | MRLFEEGHTAEDRRLKRTARRRISRRRNRLRYLQAFFEEAMTDLDENFFARLQESFLVP |
| Enterococcus | EDKKWHRHPIFAKLEDEVAYHETYPTIYHLRKKLADSSEQADLRLIYLALAHIVKYRGH |
| faecalis | FLIEGKLSTENTSVKDQFQQFMVIYNQTFVNGESRLVSAPLPESVLIEEELTEKASRTKKS |
| NCBI | EKVLQQFPQEKANGLFGQFLKLMVGNKADFKKVFGLEEEAKITYASESYEEDLEGILAK |
| Reference | VGDEYSDVFLAAKNVYDAVELSTILADSDKKSHAKLSSSMIVRFTEHQEDLKKFKRFIRE |
| Sequence: | NCPDEYDNLFKNEQKDGYAGYIAHAGKVSQLKFYQYVKKIIQDIAGAEYFLEKIAQENF |
| WP_0166310 | LRKQRTFDNGVIPHQIHLAELQAIIHRQAAYYPFLKENQEKIEQLVTFRIPYYVGPLSKGD |
| 44.1 | ASTFAWLKRQSEEPIRPWNLQETVDLDQSATAFIERMTNFDTYLPSEKVLPKHSLLYEKF |
| Wild type | MVFNELTKISYTDDRGIKANFSGKEKEKIFDYLFKTRRKVKKKDIIQFYRNEYNTEIVTLS |
| | GLEEDQFNASFSTYQDLLKCGLTRAELDHPDNAEKLEDIIKILTIFEDRQRIRTQLSTFKG |
| | QFSAEVLKKLERKHYTGWGRLSKKLINGIYDKESGKTILDYLVKDDGVSKHYNRNFMQ |
| | LINDSQLSFKNAIQKAQSSEHEETLSETVNELAGSPAIKKGIYQSLKIVDELVAIMGYAPK |
| | RIVVEMARENQTTSTGKRRSIQRLKIVEKAMAEIGSNLLKEQPTTNEQLRDTRLFLYYM |
| | QNGKDMYTGDELSLHRLSHYDIDHIIPQSFMKDDSLDNLVLVGSTENRGKSDDVPSKEV |
| | VKDMKAYWEKLYAAGLISQRKFQRLTKGEQGGLTLEDKAHFIQRQLVETRQITKNVAG |
| | ILDQRYNAKSKEKKVQIITLKASLTSQFRSIFGLYKVREVNDYHHGQDAYLNCVVATTL |
| | LKVYPNLAPEFVYGEYPKFQTFKENKATAKAIIYTNLLRFFTEDEPRFTKDGEILWSNSY |
| | LKTIKKELNYHQMNIVKKVEVQKGGFSKESIKPKGPSNKLIPVKNGLDPQKYGGFDSPV |
| | VAYTVLFTHEKGKKPLIKQEILGITIMEKTRFEQNPILFLEEKGFLRPRVLMKLPKYTLYE |
| | FPEGRRRLLASAKEAQKGNQMVLPEHLLTLLYHAKQCLLPNQSESLAYVEQHQPEFQEI |
| | LERVVDFAEVHTLAKSKVQQIVKLFEANQTADVKEIAASFIQLMQFNAMGAPSTFKFFQ |
| | KDIERARYTSIKEIFDATIIYQSPTGLYETRRKVVD |
| | (SEQ ID NO: 21) |
| Staphylococcus | KRNYILGLDIGITSVGYGIIDYETRDVIDAGVRLFKEANVENNEGRRSKRGARRLKRRRR |
| aureus | HRIQRVKKLLFDYNLLTDHSELSGINPYEARVKGLSQKLSEEEFSAALLHLAKRRGVHN |
| Cas9 | VNEVEEDTGNELSTKEQISRNSKALEEKYVAELQLERLKKDGEVRGSINRFKTSDYVKE |
| | AKQLLKVQKAYHQLDQSFIDTYIDLLETRRTYYEGPGEGSPFGWKDIKEWYEMLMGHC |
| | TYFPEELRSVKYAYNADLYNALNDLNNLVITRDENEKLEYYEKFQIIENVFKQKKKPTL |
| | KQIAKEILVNEEDIKGYRVTSTGKPEFTNLKVYHDIKDITARKEIIENAELLDQIAKILTIY |
| | QSSEDIQEELTNLNSELTQEEIEQISNLKGYTGTHNLSLKAINLILDELWHTNDNQIAIFNR |
| | LKLVPKKVDLSQQKEIPTTLVDDFILSPVVKRSFIQSIKVINAIIKKYGLPNDIIIELAREKN |
| | SKDAQKMINEMQKRNRQTNERIEEIIRTTGKENAKYLIEKIKLHDMQEGKCLYSLEAIPL |
| | EDLLNNPFNYEVDHIIPRSVSFDNSFNNKVLVKQEENSKKGNRTPFQYLSSSDSKISYETF |
| | KKHILNLAKGKGRISKTKKEYLLEERDINRFSVQKDFINRNLVDTRYATRGLMNLLRSYF |
| | RVNNLDVKVKSINGGFTSFLRRKWKFKKERNKGYKHHAEDALIIANADFIFKEWKKLD |
| | KAKKVMENQMFEEKQAESMPEIETEQEYKEIFITPHQIKHIKDFKDYKYSHRVDKKPNR |
| | ELINDTLYSTRKDDKGNTLIVNNLNGLYDKDNDKLKKLINKSPEKLLMYHHDPQTYQK |
| | LKLIMEQYGDEKNPLYKYYEETGNYLTKYSKKDNGPVIKKIKYYGNKLNAHLDITDDY |
| | PNSRNKVVKLSLKPYRFDVYLDNGVYKFVTVKNLDVIKKENYYEVNSKCYEEAKKLK |
| | KISNQAEFIASFYNNDLIKINGELYRVIGVNNDLLNRIEVNMIDITYREYLENMNDKRPPR |
| | IIKTIASKTQSIKKYSTDILGNLYEVKSKKHPQIIKKG |
| | (SEQ ID NO: 22) |
| Geobacillus | MKYKIGLDIGITSIGWAVINLDIPRIEDLGVRIFDRAENPKTGESLALPRRLARSARRRLRR |
| thermodenitrificans | RKHRLERIRRLFVREGILTKEELNKLFEKKHEIDVWQLRVEALDRKLNNDELARILLHLA |
| Cas9 | KRRGFRSNRKSERTNKENSTMLKHIEENQSILSSYRTVAEMVVKDPKFSLHKRNKEDNY |
| | TNTVARDDLEREIKLIFAKQREYGNIVCTEAFEHEYISIWASQRPFASKDDIEKKVGFCTF |
| | EPKEKRAPKATYTFQSFTVWEHINKLRLVSPGGIRALTDDERRLIYKQAFHKNKITFHDV |
| | RTLLNLPDDTRFKGLLYDRNTTLKENEKVRFLELGAYHKIRKAIDSVYGKGAAKSFRPID |
| | FDTFGYALTMFKDDTDIRSYLRNEYEQNGKRMENLADKVYDEELIEELLNLSFSKFGHL |
| | SLKALRNILPYMEQGEVYSTACERAGYTFTGPKKKQKTVLLPNIPPIANPVVMRALTQA |
| | RKVVNAIIKKYGSPVSIHIELARELSQSFDERRKMQKEQEGNRKKNETAIRQLVEYGLTL |
| | NPTGLDIVKFKLWSEQNGKCAYSLQPIEIERLLEPGYTEVDHVIPYSRSLDDSYTNKVLV |
| | LTKENREKGNRTPAEYLGLGSERWQQFETFVLTNKQFSKKKRDRLLRLHYDENEENEF |
| | KNRNLNDTRYISRFLANFIREHLKFADSDDKQKVYTVNGRITAHLRSRWNFNKNREESN |
| | LHHAVDAAIVACTTPSDIARVTAFYQRREQNKELSKKTDPQFPQPWPHFADELQARLSK |
| | NPKESIKALNLGNYDNEKLESLQPVFVSRMPKRSITGAAHQETLRRYIGIDERSGKIQTV |
| | VKKKLSEIQLDKTGHFPMYGKESDPRTYEAIRQRLLEHNNDPKKAFQEPLYKPKKNGEL |
| | GPIIRTIKIIDTTNQVIPLNDGKTVAYNSNIVRVDVFEKDGKYYCVPIYTIDMMKGILPNK |
| | AIEPNKPYSEWKEMTEDYTFRFSLYPNDLIRIEFPREKTIKTAVGEEIKIKDLFAYYQTIDS |
| | SNGGLSLVSHDNNFSLRSIGSRTLKRFEKYQVDVLGNIYKVRGEKRVGVASSSHSKAGE |
| | TIRPL |
| | (SEQ ID NO: 23) |
| ScCas9 | MEKKYSIGLDIGTNSVGWAVITDDYKVPSKKFKVLGNTNRKSIKKNLMGALLFDSGETA |
| S. canis | EATRLKRTARRRYTRRKNRIRYLQEIFANEMAKLDDSFFQRLEESFLVEEDKKNERHPIF |
| | GNLADEVAYHRNYPTIYHLRKKLADSPEKADLRLIYLALAHIIKFRGHFLIEGKLNAENS |
| | DVAKLFYQLIQTYNQLFEESPLDEIEVDAKGILSARLSKSKRLEKLIAVFPNEKKNGLFGN |
| | IIALALGLTPNFKSNFDLTEDAKLQLSKDTYDDDLDELLGQIGDQYADLFSAAKNLSDAI |
| | LLSDILRSNSEVTKAPLSASMVKRYDEHHQDLALLKTLVRQQFPEKYAEIFKDDTKNGY |
| | AGYVGIGIKHRKRTTKLATQEEFYKFIKPILEKMDGAEELLAKLNRDDLLRKQRTFDNG |
| | SIPHQIHLKELHAILRRQEEFYPFLKENREKIEKILTFRIPYYVGPLARGNSRFAWLTRKSE |
| | EAITPWNFEEVVDKGASAQSFIERMTNFDEQLPNKKVLPKHSLLYEYFTVYNELTKVKY |
| | VTERMRKPEFLSGEQKKAIVDLLFKTNRKVTVKQLKEDYFKKIECFDSVEIIGVEDRFNA |
| | SLGTYHDLLKIIKDKDFLDNEENEDILEDIVLTLTLFEDREMIEERLKTYAHLFDDKVMK |
| | QLKRRHYTGWGRLSRKMINGIRDKQSGKTILDFLKSDGFSNRNFMQLIHDDSLTFKEEIE |
| | KAQVSGQGDSLHEQIADLAGSPAIKKGILQTVKIVDELVKVMGHKPENIVIEMARENQT |
| | TTKGLQQSRERKKRIEEGIKELESQILKENPVENTQLQNEKLYLYYLQNGRDMYVDQEL |
| | DINRLSDYDVDHIVPQSFIKDDSIDNKVLTRSVENRGKSDNVPSEEVVKKMKNYWRQLL |
| | NAKLITQRKFDNLTKAERGGLSEADKAGFIKRQLVETRQITKHVARILDSRMNTKRDKN |
| | DKPIREVKVITLKSKLVSDFRKDFQLYKVRDINNYHHAHDAYLNAVVGTALIKKYPKLE |
| | SEFVYGDYKVYDVRKMIAKSEQEIGKATAKRFFYSNIMNFFKTEVKLANGEIRKRPLIET |
| | NGETGEVVWNKEKDFATVRKVLAMPQVNIVKKTEVQTGGFSKESILSKRESAKLIPRKK |
| | GWDTRKYGGFGSPTVAYSILVVAKVEKGKAKKLKSVKVLVGITIMEKGSYEKDPIGFLE |
| | AKGYKDIKKELIFKLPKYSLFELENGRRRMLASATELQKANELVLPQHLVRLLYYTQNIS |
| | ATTGSNNLGYIEQHREEFKEIFEKIIDFSEKYILKNKVNSNLKSSFDEQFAVSDSILLSNSF |
| | VSLLKYTSFGASGGFTFLDLDVKQGRLRYQTVTEVLDATLIYQSITGLYETRTDLSQLGG |
| | D |
| | (SEQ ID NO: 24) |

The napDNAbps in the prime editors used in the methods and complexes described herein may include any suitable homologs and/or orthologs or naturally occurring enzymes, such as Cas9. Cas9 homologs and/or orthologs have been described in various species, including, but not limited to, S. pyogenes and S. thermophilus. The Cas moiety may be configured (e.g., mutagenized, recombinantly engineered, or otherwise obtained from nature) as a nickase, i.e., capable of cleaving only a single strand of the target double-stranded DNA. In some embodiments, a Cas9 nuclease has an inactive (e.g., an inactivated) DNA cleavage domain; that is, the Cas9 is a nickase. In some embodiments, the Cas9 protein comprises an amino acid sequence that is at least 85%, at least 90%, at least 92%, at least 95%, at least 96%, at least 97%, at least 98%, at least 99%, or at least 99.5% identical to the amino acid sequence of a Cas9 protein as provided by any one of the Cas9 orthologs in the above tables.

Additional suitable napDNAbp sequences that can be used in prime editors used in the methods and complexes described herein will be apparent to those of skill in the art based on this disclosure, and such Cas9 nucleases and sequences include Cas9 sequences from the organisms and loci disclosed in Chylinski, Rhun, and Charpentier, “The tracrRNA and Cas9 families of type II CRISPR-Cas immunity systems” (2013) RNA Biology 10:5, 726-737; the entire contents of which are incorporated herein by reference. Additional exemplary Cas variants and homologs include, but are not limited to, Cas9 (e.g., dCas9 and nCas9), Cpf1, CasX, CasY, C2c1, C2c2, C2c3, GeoCas9, CjCas9, Cas12a, Cas12b, Cas12g, Cas12h, Cas12i, Cas13b, Cas13c, Cas13d, Cas14, Csn2, xCas9, SpCas9-NG, Nme2Cas9, circularly permuted Cas9, Argonaute (Ago), Cas9-KKH, SmacCas9, Spy-macCas9, SpCas9-VRQR, SpCas9-NRRH, SpaCas9-NRTH, SpCas9-NRCH, LbCas12a, AsCas12a, CeCas12a, MbCas12a, Cas3, CasΦ, and circularly permuted Cas9 domains, such as CP1012, CP1028, CP1041, CP1249, and CP1300, and variants and homologs thereof.

### Reverse Transcriptase Domain

In various embodiments, the prime editors used in the methods and complexes described herein comprise a reverse transcriptase domain. In some embodiments, the reverse transcriptase domain is a wild type MMLV reverse transcriptase. In some embodiments, the reverse transcriptase domain is a variant of wild type MMLV reverse transcriptase having the amino acid sequence of SEQ ID NO: 31.

For example, PE2 and PEmax comprise a variant reverse transcriptase domain of SEQ ID NO: 31, which is based on the wild type MMLV reverse transcriptase domain of SEQ ID NO: 30 (and, in particular, a Genscript codon optimized MMLV reverse transcriptase having the nucleotide sequence of SEQ ID NO: 30) and which comprises amino acid substitutions D200N, T306K, W313F, T330P, and L603W relative to the wild type MMLV RT of SEQ ID NO: 30. The amino acid sequence of the variant RT of PE2 and PEmax is SEQ ID NO: 31. In some embodiments, the RT used in the prime editors used in the methods provided herein comprises the amino acid sequence of SEQ ID NO: 31, or an amino acid sequence at least 70%, at least 75%, at least 80%, at least 85%, at least 90%, at least 95%, at least 96%, at least 97%, at least 98%, or at least 99% identical to the amino acid sequence of SEQ ID NO: 31.

Prime editors may also comprise other variant RTs as well. In various embodiments, the prime editors used in the methods described herein (with RT provided as either a fusion partner or in trans) can include a variant RT comprising one or more of the following mutations: P51L, S67K, E69K, L139P, T197A, D200N, H204R, F209N, E302K, E302R, T306K, F309N, W313F, T330P, L345G, L435G, N454K, D524G, E562Q, D583N, H594Q, L603W, E607K, or D653N in the wild type M-MLV RT of SEQ ID NO: 30, or at a corresponding amino acid position in another wild type RT polypeptide sequence.

Some exemplary reverse transcriptases that can be fused to napDNAbp proteins or provided as individual proteins according to various embodiments of this disclosure are provided below. Exemplary reverse transcriptases include variants with at least 80%, at least 85%, at least 90%, at least 95%, or at least 99% sequence identity to the following wild-type enzymes or partial enzymes:

| Description | Sequence (variant substitutions relative to wild type) |
|---|---|
| Reverse | TLNIEDEYRLHETSKEPDVSLGSTWLSDFPQAWAETGGMGLAVRQAPLIIPLKATSTP |
| transcriptase | VSIKQYPMSQEARLGIKPHIQRLLDQGILVPCQSPWNTPLLPVKKPGTNDYRPVQDLR |
| (M-MLV RT) | EVNKRVEDIHPTVPNPYNLLSGLPPSHQWYTVLDLKDAFFCLRLHPTSQPLFAFEWR |
| wild type | DPEMGISGQLTWTRLPQGFKNSPTLFDEALHRDLADFRIQHPDLILLQYVDDLLLAAT |
| Moloney | SELDCQQGTRALLQTLGNLGYRASAKKAQICQKQVKYLGYLLKEGQRWLTEARKE |
| murine | TVMGQPTPKTPRQLREFLGTAGFCRLWIPGFAEMAAPLYPLTKTGTLFNWGPDQQK |
| leukemia virus | AYQEIKQALLTAPALGLPDLTKPFELFVDEKQGYAKGVLTQKLGPWRRPVAYLSKK |
| Used in PE1 | LDPVAAGWPPCLRMVAAIAVLTKDAGKLTMGQPLVILAPHAVEALVKQPPDRWLS |
| | NARMTHYQALLLDTDRVQFGPVVALNPATLLPLPEEGLQHNCLDILAEAHGTRPDLT |
| | DQPLPDADHTWYTDGSSLLQEGQRKAGAAVTTETEVIWAKALPAGTSAQRAELIAL |
| | TQALKMAEGKKLNVYTDSRYAFATAHIHGEIYRRRGLLTSEGKEIKNKDEILALLKA |
| | LFLPKRLSIIHCPGHQKGHSAEARGNRMADQAARKAAITETPDTSTLLIENSSP |
| | (SEQ ID NO: 30) |
| M-MLV RT | TLNIEDEYRLHETSKEPDVSLGSTWLSDFPQAWAETGGMGLAVRQAPLIIPLKATSTP |
| D200N | VSIKQYPMSQEARLGIKPHIQRLLDQGILVPCQSPWNTPLLPVKKPGTNDYRPVQDLR |
| | EVNKRVEDIHPTVPNPYNLLSGLPPSHQWYTVLDLKDAFFCLRLHPTSQPLFAFEWR |
| | DPEMGISGQLTWTRLPQGFKNSPTLFNEALHRDLADFRIQHPDLILLQYVDDLLLAAT |
| | SELDCQQGTRALLQTLGNLGYRASAKKAQICQKQVKYLGYLLKEGQRWLTEARKE |
| | TVMGQPTPKTPRQLREFLGTAGFCRLWIPGFAEMAAPLYPLTKTGTLFNWGPDQQK |
| | AYQEIKQALLTAPALGLPDLTKPFELFVDEKQGYAKGVLTQKLGPWRRPVAYLSKK |
| | LDPVAAGWPPCLRMVAAIAVLTKDAGKLTMGQPLVILAPHAVEALVKQPPDRWLS |
| | NARMTHYQALLLDTDRVQFGPVVALNPATLLPLPEEGLQHNCLDILAEAHGTRPDLT |
| | DQPLPDADHTWYTDGSSLLQEGQRKAGAAVTTETEVIWAKALPAGTSAQRAELIAL |
| | TQALKMAEGKKLNVYTDSRYAFATAHIHGEIYRRRGLLTSEGKEIKNKDEILALLKA |
| | LFLPKRLSIIHCPGHQKGHSAEARGNRMADQAARKAAITETPDTSTLLIENSSP (SEQ |
| | ID NO: 32) |
| M-MLV RT | TLNIEDEYRLHETSKEPDVSLGSTWLSDFPQAWAETGGMGLAVRQAPLIIPLKATSTP |
| D200N | VSIKQYPMSQEARLGIKPHIQRLLDQGILVPCQSPWNTPLLPVKKPGTNDYRPVQDLR |
| T330P | EVNKRVEDIHPTVPNPYNLLSGLPPSHQWYTVLDLKDAFFCLRLHPTSQPLFAFEWR |
| | DPEMGISGQLTWTRLPQGFKNSPTLFNEALHRDLADFRIQHPDLILLQYVDDLLLAAT |
| | SELDCQQGTRALLQTLGNLGYRASAKKAQICQKQVKYLGYLLKEGQRWLTEARKE |
| | TVMGQPTPKTPRQLREFLGTAGFCRLWIPGFAEMAAPLYPLTKPGTLFNWGPDQQK |
| | AYQEIKQALLTAPALGLPDLTKPFELFVDEKQGYAKGVLTQKLGPWRRPVAYLSKK |
| | LDPVAAGWPPCLRMVAAIAVLTKDAGKLTMGQPLVILAPHAVEALVKQPPDRWLS |
| | NARMTHYQALLLDTDRVQFGPVVALNPATLLPLPEEGLQHNCLDILAEAHGTRPDLT |
| | DOPLPDADHTWYTDGSSLLQEGQRKAGAAVTTETEVIWAKALPAGTSAQRAELIAL |
| | TQALKMAEGKKLNVYTDSRYAFATAHIHGEIYRRRGLLTSEGKEIKNKDEILALLKA |
| | LFLPKRLSIIHCPGHQKGHSAEARGNRMADQAARKAAITETPDTSTLLIENSSP (SEQ |
| | ID NO: 33) |
| M-MLV RT | TLNIEDEYRLHETSKEPDVSLGSTWLSDFPQAWAETGGMGLAVRQAPLIIPLKATSTP |
| D200N | VSIKQYPMSQEARLGIKPHIQRLLDQGILVPCQSPWNTPLLPVKKPGTNDYRPVQDLR |
| T330P | EVNKRVEDIHPTVPNPYNLLSGLPPSHQWYTVLDLKDAFFCLRLHPTSQPLFAFEWR |
| L603W | DPEMGISGQLTWTRLPQGFKNSPTLFNEALHRDLADFRIQHPDLILLQYVDDLLLAAT |
| | SELDCQQGTRALLQTLGNLGYRASAKKAQICQKQVKYLGYLLKEGQRWLTEARKE |
| | TVMGQPTPKTPRQLREFLGTAGFCRLWIPGFAEMAAPLYPLTKPGTLFNWGPDQQK |
| | AYQEIKQALLTAPALGLPDLTKPFELFVDEKQGYAKGVLTQKLGPWRRPVAYLSKK |
| | LDPVAAGWPPCLRMVAAIAVLTKDAGKLTMGQPLVILAPHAVEALVKQPPDRWLS |
| | NARMTHYQALLLDTDRVQFGPVVALNPATLLPLPEEGLQHNCLDILAEAHGTRPDLT |
| | DQPLPDADHTWYTDGSSLLQEGQRKAGAAVTTETEVIWAKALPAGTSAQRAELIAL |
| | TQALKMAEGKKLNVYTDSRYAFATAHIHGEIYRRRGWLTSEGKEIKNKDEILALLKA |
| | LFLPKRLSIIHCPGHQKGHSAEARGNRMADQAARKAAITETPDTSTLLIENSSP (SEQ |
| | ID NO: 34) |
| M-MLV RT | TLNIEDEYRLHETSKEPDVSLGSTWLSDFPQAWAETGGMGLAVRQAPLIIPLKATSTP |
| D200N | VSIKQYPMSQKARLGIKPHIQRLLDQGILVPCQSPWNTPLLPVKKPGTNDYRPVQDL |
| T330P | REVNKRVEDIHPTVPNPYNLLSGLPPSHQWYTVLDLKDAFFCLRLHPTSQPLFAFEW |
| L603W | RDPEMGISGQLTWTRLPQGFKNSPTLFNEALHRDLADFRIQHPDLILLQYVDDLLLAA |
| E69K | TSELDCQQGTRALLQTLGNLGYRASAKKAQICQKQVKYLGYLLKEGQRWLTEARK |
| | ETVMGQPTPKTPRQLREFLGTAGFCRLWIPGFAEMAAPLYPLTKPGTLFNWGPDQQ |
| | KAYQEIKQALLTAPALGLPDLTKPFELFVDEKQGYAKGVLTQKLGPWRRPVAYLSK |
| | KLDPVAAGWPPCLRMVAAIAVLTKDAGKLTMGQPLVILAPHAVEALVKQPPDRWL |
| | SNARMTHYQALLLDTDRVQFGPVVALNPATLLPLPEEGLQHNCLDILAEAHGTRPDL |
| | TDQPLPDADHTWYTDGSSLLQEGQRKAGAAVTTETEVIWAKALPAGTSAQRAELIA |
| | LTQALKMAEGKKLNVYTDSRYAFATAHIHGEIYRRRGWLTSEGKEIKNKDEILALLK |
| | ALFLPKRLSIIHCPGHQKGHSAEARGNRMADQAARKAAITETPDTSTLLIENSSP |
| | (SEQ ID NO: 35) |
| M-MLV RT | TLNIEDEYRLHETSKEPDVSLGSTWLSDFPQAWAETGGMGLAVRQAPLIIPLKATSTP |
| D200N | VSIKQYPMSQEARLGIKPHIQRLLDQGILVPCQSPWNTPLLPVKKPGTNDYRPVQDLR |
| T330P | EVNKRVEDIHPTVPNPYNLLSGLPPSHQWYTVLDLKDAFFCLRLHPTSQPLFAFEWR |
| L603W | DPEMGISGQLTWTRLPQGFKNSPTLFNEALHRDLADFRIQHPDLILLQYVDDLLLAAT |
| E302R | SELDCQQGTRALLQTLGNLGYRASAKKAQICQKQVKYLGYLLKEGQRWLTEARKE |
| | TVMGQPTPKTPRQLRRFLGTAGFCRLWIPGFAEMAAPLYPLTKPGTLFNWGPDQQK |
| | AYQEIKQALLTAPALGLPDLTKPFELFVDEKQGYAKGVLTQKLGPWRRPVAYLSKK |
| | LDPVAAGWPPCLRMVAAIAVLTKDAGKLTMGQPLVILAPHAVEALVKQPPDRWLS |
| | NARMTHYQALLLDTDRVQFGPVVALNPATLLPLPEEGLQHNCLDILAEAHGTRPDLT |
| | DQPLPDADHTWYTDGSSLLQEGQRKAGAAVTTETEVIWAKALPAGTSAQRAELIAL |
| | TQALKMAEGKKLNVYTDSRYAFATAHIHGEIYRRRGWLTSEGKEIKNKDEILALLKA |
| | LFLPKRLSIIHCPGHQKGHSAEARGNRMADQAARKAAITETPDTSTLLIENSSP(SEQ |
| | ID NO: 36) |
| M-MLV RT | TLNIEDEYRLHETSKEPDVSLGSTWLSDFPQAWAETGGMGLAVRQAPLIIPLKATSTP |
| D200N | VSIKQYPMSQEARLGIKPHIQRLLDQGILVPCQSPWNTPLLPVKKPGTNDYRPVQDLR |
| T330P | EVNKRVEDIHPTVPNPYNLLSGLPPSHQWYTVLDLKDAFFCLRLHPTSQPLFAFEWR |
| L603W | DPEMGISGQLTWTRLPQGFKNSPTLFNEALHRDLADFRIQHPDLILLQYVDDLLLAAT |
| E607K | SELDCQQGTRALLQTLGNLGYRASAKKAQICQKQVKYLGYLLKEGQRWLTEARKE |
| | TVMGQPTPKTPRQLREFLGTAGFCRLWIPGFAEMAAPLYPLTKPGTLFNWGPDQQK |
| | AYQEIKQALLTAPALGLPDLTKPFELFVDEKQGYAKGVLTQKLGPWRRPVAYLSKK |
| | LDPVAAGWPPCLRMVAAIAVLTKDAGKLTMGQPLVILAPHAVEALVKQPPDRWLS |
| | NARMTHYQALLLDTDRVQFGPVVALNPATLLPLPEEGLQHNCLDILAEAHGTRPDLT |
| | DQPLPDADHTWYTDGSSLLQEGQRKAGAAVTTETEVIWAKALPAGTSAQRAELIAL |
| | TQALKMAEGKKLNVYTDSRYAFATAHIHGEIYRRRGWLTSKGKEIKNKDEILALLK |
| | ALFLPKRLSIIHCPGHQKGHSAEARGNRMADQAARKAAITETPDTSTLLIENSSP (SEQ |
| | ID NO: 37) |
| M-MLV RT | TLNIEDEYRLHETSKEPDVSLGSTWLSDFPQAWAETGGMGLAVRQAPLIIPLKATSTP |
| D200N | VSIKQYPMSQEARLGIKPHIQRLLDQGILVPCQSPWNTPLLPVKKPGTNDYRPVQDLR |
| T330P | EVNKRVEDIHPTVPNPYNLLSGPPPSHQWYTVLDLKDAFFCLRLHPTSQPLFAFEWR |
| L603W | DPEMGISGQLTWTRLPQGFKNSPTLFNEALHRDLADFRIQHPDLILLQYVDDLLLAAT |
| L139P | SELDCQQGTRALLQTLGNLGYRASAKKAQICQKQVKYLGYLLKEGQRWLTEARKE |
| | TVMGQPTPKTPRQLREFLGTAGFCRLWIPGFAEMAAPLYPLTKPGTLFNWGPDQQK |
| | AYQEIKQALLTAPALGLPDLTKPFELFVDEKQGYAKGVLTQKLGPWRRPVAYLSKK |
| | LDPVAAGWPPCLRMVAAIAVLTKDAGKLTMGQPLVILAPHAVEALVKQPPDRWLS |
| | NARMTHYQALLLDTDRVQFGPVVALNPATLLPLPEEGLQHNCLDILAEAHGTRPDLT |
| | DQPLPDADHTWYTDGSSLLQEGQRKAGAAVTTETEVIWAKALPAGTSAQRAELIAL |
| | TQALKMAEGKKLNVYTDSRYAFATAHIHGEIYRRRGWLTSEGKEIKNKDEILALLKA |
| | LFLPKRLSIIHCPGHQKGHSAEARGNRMADQAARKAAITETPDTSTLLIENSSP (SEQ |
| | ID NO: 38) |
| M-MLV RT | TLNIEDEYRLHETSKEPDVSLGSTWLSDFPQAWAETGGMGLAVRQAPLIIPLKATSTP |
| D200N | VSIKQYPMSQEARLGIKPHIQRLLDQGILVPCQSPWNTPLLPVKKPGTNDYRPVQDLR |
| T330P | EVNKRVEDIHPTVPNPYNLLSGLPPSHQWYTVLDLKDAFFCLRLHPTSQPLFAFEWR |
| L603W | DPEMGISGQLTWTRLPQGFKNSPTLFNEALHRDLADFRIQHPDLILLQYVDDLLLAAT |
| L435G | SELDCQQGTRALLQTLGNLGYRASAKKAQICQKQVKYLGYLLKEGQRWLTEARKE |
| | TVMGQPTPKTPRQLREFLGTAGFCRLWIPGFAEMAAPLYPLTKPGTLFNWGPDQQK |
| | AYQEIKQALLTAPALGLPDLTKPFELFVDEKQGYAKGVLTQKLGPWRRPVAYLSKK |
| | LDPVAAGWPPCLRMVAAIAVLTKDAGKLTMGQPLVIGAPHAVEALVKQPPDRWLS |
| | NARMTHYQALLLDTDRVQFGPVVALNPATLLPLPEEGLQHNCLDILAEAHGTRPDLT |
| | DQPLPDADHTWYTDGSSLLQEGQRKAGAAVTTETEVIWAKALPAGTSAQRAELIAL |
| | TQALKMAEGKKLNVYTDSRYAFATAHIHGEIYRRRGWLTSEGKEIKNKDEILALLKA |
| | LFLPKRLSIIHCPGHQKGHSAEARGNRMADQAARKAAITETPDTSTLLIENSSP (SEQ |
| | ID NO: 39) |
| M-MLV RT | TLNIEDEYRLHETSKEPDVSLGSTWLSDFPQAWAETGGMGLAVRQAPLIIPLKATSTP |
| D200N | VSIKQYPMSQEARLGIKPHIQRLLDQGILVPCQSPWNTPLLPVKKPGTNDYRPVQDLR |
| T330P | EVNKRVEDIHPTVPNPYNLLSGLPPSHQWYTVLDLKDAFFCLRLHPTSQPLFAFEWR |
| L603W | DPEMGISGQLTWTRLPQGFKNSPTLFNEALHRDLADFRIQHPDLILLQYVDDLLLAAT |
| N454K | SELDCQQGTRALLQTLGNLGYRASAKKAQICQKQVKYLGYLLKEGQRWLTEARKE |
| | TVMGQPTPKTPRQLREFLGTAGFCRLWIPGFAEMAAPLYPLTKPGTLFNWGPDQQK |
| | AYQEIKQALLTAPALGLPDLTKPFELFVDEKQGYAKGVLTQKLGPWRRPVAYLSKK |
| | LDPVAAGWPPCLRMVAAIAVLTKDAGKLTMGQPLVILAPHAVEALVKQPPDRWLS |
| | KARMTHYQALLLDTDRVQFGPVVALNPATLLPLPEEGLQHNCLDILAEAHGTRPDL |
| | TDQPLPDADHTWYTDGSSLLQEGQRKAGAAVTTETEVIWAKALPAGTSAQRAELIA |
| | LTQALKMAEGKKLNVYTDSRYAFATAHIHGEIYRRRGWLTSEGKEIKNKDEILALLK |
| | ALFLPKRLSIIHCPGHQKGHSAEARGNRMADQAARKAAITETPDTSTLLIENSSP (SEQ |
| | ID NO: 40) |
| M-MLV RT | TLNIEDEYRLHETSKEPDVSLGSTWLSDFPQAWAETGGMGLAVRQAPLIIPLKATSTP |
| D200N | VSIKQYPMSQEARLGIKPHIQRLLDQGILVPCQSPWNTPLLPVKKPGTNDYRPVQDLR |
| T330P | EVNKRVEDIHPTVPNPYNLLSGLPPSHQWYTVLDLKDAFFCLRLHPTSQPLFAFEWR |
| L603W | DPEMGISGQLTWTRLPQGFKNSPTLFNEALHRDLADFRIQHPDLILLQYVDDLLLAAT |
| T306K | SELDCQQGTRALLQTLGNLGYRASAKKAQICQKQVKYLGYLLKEGQRWLTEARKE |
| | TVMGQPTPKTPRQLREFLGKAGFCRLWIPGFAEMAAPLYPLTKPGTLFNWGPDQQK |
| | AYQEIKQALLTAPALGLPDLTKPFELFVDEKQGYAKGVLTQKLGPWRRPVAYLSKK |
| | LDPVAAGWPPCLRMVAAIAVLTKDAGKLTMGQPLVILAPHAVEALVKQPPDRWLS |
| | NARMTHYQALLLDTDRVQFGPVVALNPATLLPLPEEGLQHNCLDILAEAHGTRPDLT |
| | DQPLPDADHTWYTDGSSLLQEGQRKAGAAVTTETEVIWAKALPAGTSAQRAELIAL |
| | TQALKMAEGKKLNVYTDSRYAFATAHIHGEIYRRRGWLTSEGKEIKNKDEILALLKA |
| | LFLPKRLSIIHCPGHQKGHSAEARGNRMADQAARKAAITETPDTSTLLIENSSP (SEQ |
| | ID NO: 41) |
| M-MLV RT | TLNIEDEYRLHETSKEPDVSLGSTWLSDFPQAWAETGGMGLAVRQAPLIIPLKATSTP |
| D200N | VSIKQYPMSQEARLGIKPHIQRLLDQGILVPCQSPWNTPLLPVKKPGTNDYRPVQDLR |
| T330P | EVNKRVEDIHPTVPNPYNLLSGLPPSHQWYTVLDLKDAFFCLRLHPTSQPLFAFEWR |
| L603W | DPEMGISGQLTWTRLPQGFKNSPTLFNEALHRDLADFRIQHPDLILLQYVDDLLLAAT |
| W313F | SELDCQQGTRALLQTLGNLGYRASAKKAQICQKQVKYLGYLLKEGQRWLTEARKE |
| | TVMGQPTPKTPRQLREFLGTAGFCRLFIPGFAEMAAPLYPLTKPGTLFNWGPDQQKA |
| | YQEIKQALLTAPALGLPDLTKPFELFVDEKQGYAKGVLTQKLGPWRRPVAYLSKKL |
| | DPVAAGWPPCLRMVAAIAVLTKDAGKLTMGQPLVILAPHAVEALVKQPPDRWLSN |
| | ARMTHYQALLLDTDRVQFGPVVALNPATLLPLPEEGLQHNCLDILAEAHGTRPDLTD |
| | QPLPDADHTWYTDGSSLLQEGQRKAGAAVTTETEVIWAKALPAGTSAQRAELIALT |
| | QALKMAEGKKLNVYTDSRYAFATAHIHGEIYRRRGWLTSEGKEIKNKDEILALLKAL |
| | FLPKRLSIIHCPGHQKGHSAEARGNRMADQAARKAAITETPDTSTLLIENSSP (SEQ ID |
| | NO: 42) |
| M-MLV RT | TLNIEDEYRLHETSKEPDVSLGSTWLSDFPQAWAETGGMGLAVRQAPLIIPLKATSTP |
| D200N | VSIKQYPMSQEARLGIKPHIQRLLDQGILVPCQSPWNTPLLPVKKPGTNDYRPVQDLR |
| T330P | EVNKRVEDIHPTVPNPYNLLSGLPPSHQWYTVLDLKDAFFCLRLHPTSQPLFAFEWR |
| L603W | DPEMGISGQLTWTRLPQGFKNSPTLFNEALHRDLADFRIQHPDLILLQYVDDLLLAAT |
| D524G | SELDCQQGTRALLQTLGNLGYRASAKKAQICQKQVKYLGYLLKEGQRWLTEARKE |
| E562Q | TVMGQPTPKTPRQLREFLGTAGFCRLWIPGFAEMAAPLYPLTKPGTLFNWGPDQQK |
| D583N | AYQEIKQALLTAPALGLPDLTKPFELFVDEKQGYAKGVLTQKLGPWRRPVAYLSKK |
| | LDPVAAGWPPCLRMVAAIAVLTKDAGKLTMGQPLVILAPHAVEALVKQPPDRWLS |
| | NARMTHYQALLLDTDRVQFGPVVALNPATLLPLPEEGLQHNCLDILAEAHGTRPDLT |
| | DQPLPDADHTWYTGGSSLLQEGQRKAGAAVTTETEVIWAKALPAGTSAQRAQLIAL |
| | TQALKMAEGKKLNVYTNSRYAFATAHIHGEIYRRRGWLTSEGKEIKNKDEILALLKA |
| | LFLPKRLSIIHCPGHQKGHSAEARGNRMADQAARKAAITETPDTSTLLIENSSP (SEQ |
| | ID NO: 43) |
| M-MLV RT | TLNIEDEYRLHETSKEPDVSLGSTWLSDFPQAWAETGGMGLAVRQAPLIIPLKATSTP |
| D200N | VSIKQYPMSQEARLGIKPHIQRLLDQGILVPCQSPWNTPLLPVKKPGTNDYRPVQDLR |
| T330P | EVNKRVEDIHPTVPNPYNLLSGLPPSHQWYTVLDLKDAFFCLRLHPTSQPLFAFEWR |
| L603W | DPEMGISGQLTWTRLPQGFKNSPTLFNEALHRDLADFRIQHPDLILLQYVDDLLLAAT |
| E302R | SELDCQQGTRALLQTLGNLGYRASAKKAQICQKQVKYLGYLLKEGQRWLTEARKE |
| W313F | TVMGQPTPKTPRQLRRFLGTAGFCRLFIPGFAEMAAPLYPLTKPGTLFNWGPDQQKA |
| | YQEIKQALLTAPALGLPDLTKPFELFVDEKQGYAKGVLTQKLGPWRRPVAYLSKKL |
| | DPVAAGWPPCLRMVAAIAVLTKDAGKLTMGQPLVILAPHAVEALVKQPPDRWLSN |
| | ARMTHYQALLLDTDRVQFGPVVALNPATLLPLPEEGLQHNCLDILAEAHGTRPDLTD |
| | QPLPDADHTWYTDGSSLLQEGQRKAGAAVTTETEVIWAKALPAGTSAQRAELIALT |
| | QALKMAEGKKLNVYTDSRYAFATAHIHGEIYRRRGWLTSEGKEIKNKDEILALLKAL |
| | FLPKRLSIIHCPGHQKGHSAEARGNRMADQAARKAAITETPDTSTLLIENSSP (SEQ ID |
| | NO: 44) |
| M-MLV RT | TLNIEDEYRLHETSKEPDVSLGSTWLSDFPQAWAETGGMGLAVRQAPLIIPLKATSTP |
| D200N | VSIKQYPMSQEARLGIKPHIQRLLDQGILVPCQSPWNTPLLPVKKPGTNDYRPVQDLR |
| T330P | EVNKRVEDIHPTVPNPYNLLSGPPPSHQWYTVLDLKDAFFCLRLHPTSQPLFAFEWR |
| L603W | DPEMGISGQLTWTRLPQGFKNSPTLFNEALHRDLADFRIQHPDLILLQYVDDLLLAAT |
| E607K | SELDCQQGTRALLQTLGNLGYRASAKKAQICQKQVKYLGYLLKEGQRWLTEARKE |
| L139P | TVMGQPTPKTPRQLREFLGTAGFCRLWIPGFAEMAAPLYPLTKPGTLFNWGPDQQK |
| | AYQEIKQALLTAPALGLPDLTKPFELFVDEKQGYAKGVLTQKLGPWRRPVAYLSKK |
| | LDPVAAGWPPCLRMVAAIAVLTKDAGKLTMGQPLVILAPHAVEALVKQPPDRWLS |
| | NARMTHYQALLLDTDRVQFGPVVALNPATLLPLPEEGLQHNCLDILAEAHGTRPDLT |
| | DQPLPDADHTWYTDGSSLLQEGQRKAGAAVTTETEVIWAKALPAGTSAQRAELIAL |
| | TQALKMAEGKKLNVYTDSRYAFATAHIHGEIYRRRGWLTSKGKEIKNKDEILALLK |
| | ALFLPKRLSIIHCPGHQKGHSAEARGNRMADQAARKAAITETPDTSTLLIENSSP (SEQ |
| | ID NO: 45) |
| M-MLV RT | TLNIEDEYRLHETSKEPDVSLGSTWLSDFPQA WAETGGMGLAVRQAPLIILLKATSTP |
| P51L S67K | VSIKQYPMKQEARLGIKPHIQRLLDQGILVPCQSPWNTPLLPVKKPGTNDYRPVQDL |
| T197A H204R | REVNKRVEDIHPTVPNPYNLLSGLPPSHQWYTVLDLKDAFFCLRLHPTSQPLFAFEW |
| E302K F309N | RDPEMGISGQLTWTRLPQGFKNSPALFDEALRRDLADFRIQHPDLILLQYVDDLLLA |
| W313F T330P | ATSELDCQQGTRALLQTLGNLGYRASAKKAQICQKQVKYLGYLLKEGQRWLTEAR |
| L435G N454K | KETVMGQPTPKTPRQLRKFLGTAGNCRLFIPGFAEMAAPLYPLTKPGTLFNWGPDQ |
| D524G D583N | QKAYQEIKQALLTAPALGLPDLTKPFELFVDEKQGYAKGVLTQKLGPWRRPVAYLS |
| H594Q D653N | KKLDPVAAGWPPCLRMVAAIAVLTKDAGKLTMGQPLVIGAPHAVEALVKQPPDRW |
| | LSKARMTHYQALLLDTDRVQFGPVVALNPATLLPLPEEGLQHNCLDILAEAHGTRPD |
| | LTDQPLPDADHTWYTGGSSLLQEGQRKAGAAVTTETEVIWAKALPAGTSAQRAELI |
| | ALTQALKMAEGKKLNVYTNSRYAFATAHIQGEIYRRRGLLTSEGKEIKNKDEILALL |
| | KALFLPKRLSIIHCPGHQKGHSAEARGNRMANQAARKAAITETPDTSTLLIENSSP |
| | (SEQ ID NO: 46) |
| M-MLV RT | TLNIEDEYRLHETSKEPDVSLGSTWLSDFPQA WAETGGMGLAVRQAPLIILLKATSTP |
| D200N P51L | VSIKQYPMKQEARLGIKPHIQRLLDQGILVPCQSPWNTPLLPVKKPGTNDYRPVQDL |
| S67K T197A | REVNKRVEDIHPTVPNPYNLLSGLPPSHQWYTVLDLKDAFFCLRLHPTSQPLFAFEW |
| H204R E302K | RDPEMGISGQLTWTRLPQGFKNSPALFNEALRRDLADFRIQHPDLILLQYVDDLLLA |
| F309N W313F | ATSELDCQQGTRALLQTLGNLGYRASAKKAQICQKQVKYLGYLLKEGQRWLTEAR |
| T330P L345G | KETVMGQPTPKTPRQLRKFLGTAGNCRLFIPGFAEMAAPLYPLTKPGTLFNWGPDQ |
| N454K D524G | QKAYQEIKQALLTAPALGLPDLTKPFELFVDEKQGYAKGVLTQKLGPWRRPVAYLS |
| D583N H594Q | KKLDPVAAGWPPCLRMVAAIAVLTKDAGKLTMGQPLVIGAPHAVEALVKQPPDRW |
| D653N | LSKARMTHYQALLLDTDRVQFGPVVALNPATLLPLPEEGLQHNCLDILAEAHGTRPD |
| | LTDQPLPDADHTWYTGGSSLLQEGQRKAGAAVTTETEVIWAKALPAGTSAQRAELI |
| | ALTQALKMAEGKKLNVYTNSRYAFATAHIQGEIYRRRGLLTSEGKEIKNKDEILALL |
| | KALFLPKRLSIIHCPGHQKGHSAEARGNRMANQAARKAAITETPDTSTLLIENSSP |
| | (SEQ ID NO: 47) |
| M-MLV RT | TLNIEDEYRLHETSKEPDVSLGSTWLSDFPQAWAETGGMGLAVRQAPLIIPLKATSTP |
| D200N T330P | VSIKQYPMSQEARLGIKPHIQRLLDQGILVPCQSPWNTPLLPVKKPGTNDYRPVQDLR |
| L603W T306K | EVNKRVEDIHPTVPNPYNLLSGLPPSHQWYTVLDLKDAFFCLRLHPTSQPLFAFEWR |
| W313F | DPEMGISGQLTWTRLPQGFKNSPTLFNEALHRDLADFRIQHPDLILLQYVDDLLLAAT |
| in PE2 and | SELDCQQGTRALLQTLGNLGYRASAKKAQICQKQVKYLGYLLKEGQRWLTEARKE |
| PEmax | TVMGQPTPKTPRQLREFLGKAGFCRLFIPGFAEMAAPLYPLTKPGTLFNWGPDQQK |
| | AYQEIKQALLTAPALGLPDLTKPFELFVDEKQGYAKGVLTQKLGPWRRPVAYLSKK |
| | LDPVAAGWPPCLRMVAAIAVLTKDAGKLTMGQPLVILAPHAVEALVKQPPDRWLS |
| | NARMTHYQALLLDTDRVQFGPVVALNPATLLPLPEEGLQHNCLDILAEAHGTRPDLT |
| | DQPLPDADHTWYTDGSSLLQEGQRKAGAAVTTETEVIWAKALPAGTSAQRAELIAL |
| | TQALKMAEGKKLNVYTDSRYAFATAHIHGEIYRRRGWLTSEGKEIKNKDEILALLKA |
| | LFLPKRLSIIHCPGHQKGHSAEARGNRMADQAARKAAITETPDTSTLLIENSSP (SEQ |
| | ID NO: 31) |

In various embodiments, the prime editors utilized in the methods and complexes described herein (with RT provided as either a fusion partner or in trans) can include a variant RT comprising one or more of the following mutations: P51X, S67X, E69X, L139X, T197X, D200X, H204X, F209X, E302X, T306X, F309X, W313X, T330X, L345X, L435X, N454X, D524X, E562X, D583X, H594X, L603X, E607X, or D653X in the wild type M-MLV RT of SEQ ID NO: 30, or at a corresponding amino acid position in another wild type RT polypeptide sequence, wherein “X” can be any amino acid.

The prime editors used in the methods and complexes described herein may comprise any publicly-available reverse transcriptase described or disclosed in any of the following U.S. patents (each of which are incorporated by reference): U.S. Pat. Nos. 10,202,658; 10,189,831; 10,150,955; 9,932,567; 9,783,791; 9,580,698; 9,534,201; and 9,458,484, and any variant thereof that can be made using known methods for installing mutations or known methods for evolving proteins. The following references describe reverse transcriptases in known the art. Each of their disclosures are incorporated herein by reference.
Herzig, E., Voronin, N., Kucherenko, N. & Hizi, A. A Novel Leu92 Mutant of HIV-1 Reverse Transcriptase with a Selective Deficiency in Strand Transfer Causes a Loss of Viral Replication. J. Virol. 89, 8119-8129 (2015).
Mohr, G. et al. A Reverse Transcriptase-Cas1 Fusion Protein Contains a Cas6 Domain Required for Both CRISPR RNA Biogenesis and RNA Spacer Acquisition. Mol. Cell 72, 700-714.e8 (2018).
Zhao, C., Liu, F. & Pyle, A. M. An ultraprocessive, accurate reverse transcriptase encoded by a metazoan group II intron. RNA 24, 183-195 (2018).
Zimmerly, S. & Wu, L. An Unexplored Diversity of Reverse Transcriptases in Bacteria. Microbiol Spectr 3, MDNA3-0058-2014 (2015).
Ostertag, E. M. & Kazazian Jr, H. H. Biology of Mammalian L1 Retrotransposons. Annual Review of Genetics 35, 501-538 (2001).
Perach, M. & Hizi, A. Catalytic Features of the Recombinant Reverse Transcriptase of Bovine Leukemia Virus Expressed in Bacteria. Virology 259, 176-189 (1999).
Lim, D. et al. Crystal structure of the moloney murine leukemia virus RNase H domain. J. Virol. 80, 8379-8389 (2006).
Zhao, C. & Pyle, A. M. Crystal structures of a group II intron maturase reveal a missing link in spliceosome evolution. Nature Structural & Molecular Biology 23, 558-565 (2016).
Griffiths, D. J. Endogenous retroviruses in the human genome sequence. Genome Biol. 2, REVIEWS1017 (2001).
Baranauskas, A. et al., Generation and characterization of new highly thermostable and processive M-MuLV reverse transcriptase variants. Protein Eng Des Sel 25, 657-668 (2012).
Zimmerly, S., Guo, H., Perlman, P. S. & Lambowltz, A. M. Group II intron mobility occurs by target DNA-primed reverse transcription. Cell 82, 545-554 (1995).
Feng, Q., Moran, J. V., Kazazian, H. H. & Boeke, J. D. Human L1 retrotransposon encodes a conserved endonuclease required for retrotransposition. Cell 87, 905-916 (1996).
Berkhout, B., Jebbink, M. & Zsíros, J. Identification of an Active Reverse Transcriptase Enzyme Encoded by a Human Endogenous HERV-K Retrovirus. Journal of Virology 73, 2365-2375 (1999).
Kotewicz, M. L., Sampson, C. M., D'Alessio, J. M. & Gerard, G. F. Isolation of cloned Moloney murine leukemia virus reverse transcriptase lacking ribonuclease H activity. Nucleic Acids Res 16, 265-277 (1988).
Arezi, B. & Hogrefe, H. Novel mutations in Moloney Murine Leukemia Virus reverse transcriptase increase thermostability through tighter binding to template-primer. Nucleic Acids Res 37, 473-481 (2009).
Blain, S. W. & Goff, S. P. Nuclease activities of Moloney murine leukemia virus reverse transcriptase. Mutants with altered substrate specificities. J. Biol. Chem. 268, 23585-23592 (1993).
Xiong, Y. & Eickbush, T. H. Origin and evolution of retroelements based upon their reverse transcriptase sequences. EMBO J 9, 3353-3362 (1990).
Herschhorn, A. & Hizi, A. Retroviral reverse transcriptases. Cell. Mol. Life Sci. 67, 2717-2747 (2010).
Taube, R., Loya, S., Avidan, O., Perach, M. & Hizi, A. Reverse transcriptase of mouse mammary tumour virus: expression in bacteria, purification and biochemical characterization. Biochem. J. 329 (Pt 3), 579-587 (1998).
Liu, M. et al. Reverse Transcriptase-Mediated Tropism Switching in Bordetella Bacteriophage. Science 295, 2091-2094 (2002).
Luan, D. D., Korman, M. H., Jakubczak, J. L. & Eickbush, T. H. Reverse transcription of R2Bm RNA is primed by a nick at the chromosomal target site: a mechanism for non-LTR retrotransposition. Cell 72, 595-605 (1993).
Nottingham, R. M. et al. RNA-seq of human reference RNA samples using a thermostable group II intron reverse transcriptase. RNA 22, 597-613 (2016).
Telesnitsky, A. & Goff, S. P. RNase H domain mutations affect the interaction between Moloney murine leukemia virus reverse transcriptase and its primer-template. Proc. Natl. Acad. Sci. U.S.A. 90, 1276-1280 (1993).
Halvas, E. K., Svarovskaia, E. S. & Pathak, V. K. Role of Murine Leukemia Virus Reverse Transcriptase Deoxyribonucleoside Triphosphate-Binding Site in Retroviral Replication and In Vivo Fidelity. Journal of Virology 74, 10349-10358 (2000).
Nowak, E. et al., Structural analysis of monomeric retroviral reverse transcriptase in complex with an RNA/DNA hybrid. Nucleic Acids Res 41, 3874-3887 (2013).
Stamos, J. L., Lentzsch, A. M. & Lambowitz, A. M. Structure of a Thermostable Group II Intron Reverse Transcriptase with Template-Primer and Its Functional and Evolutionary Implications. Molecular Cell 68, 926-939.e4 (2017).
Das, D. & Georgiadis, M. M. The Crystal Structure of the Monomeric Reverse Transcriptase from Moloney Murine Leukemia Virus. Structure 12, 819-829 (2004).
Avidan, O., Meer, M. E., Oz, I. & Hizi, A. The processivity and fidelity of DNA synthesis exhibited by the reverse transcriptase of bovine leukemia virus. European Journal of Biochemistry 269, 859-867 (2002).
Gerard, G. F. et al. The role of template-primer in protection of reverse transcriptase from thermal inactivation. Nucleic Acids Res 30, 3118-3129 (2002).
Monot, C. et al. The Specificity and Flexibility of L1 Reverse Transcription Priming at Imperfect T-Tracts. PLOS Genetics 9, e1003499 (2013).
Mohr, S. et al. Thermostable group II intron reverse transcriptase fusion proteins and their use in cDNA synthesis and next-generation RNA sequencing. RNA 19, 958-970 (2013).

### Nuclear Localization Sequences (NLS)

In various embodiments, the prime editors used in the methods and complexes described herein may comprise one or more nuclear localization sequences (NLS), which help promote translocation of a protein into the cell nucleus. Such sequences are well-known in the art and can include the following examples:

| DESCRIPTION | SEQUENCE | SEQ |
|---|---|---|
| | | ID |
| | | NO: |
| NLS OF SV40 LARGE | PKKKRKV | 94 |
| T-AG | | |
| NLS | MKRTADGSEFESPKKKRKV | 95 |
| NLS | MDSLLMNRRKFLYQFKNVR | 99 |
| | WAKGRRETYLC | |
| NLS OF | AVKRPAATKKAGQAKKKKLD | 100 |
| NUCLEOPLASMIN | | |
| NLS OF EGL-13 | MSRRRKANPTKLSENAKKL | 101 |
| AKEV | EN | |
| NLS OF C-MYC | PAAKRVKLD | 98 |
| NLS OF TUS-PROTEIN | KLKIKRPVK | 102 |
| NLS OF POLYOMA | VSRKRPRP | 103 |
| LARGE T-AG | | |
| NLS OF HEPATITIS D | EGAPPAKRAR | 104 |
| VIRUS ANTIGEN | | |
| NLS OF MURINE P53 | PPQPKKKPLDGE | 105 |
| NLS OF PE1 AND PE2 | SGGSKRTADGSEFEPKKKRKV | 96 |
| BIPARTITE SV40 NLS | KRTADGSEFESPKKKRKV | 97 |

The NLS examples above are non-limiting. The prime editors used in the presently described methods and complexes may comprise any known NLS sequence, including any of those described in Cokol et al., “Finding nuclear localization signals,” EMBO Rep., 2000, 1 (5): 411-415 and Freitas et al., “Mechanisms and Signals for the Nuclear Import of Proteins,” Current Genomics, 2009, 10 (8): 550-7, each of which are incorporated herein by reference.

In various embodiments, the fusion proteins used in the methods and complexes described herein further comprise one or more (and preferably at least two) nuclear localization sequences. In certain embodiments, the fusion proteins comprise at least two NLSs. In embodiments with at least two NLSs, the NLSs can be the same NLSs or they can be different NLSs. In some embodiments, one or more of the NLSs are bipartite NLSs (“bpNLS”). In certain embodiments, the prime editors comprise two bipartite NLSs. In some embodiments, the prime editors comprise more than two bipartite NLSs.

The location of the NLS fusion can be at the N-terminus, the C-terminus, or within a sequence of a fusion protein (e.g., inserted between the encoded napDNAbp component (e.g., Cas9) and a polymerase domain (e.g., a reverse transcriptase).

The NLSs may be any known NLS sequence in the art. The NLSs may also be any future-discovered NLSs for nuclear localization. The NLSs also may be any naturally-occurring NLS, or any non-naturally occurring NLS (e.g., an NLS with one or more desired mutations).

The term “nuclear localization sequence” or “NLS” refers to an amino acid sequence that promotes import of a protein into the cell nucleus, for example, by nuclear transport. Nuclear localization sequences are known in the art and would be apparent to the skilled artisan. For example, NLS sequences are described in Plank et al., International PCT application PCT/EP2000/011690, filed Nov. 23, 2000, published as WO/2001/038547 on May 31, 2001, the contents of which are incorporated herein by reference. In some embodiments, an NLS comprises the amino acid sequence PKKKRKV (SEQ ID NO: 94), MDSLLMNRRKFLYQFKNVRWAKGRRETYLC (SEQ ID NO: 99), KRTADGSEFESPKKKRKV (SEQ ID NO: 97), or KRTADGSEFEPKKKRKV (SEQ ID NO: 106). In other embodiments, an NLS comprises the amino acid sequences NLSKRPAAIKKAGQAKKKK (SEQ ID NO: 107), PAAKRVKLD (SEQ ID NO: 98), RQRRNELKRSF (SEQ ID NO: 108), or NQSSNFGPMKGGNFGGRSSGPYGGGGQYFAKPRNQGGY (SEQ ID NO: 109).

In one aspect of the disclosure, a prime editor or other fusion protein used in the methods and complexes described herein may be modified with one or more nuclear localization sequences (NLS), preferably at least two NLSs. In certain embodiments, the prime editors are modified with two or more NLSs. The disclosure contemplates the use of any nuclear localization sequence known in the art at the time of the disclosure, or any nuclear localization sequence that is identified or otherwise made available in the state of the art after the time of the instant filing. A representative nuclear localization sequence is a peptide sequence that directs the protein to the nucleus of the cell in which the sequence is expressed. A nuclear localization signal is predominantly basic, can be positioned almost anywhere in a protein's amino acid sequence, generally comprises a short sequence of four amino acids (Autieri & Agrawal, (1998) J. Biol. Chem. 273:14731-37, incorporated herein by reference) to eight amino acids, and is typically rich in lysine and arginine residues (Magin et al., (2000) Virology 274:11-16, incorporated herein by reference). Nuclear localization sequences often comprise proline residues. A variety of nuclear localization sequences have been identified and have been used to effect transport of biological molecules from the cytoplasm to the nucleus of a cell. See, e.g., Tinland et al., (1992) Proc. Natl. Acad. Sci. U.S.A. 89:7442-46; Moede et al., (1999) FEBS Lett. 461:229-34, which is incorporated herein by reference. Translocation is currently thought to involve nuclear pore proteins.

Most NLSs can be classified in three general groups: (i) a monopartite NLS exemplified by the SV40 large T antigen NLS (PKKKRKV (SEQ ID NO: 94)); (ii) a bipartite motif consisting of two basic domains separated by a variable number of spacer amino acids and exemplified by the Xenopus nucleoplasmin NLS (KRXXXXXXXXXXKKKL (SEQ ID NO: 110)); and (iii) noncanonical sequences such as M9 of the hnRNP Al protein, the influenza virus nucleoprotein NLS, and the yeast Gal4 protein NLS (Dingwall and Laskey 1991).

Nuclear localization sequences appear at various points in the amino acid sequences of proteins. NLS have been identified at the N-terminus, the C-terminus, and in the central region of proteins. Thus, the disclosure provides fusion proteins that may be modified with one or more NLSs at the C-terminus and/or the N-terminus, as well as at internal regions of the fusion protein. The residues of a longer sequence that do not function as component NLS residues should be selected so as not to interfere, for example, tonically or sterically, with the nuclear localization signal itself. Therefore, although there are no strict limits on the composition of an NLS-comprising sequence, in practice, such a sequence can be functionally limited in length and composition.

The present disclosure contemplates any suitable means by which to modify a prime editor to include one or more NLSs. In one aspect, the prime editors may be engineered to express a prime editor that is translationally fused at its N-terminus or its C-terminus (or both) to one or more NLSs, i.e., to form a prime editor-NLS fusion construct. In other embodiments, a prime editor-encoding nucleotide sequence may be genetically modified to incorporate a reading frame that encodes one or more NLSs in an internal region of the encoded prime editor. In addition, the NLSs may include various amino acid linkers or spacer regions encoded between the prime editor and the N-terminally, C-terminally, or internally attached NLS amino acid sequence, e.g., and in the central region of proteins. Thus, the present disclosure also provides for nucleotide constructs, vectors, and host cells for expressing fusion proteins that comprise a prime editor and one or more NLSs, among other components.

The prime editors used in the methods and complexes described herein may also comprise nuclear localization sequences that are linked to a prime editor through one or more linkers, e.g., a polymeric, amino acid, nucleic acid, polysaccharide, chemical, or nucleic acid linker element. The linkers within the contemplated scope of the disclosure are not intended to have any limitations and can be any suitable type of molecule (e.g., polymer, amino acid, polysaccharide, nucleic acid, lipid, or any synthetic chemical linker domain) and can be joined to the prime editor by any suitable strategy that effectuates forming a bond (e.g., covalent linkage, hydrogen bonding) between the prime editor and the one or more NLSs.

In some embodiments, the prime editors used in the methods provided herein comprise an NLS comprising the amino acid sequence of SEQ ID NO: 95, or an amino acid sequence at least 70%, at least 75%, at least 80%, at least 85%, at least 90%, at least 95%, at least 96%, at least 97%, at least 98%, or at least 99% identical to the amino acid sequence of SEQ ID NO: 95. In some embodiments, the prime editors used in the methods provided herein comprise an NLS comprising the amino acid sequence of SEQ ID NO: 97, or an amino acid sequence at least 70%, at least 75%, at least 80%, at least 85%, at least 90%, at least 95%, at least 96%, at least 97%, at least 98%, or at least 99% identical to the amino acid sequence of SEQ ID NO: 97. In some embodiments, the prime editors used in the methods provided herein comprise an NLS comprising the amino acid sequence of SEQ ID NO: 98, or an amino acid sequence at least 70%, at least 75%, at least 80%, at least 85%, at least 90%, at least 95%, at least 96%, at least 97%, at least 98%, or at least 99% identical to the amino acid sequence of SEQ ID NO: 98. In certain embodiments, the prime editors used in the methods provided herein comprise a first NLS comprising the amino acid sequence of SEQ ID NO: 95, or an amino acid sequence at least 70%, at least 75%, at least 80%, at least 85%, at least 90%, at least 95%, at least 96%, at least 97%, at least 98%, or at least 99% identical to the amino acid sequence of SEQ ID NO: 95, a second NLS comprising the amino acid sequence of SEQ ID NO: 97, or an amino acid sequence at least 70%, at least 75%, at least 80%, at least 85%, at least 90%, at least 95%, at least 96%, at least 97%, at least 98%, or at least 99% identical to the amino acid sequence of SEQ ID NO: 97, and a third NLS comprising the amino acid sequence of SEQ ID NO: 98, or an amino acid sequence at least 70%, at least 75%, at least 80%, at least 85%, at least 90%, at least 95%, at least 96%, at least 97%, at least 98%, or at least 99% identical to the amino acid sequence of SEQ ID NO: 98.

### Linkers

The prime editors used in the methods and complexes described herein may include one or more linkers. As defined above, the term “linker,” as used herein, refers to a chemical group or a molecule linking two molecules or moieties, e.g., a binding domain and a cleavage domain of a nuclease. In some embodiments, a linker joins a gRNA binding domain of an RNA-programmable nuclease and a polymerase (e.g., a reverse transcriptase). In some embodiments, a linker joins a Cas9 nickase and a reverse transcriptase. Typically, the linker is positioned between, or flanked by, two groups, molecules, or other moieties and connected to each one via a covalent bond, thus connecting the two. In some embodiments, the linker is an amino acid or a plurality of amino acids (e.g., a peptide or protein). In some embodiments, the linker is an organic molecule, group, polymer, or chemical moiety. In some embodiments, the linker is 5-100 amino acids in length, for example, 5, 6, 7, 8, 9, 10, 11, 12, 13, 14, 15, 16, 17, 18, 19, 20, 21, 22, 23, 24, 25, 26, 27, 28, 29, 30, 30-35, 35-40, 40-45, 45-50, 50-60, 60-70, 70-80, 80-90, 90-100, 100-150, or 150-200 amino acids in length. Longer or shorter linkers are also contemplated.

The linker may be as simple as a covalent bond, or it may be a polymeric linker many atoms in length. In certain embodiments, the linker is a polypeptide, or amino acid-based. In other embodiments, the linker is not peptide-like. In certain embodiments, the linker is a covalent bond (e.g., a carbon-carbon bond, disulfide bond, carbon-heteroatom bond, etc.). In certain embodiments, the linker is a carbon-nitrogen bond of an amide linkage. In certain embodiments, the linker is a cyclic or acyclic, substituted or unsubstituted, branched or unbranched, aliphatic or heteroaliphatic linker. In certain embodiments, the linker is polymeric (e.g., polyethylene, polyethylene glycol, polyamide, polyester, etc.). In certain embodiments, the linker comprises a monomer, dimer, or polymer of aminoalkanoic acid. In certain embodiments, the linker comprises an aminoalkanoic acid (e.g., glycine, ethanoic acid, alanine, beta-alanine, 3-aminopropanoic acid, 4-aminobutanoic acid, 5-pentanoic acid, etc.). In certain embodiments, the linker comprises a monomer, dimer, or polymer of aminohexanoic acid (Ahx). In certain embodiments, the linker is based on a carbocyclic moiety (e.g., cyclopentane, cyclohexane). In other embodiments, the linker comprises a polyethylene glycol moiety (PEG). In other embodiments, the linker comprises amino acids. In certain embodiments, the linker comprises a peptide. In certain embodiments, the linker comprises an aryl or heteroaryl moiety. In certain embodiments, the linker is based on a phenyl ring. The linker may include functionalized moieties to facilitate attachment of a nucleophile (e.g., thiol, amino) from the peptide to the linker. Any electrophile may be used as part of the linker. Exemplary electrophiles include, but are not limited to, activated esters, activated amides, Michael acceptors, alkyl halides, aryl halides, acyl halides, and isothiocyanates.

In some other embodiments, the linker comprises the amino acid sequence (GGGGS) n (SEQ ID NO: 84), (G) n (SEQ ID NO: 85), (EAAAK), (SEQ ID NO: 86), (GGS) n (SEQ ID NO: 87), (SGGS) n (SEQ ID NO: 81), (XP) n (SEQ ID NO: 88), or any combination thereof, wherein n is independently an integer between 1 and 30, and wherein X is any amino acid. In some embodiments, the linker comprises the amino acid sequence (GGS) n (SEQ ID NO: 87), wherein n is 1, 3, or 7. In some embodiments, the linker comprises the amino acid sequence SGSETPGTSESATPES (SEQ ID NO: 89). In some embodiments, the linker comprises the amino acid sequence SGGSSGGSSGSETPGTSESATPESSGGSSGGS (SEQ ID NO: 90). In some embodiments, the linker comprises the amino acid sequence SGGSGGSGGS (SEQ ID NO: 91). In some embodiments, the linker comprises the amino acid sequence SGGS (SEQ ID NO: 82). In other embodiments, the linker comprises the amino acid sequence SGGSSGGSSGSETPGTSESATPESAGSYPYDVPDYAGSAAPAAKKKKLDGSGSGGSS GGS (SEQ ID NO: 83, 60AA). In some embodiments, the linker comprises the amino acid sequence GGS, GGSGGS (SEQ ID NO: 92), GGSGGSGGS (SEQ ID NO: 93), SGGSSGGSSGSETPGTSESATPESSGGSSGGSS (SEQ ID NO: 80), SGSETPGTSESATPES (SEQ ID NO: 89), or SGGSSGGSSGSETPGTSESATPESAGSYPYDVPDYAGSAAPAAKKKKLDGSGSGGSS GGS (SEQ ID NO: 83).

In certain embodiments, linkers may be used to link any of the peptides or peptide domains or moieties of the invention (e.g., a napDNAbp linked or fused to a reverse transcriptase domain, and/or a napDNAbp linked to one or more NLS). Any of the domains of the fusion proteins used in the systems and methods described herein may also be connected to one another through any of the presently described linkers.

In some embodiments, one or more linkers may be used to join one or more portions of a pegRNA or epegRNA to one another. In certain embodiments, a linker is used to join a structured motif, such as evopreq1, to the 3′ end of an epegRNA. In some embodiments, the linker has been engineered to ensure that the 3′ structured motif (e.g., evopreq1) does not disrupt the activity and/or the structure of the epegRNA. In certain embodiments, such a linker comprises the sequence 5′-AGAATAAA-3′, or a sequence at least 90%, at least 95%, at least 96%, at least 97%, at least 98%, or at least 99% identical to the sequence 5′-AGAATAAA-3′, or a fragment thereof.

In certain embodiments, the prime editors used in the methods, complexes, compositions, and kits provided herein comprise a linker of SEQ ID NO: 79, for example, joining the napDNAbp and the reverse transcriptase to one another. In certain embodiments, the prime editors used in the methods, complexes, compositions, and kits provided herein comprise a linker of SEQ ID NO: 82, for example, joining the reverse transcriptase and an NLS. In certain embodiments, the prime editors used in the methods, complexes, compositions, and kits provided herein comprise a linker of the sequence GSG, for example, joining one NLS to another NLS.

### Complexes

Other aspects of the present disclosure relate to complexes comprising a prime editor and any of the epegRNAs disclosed herein. In some embodiments, the prime editor is PE2, PE3, PE3b, PE4, PE5, or PE5b. In some embodiments, the prime editor comprises PEmax architecture. In some embodiments, the prime editor is PE2max, PE3max, PE3bmax, PE4max, PE5max, or PE5bmax.

### Pharmaceutical Compositions

Other aspects of the present disclosure relate to pharmaceutical compositions for treating sickle cell disease comprising any of the epegRNAs, nicking gRNAs, prime editors, and/or complexes described herein, or any of the polynucleotides or vectors encoding such epegRNAs, prime editors, and/or complexes described herein. The term “pharmaceutical composition”, as used herein, refers to a composition formulated for pharmaceutical use. In some embodiments, the pharmaceutical composition further comprises a pharmaceutically acceptable carrier. In some embodiments, the pharmaceutical composition comprises additional agents (e.g., for specific delivery, increasing half-life, or other therapeutic compounds).

As used herein, the term “pharmaceutically-acceptable carrier” means a pharmaceutically-acceptable material, composition, or vehicle, such as a liquid or solid filler, diluent, excipient, manufacturing aid (e.g., lubricant, talc magnesium, calcium or zinc stearate, or steric acid), or solvent encapsulating material, involved in carrying or transporting the compound from one site (e.g., the delivery site) of the body, to another site (e.g., an organ, tissue, or other part of the body). A pharmaceutically acceptable carrier is “acceptable” in the sense of being compatible with the other ingredients of the formulation and not injurious to the tissue of the subject (e.g., physiologically compatible, sterile, physiologic pH, etc.). Some examples of materials that can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil, and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids; (23) serum component, such as serum albumin, HDL, and LDL; (22) C2-C₁₂ alcohols, such as ethanol; and (23) other non-toxic compatible substances employed in pharmaceutical formulations. Wetting agents, coloring agents, release agents, coating agents, sweetening agents, flavoring agents, perfuming agents, preservatives, and antioxidants can also be present in the formulation. The terms such as “excipient,” “carrier,” “pharmaceutically acceptable carrier,” or the like are used interchangeably herein.

In some embodiments, the pharmaceutical composition is formulated for delivery to a subject, e.g., for gene editing. Suitable routes of administering the pharmaceutical composition described herein include, without limitation: topical, subcutaneous, transdermal, intradermal, intralesional, intraarticular, intraperitoneal, intravesical, transmucosal, gingival, intradental, intracochlear, transtympanic, intraorgan, epidural, intrathecal, intramuscular, intravenous, intravascular, intraosseus, periocular, intratumoral, intracerebral, and intracerebroventricular administration.

In some embodiments, the pharmaceutical composition described herein is administered locally to a diseased site (e.g., tumor site). In some embodiments, the pharmaceutical composition described herein is administered to a subject by injection, by means of a catheter, by means of a suppository, or by means of an implant, the implant being of a porous, non-porous, or gelatinous material, including a membrane, such as a sialastic membrane, or a fiber.

In other embodiments, the pharmaceutical composition described herein is delivered in a controlled release system. In one embodiment, a pump may be used (see, e.g., Langer, 1990, Science 249:1527-1533; Sefton, 1989, CRC Crit. Ref. Biomed. Eng. 14:201; Buchwald et al., 1980, Surgery 88:507; Saudek et al., 1989, N. Engl. J. Med. 321:574). In another embodiment, polymeric materials can be used. (See, e.g., Medical Applications of Controlled Release (Langer and Wise eds., CRC Press, Boca Raton, Fla., 1974); Controlled Drug Bioavailability, Drug Product Design and Performance (Smolen and Ball eds., Wiley, New York, 1984); Ranger and Peppas, 1983, Macromol. Sci. Rev. Macromol. Chem. 23:61. See also Levy et al., 1985, Science 228:190; During et al., 1989, Ann. Neurol. 25:351; Howard et al., 1989, J. Neurosurg. 71:105). Other controlled release systems are discussed, for example, in Langer, supra.

In some embodiments, the pharmaceutical composition is formulated in accordance with routine procedures as a composition adapted for intravenous or subcutaneous administration to a subject, e.g., a human. In some embodiments, pharmaceutical compositions for administration by injection are solutions in sterile isotonic aqueous buffer. Where necessary, the pharmaceutical composition can also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the pharmaceutical composition is to be administered by infusion, it can be dispensed with an infusion bottle containing sterile pharmaceutical grade water or saline. Where the pharmaceutical composition is administered by injection, an ampoule of sterile water for injection or saline can be provided so that the ingredients can be mixed prior to administration.

A pharmaceutical composition for systemic administration may be a liquid, e.g., sterile saline, lactated Ringer's or Hank's solution. In addition, the pharmaceutical composition can be in solid forms and re-dissolved or suspended immediately prior to use. Lyophilized forms are also contemplated.

The pharmaceutical composition can be contained within a lipid particle or vesicle, such as a liposome or microcrystal, which is also suitable for parenteral administration. The particles can be of any suitable structure, such as unilamellar or plurilamellar, so long as compositions are contained therein. Compounds can be entrapped in “stabilized plasmid-lipid particles” (SPLP) containing the fusogenic lipid dioleoylphosphatidylethanolamine (DOPE), low levels (5-10 mol %) of cationic lipid and stabilized by a polyethyleneglycol (PEG) coating (Zhang Y. P. et al., Gene Ther. 1999, 6:1438-47). Positively charged lipids such as N-[1-(2,3-dioleoyloxi) propyl]-N,N,N-trimethyl-amoniummethylsulfate, or “DOTAP,” are particularly preferred for such particles and vesicles. The preparation of such lipid particles is well known. See, e.g., U.S. Pat. Nos. 4,880,635; 4,906,477; 4,911,928; 4,917,951; 4,920,016; and 4,921,757; each of which is incorporated herein by reference.

The pharmaceutical compositions described herein may be administered or packaged as a unit dose, for example. The term “unit dose” when used in reference to a pharmaceutical composition of the present disclosure refers to physically discrete units suitable as unitary dosage for the subject, each unit containing a predetermined quantity of active material calculated to produce the desired therapeutic effect in association with the required diluent; i.e., carrier, or vehicle.

Further, the pharmaceutical composition can be provided as a pharmaceutical kit comprising (a) a container containing a compound of the invention in lyophilized form and (b) a second container containing a pharmaceutically acceptable diluent (e.g., sterile water) for injection. The pharmaceutically acceptable diluent can be used for reconstitution or dilution of the lyophilized compound of the invention. Optionally associated with such container(s) can be a notice in the form prescribed by a governmental agency regulating the manufacture, use, or sale of pharmaceuticals or biological products, which notice reflects approval by the agency of manufacture, use, or sale for human administration.

In another aspect, an article of manufacture containing materials useful for the treatment of the diseases described above is included. In some embodiments, the article of manufacture comprises a container and a label. Suitable containers include, for example, bottles, vials, syringes, and test tubes. The containers may be formed from a variety of materials such as glass or plastic. In some embodiments, the container holds a composition that is effective for treating a disease and may have a sterile access port. For example, the container may be an intravenous solution bag or a vial having a stopper pierce-able by a hypodermic injection needle. The active agent in the composition is a compound of the invention. In some embodiments, the label on or associated with the container indicates that the composition is used for treating the disease of choice. The article of manufacture may further comprise a second container comprising a pharmaceutically acceptable buffer, such as phosphate-buffered saline, Ringer's solution, or dextrose solution. It may further include other materials desirable from a commercial and user standpoint, including other buffers, diluents, filters, needles, syringes, and package inserts with instructions for use.

### Polynucleotides, Vectors, Kits, and Cells

In some aspects, the present disclosure provides polynucleotides and vectors encoding any of the epegRNAs, nicking gRNAs, and/or prime editors provided herein. In some aspects, the present disclosure provides one or more polynucleotides and vectors encoding any of the complexes provided herein. In some embodiments, the polynucleotides and vectors provided herein comprise DNA. In some embodiments, the polynucleotides and vectors provided herein comprise RNA. In some embodiments, the present disclosure provides mRNA polynucleotides for producing any of the prime editors described herein. In some aspects, any of the polynucleotides described herein may be provided in a vector.

The epegRNAs, nicking gRNAs, prime editors, complexes, polynucleotides, and/or vectors of the present disclosure may be assembled into kits. In some embodiments, the kit comprises polynucleotides for expression of any of the epegRNAs, nicking gRNAs, prime editors, and/or complexes provided herein. In other embodiments, the kit further comprises appropriate epegRNAs or nucleic acid vectors for the expression of such epegRNAs, to target the Cas9 protein of the prime editors to the desired target sequence, e.g., in the β-globin (HBB) gene for the treatment of sickle cell disease.

The kits described herein may include one or more containers housing components for performing the methods described herein, and optionally instructions for use. In some embodiments, the kits include instructions for editing the HBB gene. In some embodiments, the kits include instructions for editing the HBB gene in a cell (e.g., in an HSPC). Any of the kits described herein may further comprise components needed for performing any of the methods described herein (e.g., for editing HBB and/or for treating sickle cell disease, for example, by correcting an A·T:T·A transversion mutation at nucleotide position 20 in HBB). Each component of the kits, where applicable, may be provided in liquid form (e.g., in solution) or in solid form, (e.g., a dry powder). In certain cases, some of the components may be reconstitutable or otherwise processible (e.g., to an active form), for example, by the addition of a suitable solvent or other species (for example, water), which may or may not be provided with the kit.

In some embodiments, the kits may optionally include instructions and/or promotion for use of the components provided. As used herein, “instructions” can define a component of instruction and/or promotion, and typically involve written instructions on or associated with packaging of the disclosure. Instructions also can include any oral or electronic instructions provided in any manner such that a user will clearly recognize that the instructions are to be associated with the kit, for example, audiovisual (e.g., videotape, DVD, etc.), internet, and/or web-based communications, etc. The written instructions may be in a form prescribed by a governmental agency regulating the manufacture, use, or sale of pharmaceuticals or biological products, which can also reflect approval by the agency of manufacture, use, or sale for animal administration. As used herein, “promoted” includes all methods of doing business including methods of education, hospital and other clinical instruction, scientific inquiry, drug discovery or development, academic research, pharmaceutical industry activity including pharmaceutical sales, and any advertising or other promotional activity including written, oral, and electronic communication of any form, associated with the disclosure. Additionally, the kits may include other components depending on the specific application, as described herein.

The kits may contain any one or more of the components described herein in one or more containers. The components may be prepared sterilely, packaged in a syringe, and shipped refrigerated. Alternatively, they may be housed in a vial or other container for storage. A second container may have other components prepared sterilely. Alternatively, the kits may include the active agents premixed and shipped in a vial, tube, or other container.

The kits may have a variety of forms, such as a blister pouch, a shrink-wrapped pouch, a vacuum sealable pouch, a sealable thermoformed tray, or a similar pouch or tray form, with the accessories loosely packed within the pouch, one or more tubes, containers, a box, or a bag. The kits may be sterilized after the accessories are added, thereby allowing the individual accessories in the container to be otherwise unwrapped. The kits can be sterilized using any appropriate sterilization techniques, such as radiation sterilization, heat sterilization, or other sterilization methods known in the art. The kits may also include other components, depending on the specific application, for example, containers, cell media, salts, buffers, reagents, syringes, needles, a fabric, such as gauze, for applying or removing a disinfecting agent, disposable gloves, a support for the agents prior to administration, etc. Some aspects of this disclosure provide kits comprising a nucleic acid construct comprising a nucleotide sequence encoding the prime editor systems described herein, or various components thereof (e.g., the epegRNAs, prime editors, complexes, polynucleotides, and/or vectors provided herein). In some embodiments, the nucleotide sequence(s) comprises a heterologous promoter (or more than a single promoter) that drives expression of the prime editor system components.

Cells that may contain any of the epegRNAs, complexes, polynucleotides, and/or vectors described herein include prokaryotic cells and eukaryotic cells. The methods described herein may be used to deliver an epegRNA and a prime editor into a eukaryotic cell (e.g., a mammalian cell, such as a human cell). In some embodiments, mRNA for producing the prime editor is delivered to the cell. In certain embodiments, the cell is a hematopoietic stem or progenitor cell (HSPC). In some embodiments, the cell is in vitro (e.g., a cultured cell). In some embodiments, the cell is in vivo (e.g., in a subject, such as a human subject). In some embodiments, the cell is ex vivo (e.g., isolated from a subject and may be administered back to the same or a different subject). In certain embodiments, a cell (e.g., an HSPC) is isolated from a subject, edited using the methods described herein, and delivered back into the subject, for example, in order to treat sickle cell disease by editing the DNA in the cell to correct one or more mutations in the HBB gene, e.g., an A·T:T·A transversion mutation at nucleotide position 20 in HBB.

Some aspects of this disclosure provide cells comprising any of the constructs disclosed herein. In some embodiments, a host cell is transiently or non-transiently transfected or electroporated with one or more vectors described herein. In some embodiments, a cell is transfected or electroporated as it naturally occurs in a subject. In some embodiments, a cell that is transfected or electroporated is taken from a subject. In some embodiments, the cell is derived from cells taken from a subject, such as a cell line. In some embodiments, cells (e.g., HSPCs) are electroporated to introduce an epegRNA and mRNA for producing a prime editor as described herein.

Some aspects of this disclosure provide cells comprising DNA comprising the sequence 5′-ATGGTGCACCTGACTCCTGAAGAGAAG-3′ (SEQ ID NO: 78) (e.g., in the chromosomal DNA of the cell). In some embodiments, a cell comprises DNA comprising a sequence at least 90%, at least 95%, at least 96%, at least 97%, at least 98%, or at least 99% identical to the sequence 5′-ATGGTGCACCTGACTCCTGAAGAGAAG-3′ (SEQ ID NO: 78), wherein the underlined nucleotide is an A (i.e., the PAM-disrupting mutation introduced into the genome along with the corrected amino acid substitution as described herein). In certain embodiments, the cell is an HSPC. In certain embodiments, the cell is an isolated cell.

The function and advantage of these and other embodiments of the present invention will be more fully understood from the Examples below. The following Examples are intended to illustrate the benefits of the present invention and to describe particular embodiments but are not intended to exemplify the full scope of the invention. Accordingly, it will be understood that the Examples are not meant to limit the scope of the invention.

### EXAMPLES

### Example 1. Ex Vivo Prime Editing Reverts the Sickle-Cell Allele in Hematopoietic Stem Cells and Corrects Disease Phenotypes in Mice

Sickle cell disease (SCD), a monogenic disorder affecting millions of individuals, is caused by an A·T-to-T·A transversion mutation in the β-globin gene (HBB)¹. As described herein, a prime editing² system was developed and optimized to directly revert the SCD allele (HBB^{S}) to the wild-type allele (HBB^{A}) with high ratios of desired edit to indel byproducts. Sickle-cell patient hematopoietic stem and progenitor cells (HSPCs) electroporated with PEmax mRNA³, an engineered pegRNA (epegRNA) 4, and a nicking single-guide RNA (sgRNA) yielded up to 41% conversion of HBB^{S} to HBB^{A}. Seventeen weeks after transplantation of prime edited HSPCs into immunodeficient mice, high-level correction of HBB^{S} was maintained in all donor derived cells, with no alterations detected in the frequency of engraftment, hematopoietic differentiation, or lineage maturation compared to unedited healthy donor HSPCs. On average, 42% of human erythroblasts and reticulocytes isolated 17 weeks after transplantation of prime-edited HSPCs from four SCD patient donors contained monoallelic or biallelic HBB correction, exceeding the levels predicted to be required for therapeutic benefit in patients^{5,6}. Red blood cells derived from prime-edited SCD HSPCs contained significantly reduced levels of sickle hemoglobin (HbS), had a proportionate increase in normal adult hemoglobin (HbA) of up to 42%, and were resistant to hypoxia-induced sickling. An extensive analysis of over 100 candidate off-target sites nominated by an unbiased genome-wide experimental approach detected minimal off-target editing from all prime editing components used for the corrective edit. These results represent one of the first PE-based therapeutic strategies for HSCs, suggesting the potential of a one-time treatment for sickle cell disease that directly corrects pathogenic HBB^{S} to wild-type HBB^{A}, does not require delivery of any viral or non-viral DNA template, and minimizes undesired consequences associated with DNA double-strand breaks.

### Results

### Optimizing Prime Editing Systems for HSPCs

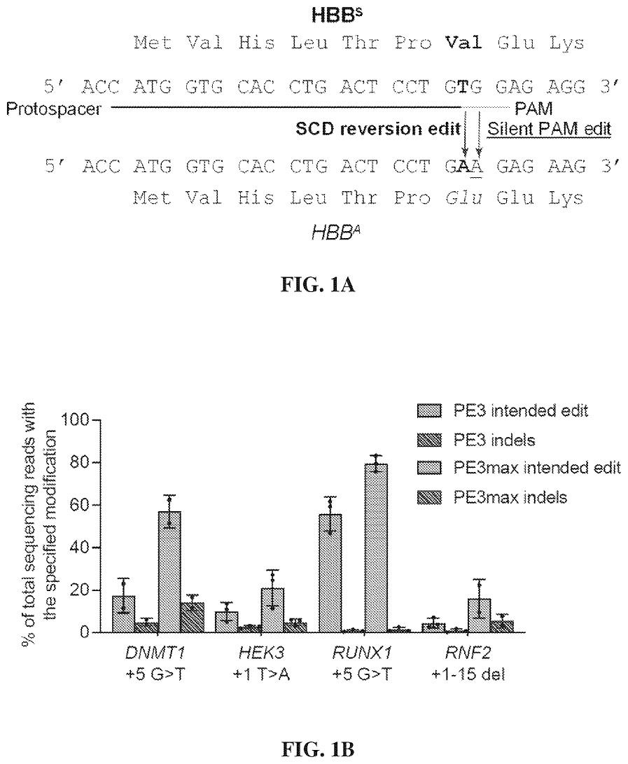

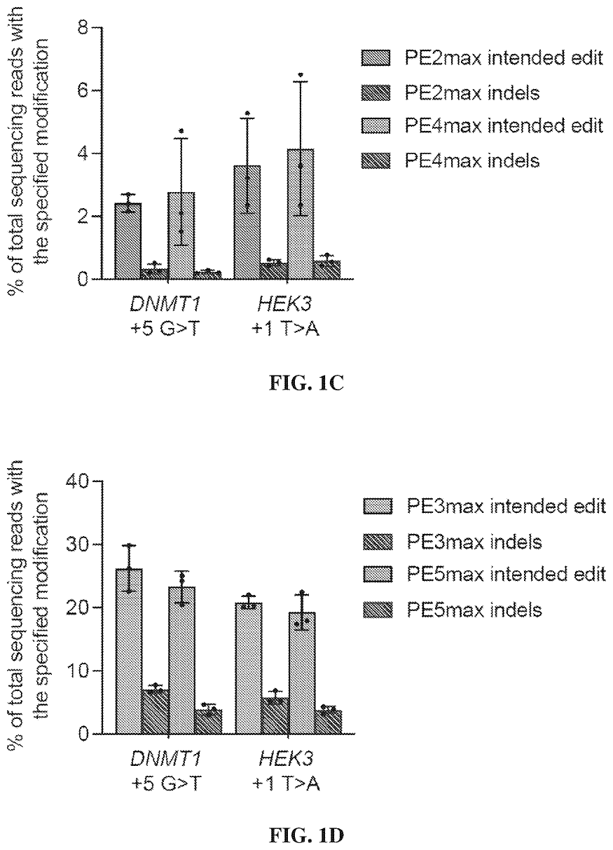

The use of prime editing to correct HBB^{S} by plasmid transfection in HEK293T cells containing the SCD mutation has previously been reported, reaching up to 58% efficiency (FIG. 1A) 2. In contrast to HEK293T cells, HSPCs are difficult to transfect with plasmid DNA but are amenable to RNA electroporation, an ex vivo delivery method that has been used in the clinic to manipulate HSPCs prior to transplantation^{11,12,33}. To that end, an RNA electroporation-based prime editing strategy for HSPCs that incorporates recent prime editing advances was sought. Recently, improved engineered pegRNAs (epegRNAs) 4 that incorporate a 3′ structured motif to protect the reverse transcriptase template (RTT) from exonuclease degradation and an 8-nt linker that hinders potential interference between the motif and the RTT were designed. In addition, PEmax, an improved prime editor architecture with an optimized Cas9 sequence and nuclear localization sequence, and codon usage, was developed³. Editing outcomes were compared following the electroporation of in vitro transcribed PE and PEmax mRNA with synthetic epegRNAs in healthy human donor HSPCs to install various edits across four genomic loci—DNMT1, HEK293T genomic site 3 (hereafter referred to as HEK3), RUNX1, and RNF2 (Tables 1-2). Both PE and PEmax mRNA together with epegRNAs and nicking single-guide (sgRNAs) supported substantial prime editing efficiencies in HSPCs (up to 80%), with PEmax conferring 1.3-to-3.5-fold average increases in editing efficiency over PE (FIG. 1B). Thus, PEmax mRNA can be electroporated together with synthetic epegRNAs and nicking sgRNAs to robustly edit primary human HSPCs.

In addition to the improved PEmax architecture, it was previously reported that prime editing outcomes can be improved by inhibiting mismatch repair using transient co-expression of MLH1dn, a dominant negative MLH1 variant³, or by installing benign or silent mutations near the desired edit that cause the prime editing intermediate to naturally evade mismatch repair. PE4 and PE5 systems transiently coexpress MLH1dn with PE2 and PE3, respectively³. Impeding mismatch repair increases prime editing efficiencies for small substitutions, insertions, and deletions and decreases the formation of indel byproducts in a wide variety of cells^{3,34}. To determine if PE4max or PE5max might increase desired editing outcomes over PE2max and PE3max in primary human HSPCs, in vitro-transcribed RNA encoding all prime editing components and MLH1dn were electroporated into healthy donor cells. Surprisingly, transient MLH1 expression did not improve prime editing over PE2max or PE3max in HSPCs (FIGS. 1C-1D). The +5 G>T edit at DNMT1 is a silent PAM alteration that may help the prime editing intermediate natively evade mismatch repair, without MLH1dn.

However, the intermediate mismatch to install the +1 HEK3 T>A is a known substrate for MMR3, and the prime editing efficiency in HSPCs of +1 HEK3 T>A also does not significantly benefit from the addition of MLH1dn (FIGS. 1C-1D). These observations suggest that cellular mismatch repair may not limit prime editing in HSPCs. Therefore, optimizations using prime editing systems with epegRNAs and PE3max were continued without MLH1dn.

Optimization of Prime Editing Agents to Revert HBB^{S} to HBB^{A}

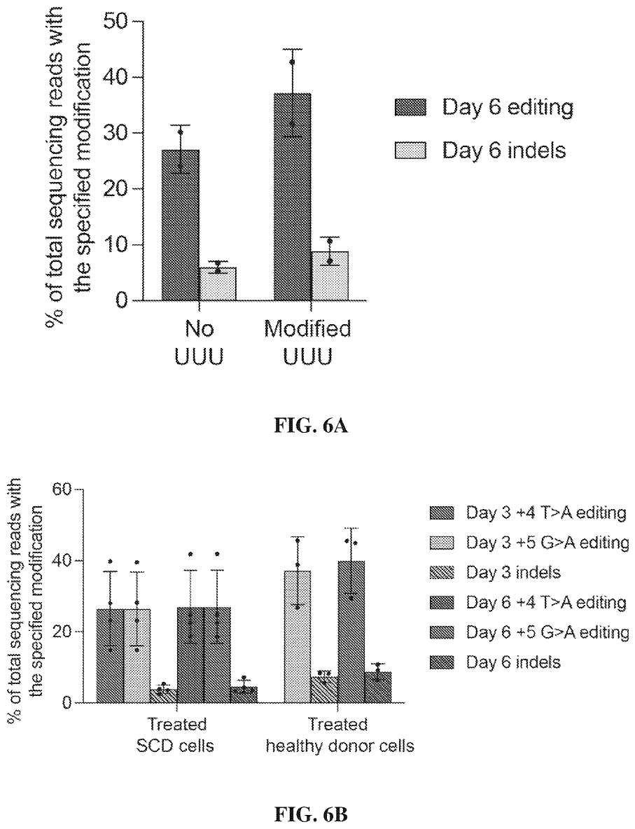

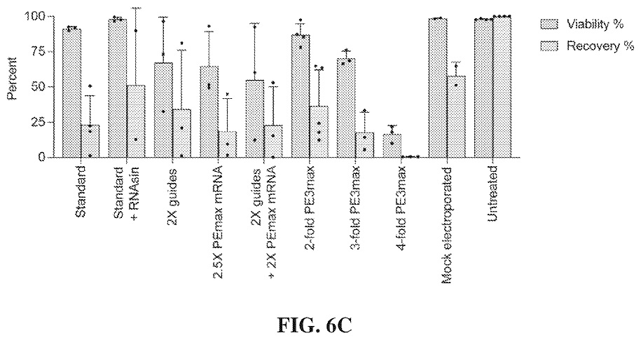

Next, editing outcomes were optimized at the HBB^{S} locus in primary human CD34⁺ cells. An epegRNA was designed that would both revert the pathogenic sickle mutation and also allow optimization in healthy (homozygous HBB^{A}) donor CD34⁺ cells by including a silent PAM edit. First, the 5′ G required for efficient guide expression from plasmids was removed, and an 8-nt linker designed by pegLIT, a computational tool that identifies non-interfering nucleotide linkers between pegRNAs and 3′ epegRNA motifs, was included⁴. Additionally, a UUU trinucleotide was appended to the 3′ end of the epegRNA for additional protection from degradation, with each uracil harboring a 2′-O-methyl modification and with three phosphorothioate linkages, one before each 3′ uracil nucleotide⁴. On average, epegRNAs containing this modified trinucleotide conferred a 1.4-fold increase in prime editing efficiency with a similar product purity compared to epegRNAs lacking the 3′ modified UUU (FIG. 6A). A silent, PAM-disrupting edit was also included in the epegRNA (+5 G>A), which prevents the reengagement of target DNA after prime editing and serves as a marker to assess prime editing efficiencies in healthy donor CD34⁺ cells that lack the pathogenic+4 T>A HBB^{S} mutation.

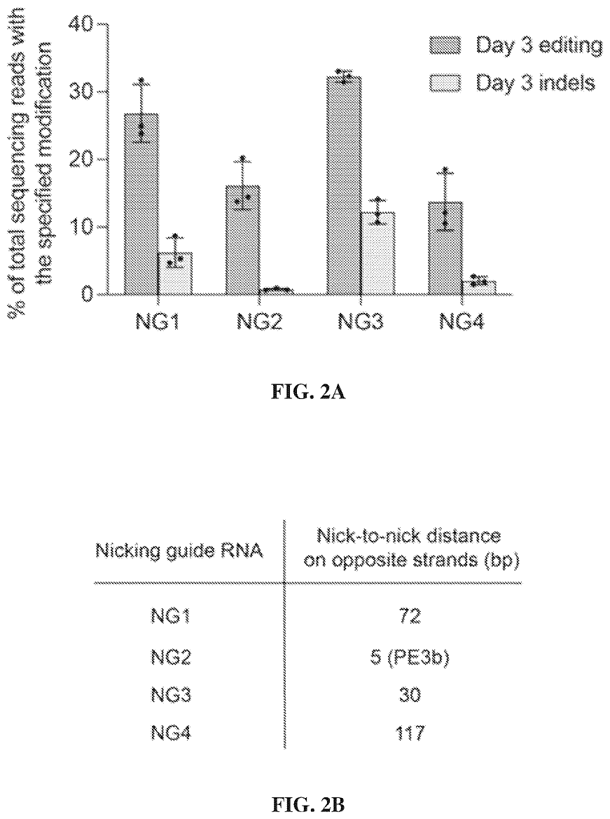

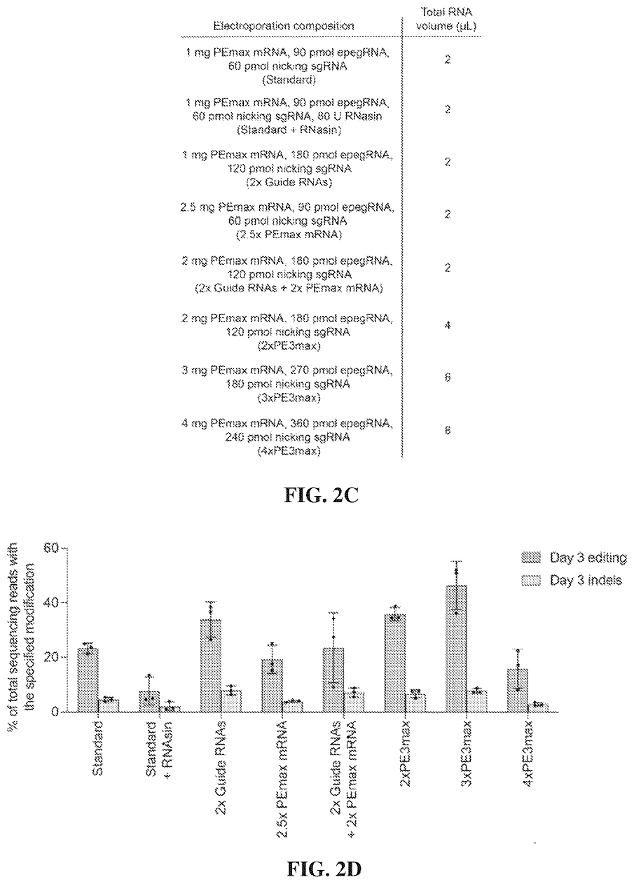

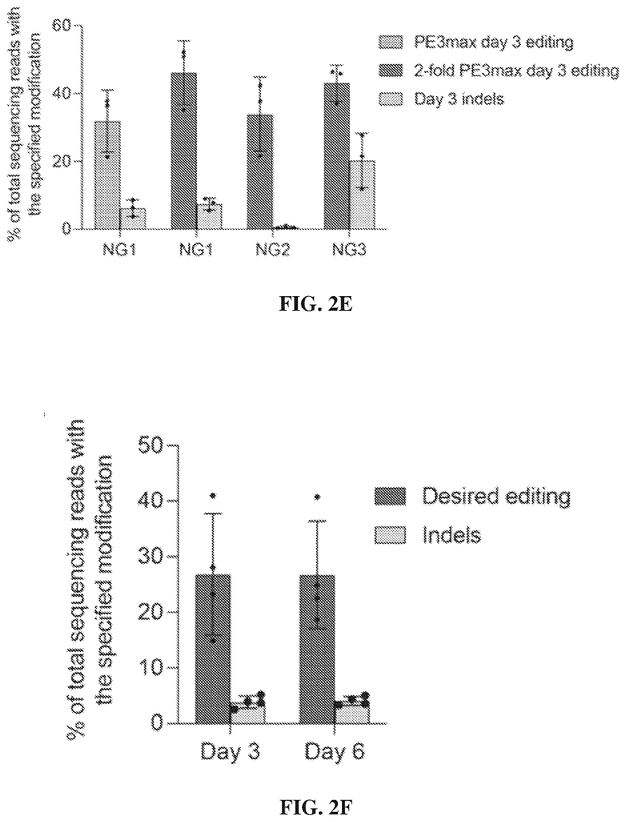

The newly designed synthetic epegRNA was electroporated together with in vitro-transcribed PEmax mRNA and synthetic nicking sgRNA NG1 (constituting a PE3max system) into healthy human donor HSPCs (FIG. 2A). Genomic DNA was harvested from treated cells (without enriching for transfected cells) three days following electroporation, and on-target editing was assessed by high-throughput sequencing (HTS). The desired precise prime editing outcome was observed without any indels or other unwanted target site changes at an average efficiency of 27% (FIG. 2A). This observation demonstrated that synthetic epegRNAs containing a modified 3′ UUU trinucleotide support substantial prime editing efficiencies in primary human CD34⁺ cells.

To further optimize the PE3max system, multiple HBB-nicking sgRNAs were screened (NGs, FIGS. 2A-2B). Three additional NGs were tested, including a PE3b nicking sgRNA that cannot nick the unedited strand until after the desired prime edit has occurred on the opposite strand. It was previously established that PE3b editing strategies minimize indel byproducts from prime editing by reducing the frequency of intermediates containing simultaneous nicks in both DNA strands². Indel formation at this target site can eliminate HBB expression or create non-functional mutant proteins. Indeed, the PE3b nicking sgRNA (NG2), yielded the best ratio of desired edit to indel byproducts, with 16±3.5% desired editing and only 0.75±0.16% indels 3 days post-electroporation (FIG. 2A). However, desired on-target editing using NG2 was lower than the original nicking sgRNA (NG1), which resulted in 27±4.3% desired editing and 6.2±2.2% indels. While NG3 resulted in higher on-target editing (32±0.81%), this increased editing efficiency was accompanied by higher levels of indels (12±1.8%). NG4 achieved only 14±4.3% editing with 2.0±0.58% indels. Together, these results establish that the choice of the nicking sgRNA substantially impacts HBB^{S} prime editing outcomes.

### Optimizing Ratios and Volumes of Electroporation Editing Reagents

Next, efficiency bottlenecks were identified in ex vivo prime editing of HSPCs, and the optimal ratio of PEmax mRNA to synthetic epegRNA and nicking sgRNA was determined. Keeping the guide RNAs and PEmax mRNA at 10% of the total electroporation volume following to the Lonza 4-D manufacturer's recommendation, the concentration of guide RNAs, PEmax mRNA, or both was doubled (FIG. 2C). Additionally, the total volume of all RNAs added per electroporation was doubled, tripled, or quadrupled beyond the manufacturer's suggestion. Finally, the addition of RNasin was tested, which has been previously reported to increase the efficiency of RNA-based electroporations by inhibiting endogenous RNase activity³⁵ (FIG. 2C).

Among these variables, it was found that increasing the total volume of editing reagents, but not changing the ratio of PEmax mRNA: guide RNAs, or adding RNasin, had the largest effect on prime editing outcomes. The standard volumes of PEmax mRNA and guide RNAs resulted in 23% desired editing without other target site changes. However, increasing the volume of the delivered RNAs to 2-fold higher than the amount recommended by Lonza increased on-target editing to 36% (FIG. 2D). A 3-fold increase of RNA editing reagents over the recommended volumes further increased on-target editing to 46%. However, cell viability decreased from 87% to 70% (FIG. 6B). A 4-fold increase over the suggested volume of RNAs substantially reduced editing efficiency to 16% and greatly reduced cell viability to 17%. All other conditions tested failed to outperform the 2-fold increase of delivered RNAs. These results show that editing efficiency can be enhanced by optimizing the ratio between the in vitro transcribed PEmax mRNA and the synthetic guide RNAs, and that increasing the volume of editor reagents above 20% of the total volume of electroporation harms cell viability. In light of these results, 20% by volume PEmax mRNA and guide RNAs were used for all subsequent electroporation experiments.

To determine whether 20% by volume of PE3max could improve editing with different nicking guides (NG1-3), each nicking sgRNA was tested and compared (FIG. 2E). Increased desired editing was observed using the original nicking sgRNA, NG1 (46±9.5% desired editing with no other target site changes) with modest indel frequency (7.4±1.8%); NG2, the PE3b nicking sgRNA, (34±11%) with minimal indels (0.61±0.36%); and NG3 (43±5.3%) with high indel levels (20±8%). Together, these results indicate that the improvement in editing efficiency from using a 2× volume of reagents in the electroporation reaction is applicable across multiple nicking sites. Since the frequency of indel-free on-target editing (46%) was the highest for NG1, this nicking sgRNA, the epegRNA optimized above, and in vitro transcribed PEmax mRNA were used to directly revert the HBB^{S} allele in HSPCs from SCD patients.

### HBB^{S} Correction in SCD Patient HSPCs

For prime editing of patient HSPCs, cryopreserved Plerixafor-mobilized CD34⁺ cells from three SCD patient donors, or CD34⁺ cells isolated from cryopreserved bone marrow from two additional SCD patient donors were thawed and then allowed to recover for 24 hours. Next, the patient cells and healthy donor HSPCs were electroporated in parallel using the optimized PE3max system. 100,000 cells were maintained in culture to extract genomic DNA at days 3 and 6, and the remaining cells were cryopreserved for mouse engraftment experiments. Edited CD34⁺ cells from four different SCD patient donors showed an average of 26±10% desired prime editing of HBB^{S} to wild-type HBB^{A} by day 3 and 27±10% editing by day 6 (FIG. 2F). Compared to these samples, CD34⁺ HSPCs from a fifth SCD patient exhibited poor editing with extensive cell aggregation at day 3 and were not carried forward into subsequent experiments.

Indel frequencies in prime-edited HSPCs remained low, averaging 3.9±1.2% and 4.6±1.8% on days 3 and 6 respectively, reinforcing that prime editing leads to much fewer indels and much higher desired edit-to-indel ratios (5.9-6.7) than current HBB^{S} correction strategies that utilize Cas9 nuclease-HDR, which have reported indel frequencies of 28-45% and edit-to-indel ratios of 0.74-1.6^{2,17,18}. The most efficiently edited patient HSPCs exhibited 41% HBB^{S}-to-HBB^{A} correction, with 5.2% indels at day 3, representing an edit-to-indel ratio of 7.9. Editing efficiency for the silent, PAM-disrupting +5 G>A edit was nearly identical to that of the +4 T>A reversion edit for all SCD patient donors, consistent with the processive mechanism of prime editing² (FIG. 6C). Thus, the optimized prime editing system described herein robustly reverts the HBB^{S} allele back to wild-type HBB^{A} in SCD patient HSPCs with high reversion to indel ratios.

Transplantation of Prime-Edited Human HSPCs into Mice

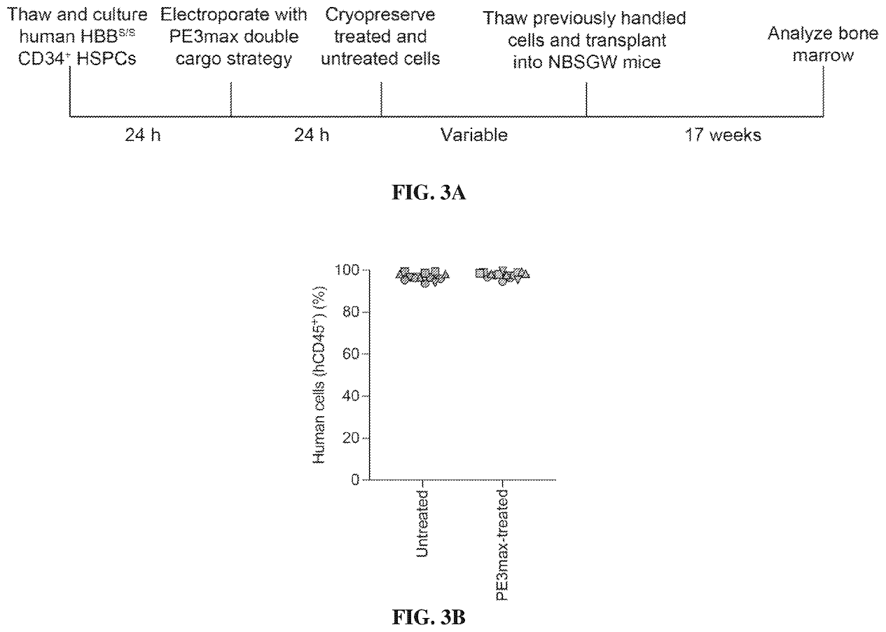

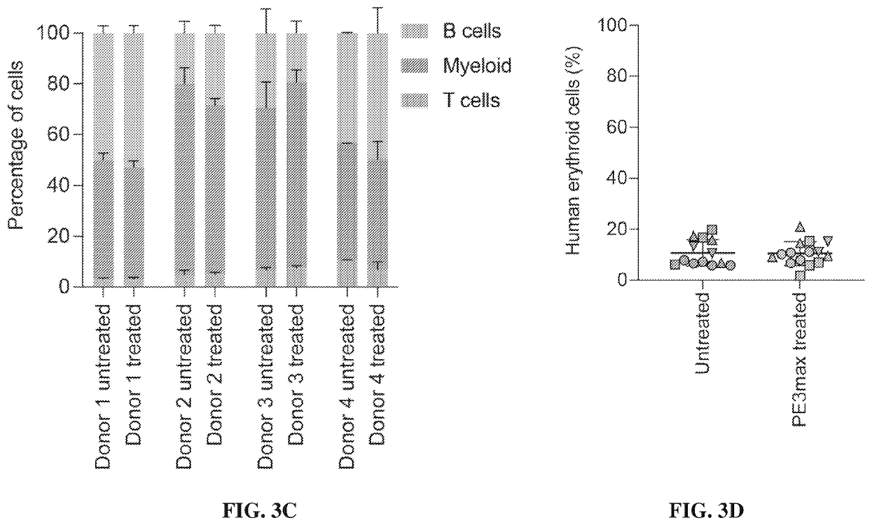

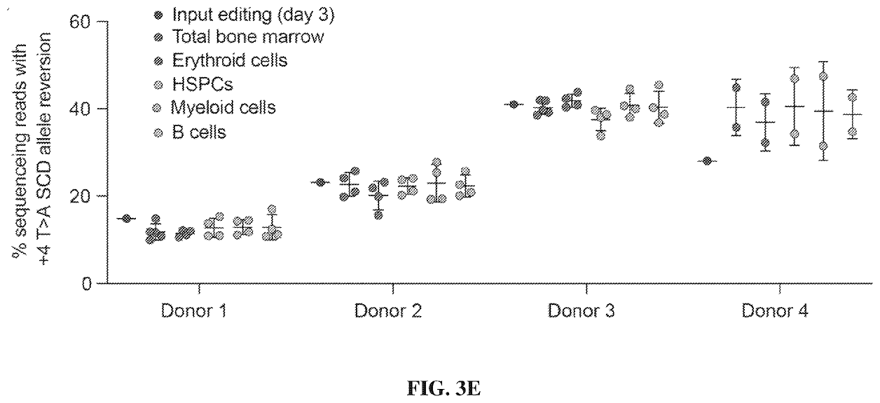

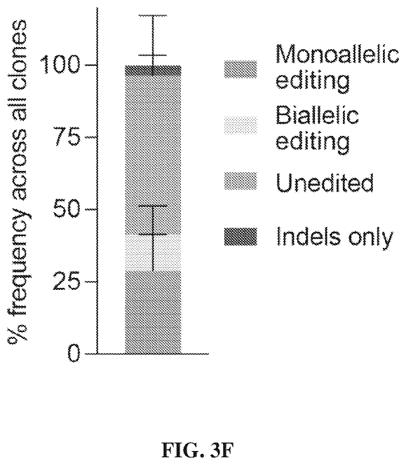

To determine whether prime-edited HSPCs from SCD donors can repopulate bone marrow in vivo, cryopreserved prime-edited and untreated HSPCs from four SCD donors were thawed, then each was transplanted via tail-vein injection into 2-5 immunodeficient NOD B6.SCID Il2rγ^{-/-}Kit^{W41/W41} (NBSGW) mice, which were pretreated with low-dose busulfan 2 days prior to injection to enhance donor HSC engraftment³⁶ (FIG. 3A). The bone marrow of the mice was harvested for analysis 17 weeks post-injection, a time when most or all remaining human cells have been demonstrated to be derived from bone marrow-repopulating HSCs³⁷.

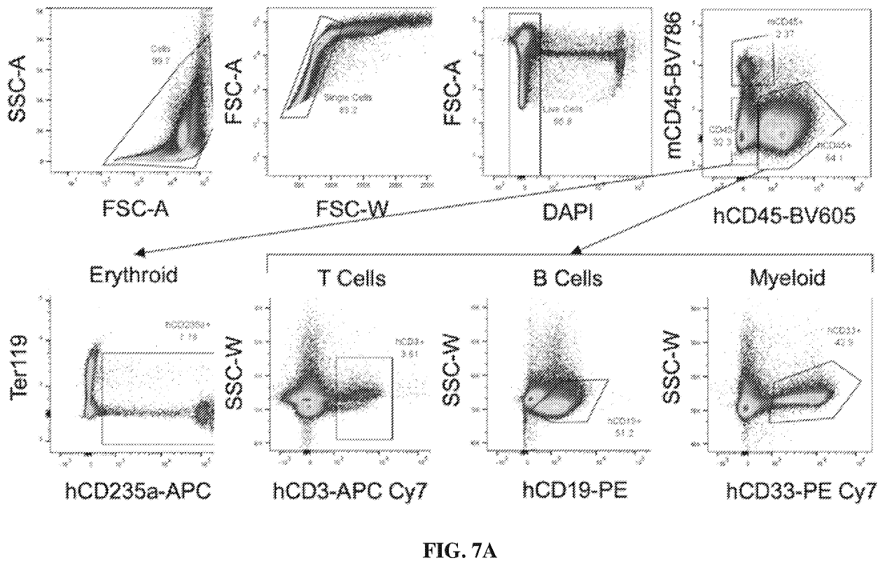

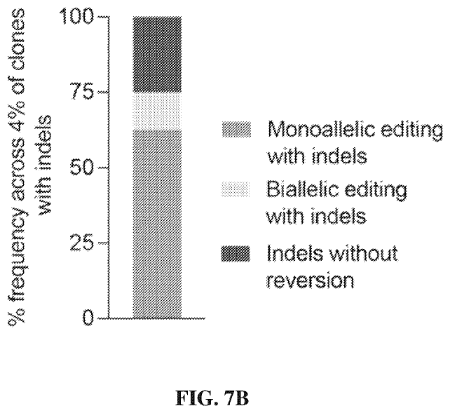

The engraftment, expansion, and differentiation of human HSCs can be altered by genome editing^{38,39}. To investigate whether these parameters were affected by prime editing of HBB^{S}, flow cytometry with human-specific antibodies was used to quantify human donor cells in recipient mouse bone marrow. Human cells expressing the CD45 hematopoietic antigen represented approximately 98% of all bone marrow cells in recipient mice (FIG. 3B), in which the prime-edited cells engrafted with efficiencies comparable to untreated cells, indicating that there was no engraftment impairment, in contrast with nuclease-initiated HDR methods¹⁸. The frequencies of human B-cells (CD19⁺), myeloid cells (CD33⁺), T cells (CD3⁺), and erythroid cells (CD235a⁺) were similar in bone marrow reconstituted with prime-edited HSPCs and untreated control HSPCs (FIGS. 3C-3D, Table 3). Next, lineage-specific antibodies were used to purify SCD patient donor mononuclear cells (CD45⁺, “total bone marrow”), erythroblasts (CD235a⁺), HSPCs (CD34⁺), myeloid cells (CD33⁺), and B cells (CD19⁺) (Table 3, FIG. 7A), and the frequency of HBB^{S} reversion across all lineages was quantified for each of the four donors. The prime editing frequencies of the injected cell population (15 to 41%) largely matched the levels of editing across all lineages (12±0.62 to 40±1.6%) recovered at 17 weeks post-transplantation (FIG. 3E). Together, these findings indicated that prime editing is retained at high frequency in bone marrow-repopulating HSCs, which remains a challenge for some editing strategies that require DSBs¹⁸. Moreover, prime editing did not appear to alter HSC differentiation or maintenance of the lineages examined after bone marrow transplantation.

Of note, one donor achieved higher editing levels in all human cell populations harvested from mouse bone marrow compared to input editing levels at day 3 (donor 4, FIG. 3E). This engrafted donor was the only one in which CD34⁺ cells were isolated from cryopreserved bone marrow, while all HSPC populations were collected from Plerixafor-mobilized blood. It is tempting to speculate that the propensity of HSCs for prime editing may vary according to the HSPC source. Although the editing frequencies of repopulating cells was higher than that of input cells for this donor, all donor-derived lineages present in recipient bone marrow exhibited similar editing frequencies (FIG. 3E), consistent with the findings that PE-mediated conversion of HBB^{S} to HBB^{A} in bone marrow-repopulating HSCs can be as efficient as what occurs in the bulk HSPC population, and that prime editing does not impact HSC lineage outcomes.

Next, clonal editing outcomes among engrafted cells in mice were determined at 17 weeks post-transplantation. Human HSPCs (CD34⁺) and CD235a⁺ cells were isolated from the bone marrow of PE3max treated or untreated mice via magnetic-activated cell sorting (MACS) and seeded into semi-solid methylcellulose medium to generate clonal burst-forming unit-erythroid (BFU-E) colonies. 454 colonies distributed approximately equally from mice transplanted were isolated with four different HSPC donors. On average, 42% of clones harbored the reversion edit in one (29%) or both (13%) HBB^{S} alleles without any target site indels, and 54% were unedited (FIG. 3F). The remaining 4% of clones had indels in at least one allele, with 75% of those colonies also containing an intact allele with the desired edit (FIG. 7B). These clonal editing outcomes reveal that PE3max treated cells are corrected at a level that substantially exceeds the 20% correction thought to be therapeutic in SCD patients^{5,6}. Overall, these results demonstrate that prime-edited cells support hematopoietic repopulation and sustain predicted therapeutic levels of editing in long-term HSC populations.

Prime Editing Corrects SCD Characteristics in Red Blood Cells from Transplanted Human HSCs

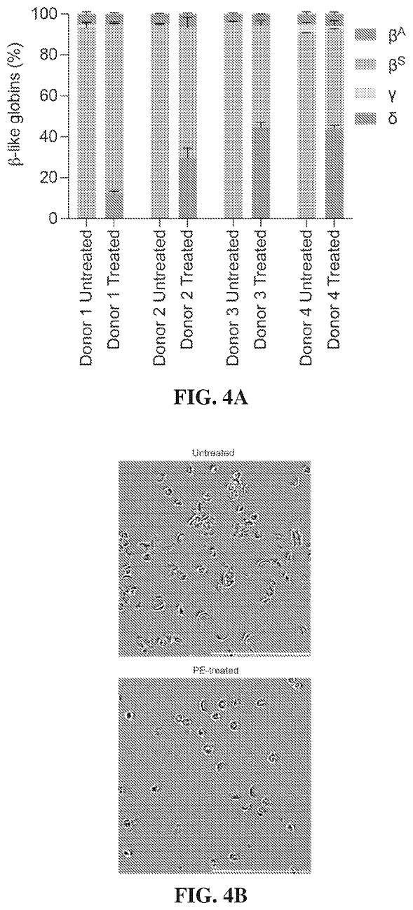

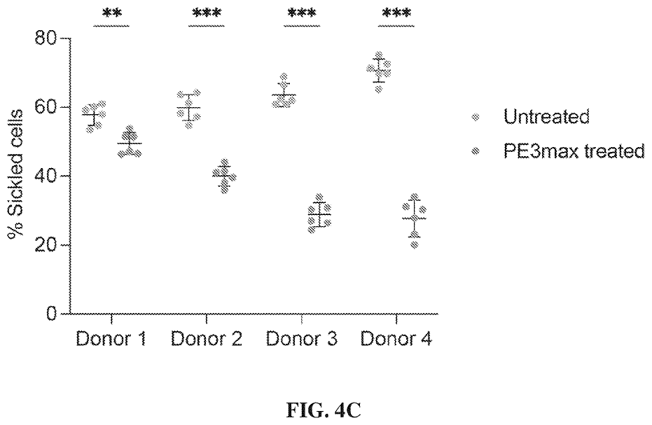

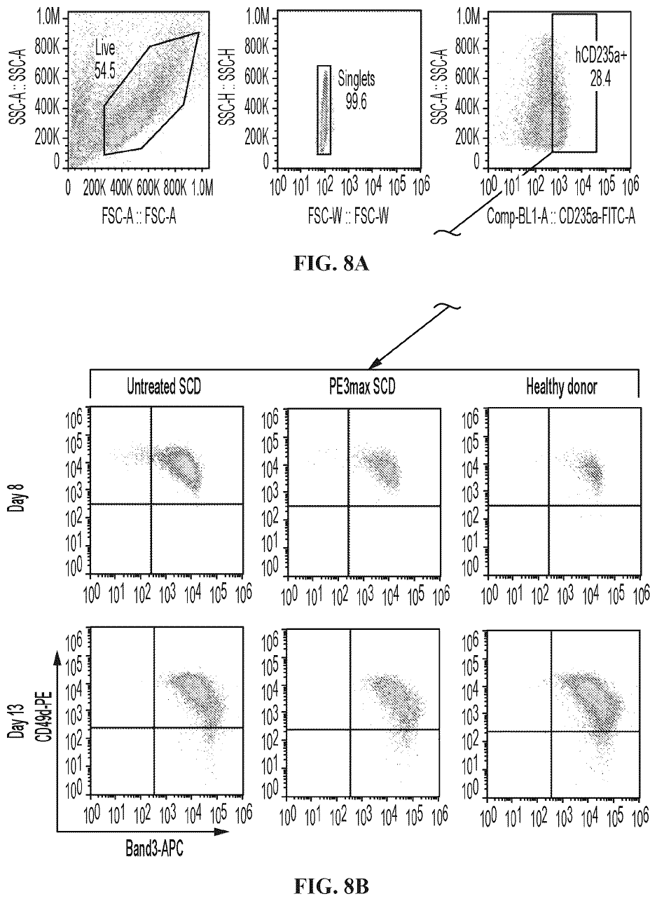

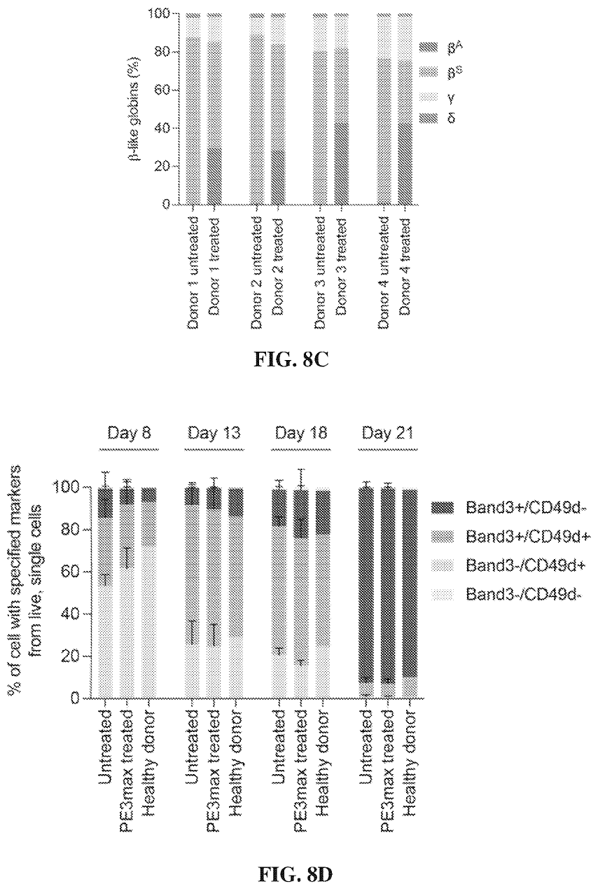

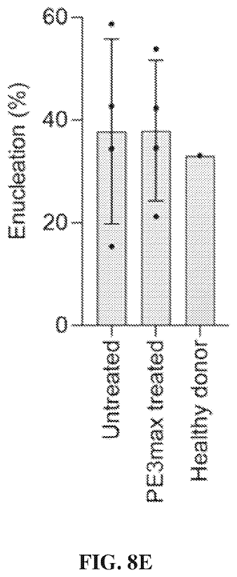

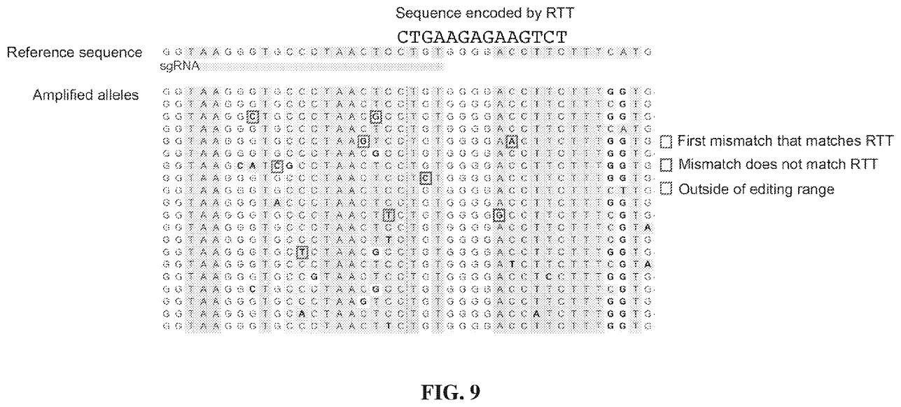

To determine the phenotypic impact of prime editing-mediated reversion of the sickle-cell mutation, CD235a⁺ cells were isolated from the bone marrow of transplanted mice, and the relative fractions of β-like globin proteins were quantified by HPLC. A decrease in HbS and an increase in HbA, proportional to the frequency of editing observed in total bone marrow and other lineages, were observed (FIG. 4A). Three of the four donors were edited with over 20% efficiency, with 28-43% HbA in bone marrow-derived CD235a⁺ cells. A similar result in SCD patient HSPCs differentiated toward erythroid cells in vitro was observed (FIG. 8A), with HbA production ranging from 28-43% (FIGS. 8B-8C). On average, the maturation stages of erythroid precursors, including anucleate reticulocytes, was similar between mice that received prime-edited SCD HSPCs or those that received unedited healthy donor HSPCs (FIGS. 8D-8E). These results reveal that direct reversion of HBB^{S} with the optimized prime editing strategy rescued HbA production, proportionately reduced pathogenic HbS levels, and did not alter erythroid maturation.

The hallmark phenotype of SCD is sickling of RBCs under hypoxic conditions. To determine if prime editing of SCD patient HSPCs to revert HBB^{S} to wild-type HBB^{A} reduces sickling in hypoxic conditions, purified reticulocytes from mice 17 weeks after transplantation were incubated in 2% O₂. All reticulocytes from mice receiving prime-edited cells showed a substantial reduction in sickling from an average of 63% sickled cells in untreated controls to 37% sickled cells in cells derived from prime-edited mice (FIGS. 4B-4C). The reduction of sickling was approximately proportional to the level of HbA in edited cells. These data together establish that prime editing can durably modify repopulating human HSCs, resulting in the production of HbA-expressing reticulocytes that resist hypoxia-induced sickling.

### Genome-Wide Off-Target Editing Analysis

Previous studies have reported that prime editing causes substantially lower levels of off-target editing compared to other CRISPR gene editing methods, consistent with the mechanism of prime editing, which requires three separate base pairing events between a target DNA strand and either the pegRNA or the pegRNA-derived flap, each of which provides an opportunity to reject an off-target sequence without modification^{2,29,40-48}.

Off-target prime editing outcomes from the disclosed HBB^{S} correction strategy were assessed in CD34⁺ cells. The experimental genome-wide off-target identification method CIRCLE-seq⁴⁹ was used to nominate potential off-target loci engaged by the Cas9 domain and guide RNAs used in the prime editing experiments above, and then off-target editing was measured by high-throughput sequencing of the nominated sites in prime-edited HSPCs from SCD patient donors. Since Cas9 nuclease activity when complexed with epegRNAs is modestly decreased compared to Cas9 with the corresponding sgRNA4, CIRCLE-seq was performed using the optimized epegRNA, using an sgRNA surrogate containing the identical protospacer, or using the NG1 nicking sgRNA.

CIRCLE-seq nominated 516 off-target sites when using the epegRNA, 437 sites when using an sgRNA with the epegRNA spacer, and 281 sites when using the nicking sgRNA. rhAmpSeq was used to perform multiplex-targeted DNA sequencing of the top 50 sites for each of these three categories, excluding the on-target sites and sites not amenable to pooling with the other loci (Table 4). Only 13 of the top 50 sites nominated using the surrogate sgRNA version of the epegRNA were not also nominated in the top 50 sites using the epegRNA. Of those 13 sites, all of them were within the top 173 sites for the epegRNA. In total, from the epegRNA and surrogate sgRNA CIRCLE-seq results, 63 nominated off-target candidates were examined in depth, including the top 50 sites from both lists.

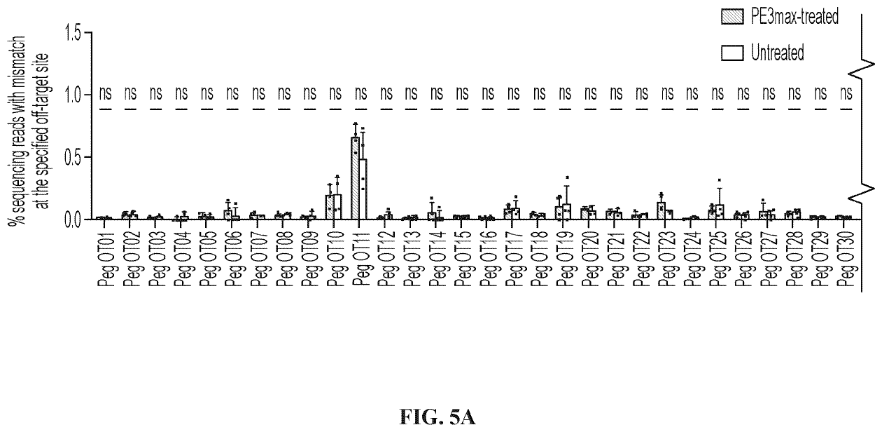

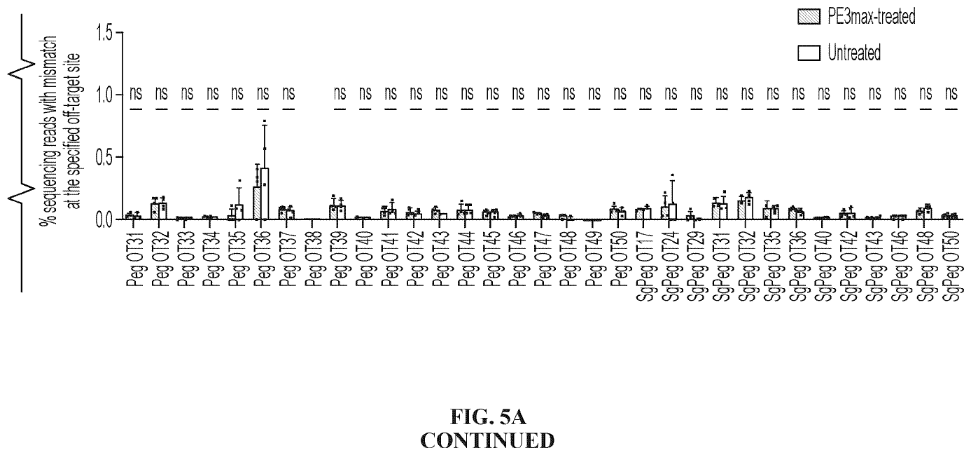

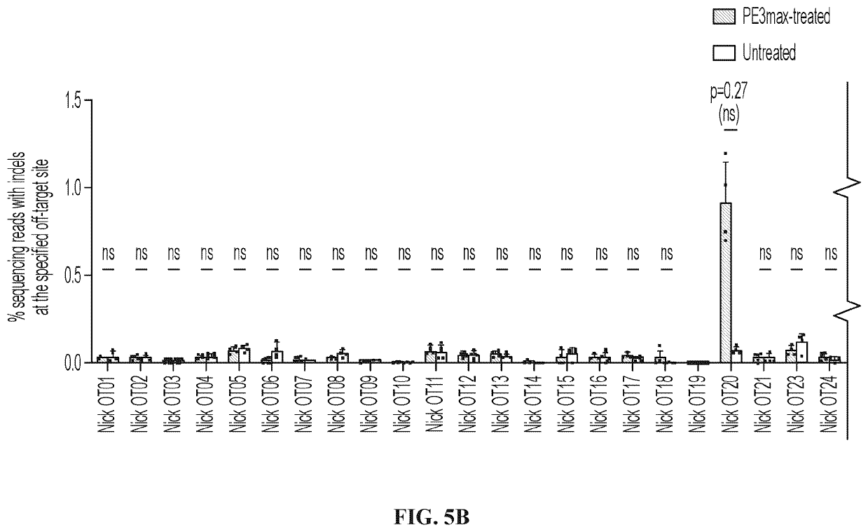

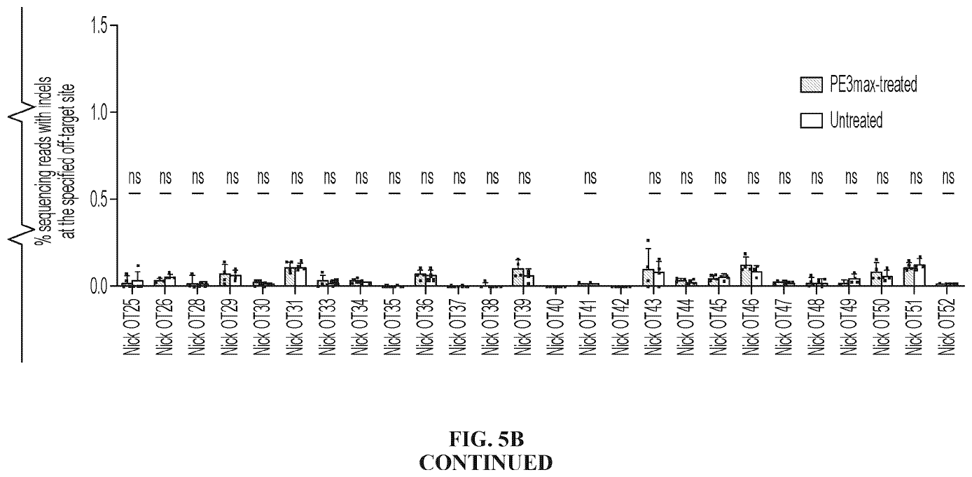

To assess off-target editing at CIRCLE-seq-detected sites of engagement by Cas9·epegRNA or Cas9·surrogate sgRNA complexes, the mutation frequency was quantified at the position of the first nucleotide change that would be introduced by the epegRNA RTT at each off-target site (FIG. 9). This position is the most likely nucleotide to be modified during prime editing². One nominated off-target site, pegOT49, was not compatible with rhAmpSeq amplification and was analyzed separately. No epegRNA-dependent off-target prime editing was detected in treated cells compared to untreated controls at any of the 63 CIRCLE-seq-nominated sites (FIG. 5A). This high degree of DNA specificity is consistent with previous reports of low off-target prime editing, and likely arises from the three distinct DNA hybridization events that must take place to result in productive prime editing^{2,29,40-48}.

To determine off-target editing at CIRCLE-seq-nominated off-target sites for the nicking sgRNA, the frequency of indels at the top 50 sites was quantified. One nominated off-target site, Nick OT32, was not compatible with rhAmpSeq or PCR amplification and could not be analyzed. Among the remaining 49 loci, the highest observed level of any editing (averaging 0.91%) was at NickOT20, a genomic site that contains the identical protospacer targeted by NG1. This level of editing was not statistically significant (p=0.27) in treated cells compared to untreated controls when analyzed using one-sided, paired multiple comparisons t-tests correcting for multiple comparisons using the Holm-Šídák method (FIG. 5B).

Collectively, the analysis of prime edited HSPCs at a total of 112 CIRCLE-seq-nominated candidate off-target sites engaged by Cas9 complexed with the HBB^{S}-targeting epegRNA, a corresponding sgRNA, or the NG1 nicking sgRNA did not detect any significant off-target editing. Though it was not statistically validated as a bona fide off-target locus, the nominated NickOT20 site contains the same NG1 protospacer sequence that occurs within the HBD gene encoding hemoglobin delta, which makes up only 3% of adult globin content⁵⁰. It is noted that the PE3b nicking sgRNA has a spacer sequence that does not match any sequence in HBD or any other region of human genome sequence hg38. Overall, these results suggest that the prime editing strategy used to revert HBB^{S} causes minimal off-target edits in the human genome.

### Discussion

Advancements in genome editing technologies have provided many options for treating SCD. While the best strategy has not yet been determined, and multiple strategies may offer clinical benefit, reverting the SCD allele back to wild-type is the most physiological approach. Correction strategies using nuclease-initiated HDR face several challenges including donor DNA template delivery, perturbation of engraftment potential^{18,27}, and low ratios of desired editing to indel byproducts^{17,18}. Described herein is a prime editing strategy that directly reverts the SCD allele to wild-type HBB^{A} without requiring double-strand DNA breaks, viral transduction, or any donor DNA templates. Following extensive optimization of key parameters including the choice of PE3max, the design of the pegRNA, the total volume of RNAs electroporated, and the position of the nicking guide, the strategy disclosed herein yields up to 41% prime editing in SCD patient HSPCs by day 3 that was maintained in bone-marrow repopulating HSCs in transplanted mice after 17 weeks. Red blood cells derived from repopulated HSCs after prime editing and transplantation in mice showed a reduction in HbS and sickling, and a rise in HbA, proportional to on-target editing. Ratios of HBB^{S} reversion to indels were high, and off-target editing was minimal after investigating 112 CIRCLE-seq-nominated candidate off-target sites.

There are several autologous transplantation approaches being developed or in clinical trials towards a treatment for SCD^{8-11,15,16,26}, and it is not yet known which strategy will be the safest and most effective for patients. The prime editing strategy described herein offers several potential advantages. It directly eliminates the pathogenic HBB^{S} allele and converts it back to the wild-type allele, in contrast with strategies that rely on lentiviral expression of non-sickling globin or induction of fetal hemoglobin (HbF).

Current strategies to correct HBB^{S} in SCD patient HSCs using nuclease-initiated HDR electroporate wild-type or a high-fidelity (HiFi) Cas9 nuclease complexed with a chemically-modified sgRNA, and a donor DNA template either delivered as single-stranded oligodeoxynucleotide donor (ssODN) 17 or through recombinant adeno-associated virus serotype 6 (rAAV6)-mediated deliveryl⁸. Compared to non-viral donor template deliveryl⁷, the prime editing strategy described herein leads to similar levels of desired correction in patient HSCs with a much higher desired edit-to-indel ratio and far fewer off-target indels. Similarly, compared to an HDR strategy that uses rAAV6-mediated donor template deliveryl⁸, the prime editing strategy described here results in a higher desired edit-to-indel ratio, and more efficient targeting of long-term HSCs with similar levels of pre-transplantation and post-transplantation HBB^{S}-to-HBB^{A} correction. The untreated control patient HSCs engrafted at the same level as prime-edited patient HSCs. In contrast, HSCs with rAAV6-delivered donor template engrafted significantly less efficiently than mock-electroporated cells¹⁸, consistent with a recent report that long-term HSCs engraft more poorly following HDR editing with rAAV6-delivered donor templates²⁷. These observations suggest that prime-edited HSCs engraft at higher rates.

Another important advantage of the prime editing strategy described here is that it does not require DSBs. Therefore, compared to nuclease-dependent approaches, prime editing can reduce the likelihood of undesired DSB outcomes such as uncontrolled mixtures of indels-which in HBB can lead to β-thalassemia-like loss-of-function^{17,18}—as well as large deletions, chromosomal loss, translocations, chromothripsis, and other undesired cell state changes¹⁹⁻²³. Several studies have used whole-genome sequencing, whole transcriptome sequencing, and other broad analytical methods following prime editing to assess potential genome- or transcriptome-wide changes in mammalian cells, and thus far no changes in single-nucleotide variants (SNPs), indels, telomere length, endogenous retrotransposon activity, gene expression, or splicing have been reported, nor any off-target RNA editing^{43,44,46-48}.

An additional feature of the prime editing strategy disclosed herein is that it does not require DNA delivery, viral transduction, or drug selection to enrich edited cells. DNA delivery is required for homology-directed repair and gene therapy, but can also lead to increased toxicity, lower engraftment frequency, or insertional mutagenesis^{18,25,27,51-53}.

CIRCLE-seq off-target site nomination using an sgRNA containing the epegRNA spacer sequence was compared to CIRCLE-seq using the epegRNA directly. The overlap between top hits was substantial. Of the 112 candidate off-target sites that were assessed with HTS, only one site (NickOT20) showed off-target editing consistently above that of untreated cells. While the observed level of off-target editing at NickOT20 (0.91%) was not statistically significant, this candidate off-target site contains an identical protospacer sequence to the nicking sgRNA.

Converting HBB^{S} to the benign, naturally-occurring β-globin Makassar (HBB^{G}) variant with an adenine base editor^{16,54} also offers advantages over Cas nuclease-based approaches and occurs more efficiently with fewer indels than reverting HBB^{S} to HBB^{A} with PE3max^{16,54}. However, the prime editing approach generates the natural adult β-globin allele and produces fewer off target edits than previously described HBB^{S}-to-HBB^{G} adenine base editing^{16,54}. Cas-independent off-target editing of DNA or RNA can occur with some base editors⁵⁵⁻⁵⁷ but was not detected with prime editing in several studies that investigated this possibility^{2,29,40-48}.

The ex vivo mRNA delivery method used in this Example is similar to current methods used for HSC editing in clinical trials^{11,12,33}. Much like ribonucleoproteins (RNPs), mRNA delivery reduces off-target editing compared to DNA delivery due to its transient expression^{16,59,60}. With a single electroporation, cells could be efficiently edited and cryopreserved. After being injected upon thawing to minimize loss of multipotency in vitro, edited cells efficiently engrafted into animal recipients with no loss of target prime editing efficiency after 17 weeks. The observed reduction in HbS levels, increase in HbA levels, and reduction in sickling propensity are suggestive of exceeding the predicted levels required for therapeutic benefit in SCD patients^{5,6}. These findings collectively suggesting that prime editing and transplanting patient HSPCs may represent a promising therapeutic strategy as a one-time autologous treatment for SCD.

### Methods

### High-Throughput Sequencing

High-throughput sequencing of genomic DNA extracted from human CD34⁺ cells was performed as previously described¹⁶. Genomic DNA was isolated from cells using lysis buffer lysis buffer (10 mM Tris-HCl, pH 7.5, 0.05% SDS, 25 μg/ml proteinase K (ThermoFisher Scientific)). Genomic DNA lysis was incubated at 37° C. for 1 hour followed by heat inactivation at 80° C. for 30 min. Primers for amplification of the DNMT1, HEK3, RNF2, RUNX1, HBB, and BCL11A loci are provided in Table 1. Primers include adapters for Illumina sequencing. Following Illumina barcoding, PCR products were pooled and purified by electrophoresis with a 2% agarose gel and a QIAquick Gel Extraction Kit (Qiagen), eluting with 30 μL of warm water. DNA concentration was determined using a Qubit dsDNA High-Sensitivity Assay Kit (ThermoFisher Scientific) and sequenced on an Illumina MiSeq instrument (single-end read, 280 cycles) according to the manufacturers' protocols. Alignment of fasq files and quantification of editing frequency was performed using CRISPResso2 in batch mode with a window width spanning at least 10 nt past each nick site.

PE, PEmax, and MLH1dn mRNA In Vitro Transcription

In vitro transcription of PE2 and PEmax mRNA was performed as previously described^{3,4}. Briefly, the 5′ untranslated region (UTR), Kozak sequence, PE2, or PEmax open reading frame (ORF) and 3′ UTR were cloned into a plasmid containing an inactive T7 (dT7) promoter. The mRNA transcription template was generated via PCR with primers that correct the dT7 promoter sequence into a functional one and install a poly (A) tail. The mRNA was transcribed using the T7 High-Yield RNA Kit (New England Biolabs) according to the manufacturer's instructions with the exception of full substitution of N1-methylpseudouridine (Trilink) for uridine and co-transcriptional capping with CleanCap AG (Trilink). The resulting mRNA was purified via lithium chloride precipitation and resuspended in TE buffer (10 nM Tris, 1 mM EDTA, pH 8.0 at room temperature). MLH1 dominant negative mRNA (MLH1dn) was transcribed analogously.

Synthetic epegRNA and Nicking Single-Guide RNA Generation

Synthetic epegRNAs were ordered from Integrated DNA Technologies. Each contained 2′-O-methyl modifications at the first and last three nucleotides and phosphorothioate linkages between the three first and last nucleotides. For all epegRNAs, the pegLIT program was used as previously described⁴. Synthetic nicking sgRNAs were obtained from Synthego and included 2′-O-methyl modifications at the first and last three bases as well as phosphorothioate bonds between the first three and last two bases.

### Isolation and Culture of CD34+ Human HSPCs

Circulating G-CSF-mobilized human mononuclear cells were obtained from deidentified healthy adult donors (Fred Hutchinson Research Center). Plerixafor-mobilized CD34⁺ cells or harvested bone marrow from deidentified SCD patient donors were collected according to the protocol “Peripheral Blood Stem Cell Collection for Sickle Cell Disease Patients” (ClinicalTrials.gov identifier NCT03226691), which was approved by the human subject research institutional review boards at the National Institutes of Health and St. Jude Children's Research Hospital. HSPCs were maintained in stem cell culture media: X-VIVO-15 (Lonza, 04-418Q) media supplemented with 100 ng/μL human SCF (R&D systems, 255-SC/CF), 100 ng/μL human TPO (R&D systems, 288-TP/CF), and 100 ng/μL human Flt-3 ligand (R&D systems, 308-FK/CF). Cells were seeded and maintained at a density of 1-2×10⁶ cells/mL.

### PE Electroporation of Human HSPC

Electroporations were performed with the Lonza 4-D Nucleofector System using program DS-130. All electroporations were performed in 20 μL reactions using the P3 Primary Cell X Kit S (Lonza, V4XP) with 15 μL of supplemented P3 buffer according to the manufacturer's instructions. For a standard PE3 electroporation, 1000 ng of in vitro transcribed PEmax mRNA was mixed with 90 μmol of synthetic epegRNA and 60 μmol of synthetic nicking sgRNA in 2 μL. For PE4 and PE5 electroporations, 1500 ng of in vitro transcribed MLH1dn mRNA was also used. For electroporations in which either the PEmax mRNA, the guide RNAs, or both had increased concentrations, the concentrations were increased without increasing the standard volume of 2 μL. For 2-fold, 3-fold, and 4-fold electroporations, the volume of the editing reagents was increased to 2 μL, 4 μL, or 6 μL while keeping the standard concentrations. Cells were thawed and allowed to recover in X-VIVO 15 cytokine-supplemented media for 24 hours before electroporation. 5×10⁵-1×10⁶ cells were electroporated per reaction and cultured at a density of 2×10⁶ cells per mL. For SCD patient HSPCs to be transplanted into NBSGW mice, the cells were cryopreserved 24 hours post-electroporation. All epegRNA and nicking sgRNA sequences can be found in Table 2.

### Cryopreservation of Edited HSPCs

1×10⁶-2×10⁶ edited cells were allowed to recover in cytokine-supplemented X-VIVO 15 media for 24 hours before cryopreservation. Cell pellets were collected and resuspended in equal volumes of Plasma-lyte-A media (Baxter International Inc) supplemented with 25% human serum albumin (HSA, Grifols Biologicals, LLC) and pentastarch media (Preservation Solutions Inc.) supplemented with DMSO (ATCC) and HSA.

### Mouse Experiments

No statistical test was used to predetermine sample size. All recipient mice were randomly selected for transplantation conditions by a blinded investigator who determined which mice would receive which cells. Investigators were blinded to the conditions each mouse was assigned with identification numbers. All assays were performed before identification numbers were matched to each experimental group.

Transplantation of Gene-Edited CD34+ HSPCs into NOD.Cg^{-KitW-41J} Tyr+Prkdc^{scid} Il2rg^{tmlWjl}/ThomJ (NBSGW) Mice

Transplantation experiments were performed as previously described¹⁶ with the following exception: cryopreserved cells were thawed, counted, and immediately injected into recipients. All antibodies used in the study can be found in Table 3.

### Erythroid Culture

Erythroid differentiation was completed as previously described¹⁶.

### Colony Forming Assay and Analysis of Clonal Editing Outcomes

BFU-E assays were performed as previously described¹⁶.

### Hemoglobin Quantification

Hemoglobin was quantified via HPLC using ion exchange columns as previously described¹⁶.

### In Vitro Sickling Assay

The in vitro sickling assay was performed as previously described¹⁶. Briefly, erythroid cells were seeded into 96-well plates with 100 μL of phase 3 erythroid differentiation medium under hypoxic conditions (2% oxygen) for 24 hours. Cells were monitored for 8 hours with the IncuCyte S3 Live-Cell Analysis system, and images of the cells were taken at 20× objective. A blinded researcher quantified sickling of each condition by counting over 300 cells per condition.

### CIRCLE-Seq Off-Target Editing Analysis

CIRCLE-seq off-target nomination and analysis was conducted as previously described¹⁶.

Targeted Off-Target Amplicon Sequencing and Analysis by rhAmpSeq

Off-target sites nominated by CIRCLE-seq were amplified from PE3max-treated or untreated HSPCs from sickle-cell donors 3 days post-electroporation using the rhAmpSeq system (IDT). A pooled sequencing library was generated using the rhAmpSeq design tool (primers located in Table 4). Genomic DNA was amplified using the pooled library according to the manufacturer's instructions and sequenced on an Illumina MiSeq instrument with 270 single-end reads.

For epegRNA off-target analysis, the percentage of mismatches that could be encoded by the epegRNA was quantified. For nicking sgRNA off-target analysis, the percentage of indels at the off-target loci was quantified. Both epegRNA OT49 and Nick OT32 were not compatible with the pooled rhAmpSeq analysis. EpegRNA OT49 was analyzed via HTS with forward primer 5′-ACACTCTTTCCCTACACGACGCTCTTCCGATCTNNNNTGGGTGTTATGGCCATCAT GA-3′ (SEQ ID NO: 136) and reverse primer 5′-TGGAGTTCAGACGTGTGCTCTTCCGATCTCCTCAACAAACTGAGGCATAC-3′ (SEQ ID NO: 254). Nick OT32 could not be analyzed because it was not amenable to PCR amplification.

### Tables

TABLE 1
Sequencing primers.
Sequences of primers for genomic amplification are shown. Each included the
relevant overhang for HTS as follows: Forward primer 5′ extension:
ACACTCTTTCCCTACACGACGCTCTTCCGATCTNNNN (SEQ ID NO: 137); Reverse
primer 5′ extension: TGGAGTTCAGACGTGTGCTCTTCCGATCT (SEQ ID NO: 255)

| | | FORWARD | | REVERSE |
|---|---|---|---|---|
| | | SEQ ID | | SEQ ID |
| SITE | FORWARD PRIMER | NO : | REVERSE PRIMER | NO: |
| DNMT1 | CACAACAGCTTCATGTCAGCC | 138 | ACGTTAATGTTTCCTGATGGTCC | 256 |
| HEK3 | CAGGGAAACGCCCATGCAATTAG | 140 | CTCTGTTGAGCTCGACCCTGAA | 258 |
| RNF2 | ACGTCTCATATGCCCCTTGG | 239 | ACGTAGGAATTTTGGTGGGACA | 357 |
| RUNX1 | ACAAACAAGACAGGGAACTGG | 240 | CTAGAGGGGTGAGGCTGAAAC | 358 |
| HBB | GCCACACCCTAGGGTTG | 139 | GGGAAAATAGACCAATAGGCAG | 257 |

TABLE 2
Sequences of epegRNAs, nicking guide RNAs, and sgRNAs.

| | | SEQ |
|---|---|---|
| | | ID |
| | | NO: |
| Site | Sequence | |
| DNMT1 (FIG. 1B) | GATTCCTGGTGCCAGAAACAGTTTTAGAGCTAGAAATAGCAAGTTAAAAT | 117 |
| | AAGGCTAGTCCGTTATCAACTTGAAAAAGTGGCACCGAGTCGGTGCGTCA | |
| | CCACTGTTTCTGGCACCAGGATAAATAACCGCGGTTCTATCTAGTTACGC | |
| | GTTAAACCAACTAGAA | |
| DNMT1 (FIG. 1C) | GATTCCTGGTGCCAGAAACAGTTTTAGAGCTAGAAATAGCAAGTTAAAAT | 118 |
| | AAGGCTAGTCCGTTATCAACTTGAAAAAGTGGCACCGAGTCGGTGCGTCA | |
| | CCACTGTTTCTGGCACCAGGACCTCTTCTTTGACGCGGTTCTATCTAGTT | |
| | ACGCGTTAAACCAACTAGAAA | |
| HEK3 | GGCCCAGACTGAGCACGTGAGTTTTAGAGCTAGAAATAGCAAGTTAAAAT | 119 |
| | AAGGCTAGTCCGTTATCAACTTGAAAAAGTGGCACCGAGTCGGTGCTTCC | |
| | TCTGCCATCTCGTGCTCAGTCTGTTATAAACCGCGGTTCTATCTAGTTAC | |
| | GCGTTAAACCAACTAGAA | |
| RNF2 | GTCATCTTAGTCATTACCTGGTTTTAGAGCTAGAAATAGCAAGTTAAAAT | 120 |
| | AAGGCTAGTCCGTTATCAACTTGAAAAAGTGGCACCGAGTCGGTGCATGG | |
| | GAACTCAGTTTATATGAGTTAGTAATGACTAAGATGTAAAGGAACGCGGT | |
| | TCTATCTAGTTACGCGTTAAACCAACTAGAA | |
| RUNX1 | GCATTTTCAGGAGGAAGCGAGTTTTAGAGCTAGAAATAGCAAGTTAAAAT | 121 |
| | AAGGCTAGTCCGTTATCAACTTGAAAAAGTGGCACCGAGTCGGTGCTGTC | |
| | TGAAGCAATCGCTTCCTCCTGAAAATTATACACGCGCGGTTCTATCTAGT | |
| | TACGCGTTAAACCAACTAGAA | |
| HBB (WITH 3′ UUU) | CATGGTGCACCTGACTCCTGGTTTTAGAGCTAGAAATAGCAAGTTAAAAT | 116 |
| | AAGGCTAGTCCGTTATCAACTTGAAAAAGTGGCACCGAGTCGGTGCAGAC | |
| | TTCTCTTCAGGAGTCAGGTGCACAGAATAAACGCGGTTCTATCTAGTTAC | |
| | GCGTTAAACCAACTAGAATTT | |
| HBB (WITHOUT 3′ UUU) | CATGGTGCACCTGACTCCTGGTTTTAGAGCTAGAAATAGCAAGTTAAAAT | 115 |
| | AAGGCTAGTCCGTTATCAACTTGAAAAAGTGGCACCGAGTCGGTGCAGAC | |
| | TTCTCTTCAGGAGTCAGGTGCACAGAATAAACGCGGTTCTATCTAGTTAC | |
| | GCGTTAAACCAACTAGAA | |

Nicking guideRNA (s)

| | | |
|---|---|---|
| DNMT1 | GCCCTTCAGCTAAAATAAAGG | 122 |
| HEK3 | GTCAACCAGTATCCCGGTGC | 123 |
| RNF2 | TCAACCATTAAGCAAAACAT | 124 |
| RUNX1 | ATGAAGCACTGTGGGTACGA | 125 |
| HBB | NG1: CCTTGATACCAACCTGCCCA | 128 |
| | NG2: GTAACGGCAGACTTCTCTTC | 129 |
| | NG3: CACGTTCACCTTGCCCCACA | 130 |
| | NG4: TCCACATGCCCAGTTTCTAT | 131 |

TABLE 3
Antibodies used in this Example.

| | | | | | |
|---|---|---|---|---|---|
| | | | | | PMID |
| | | | | | reference |
| Antibody and | | Dilutions | | | using this |
| fluorophore | Clone | used | Vendor | Catalog # | antibody |

Erythropoeisis

| | | | | | |
|---|---|---|---|---|---|
| Anti-Human | GA-R2 | 1:100 | BD Pharmingen ™ | 559943 | 31698466 |
| CD235a FITC | (HIR2) | | | | |
| Hoechst 33342 | Stock 10 | 1:1000 | Millipore Sigma | B2261 | 31698466 |
| | mM = 2000× | | | | |
| Anti-Human | 9F10 | 1:20 | BioLegend | 304304 | 31698466 |
| CD49d PE | | | | | |
| Anti-Human | | 1:100 | New York | Gift from | 31698466 |
| Band3 APC | | | Blood Center | X. An | |

Chimerism after human/NBSGW xenotransplantation

| | | | | | |
|---|---|---|---|---|---|
| Anti-Mouse CD45 | 30-F11 (RUO)/ | 1:40 | BD Pharmingen ™/ | 561088/ | 11062533/ |
| FITC/BV786 | 30-F11 (RUO) | | BD Horizon ™ | 564225 | 31698466 |
| Anti-Human CD45 | HI30 | 0.055555556 | BD Horizon ™ | 564047 | 31698466 |
| BV605 | | | | | |
| Anti-Human CD33 | P67.6 | 0.055555556 | BD Biosciences | 333946 | 31698466 |
| PE-Cy ™7 | | | | | |
| Anti-Human CD3 | SK7 (Leu-4) | 0.055555556 | BD Pharmingen ™ | 557832 | 31698466 |
| APC-Cy ™7 | (RUO) | | | | |
| Anti-Human CD19 | 4G7 (IVD)/ | 0.055555556 | BD Biosciences/ | 349209/ | 31698466/ |
| (Leu ™-12) PE/FITC | HIB19 (RUO) | | BD Pharmingen ™ | 555412 | 7690791 |
| Anti-Human CD34 | 581 (RUO)/ | 0.055555556 | BD Pharmingen ™/ | 561440/ | 7680152 |
| Alexa Flour 700/PE | 581 (RUO) | | BD Pharmingen ™ | 555822 | |
| Anti-Human CD235a | GA-R2 | 0.055555556 | BD Pharmingen ™ | 551336 | 31698466 |
| APC | (HIR2) | | | | |
| | (RUO) | | | | |
| DAPI | | | ThermoFisher | R37606 | |

TABLE 4
Pooled DNA sequencing primers for rhAmpSeq assay.
Primers were designed using IDT's online rhAmpSeq assay design tool.
Design run ID: RHC.06B5E2470E054BE.

| | | | SEQ | | SEQ |
|---|---|---|---|---|---|
| Target | | Component Forward | ID | | ID |
| Name | Component Name | Sequence | NO: | Component Sequence | NO: |
| NickOT01 | RH.62B6A820FDF | /rhSeq- | 141 | /rhSeq- | 259 |
| | 94C2Z0Z.F | f/ATGTTTAATATCCCAGG | | r/CCTGTGTCCTCAAAATGC | |
| | | CCCrCTGCT/GT2/ | | ArGTAGA/GT3/ | |
| NickOT02 | RH.35BC5AF030D | /rhSeq- | 142 | /rhSeq- | 260 |
| | 34C7Z0Z.F | f/AGGGTTAGAGCTAGGA | | r/AAATTGGCAGGACACCTT | |
| | | CAArGTGGG/GT1/ | | TTrCAGGC/GT1/ | |
| NickOT03 | RH.DD6B627905E | /rhSeq- | 143 | /rhSeq- | 261 |
| | 0458Z0Z.F | f/ACATTGACTTTGGAGA | | r/GTATTGCCAAAAGAGAA | |
| | | GTCTGrATGAC/GT1/ | | GGAGArCAAAC/GT4/ | |
| NickOT04 | RH.071B887871A8 | /rhSeq- | 144 | /rhSeq- | 262 |
| | 45EZOZ.F | f/TCTAAGTAGCTAGATTA | | r/CCACAGTCATGTAACAAA | |
| | | CAGGCArCCTGC/GT3/ | | CCTGrCACAT/GT1/ | |
| NickOT05 | RH.485B7F527270 | /rhSeq- | 145 | /rhSeq- | 263 |
| | 44CZOZ.F | f/GCAAGTAGAATCATCA | | r/CAGCCTTTGGGTCTGTAA | |
| | | GAGTGACIGGCTT/GT1/ | | AATTrCTCTG/GT1/ | |
| NickOT06 | RH.843EDB16D13 | /rhSeq- | 146 | /rhSeq- | 264 |
| | 4459Z0Z.F | f/GTATGCACTGCAAGAG | | r/ACTGAGAGTGAAGACAA | |
| | | AGTTTrGTACA/GT2/ | | CGArAGAAG/GT4/ | |
| NickOT07 | RH.BAC613B7B4F | /rhSeq- | 147 | /rhSeq- | 265 |
| | 5408ZOZ.F | f/AAGCAGTACTCCCAGA | | r/TCATCTTTATGCCACCCA | |
| | | AGAArGTTTG/GT1/ | | GArCAGTT/GT1/ | |
| NickOT08 | RH.A4470941BD3 | /rhSeq- | 148 | /rhSeq- | 266 |
| | D458Z0Z.F | f/ACAAATGGGAGAAAGA | | r/GTAATCACCTTCCTCCTC | |
| | | TCATACTrAGAGG/GT4/ | | CATrGCCTG/GT3/ | |
| NickOT09 | RH.918E7D3A8A7 | /rhSeq- | 149 | /rhSeq- | 267 |
| | 843BZOZ.F | f/CCTGTACCTCATCACTT | | r/GATGATATTAGGTCCTCT | |
| | | CAATACICACAG/GT4/ | | TCTGCrCTTAC/GT1/ | |
| NickOT10 | RH.2FC8D1855751 | /rhSeq- | 150 | /rhSeq- | 268 |
| | 4C0ZOZ.F | f/CACCATGTTAAGTGCTA | | r/TCAGTGAGTGAGACAGG | |
| | | GCGrGCTTT/GT1/ | | AArCAGTG/GT4/ | |
| NickOT11 | RH.486554E8427E | /rhSeq- | 151 | /rhSeq- | 269 |
| | 4F1Z0Z.F | f/CTCTTTTGTGCACACTC | | r/GACTCTGTGATTCTGCAA | |
| | | TAAGTrCTTCC/GT3/ | | GATIGAGCT/GT3/ | |
| NickOT12 | RH.DDODFFFBE6 | /rhSeq- | 152 | /rhSeq- | 270 |
| | 6B40BZOZ.F | f/CAGGTTGCACATGATTT | | r/AGAAATCAAAGAGGATG | |
| | | CTATTGArCATAC/GT1/ | | GGAATGrGGACA/GT1/ | |
| NickOT13 | RH.633B440D185 | /rhSeq- | 153 | /rhSeq- | 271 |
| | C4F9Z0Z.F | f/CTACTCAAGACTTGTGG | | r/GAAGCTTTTTCAAGCACT | |
| | | CTrGATGT/GT3/ | | GTTrCAACT/GT3/ | |
| NickOT14 | RH.1F6FFA8E1E0 | /rhSeq- | 154 | /rhSeq- | 272 |
| | 847FZ0Z.F | f/CAGGATTACAGTTGGA | | r/TTGGGTGACAGCATAAAT | |
| | | AAAGGTICTAGC/GT1/ | | ACATrGCAAG/GT4/ | |
| NickOT15 | RH.B81EB3F2A3F | /rhSeq- | 155 | /rhSeq- | 273 |
| | C4EFZOZ.F | f/CACCAACAGCTTGTACC | | r/GAAGGCAAAAGGGATGGr | |
| | | rCTCAG/GT1/ | | GTAGG/GT4/ | |
| NickOT16 | RH.BAA8CE4BBE | /rhSeq- | 156 | /rhSeq- | 274 |
| | 12445Z0Z.F | f/TGCTATATTGCCCAGAC | | r/CAGAACTACTCTCGTCCT | |
| | | TATTCTrCGAAC/GT1/ | | AACArCCTGT/GT1/ | |
| NickOT17 | RH.5FBF949B1FE | /rhSeq- | 157 | /rhSeq- | 275 |
| | 6486Z0Z.F | f/CTCACAAGGATACACA | | r/CTGATGATCATTTGCCAG | |
| | | GAGTTCrCCACT/GT3/ | | TCTrCAGGA/GT2/ | |
| NickOT18 | RH.27D5C64EAE8 | /rhSeq- | 158 | /rhSeq- | 276 |
| | 846EZ0Z.F | f/GTACATTCAAAAGTTTT | | r/GTGACCTGATATTTGGGT | |
| | | GCATTAAGCICAGGA/GT1 | | TAATTCArATTTG/GT1/ | |
| | | / | | | |
| NickOT19 | RH.2D095AFBA5 | /rhSeq- | 159 | /rhSeq- | 277 |
| | B84B3Z0Z.F | f/TGCTGATGCTACTCTAC | | r/TGCTCTATGCTTCTTTGG | |
| | | CTTTrGGATG/GT3/ | | AGArGAGGA/GT2/ | |
| NickOT20 | RH.3417DD3BC44 | /rhSeq- | 160 | /rhSeq- | 278 |
| | C48AZOZ.F | f/ACATGCCCAGTTTCCAT | | r/CTAGCAACCTCAAACAGA | |
| | | TTGrCCTCC/GT1/ | | CACrCATGG/GT1/ | |
| NickOT21 | RH.CFE0A0AD68 | /rhSeq- | 161 | /rhSeq- | 279 |
| | AB464Z0Z.F | f/GCAATACACTAGGTCA | | r/AAGTATATGGGACAATGT | |
| | | TCTACTTrCATTT/GT3/ | | GCATArGGTTA/GT4/ | |
| NickOT23 | RH.D8A6D93501C | /rhSeq- | 162 | /rhSeq- | 280 |
| | 04EAZOZ.F | f/CAGAATCCAGAGGCAA | | r/GGTGTCCCACATTTGTGAr | |
| | | GATrGAAGC/GT1/ | | CTTCC/GT3/ | |
| NickOT24 | RH.7392BED4418 | /rhSeq- | 163 | /rhSeq- | 281 |
| | 448FZ0Z.F | f/TGCCAAAAATGAGGTA | | r/AGACCCTTAGGTAAGAG | |
| | | TAAGATTTGATTrGCTTT/ | | ATAAACATAArCAAAC/GT2 | |
| | | GT1/ | | / | |
| NickOT25 | RH.86BA87C0E80 | /rhSeq- | 164 | /rhSeq- | 282 |
| | A429Z0Z.F | f/AGAATGTTTCTGTCATT | | r/TCATGAGAATAACATAGG | |
| | | CCAGAArAAGTC/GT3/ | | TGACTrCTCAA/GT1/ | |
| NickOT26 | RH.6D55E228D08 | /rhSeq- | 165 | /rhSeq- | 283 |
| | 145AZOZ.F | f/TTGAGAGGTAAAATTC | | r/GAAGCAACGTGATTCTAC | |
| | | CAGGCICAGAG/GT2/ | | GTIGTCTT/GT1/ | |
| NickOT28 | RH.6D36DC442D1 | /rhSeq- | 166 | /rhSeq- | 284 |
| | E438Z0Z.F | f/CCTTGTGTCCTTTTGAC | | r/CAGGGTTGACAACATAA | |
| | | ATGTrGCTTA/GT1/ | | GCTArCAAGA/GT2/ | |
| NickOT29 | RH.95C15E59C219 | /rhSeq- | 167 | /rhSeq- | 285 |
| | 421Z0Z.F | f/CTCTCAGTCACGTGGAA | | r/CCCTTGCCATATCCTGAT | |
| | | CTrGTATG/GT1/ | | CArCATGT/GT3/ | |
| NickOT30 | RH.FFCDC6A40E | /rhSeq- | 168 | /rhSeq- | 286 |
| | 994DOZOZ.F | f/ACTTATCATCAGGCATC | | r/TTCCCTGAAAATGTAAAG | |
| | | TGGTATATrGCTCA/GT4/ | | TATCAGArGCAAT/GT1/ | |
| NickOT31 | RH.ACB09C92E60 | /rhSeq- | 169 | /rhSeq- | 287 |
| | D4A3Z0Z.F | f/GTTTCAGTTAGTTGGGT | | r/CATTAAAAACCAGCATTT | |
| | | GACArCTACC/GT3/ | | GATTGGrATGTC/GT1/ | |
| NickOT33 | RH.DD623AEB119 | /rhSeq- | 170 | /rhSeq- | 288 |
| | 045CZOZ.F | f/TAGAAATGCTCTGTTTG | | r/CACCATGCCCAGCTAATT | |
| | | GGCrCTGAC/GT2/ | | TTGrCATTT/GT1/ | |
| NickOT34 | RH.EB296E9558E | /rhSeq- | 171 | /rhSeq- | 289 |
| | 741CZOZ.F | f/CACTCACAGGTTGCAG | | r/GCTGCCCAAGATTTTAGA | |
| | | ATAATCrCAGAT/GT3/ | | CTTTrGTGTT/GT1/ | |
| NickOT35 | RH.31F6A4241DF | /rhSeq- | 172 | /rhSeq- | 290 |
| | 54FDZOZ.F | f/AGCAACCCTCCAATATT | | r/GGACATGGAAAGCCTAT | |
| | | ATCACrCACTC/GT1/ | | ACTTGrAAATG/GT1/ | |
| NickOT36 | RH.F480ED73FAF | /rhSeq- | 173 | /rhSeq- | 291 |
| | 049EZOZ.F | f/TCAGACCTCGCCTTGTC | | r/CAAACTGTCCTGGCAACT | |
| | | rCAACC/GT1/ | | rCTGAC/GT1/ | |
| NickOT37 | RH.9B5A42A1FB1 | /rhSeq- | 174 | /rhSeq- | 292 |
| | C45AZOZ.F | f/GTTCAACAAGGCCTTCA | | r/AGATGCCAGGAACAGCTT | |
| | | GArGTTCC/GT3/ | | rGTGTG/GT1/ | |
| NickOT38 | RH.C9568D1B608 | /rhSeq- | 175 | /rhSeq- | 293 |
| | 0495ZOZ.F | f/ATTTGGAGTGGTCCTCT | | r/TCTCCATCCTTTGCAGAC | |
| | | GTGrCTGTT/GT2/ | | CTrGTACT/GT3/ | |
| NickOT39 | RH.3F7B9D88A44 | /rhSeq- | 176 | /rhSeq- | 294 |
| | A46AZOZ.F | f/CTGTTAACATACTGTAC | | r/GGGAAATTACAATCTTAG | |
| | | ATCCTCATAArCTGCA/GT | | TTCTACTTTTCrCACCT/GT1/ | |
| | | 1/ | | | |
| NickOT40 | RH.3DAC64CA60 | /rhSeq- | 177 | /rhSeq- | 295 |
| | 974A4Z0Z.F | f/GCATAGGGAGCCTCCTT | | r/AAGCCAGCATCCAGAGAr | |
| | | rGAGTT/GT1/ | | GACAG/GT4/ | |
| NickOT41 | RH.7372373A1547 | /rhSeq- | 178 | /rhSeq- | 296 |
| | 415Z0Z.F | f/TCTGTGTACGTACTGAT | | r/AAACAACGCAGCTGTGTG | |
| | | CCCrCAACA/GT3/ | | rGAAGC/GT1/ | |
| NickOT42 | RH.DD88E29AE44 | /rhSeq- | 179 | /rhSeq- | 297 |
| | 24F0Z0Z.F | f/TTTCAGTCCCTTGATTG | | r/GGCCACTAGTATATTGCC | |
| | | AGTCrCACGG/GT4/ | | TAArAAGCG/GT1/ | |
| NickOT43 | RH.07B3C758A45 | /rhSeq- | 180 | /rhSeq- | 298 |
| | 2415Z0Z.F | f/TGAACTCATAGGGCTTG | | r/TGGCCAGGACTCTTTAAG | |
| | | CTICAGTC/GT1/ | | TrCGTGA/GT2/ | |
| NickOT44 | RH.55878713FEA3 | /rhSeq- | 181 | /rhSeq- | 299 |
| | 459Z0Z.F | f/AAGAACCAGAGTTGGG | | r/AGGAACTGTGTGCCATCT | |
| | | CTTrCACCA/GT3/ | | GrCTCCA/GT2/ | |
| NickOT45 | RH.FAFD133CE81 | /rhSeq- | 182 | /rhSeq- | 300 |
| | 0441Z0Z.F | f/GATGGCTCACTCTCCTG | | r/GAATTTGGCTGGGCTTGT | |
| | | AATrGTGTT/GT3/ | | TrCCAAA/GT2/ | |
| NickOT46 | RH.23B3575E698C | /rhSeq- | 183 | /rhSeq- | 301 |
| | 474Z0Z.F | f/TTCAAGCAATTGTCTTG | | r/AAGCCAAGTCACACCTGT | |
| | | CGArCAGCC/GT1/ | | AATrCCCAG/GT3/ | |
| NickOT47 | RH.4242BA3B883 | /rhSeq- | 184 | /rhSeq- | 302 |
| | 94FEZOZ.F | f/CAGAGTACACTTTGCCC | | r/ACACTCTGAGAGATGCAG | |
| | | AArGCAAC/GT4/ | | GrCCAGG/GT4/ | |
| NickOT48 | RH.4FF4210F3760 | /rhSeq- | 185 | /rhSeq- | 303 |
| | 4C0ZOZ.F | f/GCTGTTTGGATGCCTTG | | r/CAGAGCAGTGGAGGGAA | |
| | | AArCTGAG/GT1/ | | ArAGCTT/GT3/ | |
| NickOT49 | RH.CCFEDC49EE | /rhSeq- | 186 | /rhSeq- | 304 |
| | BD497Z0Z.F | f/GCTAGCCTTCCCTCAAC | | r/GCAACAAGAGCGAAACT | |
| | | AArCACAT/GT3/ | | CCATrCTAAA/GT4/ | |
| NickOT50 | RH.27766B9776D9 | /rhSeq- | 187 | /rhSeq- | 305 |
| | 41FZ0Z.F | f/TCTAGACAACTGAGCTC | | r/TTGGTCAACAGCAATGAC | |
| | | CATGrGTAGG/GT2/ | | ArGATTC/GT3/ | |
| NickOT51 | RH.000B07590656 | /rhSeq- | 188 | /rhSeq- | 306 |
| | 4E2Z0Z.F | f/ACTAGTGGGCCTTTCTG | | r/TGATTTTGAGCAACACTC | |
| | | TTArCCTCC/GT1/ | | CCrCACCC/GT4/ | |
| NickOT52 | RH.DE949C7EEE7 | /rhSeq- | 189 | /rhSeq- | 307 |
| | B4E5Z0Z.F | f/GTAAACACTACCAATA | | r/GATCCCAAATCTGTTACC | |
| | | TGCTGCrCTCCC/GT1/ | | TCTGrCCTCT/GT2/ | |
| PegOT01 | RH.4D0B3A56DF8 | /rhSeq- | 190 | /rhSeq- | 308 |
| | B48AZOZ.F | f/GTGCCCTGTTTCAACTA | | r/TGAGTAAAAGCTACCCTA | |
| | | CAATrGGACA/GT2/ | | AATGCrCTGGG/GT2/ | |
| PegOT02 | RH.80F5BC9E152 | /rhSeq- | 191 | /rhSeq- | 309 |
| | 04D6Z0Z.F | f/GTCACAGTCTCTGGAAC | | r/CTTTCTTCCTGCCTGAACT | |
| | | TACrGCTCC/GT4/ | | AArGCCAC/GT3/ | |
| PegOT03 | RH.9F88B74678F1 | /rhSeq- | 192 | /rhSeq- | 310 |
| | 42AZ0Z.F | f/TTCAGGTGCATGATCCA | | r/AGTGTCCAATCAGGATTA | |
| | | TTTrCCAGT/GT1/ | | GGArGCTGT/GT2/ | |
| PegOT04 | RH.5EFCB035711 | /rhSeq- | 193 | /rhSeq- | 311 |
| | 0466Z0Z.F | f/GCATTGTGGTCGAGGTC | | r/GTGCACCTGACCTTCCGr | |
| | | TrGAACA/GT2/ | | GAACT/GT2/ | |
| PegOT05 | RH.731BA5E34E1 | /rhSeq- | 194 | /rhSeq- | 312 |
| | 543CZOZ.F | f/AAGTTTCATTCCTCAGT | | r/AAGCCAAACATTTACAAA | |
| | | CTCCTICTACC/GT1/ | | AGGGArCAGGA/GT4/ | |
| PegOT06 | RH.3FD344CA2C6 | /rhSeq- | 195 | /rhSeq- | 313 |
| | 349BZOZ.F | f/CTTCCTCCAAGATCTCA | | r/TCTGAGGTCTGTATGTGC | |
| | | GTCTrCTCAG/GT1/ | | TAArGCCAC/GT4/ | |
| PegOT07 | RH.01D9BD680E1 | /rhSeq- | 196 | /rhSeq- | 314 |
| | C439Z0Z.F | f/TCTCATCTCCTAATGCC | | r/TTGGTCAAATTCTCTCTTC | |
| | | ATCArCACTG/GT4/ | | TGArGGAGG/GT3/ | |
| PegOT08 | RH.2626A04F7C39 | /rhSeq- | 197 | /rhSeq- | 315 |
| | 432Z0Z.F | f/ACCGGTCTGATCAAAA | | r/GAATTCTTTTTCTCAGCA | |
| | | TTTATTTrGGCAG/GT2/ | | TGGAArCATCC/GT3/ | |
| PegOT09 | RH.BCE37F58B72 | /rhSeq- | 198 | /rhSeq- | 316 |
| | 0440ZOZ.F | f/TATGCATCTGTGAGCTC | | r/TTACTCCTCCAACCACCC | |
| | | CGrGAACA/GT1/ | | AArCAGAA/GT2/ | |
| PegOT10 | RH.492564EDE38 | /rhSeq- | 199 | /rhSeq- | 317 |
| | F404Z0Z.F | f/AGATCCCTTCTGCCTCTr | | r/GTGGGCAAAGTCACAAC | |
| | | CCCAG/GT3/ | | ArCCTGT/GT2/ | |
| PegOT11 | RH.887CD7219064 | /rhSeq- | 200 | /rhSeq- | 318 |
| | 45FZ0Z.F | f/GCTTGGGTGAGAGTGA | | r/CTGCATGTATCAATTTTG | |
| | | GACrCTTGT/GT3/ | | AGAGArATTCA/GT2/ | |
| PegOT12 | RH.38B6DBC2887 | /rhSeq- | 201 | /rhSeq- | 319 |
| | 2477Z0Z.F | f/ATGGACGCCGCATATA | | r/ACCTTGTGATCCTCCCGTr | |
| | | CGrCGTGA/GT3/ | | CTCAG/GT3/ | |
| PegOT13 | RH.450C52431CC | /rhSeq- | 202 | /rhSeq- | 320 |
| | 8476Z0Z.F | f/ATTTCCTGCCTGTGGTT | | r/GCGCCTTGGGTAATTAAT | |
| | | TGArCTCAC/GT2/ | | TCCrCAAAG/GT3/ | |
| PegOT14 | RH.72498897C44B | /rhSeq- | 203 | /rhSeq- | 321 |
| | 411Z0Z.F | f/CCTGCTAAGCCAGACA | | r/GGTATGGATGACCCAGTr | |
| | | TrCCTGC/GT3/ | | GGGTG/GT2/ | |
| PegOT15 | RH.C5D271FD6A | /rhSeq- | 204 | /rhSeq- | 322 |
| | BA43DZOZ.F | f/TCATTTTGTATAGACCA | | r/ATTCTCCTTTGGAAACAT | |
| | | GTGCTGrCCCAT/GT1/ | | ACCTCrGCACT/GT2/ | |
| PegOT16 | RH.034D06511A19 | /rhSeq- | 205 | /rhSeq- | 323 |
| | 4E9Z0Z.F | f/TGCTACTCTCATTCTCC | | r/TGCTTCCTCTGACTGAAC | |
| | | TCCrCTGCA/GT2/ | | ArCAAAG/GT4/ | |
| PegOT17 | RH.EA1A967E442 | /rhSeq- | 206 | /rhSeq- | 324 |
| | 745AZ0Z.F | f/GAGAACGTGACTCATC | | r/TGTTTTATGCTCCCCAAG | |
| | | CAGrCTCCA/GT2/ | | TGrCCTCA/GT3/ | |
| PegOT18 | RH.F8A82B7EDD | /rhSeq- | 207 | /rhSeq- | 325 |
| | 4C49CZOZ.F | f/CAAAGTTGAAGGTGCT | | r/ATAAACAAACACGTGGC | |
| | | CACArAAACT/GT1/ | | AGACrGGTTA/GT2/ | |
| PegOT19 | RH.CAODBOEEDC | /rhSeq- | 208 | /rhSeq- | 326 |
| | C54A4Z0Z.F | f/AGAGAGGACTGAAGGG | | r/GTCTTAGTGGATCTTGAG | |
| | | TGrCAGTC/GT1/ | | CArGAGCT/GT2/ | |
| PegOT20 | RH.56736BC330F2 | /rhSeq- | 209 | /rhSeq- | 327 |
| | 462Z0Z.F | f/CAAACTGCCCTGCATGT | | r/AGTACTGGGCTCCCTTAC | |
| | | AATrCTTCA/GT4/ | | TrGATGT/GT3/ | |
| PegOT21 | RH.2CF3FDAA244 | /rhSeq- | 210 | /rhSeq- | 328 |
| | B414Z0Z.F | f/GCAATTGTTAAGAAAT | | r/GAAAGACCTCCTACGTAA | |
| | | AATTCAGAGAGCrCAGA | | TTGCArCCCAA/GT4/ | |
| | | A/GT1/ | | | |
| PegOT22 | RH.29A50A61F0E | /rhSeq- | 211 | /rhSeq- | 329 |
| | 1412Z0Z.F | f/CCCAAGCTAGACTCATC | | r/GGTTTGAGCTGATTCAGG | |
| | | TTCTrCACTC/GT1/ | | GArGGAAA/GT2/ | |
| PegOT23 | RH.B7C1378C30E | /rhSeq- | 212 | /rhSeq- | 330 |
| | 94E5Z0Z.F | f/ATAAGCAAAAAGGAAT | | r/TCTCTTCTACAGATGAAG | |
| | | CATCGCTArGGACA/GT4/ | | AGTCTrGGGTC/GT3/ | |
| PegOT24 | RH.CE26C5910306 | /rhSeq- | 213 | /rhSeq- | 331 |
| | 40AZOZ.F | f/GCTTAGGTTACCCCAAT | | r/GTTGCCCTATATGACTTC | |
| | | TAGCrCAGAC/GT1/ | | CAAGIGCAGA/GT4/ | |
| PegOT25 | RH.6E609636E2B4 | \|/rhSeq- | 214 | /rhSeq- | 332 |
| | 43BZOZ.F | f/CAGCTCACTGGAACTA | | r/AAAGTTTTTATAGGCCCG | |
| | | CAGrGCACA/GT1/ | | GAGrCGGTG/GT2/ | |
| PegOT26 | RH.CB4A29CC056 | /rhSeq- | 215 | /rhSeq- | 333 |
| | 346EZOZ.F | f/CTTGCTTTTTCATGTTTC | | r/GCTTGTTCTGAGCCCATA | |
| | | TGTGGrGTTGG/GT2/ | | GATAArGGATC/GT1/ | |
| PegOT27 | RH.EA4FC647D0 | /rhSeq- | 216 | /rhSeq- | 334 |
| | AA413ZOZ.F | f/GATGAAGAAGTACATA | | r/GATGGTCAGTAAGTTAAT | |
| | | GGGTGAGrGTCTG/GT3/ | | CATGCrCCATG/GT1/ | |
| PegOT28 | RH.21E9F9715FE8 | /rhSeq- | 217 | /rhSeq- | 335 |
| | 4C4ZOZ.F | f/AGGAGCTGGGACCAAG | | r/GTCAGCCTTGCTACCTCrC | |
| | | rGGATG/GT2/ | | AGGA/GT3/ | |
| PegOT29 | RH.3B3F42EF4D0 | /rhSeq- | 218 | /rhSeq- | 336 |
| | 5485Z0Z.F | f/CAGACCAGAAAGGAAG | | r/ACCCACTTGAGATGTCAC | |
| | | TGGrCACAG/GT2/ | | ArGTGGA/GT1/ | |
| PegOT30 | RH.17861622FB1F | /rhSeq- | 219 | /rhSeq- | 337 |
| | 429Z0Z.F | f/ATAACGAGGTTGAGAA | | r/GATGAAACAGTGCAACA | |
| | | GGTGAArCTGAG/GT3/ | | TGTGTrGTGAA/GT4/ | |
| PegOT31 | RH.2471DCF94B8 | /rhSeq- | 220 | /rhSeq- | 338 |
| | 6429Z0Z.F | f/GACCCTGGATCCTCTGA | | r/CGATGCTATTTCCATGAG | |
| | | TAAATICTCTG/GT1/ | | GATrCTTGG/GT4/ | |
| PegOT32 | RH.0B3AE3C58D1 | /rhSeq- | 22 | /rhSeq- | 339 |
| | B494Z0Z.F | f/ACAGTTGGACATGGTG | | r/CAAGTCAAGCTGATCGAG | |
| | | CTArGAGCT/GT2/ | | AGrGTACA/GT2/ | |
| PegOT33 | RH.851EF8935C3 | /rhSeq- | 222 | /rhSeq- | 340 |
| | A4B8ZOZ.F | f/ATGCTGAGGACCCATTC | | r/CTGTTCAGCCGTACCATG | |
| | | TrCACCT/GT3/ | | ArGATGT/GT3/ | |
| PegOT34 | RH.EF27571CDD8 | /rhSeq- | 223 | /rhSeq- | 341 |
| | F477Z0Z.F | f/CCACTGCCCTTACAAAT | | r/GCTGCAGAGATTTTGTTT | |
| | | GTATArGCAAT/GT1/ | | ATGGrCCAGT/GT1/ | |
| PegOT35 | RH.6B42DDD218E | /rhSeq- | 224 | /rhSeq- | 342 |
| | 046EZOZ.F | f/GCCGGTCTTTGAATTTC | | r/CACTCACCAGTCTTTGTG | |
| | | TGArCCTCA/GT2/ | | AAArACACT/GT2/ | |
| PegOT36 | RH.4F7546848A64 | /rhSeq- | 225 | /rhSeq- | 343 |
| | 487Z0Z.F | f/TCACTTGAACCCGGAG | | r/TAATGATGCTGGGCCACA | |
| | | GrCGAAG/GT2/ | | rGGCAT/GT4/ | |
| PegOT37 | RH.C404BD2AC9 | /rhSeq- | 226 | /rhSeq- | 344 |
| | A74E8Z0Z.F | f/TTGGATTTGTTTCTACA | | r/ACACTTAGATTAGTGCCC | |
| | | GCCArGAGAC/GT1/ | | ACrCAGGA/GT2/ | |
| PegOT38 | RH.7EAAD8FD4E | /rhSeq- | 227 | /rhSeq- | 345 |
| | DC46CZOZ.F | f/TGAAAGTTCCTGCAGTC | | r/GGTTGGTATATGCTGTTC | |
| | | TTrCCCAC/GT3/ | | CAArGGAAC/GT1/ | |
| PegOT39 | RH.01361C97532F | /rhSeq- | 228 | /rhSeq- | 346 |
| | 4B3Z0Z.F | f/TGAGTATCTGGTTAGAG | | r/AAATCTCTGGGATCATGA | |
| | | GTCTTTrCAGAC/GT4/ | | TAGGGrCTTTG/GT2/ | |
| PegOT40 | RH.EOD5EBBAC1 | /rhSeq- | 229 | /rhSeq- | 347 |
| | 164EEZ0Z.F | f/GCTGTCTCCCCAAATTG | | r/ACTTCCCAGCCTTTCCAGr | |
| | | GrCAATC/GT1/ | | GAGCT/GT3/ | |
| PegOT41 | RH.636F96A38051 | /rhSeq- | 230 | /rhSeq- | 348 |
| | 4BEZOZ.F | f/AGAAGCAAAGGCCAAA | | r/ATGTCTGGGTATGGAGTT | |
| | | CTCTrGTCTT/GT4/ | | TGArGAAAC/GT2/ | |
| PegOT42 | RH.066A73D77867 | /rhSeq- | 231 | /rhSeq- | 349 |
| | 477Z0Z.F | f/GGATAACATCGGGCAC | | r/GATTCAACGGGCAGGAG | |
| | | ATGrGTATG/GT1/ | | ArGAAGA/GT2/ | |
| PegOT43 | RH.96959E1A9DE | /rhSeq- | 232 | /rhSeq- | 350 |
| | 84ECZ0Z.F | f/TGAAGGCAAAAGGAGT | | r/GCCTTTTGGTTGGTGAAC | |
| | | GGGrCTCTG/GT2/ | | ArCCAGA/GT4/ | |
| PegOT44 | RH.02EF30E8A77 | /rhSeq- | 233 | /rhSeq- | 351 |
| | E4EFZ0Z.F | f/ATAATGGATTTTGGAG | | r/TTTGGGAGTACAAGTGAT | |
| | | ATTCAGArATGGG/GT4/ | | TTTGrGGTAC/GT4/ | |
| PegOT45 | RH.79AD8395ED | /rhSeq- | 234 | /rhSeq- | 352 |
| | DB4F9Z0Z.F | f/TCCGACATAGTATGAA | | r/CGTGGGATTGTATGTTTA | |
| | | ACTGCArGCTGC/GT4/ | | GCATIGCCTC/GT4/ | |
| PegOT46 | RH.5F9FEFD3873 | /rhSeq- | 235 | /rhSeq- | 353 |
| | 046DZOZ.F | f/AATTGGGCTTGTTTGGT | | r/GGCCAAGGATCTCTGTAA | |
| | | ACTTrCCTCA/GT1/ | | AArCCCTT/GT4/ | |
| PegOT47 | RH.65B6C9A5FA0 | /rhSeq- | 236 | /rhSeq- | 354 |
| | 84FFZ0Z.F | f/AGGACTGAAAAACTTC | | r/TGGAAAGTTTCCTGTCAC | |
| | | CTATCCTrGTACA/GT4/ | | TATTTTrCTTCC/GT1/ | |
| PegOT48 | RH.0B1C67CF94B | /rhSeq- | 237 | /rhSeq- | 355 |
| | 6472Z0Z.F | f/TCTCCATTTCCAGCACC | | r/GCAGGGATCACAAATTCC | |
| | | TTrCTCCT/GT2/ | | TTrCCTCT/GT1/ | |
| PegOT50 | RH.40A3A38BOB5 | /rhSeq- | 238 | /rhSeq- | 356 |
| | 74CEZOZ.F | f/AAAGCTTCCTGGTGCTC | | r/CACGAGCAGGGAGAATGr | |
| | | rCTCCC/GT3/ | | GTGCT/GT3/ | |
| SgOT17 | RH.C005E7241F5 | /rhSeq- | 241 | /rhSeq- | 359 |
| | A408ZOZ.F | f/TCTTCACTGACACTCCC | | r/TTAAGGTCTCTCTCCCAC | |
| | | AGrCTCTG/GT1/ | | CArCCAGA/GT3/ | |
| SgOT24 | RH.068682E0B6C7 | /rhSeq- | 242 | /rhSeq- | 360 |
| | 412Z0Z.F | f/CTAATTCCCTCCCAAGT | | r/AATTAGCTAGGTGAGGCA | |
| | | CCArGGAAA/GT4/ | | GTrGGGAT/GT2/ | |
| SgOT29 | RH.A377C1890FA | /rhSeq- | 243 | /rhSeq- | 361 |
| | 546CZOZ.F | f/GGTCTCCAATACCGGG | | r/CAGCATTAACCACTGGGC | |
| | | CrCTAAG/GT2/ | | rAAATG/GT4/ | |
| SgOT31 | RH.B83DD2512D7 | /rhSeq- | 244 | /rhSeq- | 362 |
| | 9488Z0Z.F | f/GGTACCTCGCTATCCAC | | r/CACTGTGAAACCTGCAAC | |
| | | TTTrGTAAG/GT2/ | | ArCACAA/GT2/ | |
| SgOT32 | RH.97C5FEA1EF9 | /rhSeq- | 245 | /rhSeq- | 363 |
| | 9408Z0Z.F | f/TAGCAATAGGTGGTTG | | r/TAGCCAGGTGCTGAACAT | |
| | | GCCrCCAGT/GT4/ | | TrCTGGT/GT1/ | |
| SgOT35 | RH.6DBB94C7B9F | /rhSeq- | 246 | /rhSeq- | 364 |
| | 845EZ0Z.F | f/GGCATCATGAGGGTTC | | r/AGAAGGACATCAGAGAA | |
| | | ATAArCTCAG/GT4/ | | GCrCCTGG/GT2/ | |
| SgOT36 | RH.7AED8AAB18 | /rhSeq- | 247 | /rhSeq- | 365 |
| | A6434Z0Z.F | f/AGGTGAGGAGCAGCAG | | r/CGCCCCTCCTTAAACAGTr | |
| | | ArCCACA/GT1/ | | CTCTG/GT3/ | |
| SgOT40 | RH.C4CBDD69B1 | /rhSeq- | 248 | /rhSeq- | 366 |
| | 6D4F8Z0Z.F | f/TGGCTGGCTAGATTTTC | | r/CAGCAGAACATGTGTCTT | |
| | | TAAACrCCTTC/GT1/ | | TGTrCAAAT/GT3/ | |
| SgOT42 | RH.15C6A0899E6 | /rhSeq- | 249 | /rhSeq- | 367 |
| | 2401Z0Z.F | f/AGCCTAAGCCGCTCTTT | | r/GAAGGAGGGACAGAGTC | |
| | | rGAACA/GT2/ | | ArAACAA/GT2/ | |
| SgOT43 | RH.D1281FACCD | /rhSeq- | 250 | /rhSeq- | 368 |
| | 2D4FEZOZ.F | f/GAAAACTTAAGTGTAA | | r/TAGGACAAAGGAATCTTA | |
| | | GCAGCAGATIGGAAT/GT | | AGTAGTCArCTCTG/GT3/ | |
| | | 3/ | | | |
| SgOT46 | RH.3435C6DA25E | /rhSeq- | 251 | /rhSeq- | 369 |
| | 04A3Z0Z.F | f/AGACCTTTTGATTCTGT | | r/CATTTTGGAATTCCATGA | |
| | | GTCTTCrCTGTG/GT4/ | | GAAACCrCACAA/GT2/ | |
| SgOT48 | RH.1F33DB76216 | /rhSeq- | 252 | /rhSeq- | 370 |
| | A4FFZ0Z.F | f/CATTGCTTTCTTCACTG | | r/TGAGTGCTTCCAAAATCC | |
| | | GGAArCACAT/GT4/ | | TTAArGGGAA/GT4/ | |
| SgOT50 | RH.B5205F4C00E | /rhSeq- | 253 | /rhSeq- | 371 |
| | D488Z0Z.F | f/CAGATGGACTATGCTAT | | r/CATACACATTTGAGTACC | |
| | | GTTTTTGArGGACC/GT1/ | | AGACTAArCACAC/GT1/ | |

### REFERENCES

1. Piel, F. B., Steinberg, M. H., & Rees, D. C. Sickle Cell Disease. N. Engl. J. Med. 376, 1561-1573 (2017).
2. Anzalone, A. V. et al. Search-and-replace genome editing without double-strand breaks or donor DNA. Nature (2019) doi: 10.1038/s41586-019-1711-4.
3. Chen, P. J. et al. Enhanced prime editing systems by manipulating cellular determinants of editing outcomes. Cell 184, 5635-5652.e29 (2021).
4. Nelson, J. W. et al. Engineered pegRNAs improve prime editing efficiency. Nat. Biotechnol. 40, 402-410 (2022).
5. Fitzhugh, C. D. et al. At least 20% donor myeloid chimerism is necessary to reverse the sickle phenotype after allogeneic HSCT. Blood 130, 1946-1948 (2017).
6. Walters, M. C. et al. Stable mixed hematopoietic chimerism after bone marrow transplantation for sickle cell anemia. Biol. Blood Marrow Transplant. 7, 665-673 (2001).
7. Khemani, K., Katoch, D., & Krishnamurti, L. Curative Therapies for Sickle Cell Disease. Ochsner J. 19, 131-137 (2019).
8. Kanter, J. et al. Biologic and Clinical Efficacy of LentiGlobin for Sickle Cell Disease. N. Engl. J. Med. 386, 617-628 (2022).
9. Zeng, J. et al. Therapeutic base editing of human hematopoietic stem cells. Nat. Med. (2020) doi: 10.1038/s41591-020-0790-y.
10. Esrick, E. B. et al. Post-Transcriptional Genetic Silencing of BCL11A to Treat Sickle Cell Disease. N. Engl. J. Med. 384, 205-215 (2021).
11. Frangoul, H. et al. CRISPR-Cas9 Gene Editing for Sickle Cell Disease and β-Thalassemia. N. Engl. J. Med. 384, 252-260 (2021).
12. Lessard, S. et al. Zinc Finger Nuclease-Mediated Disruption of the BCL11A Erythroid Enhancer Results in Enriched Biallelic Editing, Increased Fetal Hemoglobin, and Reduced Sickling in Erythroid Cells Derived from Sickle Cell Disease Patients. Blood 134, 974-974 (2019).
13. Traxler, E. A. et al. A genome-editing strategy to treat β-hemoglobinopathies that recapitulates a mutation associated with a benign genetic condition. Nat. Med. 22, 987-990 (2016).
14. Métais, J.-Y. et al. Genome editing of HBG1 and HBG2 to induce fetal hemoglobin. Blood Adv. 3, 3379-3392 (2019).
15. Wilkinson, A. C. et al. Cas9-AAV6 gene correction of beta-globin in autologous HSCs improves sickle cell disease erythropoiesis in mice. Nat. Commun. 12, 686 (2021).
16. Newby, G. A. et al. Base editing of haematopoietic stem cells rescues sickle cell disease in mice. Nature 595, 295-302 (2021).
17. Magis, W. et al. High-level correction of the sickle mutation is amplified in vivo during erythroid differentiation. iScience 25, 104374 (2022).
18. Lattanzi, A. et al. Development of β-globin gene correction in human hematopoietic stem cells as a potential durable treatment for sickle cell disease. Sci. Transl. Med. 13, eabf2444 (2021).
19. Zuccaro, M. V. et al. Allele-Specific Chromosome Removal after Cas9 Cleavage in Human Embryos. Cell 183, 1650-1664.e15 (2020).
20. Enache, O. M. et al. Cas9 activates the p53 pathway and selects for p53-inactivating mutations. Nat. Genet. 52, 662-668 (2020).
21. Ihry, R. J. et al. p53 inhibits CRISPR-Cas9 engineering in human pluripotent stem cells. Nat. Med. 24, 939-946 (2018).
22. Kosicki, M., Tomberg, K., & Bradley, A. Repair of double-strand breaks induced by CRISPR-Cas9 leads to large deletions and complex rearrangements. Nat. Biotechnol. 36, 765-771 (2018).
23. Haapaniemi, E., Botla, S., Persson, J., Schmierer, B., & Taipale, J. CRISPR-Cas9 genome editing induces a p53-mediated DNA damage response. Nat. Med. 24, 927-930 (2018).
24. Ferrari, S. et al. Choice of template delivery mitigates the genotoxic risk and adverse impact of editing in human hematopoietic stem cells. Cell Stem Cell 29, 1428-1444.e9 (2022).
25. Schiroli, G. et al. Precise Gene Editing Preserves Hematopoietic Stem Cell Function following Transient p53-Mediated DNA Damage Response. Cell Stem Cell 24, 551-565.e8 (2019).
26. Dever, D. P. et al. CRISPR/Cas9 β-globin gene targeting in human haematopoietic stem cells. Nature 539, 384-389 (2016).
27. Romero, Z. et al. Editing the Sickle Cell Disease Mutation in Human Hematopoietic Stem Cells: Comparison of Endonucleases and Homologous Donor Templates. Mol. Ther. 27, 1389-1406 (2019).
28. Chen, P. J. & Liu, D. R. Prime editing for precise and highly versatile genome manipulation. Nat. Rev. Genet. (2022) doi: 10.1038/s41576-022-00541-1.
29. Jin, S. et al. Genome-wide specificity of prime editors in plants. Nat. Biotechnol. 39, 1292-1299 (2021).
30. Anzalone, A. V. et al. Programmable deletion, replacement, integration and inversion of large DNA sequences with twin prime editing. Nat. Biotechnol. 40, 731-740 (2022).
31. Choi, J. et al. Precise genomic deletions using paired prime editing. Nat. Biotechnol. 40, 218-226 (2022).
32. Lin, Q. et al. High-efficiency prime editing with optimized, paired pegRNAs in plants. Nat. Biotechnol. 39, 923-927 (2021).
33. De Dreuzy, E. et al. EDIT-301: An Experimental Autologous Cell Therapy Comprising Cas12a-RNP Modified mPB-CD34+ Cells for the Potential Treatment of SCD. Blood 134, 4636-4636 (2019).
34. Ferreira da Silva, J. et al. Prime editing efficiency and fidelity are enhanced in the absence of mismatch repair. Nat. Commun. 13, 760 (2022).
35. Peterson, C. W. et al. Intracellular RNase activity dampens zinc finger nuclease-mediated gene editing in hematopoietic stem and progenitor cells. Mol. Ther. —Methods Clin. Dev. 24, 30-39 (2022).
36. Leonard, A. et al. Low-Dose Busulfan Reduces Human CD34+ Cell Doses Required for Engraftment in c-kit Mutant Immunodeficient Mice. Mol. Ther.—Methods Clin. Dev. 15, 430-437 (2019).
37. McIntosh, B. E. et al. Nonirradiated NOD,B6.SCID I12rγ−/− KitW41/W41 (NBSGW) Mice Support Multilineage Engraftment of Human Hematopoietic Cells. Stem Cell Rep. 4, 171-180 (2015).
38. Luc, S. et al. Bcl11a Deficiency Leads to Hematopoietic Stem Cell Defects with an Aging-like Phenotype. Cell Rep. 16, 3181-3194 (2016).
39. Kurup, S. P., Moioffer, S. J., Pewe, L. L., & Harty, J. T. p53 Hinders CRISPR/Cas9-Mediated Targeted Gene Disruption in Memory CD8 T Cells In Vivo. J. Immunol. 205, 2222-2230 (2020).
40. Schene, I. F. et al. Prime editing for functional repair in patient-derived disease models. Nat. Commun. 11, 5352 (2020).
41. Kim, D. Y., Moon, S. B., Ko, J.-H., Kim, Y.-S., & Kim, D. Unbiased investigation of specificities of prime editing systems in human cells. Nucleic Acids Res. 48, 10576-10589 (2020).
42. Liu, Y. et al. Efficient generation of mouse models with the prime editing system. Cell Discov. 6, 27 (2020).
43. Geurts, M. H. et al. Evaluating CRISPR-based prime editing for cancer modeling and CFTR repair in organoids. Life Sci. Alliance 4, e202000940 (2021).
44. Park, S.-J. et al. Targeted mutagenesis in mouse cells and embryos using an enhanced prime editor. Genome Biol. 22, 170 (2021).
45. Gao, P. et al. Prime editing in mice reveals the essentiality of a single base in driving tissue-specific gene expression. Genome Biol. 22, 83 (2021).
46. Lin, J. et al. Modeling a cataract disorder in mice with prime editing. Mol. Ther. —Nucleic Acids 25, 494-501 (2021).
47. Habib, O., Habib, G., Hwang, G.-H., & Bae, S. Comprehensive analysis of prime editing outcomes in human embryonic stem cells. Nucleic Acids Res. 50, 1187-1197 (2022).
48. Gao, R. et al. Genomic and Transcriptomic Analyses of Prime Editing Guide RNA-Independent Off-Target Effects by Prime Editors. CRISPR J. 5, 276-293 (2022).
49. Tsai, S. Q. et al. CIRCLE-seq: a highly sensitive in vitro screen for genome-wide CRISPR-Cas9 nuclease off-targets. Nat. Methods 14, 607-614 (2017).
50. Sankaran, V. G. & Orkin, S. H. The Switch from Fetal to Adult Hemoglobin. Cold Spring Harb. Perspect. Med. 3, a011643-a011643 (2013).
51. Pattabhi, S. et al. In Vivo Outcome of Homology-Directed Repair at the HBB Gene in HSC Using Alternative Donor Template Delivery Methods. Mol. Ther. —Nucleic Acids 17, 277-288 (2019).
52. Howe, S. J. et al. Insertional mutagenesis combined with acquired somatic mutations causes leukemogenesis following gene therapy of SCID-X1 patients. J. Clin. Invest. 118, 3143-3150 (2008).
53. Stein, S. et al. Genomic instability and myelodysplasia with monosomy 7 consequent to EVI1 activation after gene therapy for chronic granulomatous disease. Nat. Med. 16, 198-204 (2010).
54. Chu, S. H. et al. Rationally Designed Base Editors for Precise Editing of the Sickle Cell Disease Mutation. CRISPR J. 4, 169-177 (2021).
55. Doman, J. L., Raguram, A., Newby, G. A., & Liu, D. R. Evaluation and minimization of Cas9-independent off-target DNA editing by cytosine base editors. Nat. Biotechnol. 38, 620-628 (2020).
56. Jin, S. et al. Cytosine, but not adenine, base editors induce genome-wide off-target mutations in rice. Science 364, 292-295 (2019).
57. Zuo, E. et al. Cytosine base editor generates substantial off-target single-nucleotide variants in mouse embryos. Science 364, 289-292 (2019).
58. Banskota, S. et al. Engineered virus-like particles for efficient in vivo delivery of therapeutic proteins. Cell 185, 250-265.e16 (2022).
59. Kaczmarek, J. C., Kowalski, P. S., & Anderson, D. G. Advances in the delivery of RNA therapeutics: from concept to clinical reality. Genome Med. 9, 60 (2017).
60. Hendel, A. et al. Chemically modified guide RNAs enhance CRISPR-Cas genome editing in human primary cells. Nat. Biotechnol. 33, 985-989 (2015).
61. Hu, J. et al. Isolation and functional characterization of human erythroblasts at distinct stages: implications for understanding of normal and disordered erythropoiesis in vivo. Blood 121, 3246-3253 (2013).

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims are introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

What is claimed is:

1. A method of treating sickle cell disease comprising contacting a target nucleotide sequence with a prime editor and an engineered prime editing guide RNA (epegRNA), wherein the epegRNA comprises the structure 5′-[spacer sequence]-[single guide RNA (sgRNA) scaffold sequence]-[extension arm sequence]-[optional engineered linker sequence]-[3′ structured motif]-3′, wherein each instance of]-[comprises an optional linker sequence.

2. The method of claim 1, wherein the epegRNA comprises the spacer sequence 5′-CATGGTGCACCTGACTCCTG-3′ (SEQ ID NO: 127), or a sequence at least 90%, at least 95%, at least 96%, at least 97%, at least 98%, or at least 99% identical to the sequence 5′-CATGGTGCACCTGACTCCTG-3′ (SEQ ID NO: 127) or a fragment thereof.

3. The method of claim 1 or 2, wherein the epegRNA comprises the sgRNA scaffold sequence 5′-GTTTTAGAGCTAGAAATAGCAAGTTAAAATAAGGCTAGTCCGTTATCAACTTGAA AAAGTGGCACCGAGTCGGTGC-3′ (SEQ ID NO: 126), or a sequence at least 90%, at least 95%, at least 96%, at least 97%, at least 98%, or at least 99% identical to the sequence 5′-GTTTTAGAGCTAGAAATAGCAAGTTAAAATAAGGCTAGTCCGTTATCAACTTGAA AAAGTGGCACCGAGTCGGTGC-3′ (SEQ ID NO: 126) or a fragment thereof.

4. The method of any one of claims 1-3, wherein the epegRNA comprises the extension arm sequence 5′-AGACTTCTCTTCAGGAGTCAGGTGCAC-3′ (SEQ ID NO: 111), or a sequence at least 90%, at least 95%, at least 96%, at least 97%, at least 98%, or at least 99% identical to the sequence 5′-AGACTTCTCTTCAGGAGTCAGGTGCAC-3′ (SEQ ID NO: 111) or a fragment thereof.

5. The method of any one of claims 1-4, wherein the epegRNA comprises the engineered linker sequence 5′-AGAATAAA-3′ between the extension arm sequence and the 3′ structured motif, or a sequence at least 90%, at least 95%, at least 96%, at least 97%, at least 98%, or at least 99% identical to the sequence 5′-AGAATAAA-3′ or a fragment thereof.

6. The method of any one of claims 1-5, wherein the 3′ structured motif comprises a toe-loop, a hairpin, a stem-loop, a pseudoknot, an aptamer, a G-quadruplex, a tRNA, a riboswitch, or a ribozyme, optionally wherein the structured motif is a pseudoknot (e.g., evopreq1).

7. The method of any one of claims 1-6, wherein the epegRNA comprises a 3′ structured motif comprising a nucleotide sequence of any one of SEQ ID NOs: 48-77 or a sequence at least 90%, at least 95%, at least 96%, at least 97%, at least 98%, or at least 99% identical to the sequence of any one of SEQ ID NOs: 48-77 or a fragment thereof.

8. The method of any one of claims 1-7, wherein the epegRNA comprises a 3′ structured motif comprising the sequence 5′-CGCGGTTCTATCTAGTTACGCGTTAAACCAACTAGAA-3′ (SEQ ID NO: 72), or a sequence at least 90%, at least 95%, at least 96%, at least 97%, at least 98%, or at least 99% identical to the sequence 5′-CGCGGTTCTATCTAGTTACGCGTTAAACCAACTAGAA-3′ (SEQ ID NO: 72) or a fragment thereof.

9. The method of any one of claims 1-8, wherein the epegRNA comprises the sequence 5′-CATGGTGCACCTGACTCCTGGTTTTAGAGCTAGAAATAGCAAGTTAAAATAAGG CTAGTCCGTTATCAACTTGAAAAAGTGGCACCGAGTCGGTGCAGACTTCTCTTCA GGAGTCAGGTGCACAGAATAAACGCGGTTCTATCTAGTTACGCGTTAAACCAAC TAGAA-3′ (SEQ ID NO: 115), or a sequence at least 90%, at least 95%, at least 96%, at least 97%, at least 98%, or at least 99% identical to the sequence of SEQ ID NO: 115.

10. The method of any one of claims 1-9, wherein the epegRNA further comprises the sequence 5′-TTT-3′ on the 3′ end.

11. The method of any one of claims 1-10, wherein the epegRNA comprises the sequence 5′-CATGGTGCACCTGACTCCTGGTTTTAGAGCTAGAAATAGCAAGTTAAAATAAGG CTAGTCCGTTATCAACTTGAAAAAGTGGCACCGAGTCGGTGCAGACTTCTCTTCA GGAGTCAGGTGCACAGAATAAACGCGGTTCTATCTAGTTACGCGTTAAACCAAC TAGAATTT-3′ (SEQ ID NO: 116), or a sequence at least 90%, at least 95%, at least 96%, at least 97%, at least 98%, or at least 99% identical to the sequence of SEQ ID NO: 116.

12. The method of claim 10 or 11, wherein at least one nucleotide of the 5′-TTT-3′ sequence comprises a 2′-O-methyl modification, optionally wherein all three of the nucleotides of the 5′-TTT-3′ sequence comprise a 2′-O-methyl modification.

13. The method of any one of claims 10-12, wherein at least one nucleotide of the 5′-TTT-3′ sequence is connected by a phosphorothioate linkage, optionally wherein all three of the nucleotides of the 5′-TTT-3′ sequence are connected by phosphorothioate linkages.

14. The method of any one of claims 1-13, wherein the method results in the correction of an A·T:T·A transversion mutation at nucleotide position 20 in the β-globin gene (HBB).

15. The method of claim 14, wherein correction of the A·T:T·A transversion mutation in HBB reverts the sickle cell disease allele to the wild type allele.

16. The method of claim 14 or 15, wherein correction of the A·T:T·A transversion mutation in HBB results in the correction of a valine mutation in the β-globin protein to a glutamic acid residue.

17. The method of any one of claims 14-16, wherein the method further results in the introduction of a G→A silent PAM-disrupting edit at the nucleotide position following the A·T:T·A transversion mutation.

18. The method of any one of claims 1-17 further comprising nicking the non-PAM-containing strand of the target nucleotide sequence using a nicking sgRNA.

19. The method of claim 18, wherein the spacer sequence of the nicking sgRNA comprises the nucleotide sequence 5′-CCTTGATACCAACCTGCCCA-3′ (SEQ ID NO: 128), 5′-GTAACGGCAGACTTCTCTTC-3′ (SEQ ID NO: 129), 5′-CACGTTCACCTTGCCCCACA-3′ (SEQ ID NO: 130), or 5′-TCCACATGCCCAGTTTCTAT-3′ (SEQ ID NO: 131), or a sequence at least 90%, at least 95%, at least 96%, at least 97%, at least 98%, or at least 99% identical to the sequence 5′-CCTTGATACCAACCTGCCCA-3′ (SEQ ID NO: 128), 5′-GTAACGGCAGACTTCTCTTC-3′ (SEQ ID NO: 129), 5′-CACGTTCACCTTGCCCCACA-3′ (SEQ ID NO: 130), or 5′-TCCACATGCCCAGTTTCTAT-3′ (SEQ ID NO: 131) or a fragment thereof.

20. The method of claim 18 or 19, wherein the spacer sequence of the nicking sgRNA comprises the nucleotide sequence 5′-CCTTGATACCAACCTGCCCA-3′ (SEQ ID NO: 128), or a sequence at least 90%, at least 95%, at least 96%, at least 97%, at least 98%, or at least 99% identical to the sequence 5′-CCTTGATACCAACCTGCCCA-3′ (SEQ ID NO: 128) or a fragment thereof.

21. The method of claim 18 or 19, wherein the spacer sequence of the nicking sgRNA comprises the nucleotide sequence 5′-GTAACGGCAGACTTCTCTTC-3′ (SEQ ID NO: 129), 5′-CACGTTCACCTTGCCCCACA-3′ (SEQ ID NO: 130), or 5′-TCCACATGCCCAGTTTCTAT-3′ (SEQ ID NO: 131), or a sequence at least 90%, at least 95%, at least 96%, at least 97%, at least 98%, or at least 99% identical to the sequence 5′-GTAACGGCAGACTTCTCTTC-3′ (SEQ ID NO: 129), 5′-CACGTTCACCTTGCCCCACA-3′ (SEQ ID NO: 130), or 5′-TCCACATGCCCAGTTTCTAT-3′ (SEQ ID NO: 131) or a fragment thereof.

22. The method of any one of claims 1-21, wherein the prime editor comprises PEmax architecture.

23. The method of any one of claims 1-22, wherein the prime editor is PE3max, PE3bmax, PE4max, PE5max, or PE5bmax.

24. The method of any one of claims 1-23, wherein the prime editor is PE3max or PE3bmax.

25. The method of any one of claims 1-24, wherein the contacting is performed in a cell.

26. The method of claim 25, wherein the cell is a eukaryotic cell.

27. The method of claim 25 or 26, wherein the cell is a human cell.

28. The method of any one of claims 25-27, wherein the cell is a hematopoietic stem or progenitor cell (HSPC) or a hematopoietic stem cell (HSC).

29. The method of claim 28, wherein the cell is an HSC, and wherein the HSC retains the edits introduced in its genome in its sub-lineage cells.

30. The method of any one of claims 25-27, wherein the cell is a CD34⁺ cell, a CD235a⁺ cell, a CD33⁺ cell, or a CD19⁺ cell.

31. The method of any one of claims 25-30, wherein the prime editor is delivered to the cell as a protein and the epegRNA is delivered to the cell as RNA, optionally wherein a nicking sgRNA is also delivered to the cell as RNA.

32. The method of any one of claims 25-30, wherein the prime editor is delivered to the cell as mRNA and the epegRNA is delivered to the cell as RNA, optionally wherein a nicking sgRNA is also delivered to the cell as RNA.

33. The method of claim 32, wherein the prime editor is delivered to the cell as mRNA by electroporation and the epegRNA is delivered to the cell as RNA by electroporation, optionally wherein a nicking sgRNA is also delivered to the cell as RNA by electroporation.

34. The method of claim 33, wherein the prime editor mRNA, epegRNA, and nicking sgRNA comprise approximately 20% of the total electroporation volume.

35. The method of claim 33 or 34, wherein the molar ratio of the amount of epegRNA to the amount of nicking sgRNA delivered to the cell is approximately 1.5:1.

36. The method of any one of claims 1-35, wherein the method is performed in vitro.

37. The method of any one of claims 1-35, wherein the method is performed ex vivo.

38. The method of claim 37, wherein the method is performed in a cell ex vivo, and wherein the edited cell is subsequently transplanted into a subject to be treated.

39. The method of any one of claims 1-35, wherein the method is performed in vivo.

40. The method of any one of claim 1-35 or 39, wherein the method is performed in a subject.

41. The method of claim 38 or 40, wherein the subject is a human.

42. The method of claim 38, 40, or 41, wherein the subject has or is suspected of having sickle cell disease.

43. The method of any one of claims 1-42, wherein the method results in a greater than 20%, greater than 25%, greater than 30%, greater than 35%, or greater than 40% efficiency of conversion of an A·T:T·A transversion mutation at nucleotide position 20 in the β-globin gene (HBB) to the wild type sequence.

44. The method of any one of claims 1-43, wherein the method results in an edit-to-indel ratio of greater than 5, greater than 5.5, greater than 6, greater than 6.5, greater than 7, or greater than 7.5.

45. The method of any one of claims 1-44, wherein the method results in at least 30%, at least 35%, or at least 40% of cells edited using the method retaining the edit following transplantation into a subject.

46. The method of any one of claims 1-45, wherein the method is performed with a prime editing efficiency of at least 60%, at least 65%, at least 70%, at least 75%, or at least 80%.

47. The method of any one of claims 1-46, wherein the method results in an indel frequency of less than 10%, less than 9%, less than 8%, less than 7%, less than 6%, less than 5%, or less than 4%.

48. A method of treating sickle cell disease comprising contacting a target nucleotide sequence with a prime editor and a prime editing guide RNA (pegRNA), wherein the prime editor is PE3max or PE3bmax, and wherein the pegRNA comprises the structure 5′-[spacer sequence]-[single guide RNA (sgRNA) scaffold sequence]-[extension arm sequence]-3′, wherein each instance of]-[comprises an optional linker sequence.

49. A method of treating sickle cell disease comprising contacting a target nucleotide sequence with a prime editor, a prime editing guide RNA (pegRNA), and a nicking single guide RNA (sgRNA), wherein the pegRNA comprises the structure 5′-[spacer sequence]-[sgRNA scaffold sequence]-[extension arm sequence]-3′, wherein each instance of]-[comprises an optional linker sequence, and wherein the spacer sequence of the nicking sgRNA comprises the nucleotide sequence 5′-GTAACGGCAGACTTCTCTTC-3′ (SEQ ID NO: 129), 5′-CACGTTCACCTTGCCCCACA-3′ (SEQ ID NO: 130), or 5′-TCCACATGCCCAGTTTCTAT-3′ (SEQ ID NO: 131), or a sequence at least 90%, at least 95%, at least 96%, at least 97%, at least 98%, or at least 99% identical to the sequence 5′-GTAACGGCAGACTTCTCTTC-3′ (SEQ ID NO: 129), 5′-CACGTTCACCTTGCCCCACA-3′ (SEQ ID NO: 130), or 5′-TCCACATGCCCAGTTTCTAT-3′ (SEQ ID NO: 131) or a fragment thereof.

50. An engineered prime editing guide RNA (epegRNA) targeting the β-globin gene (HBB), wherein the epegRNA comprises the structure 5′-[spacer sequence]-[single guide RNA (sgRNA) scaffold sequence]-[extension arm sequence]-[optional engineered linker sequence]-[3′ structured motif]-3′, wherein each instance of]-[comprises an optional linker sequence.

51. The epegRNA of claim 50, wherein the epegRNA comprises the spacer sequence 5′-CATGGTGCACCTGACTCCTG-3′ (SEQ ID NO: 127), or a sequence at least 90%, at least 95%, at least 96%, at least 97%, at least 98%, or at least 99% identical to the sequence 5′-CATGGTGCACCTGACTCCTG-3′ (SEQ ID NO: 127) or a fragment thereof.

52. The epegRNA of claim 50 or 51, wherein the epegRNA comprises the sgRNA scaffold sequence 5′-GTTTTAGAGCTAGAAATAGCAAGTTAAAATAAGGCTAGTCCGTTATCAACTTGAA AAAGTGGCACCGAGTCGGTGC-3′ (SEQ ID NO: 126), or a sequence at least 90%, at least 95%, at least 96%, at least 97%, at least 98%, or at least 99% identical to the sequence 5′-GTTTTAGAGCTAGAAATAGCAAGTTAAAATAAGGCTAGTCCGTTATCAACTTGAA AAAGTGGCACCGAGTCGGTGC-3′ (SEQ ID NO: 126) or a fragment thereof.

53. The epegRNA of any one of claims 50-52, wherein the epegRNA comprises the extension arm sequence 5′-AGACTTCTCTTCAGGAGTCAGGTGCAC-3′ (SEQ ID NO: 111), or a sequence at least 90%, at least 95%, at least 96%, at least 97%, at least 98%, or at least 99% identical to the sequence 5′-AGACTTCTCTTCAGGAGTCAGGTGCAC-3′ (SEQ ID NO: 111) or a fragment thereof.

54. The epegRNA of any one of claims 50-53, wherein the epegRNA comprises the engineered linker sequence 5′-AGAATAAA-3′ between the extension arm sequence and the 3′ structured motif, or a sequence at least 90%, at least 95%, at least 96%, at least 97%, at least 98%, or at least 99% identical to the sequence 5′-AGAATAAA-3′ or a fragment thereof.

55. The epegRNA of any one of claims 50-54, wherein the 3′ structured motif comprises a toe-loop, a hairpin, a stem-loop, a pseudoknot, an aptamer, a G-quadruplex, a tRNA, a riboswitch, or a ribozyme.

56. The epegRNA of any one of claims 50-55, wherein the epegRNA comprises a 3′ structured motif of any one of SEQ ID NOs: 48-77, or a sequence at least 90%, at least 95%, at least 96%, at least 97%, at least 98%, or at least 99% identical to the sequence of any one of SEQ ID NOs: 48-77 or a fragment thereof.

57. The epegRNA of any one of claims 50-56, wherein the epegRNA comprises a 3′ structured motif comprising the sequence 5′-CGCGGTTCTATCTAGTTACGCGTTAAACCAACTAGAA-3′ (SEQ ID NO: 72), or a sequence at least 90%, at least 95%, at least 96%, at least 97%, at least 98%, or at least 99% identical to the sequence 5′-CGCGGTTCTATCTAGTTACGCGTTAAACCAACTAGAA-3′ (SEQ ID NO: 72) or a fragment thereof.

58. The epegRNA of any one of claims 50-57, wherein the epegRNA comprises the sequence 5′-CATGGTGCACCTGACTCCTGGTTTTAGAGCTAGAAATAGCAAGTTAAAATAAGG CTAGTCCGTTATCAACTTGAAAAAGTGGCACCGAGTCGGTGCAGACTTCTCTTCA GGAGTCAGGTGCACAGAATAAACGCGGTTCTATCTAGTTACGCGTTAAACCAAC TAGAA-3′ (SEQ ID NO: 115), or a sequence at least 90%, at least 95%, at least 96%, at least 97%, at least 98%, or at least 99% identical to the sequence of SEQ ID NO: 115.

59. The epegRNA of any one of claims 50-58, wherein the epegRNA further comprises the sequence 5′-TTT-3′ at the 3′ end.

60. The epegRNA of any one of claims 50-59, wherein the epegRNA comprises the sequence 5′-CATGGTGCACCTGACTCCTGGTTTTAGAGCTAGAAATAGCAAGTTAAAATAAGG CTAGTCCGTTATCAACTTGAAAAAGTGGCACCGAGTCGGTGCAGACTTCTCTTCA GGAGTCAGGTGCACAGAATAAACGCGGTTCTATCTAGTTACGCGTTAAACCAAC TAGAATTT-3′ (SEQ ID NO: 116), or a sequence at least 90%, at least 95%, at least 96%, at least 97%, at least 98%, or at least 99% identical to the sequence of SEQ ID NO: 116.

61. The epegRNA of claim 59 or 60, wherein at least one nucleotide of the 5′-TTT-3′ sequence comprises a 2′-O-methyl modification, optionally wherein all three of the nucleotides of the 5′-TTT-3′ sequence comprise a 2′-O-methyl modification.

62. The epegRNA of any one of claims 59-61, wherein at least one nucleotide of the 5′-TTT-3′ sequence is connected by a phosphorothioate linkage, optionally wherein all three of the nucleotides of the 5′-TTT-3′ sequence are connected by phosphorothioate linkages.

63. A complex comprising a prime editor and an epegRNA of any one of claims 50-62.

64. The complex of claim 63, wherein the prime editor comprises PEmax architecture.

65. The complex of claim 63 or 64, wherein the prime editor comprises PE3max, PE3bmax, PE4max, PE5max, or PE5bmax.

66. The complex of any one of claims 63-65, wherein the prime editor comprises PE3max or PE3bmax.

67. A polynucleotide encoding the epegRNA of any one of claims 50-62.

68. One or more polynucleotides encoding the prime editor and the epegRNA of the complex of any one of claims 63-66.

69. A vector comprising the polynucleotide of claim 67.

70. One or more vectors comprising the one or more polynucleotides of claim 68.

71. A pharmaceutical composition comprising the epegRNA of any one of claims 50-62, the complex of any one of claims 63-66, the one or more polynucleotides of claim 67 or 68, or the one or more vectors of claim 69 or 70.

72. A pharmaceutical composition comprising the epegRNA of any one of claims 50-62 and an mRNA encoding a prime editor.

73. A pharmaceutical composition comprising a cell edited using the method of any one of claims 1-49.

74. A cell comprising the epegRNA of any one of claims 50-62, the complex of any one of claims 63-66, the one or more polynucleotides of claim 67 or 68, or the one or more vectors of claim 69 or 70.

75. The cell of claim 74, wherein the cell is a hematopoietic stem or progenitor cell (HSPC) or a hematopoietic stem cell (HSC).

76. A kit comprising the epegRNA of any one of claims 50-62, the complex of any one of claims 63-66, the one or more polynucleotides of claim 67 or 68, or the one or more vectors of claim 69 or 70.

77. Use of the epegRNA of any one of claims 50-62, the complex of any one of claims 63-66, the one or more polynucleotides of claim 67 or 68, the one or more vectors of claim 69 or 70, or the pharmaceutical composition of any one of claims 71-73 for the treatment of sickle cell disease.

78. Use of the epegRNA of any one of claims 50-62, the complex of any one of claims 63-66, the one or more polynucleotides of claim 67 or 68, the one or more vectors of claim 69 or 70, or the pharmaceutical composition of any one of claims 71-73 in the manufacture of a medicament for the treatment of sickle cell disease.

79. A cell comprising DNA comprising the sequence 5′-ATGGTGCACCTGACTCCTGA AGAGAAG-3′ (SEQ ID NO: 78), or a sequence at least 90%, at least 95%, at least 96%, at least 97%, at least 98%, or at least 99% identical to the sequence 5′-ATGGTGCACCTGACTCCTGAAGAGAAG-3′ (SEQ ID NO: 78), wherein the underlined nucleotide is an A.

80. The cell of claim 79, wherein the DNA is chromosomal DNA.

81. The cell of claim 79 or 80, wherein the cell is an HSPC or an HSC.


SEQUENCE LISTING (397 sequences)

SEQ ID NO: 1 - protein, 147 aa, synthetic construct
MVHLTPEEKSAVTALWGKVNVDEVGGEALGRLLVVYPWTQRFFESFGDLSTPDAVMGNPKVKAHGKKVLGAFSDGLAHLDNLKGTFATLSELHCDKLHVDPENFRLLGNVLVCVLAHHFGKEFTPPVQAAYQKVVAGVANALAHKYH

SEQ ID NO: 2 - DNA, 444 bp, Homo sapiens
  source 1..444: mol_type = genomic DNA
atggtgcacctgactcctgaggagaagtctgccgttactgccctgtggggcaaggtgaacgtggatgaagttggtggtgaggccctgggcaggctgctggtggtctacccttggacccagaggttctttgagtcctttggggatctgtccactcctgatgctgttatgggcaaccctaaggtgaaggctcatggcaagaaagtgctcggtgcctttagtgatggcctggctcacctggacaacctcaagggcacctttgccacactgagtgagctgcactgtgacaagctgcacgtggatcctgagaacttcaggctcctgggcaacgtgctggtctgtgtgctggcccatcactttggcaaagaattcaccccaccagtgcaggctgcctatcagaaagtggtggctggtgtggctaatgccctggcccacaagtatcactaa

SEQ ID NO: 3 - protein, 2117 aa, synthetic construct
MKRTADGSEFESPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSSGSETPGTSESATPESSGGSSGGSSTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGLLTSEGKEIKNKDEILALLKALFLPKRLSIIHCPGHQKGHSAEARGNRMADQAARKAAITETPDTSTLLIENSSPSGGSKRTADGSEFEPKKKRKV

SEQ ID NO: 4 - protein, 2117 aa, synthetic construct
MKRTADGSEFESPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSSGSETPGTSESATPESSGGSSGGSSTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSPSGGSKRTADGSEFEPKKKRKV

SEQ ID NO: 5 - protein, 2131 aa, synthetic construct
MKRTADGSEFESPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRKLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLKREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSKRTADGSEFESPKKKRKVSGGSSGGSTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSPSGGSKRTADGSEFESPKKKRKVGSGPAAKRVKLD

SEQ ID NO: 6 - protein, 1368 aa, Streptococcus pyogenes [sequence omitted]

SEQ ID NO: 7 - protein, 1368 aa, synthetic construct
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 8 - protein, 1367 aa, Streptococcus pyogenes
MDKKYSIGLDIGTNSVGWAVITDDYKVPSKKFKVLGNTDRHSIKKNLIGALLFGSGETAEATRLKRTARRRYTRRKNRICYLQEIFSNEMAKVDDSFFHRLEESFLVEEDKKHERHPIFGNIVDEVAYHEKYPTIYHLRKKLADSTDKADLRLIYLALAHMIKFRGHFLIEGDLNPDNSDVDKLFIQLVQIYNQLFEENPINASRVDAKAILSARLSKSRRLENLIAQLPGEKRNGLFGNLIALSLGLTPNFKSNFDLAEDAKLQLSKDTYDDDLDNLLAQIGDQYADLFLAAKNLSDAILLSDILRVNSEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGAYHDLLKIIKDKDFLDNEENEDILEDIVLTLTLFEDRGMIEERLKTYAHLFDDKVMKQLKRRRYTGWGRLSRKLINGIRDKQSGKTILDFLKSDGFANRNFMQLIHDDSLTFKEDIQKAQVSGQGHSLHEQIANLAGSPAIKKGILQTVKIVDELVKVMGHKPENIVIEMARENQTTQKGQKNSRERMKRIEEGIKELGSQILKEHPVENTQLQNEKLYLYYLQNGRDMYVDQELDINRLSDYDVDHIVPQSFIKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 9 - protein, 1404 aa, Streptococcus pyogenes
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGSPKKKRKVSSDYKDHDGDYKDHDIDYKDDDDKAAG

SEQ ID NO: 10 - protein, 1367 aa, synthetic construct
DKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 11 - protein, 1367 aa, synthetic construct
DKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRKLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLKREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 12 - protein, 1378 aa, Lactobacillus fermentum
MKEYHIGLDIGTSSIGWAVTDSQFKLMRIKGKTAIGVRLFEEGKTAAERRTFRTTRRRLKRRKWRLHYLDEIFAPHLQEVDENFLRRLKQSNIHPEDPTKNQAFIGKLLFPDLLKKNERGYPTLIKMRDELPVEQRAHYPVMNIYKLREAMINEDRQFDLREVYLAVHHIVKYRGHFLNNASVDKFKVGRIDFDKSFNVLNEAYEELQNGEGSFTIEPSKVEKIGQLLLDTKMRKLDRQKAVAKLLEVKVADKEETKRNKQIATAMSKLVLGYKADFATVAMANGNEWKIDLSSETSEDEIEKFREELSDAQNDILTEITSLFSQIMLNEIVPNGMSISESMMDRYWTHERQLAEVKEYLATQPASARKEFDQVYNKYIGQAPKERGFDLEKGLKKILSKKENWKEIDELLKAGDFLPKQRTSANGVIPHQMHQQELDRIIEKQAKYYPWLATENPATGERDRHQAKYELDQLVSFRIPYYVGPLVTPEVQKATSGAKFAWAKRKEDGEITPWNLWDKIDRAESAEAFIKRMTVKDTYLLNEDVLPANSLLYQKYNVLNELNNVRVNGRRLSVGIKQDIYTELFKKKKTVKASDVASLVMAKTRGVNKPSVEGLSDPKKFNSNLATYLDLKSIVGDKVDDNRYQTDLENIIEWRSVFEDGEIFADKLTEVEWLTDEQRSALVKKRYKGWGRLSKKLLTGIVDENGQRIIDLMWNTDQNFKEIVDQPVFKEQIDQLNQKAITNDGMTLRERVESVLDDAYTSPQNKKAIWQVVRVVEDIVKAVGNAPKSISIEFARNEGNKGEITRSRRTQLQKLFEDQAHELVKDTSLTEELEKAPDLSDRYYFYFTQGGKDMYTGDPINFDEISTKYDIDHILPQSFVKDNSLDNRVLTSRKENNKKSDQVPAKLYAAKMKPYWNQLLKQGLITQRKFENLTKDVDQNIKYRSLGFVKRQLVETRQVIKLTANILGSMYQEAGTEIIETRAGLTKQLREEFDLPKVREVNDYHHAVDAYLTTFAGQYLNRRYPKLRSFFVYGEYMKFKHGSDLKLRNFNFFHELMEGDKSQGKVVDQQTGELITTRDEVAKSFDRLLNMKYMLVSKEVHDRSDQLYGATIVTAKESGKLTSPIEIKKNRLVDLYGAYTNGTSAFMTIIKFTGNKPKYKVIGIPTTSAASLKRAGKPGSESYNQELHRIIKSNPKVKKGFEIVVPHVSYGQLIVDGDCKFTLASPTVQHPATQLVLSKKSLETISSGYKILKDKPAIANERLIRVFDEVVGQMNRYFTIFDQRSNRQKVADARDKFLSLPTESKYEGAKKVQVGKTEVITNLLMGLHANATQGDLKVLGLATFGFFQSTTGLSLSEDTMIVYQSPTGLFERRICLKDI

SEQ ID NO: 13 - protein, 1053 aa, Staphylococcus aureus
MGKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRKLINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYKNDLIKINGELYRVIGVNNDLLNRIEVNMIDITYREYLENMNDKRPPHIIKTIASKTQSIKKYSTDILGNLYEVKSKKHPQIIKK

SEQ ID NO: 14 - protein, 1409 aa, Streptococcus thermophilus
MLFNKCIIISINLDFSNKEKCMTKPYSIGLDIGTNSVGWAVITDNYKVPSKKMKVLGNTSKKYIKKNLLGVLLFDSGITAEGRRLKRTARRRYTRRRNRILYLQEIFSTEMATLDDAFFQRLDDSFLVPDDKRDSKYPIFGNLVEEKVYHDEFPTIYHLRKYLADSTKKADLRLVYLALAHMIKYRGHFLIEGEFNSKNNDIQKNFQDFLDTYNAIFESDLSLENSKQLEEIVKDKISKLEKKDRILKLFPGEKNSGIFSEFLKLIVGNQADFRKCFNLDEKASLHFSKESYDEDLETLLGYIGDDYSDVFLKAKKLYDAILLSGFLTVTDNETEAPLSSAMIKRYNEHKEDLALLKEYIRNISLKTYNEVFKDDTKNGYAGYIDGKTNQEDFYVYLKNLLAEFEGADYFLEKIDREDFLRKQRTFDNGSIPYQIHLQEMRAILDKQAKFYPFLAKNKERIEKILTFRIPYYVGPLARGNSDFAWSIRKRNEKITPWNFEDVIDKESSAEAFINRMTSFDLYLPEEKVLPKHSLLYETFNVYNELTKVRFIAESMRDYQFLDSKQKKDIVRLYFKDKRKVTDKDIIEYLHAIYGYDGIELKGIEKQFNSSLSTYHDLLNIINDKEFLDDSSNEAIIEEIIHTLTIFEDREMIKQRLSKFENIFDKSVLKKLSRRHYTGWGKLSAKLINGIRDEKSGNTILDYLIDDGISNRNFMQLIHDDALSFKKKIQKAQIIGDEDKGNIKEVVKSLPGSPAIKKGILQSIKIVDELVKVMGGRKPESIVVEMARENQYTNQGKSNSQQRLKRLEKSLKELGSKILKENIPAKLSKIDNNALQNDRLYLYYLQNGKDMYTGDDLDIDRLSNYDIDHIIPQAFLKDNSIDNKVLVSSASNRGKSDDFPSLEVVKKRKTFWYQLLKSKLISQRKFDNLTKAERGGLLPEDKAGFIQRQLVETRQITKHVARLLDEKFNNKKDENNRAVRTVKIITLKSTLVSQFRKDFELYKVREINDFHHAHDAYLNAVIASALLKKYPKLEPEFVYGDYPKYNSFRERKSATEKVYFYSNIMNIFKKSISLADGRVIERPLIEVNEETGESVWNKESDLATVRRVLSYPQVNVVKKVEEQNHGLDRGKPKGLFNANLSSKPKPNSNENLVGAKEYLDPKKYGGYAGISNSFAVLVKGTIEKGAKKKITNVLEFQGISILDRINYRKDKLNFLLEKGYKDIELIIELPKYSLFELSDGSRRMLASILSTNNKRGEIHKGNQIFLSQKFVKLLYHAKRISNTINENHRKYVENHKKEFEELFYYILEFNENYVGAKKNGKLLNSAFQSWQNHSIDELCSSFIGPTGSERKGLFELTSRGSAADFEFLGVKIPRYRDYTPSSLLKDATLIHQSVTGLYETRIDLAKLGEG

SEQ ID NO: 15 - protein, 1402 aa, Lactobacillus crispatus
MKIKNYNLALTPSTSAVGHVEVDDDLNILEPVHHQKAIGVAKFGEGETAEARRLARSARRTTKRRANRINHYFNEIMKPEIDKVDPLMFDRIKQAGLSPLDERKEFRTVIFDRPNIASYYHNQFPTIWHLQKYLMITDEKADIRLIYWALHSLLKHRGHFFNTTPMSQFKPGKLNLKDDMLALDDYNDLEGLSFAVANSPEIEKVIKDRSMHKKEKIAELKKLIVNDVPDKDLAKRNNKIITQIVNAIMGNSFHLNFIFDMDLDKLTSKAWSFKLDDPELDTKFDAISGSMTDNQIGIFETLQKIYSAISLLDILNGSSNVVDAKNALYDKHKRDLNLYFKFLNTLPDEIAKTLKAGYTLYIGNRKKDLLAARKLLKVNVAKNFSQDDFYKLINKELKSIDKQGLQTRFSEKVGELVAQNNFLPVQRSSDNVFIPYQLNAITFNKILENQGKYYDFLVKPNPAKKDRKNAPYELSQLMQFTIPYYVGPLVTPEEQVKSGIPKTSRFAWMVRKDNGAITPWNFYDKVDIEATADKFIKRSIAKDSYLLSELVLPKHSLLYEKYEVFNELSNVSLDGKKLSGGVKQILFNEVFKKTNKVNTSRILKALAKHNIPGSKITGLSNPEEFTSSLQTYNAWKKYFPNQIDNFAYQQDLEKMIEWSTVFEDHKILAKKLDEIEWLDDDQKKFVANTRLRGWGRLSKRLLTGLKDNYGKSIMQRLETTKANFQQIVYKPEFREQIDKISQAAAKNQSLEDILANSYTSPSNRKAIRKTMSVVDEYIKLNHGKEPDKIFLMFQRSEQEKGKQTEARSKQLNRILSQLKADKSANKLFSKQLADEFSNAIKKSKYKLNDKQYFYFQQLGRDALTGEVIDYDELYKYTVLHIIPRSKLTDDSQNNKVLTKYKIVDGSVALKFGNSYSDALGMPIKAFWTELNRLKLIPKGKLLNLTTDFSTLNKYQRDGYIARQLVETQQIVKLLATIMQSRFKHTKIIEVRNSQVANIRYQFDYFRIKNLNEYYRGFDAYLAAVVGTYLYKVYPKARRLFVYGQYLKPKKTNQENQDMHLDSEKKSQGFNFLWNLLYGKQDQIFVNGTDVIAFNRKDLITKMNTVYNYKSQKISLAIDYHNGAMFKATLFPRNDRDTAKTRKLIPKKKDYDTDIYGGYTSNVDGYMLLAEIIKRDGNKQYGFYGVPSRLVSELDTLKKTRYTEYEEKLKEIIKPELGVDLKKIKKIKILKNKVPFNQVIIDKGSKFFITSTSYRWNYRQLILSAESQQTLMDLVVDPDFSNHKARKDARKNADERLIKVYEEILYQVKNYMPMFVELHRCYEKLVDAQKTFKSLKISDKAMVLNQILILLHSNATSPVLEKLGYHTRFTLGKKHNLISENAVLVTQSITGLKENHVSIKQML

SEQ ID NO: 16 - protein, 1385 aa, unidentified
  source 1..1385: note = Pedicoccus damnosus
MTNEKYSIGLDIGTSSIGFAVVNDNNRVIRVKGKNAIGVRLFDEGKAAADRRSFRTTRRSFRTTRRRLSRRRWRLKLLREIFDAYITPVDEAFFIRLKESNLSPKDSKKQYSGDILFNDRSDKDFYEKYPTIYHLRNALMTEHRKFDVREIYLAIHHIMKFRGHFLNATPANNFKVGRLNLEEKFEELNDIYQRVFPDESIEFRTDNLEQIKEVLLDNKRSRADRQRTLVSDIYQSSEDKDIEKRNKAVATEILKASLGNKAKLNVITNVEVDKEAAKEWSITFDSESIDDDLAKIEGQMTDDGHEIIEVLRSLYSGITLSAIVPENHTLSQSMVAKYDLHKDHLKLFKKLINGMTDTKKAKNLRAAYDGYIDGVKGKVLPQEDFYKQVQVNLDDSAEANEIQTYIDQDIFMPKQRTKANGSIPHQLQQQELDQIIENQKAYYPWLAELNPNPDKKRQQLAKYKLDELVTFRVPYYVGPMITAKDQKNQSGAEFAWMIRKEPGNITPWNFDQKVDRMATANQFIKRMTTTDTYLLGEDVLPAQSLLYQKFEVLNELNKIRIDHKPISIEQKQQIFNDLFKQFKNVTIKHLQDYLVSQGQYSKRPLIEGLADEKRFNSSLSTYSDLCGIFGAKLVEENDRQEDLEKIIEWSTIFEDKKIYRAKLNDLTWLTDDQKEKLATKRYQGWGRLSRKLLVGLKNSEHRNIMDILWITNENFMQIQAEPDFAKLVTDANKGMLEKTDSQDVINDLYTSPQNKKAIRQILLVVHDIQNAMHGQAPAKIHVEFARGEERNPRRSVQRQRQVEAAYEKVSNELVSAKVRQEFKEAINNKRDFKDRLFLYFMQGGIDIYTGKQLNIDQLSSYQIDHILPQAFVKDDSLTNRVLTNENQVKADSVPIDIFGKKMLSVWGRMKDQGLISKGKYRNLTMNPENISAHTENGFINRQLVETRQVIKLAVNILADEYGDSTQIISVKADLSHQMREDFELLKNRDVNDYHHAFDAYLAAFIGNYLLKRYPKLESYFVYGDFKKFTQKETKMRRFNFIYDLKHCDQVVNKETGEILWTKDEDIKYIRHLFAYKKILVSHEVREKRGALYNQTIYKAKDDKGSGQESKKLIRIKDDKETKIYGGYSGKSLAYMTIVQITKKNKVSYRVIGIPTLALARLNKLENDSTENNGELYKIIKPQFTHYKVDKKNGEIIETTDDFKIVVSKVRFQQLIDDAGQFFMLASDTYKNNAQQLVISNNALKAINNTNITDCPRDDLERLDNLRLDSAFDEIVKKMDKYFSAYDANNFREKIRNSNLIFYQLPVEDQWENNKITELGKRTVLTRILQGLHANATTTDMSIFKIKTPFGQLRQRSGISLSENAQLIYQSPTGLFERRVQLNKIK

SEQ ID NO: 17 - protein, 1367 aa, Fusobacterium nucleatum
MKKQKFSDYYLGFDIGTNSVGWCVTDLDYNVLRFNKKDMWGSRLFEEAKTAAERRVQRNSRRRLKRRKWRLNLLEEIFSNEILKIDSNFFRRLKESSLWLEDKSSKEKFTLFNDDNYKDYDFYKQYPTIFHLRNELIKNPEKKDIRLVYLAIHSIFKSRGHFLFEGQNLKEIKNFETLYNNLIAFLEDNGINKIIDKNNIEKLEKIVCDSKKGLKDKEKEFKEIFNSDKQLVAIFKLSVGSSVSLNDLFDTDEYKKGEVEKEKISFREQIYEDDKPIYYSILGEKIELLDIAKTFYDFMVLNNILADSQYISEAKVKLYEEHKKDLKNLKYIIRKYNKGNYDKLFKDKNENNYSAYIGLNKEKSKKEVIEKSRLKIDDLIKNIKGYLPKVEEIEEKDKAIFNKILNKIELKTILPKQRISDNGTLPYQIHEAELEKILENQSKYYDFLNYEENGIITKDKLLMTFKFRIPYYVGPLNSYHKDKGGNSWIVRKEEGKILPWNFEQKVDIEKSAEEFIKRMTNKCTYLNGEDVIPKDTFLYSEYVILNELNKVQVNDEFLNEENKRKIIDELFKENKKVSEKKFKEYLLVKQIVDGTIELKGVKDSFNSNYISYIRFKDIFGEKLNLDIYKEISEKSILWKCLYGDDKKIFEKKIKNEYGDILTKDEIKKINTFKFNNWGRLSEKLLTGIEFINLETGECYSSVMDALRRTNYNLMELLSSKFTLQESINNENKEMNEASYRDLIEESYVSPSLKRAIFQTLKIYEEIRKITGRVPKKVFIEMARGGDESMKNKKIPARQEQLKKLYDSCGNDIANFSIDIKEMKNSLISYDNNSLRQKKLYLYYLQFGKCMYTGREIDLDRLLQNNDTYDIDHIYPRSKVIKDDSFDNLVLVLKNENAEKSNEYPVKKEIQEKMKSFWRFLKEKNFISDEKYKRLTGKDDFELRGFMARQLVNVRQTTKEVGKILQQIEPEIKIVYSKAEIASSFREMFDFIKVRELNDTHHAKDAYLNIVAGNVYNTKFTEKPYRYLQEIKENYDVKKIYNYDIKNAWDKENSLEIVKKNMEKNTVNITRFIKEKKGQLFDLNPIKKGETSNEIISIKPKVYNGKDDKLNEKYGYYKSLNPAYFLYVEHKEKNKRIKSFERVNLVDVNNIKDEKSLVKYLIENKKLVEPRVIKKVYKRQVILINDYPYSIVTLDSNKLMDFENLKPLFLENKYEKILKNVIKFLEDNQGKSEENYKFIYLKKKDRYEKNETLESVKDRYNLEFNEMYDKFLEKLDSKDYKNYMNNKKYQELLDVKEKFIKLNLFDKAFTLKSFLDLFNRKTMADFSKVGLTKYLGKIQKISSNVLSKNELYLLEESVTGLFVKKIKL

SEQ ID NO: 18 - protein, 1286 aa, Enterococcus cecorum
RRKQRIQILQELLGEEVLKTDPGFFHRMKESRYVVEDKRTLDGKQVELPYALFVDKDYTDKEYYKQFPTINHLIVYLMTTSDTPDIRLVYLALHYYMKNRGNFLHSGDINNVKDINDILEQLDNVLETFLDGWNLKLKSYVEDIKNIYNRDLGRGERKKAFVNTLGAKTKAEKAFCSLISGGSTNLAELFDDSSLKEIETPKIEFASSSLEDKIDGIQEALEDRFAVIEAAKRLYDWKTLTDILGDSSSLAEARVNSYQMHHEQLLELKSLVKEYLDRKVFQEVFVSLNVANNYPAYIGHTKINGKKKELEVKRTKRNDFYSYVKKQVIEPIKKKVSDEAVLTKLSEIESLIEVDKYLPLQVNSDNGVIPYQVKLNELTRIFDNLENRIPVLRENRDKIIKTFKFRIPYYVGSLNGVVKNGKCTNWMVRKEEGKIYPWNFEDKVDLEASAEQFIRRMTNKCTYLVNEDVLPKYSLLYSKYLVLSELNNLRIDGRPLDVKIKQDIYENVFKKNRKVTLKKIKKYLLKEGIITDDDELSGLADDVKSSLTAYRDFKEKLGHLDLSEAQMENIILNITLFGDDKKLLKKRLAALYPFIDDKSLNRIATLNYRDWGRLSERFLSGITSVDQETGELRTIIQCMYETQANLMQLLAEPYHFVEAIEKENPKVDLESISYRIVNDLYVSPAVKRQIWQTLLVIKDIKQVMKHDPERIFIEMAREKQESKKTKSRKQVLSEVYKKAKEYEHLFEKLNSLTEEQLRSKKIYLYFTQLGKCMYSGEPIDFENLVSANSNYDIDHIYPQSKTIDDSFNNIVLVKKSLNAYKSNHYPIDKNIRDNEKVKTLWNTLVSKGLITKEKYERLIRSTPFSDEELAGFIARQLVETRQSTKAVAEILSNWFPESEIVYSKAKNVSNFRQDFEILKVRELNDCHHAHDAYLNIVVGNAYHTKFTNSPYRFIKNKANQEYNLRKLLQKVNKIESNGVVAWVGQSENNPGTIATVKKVIRRNTVLISRMVKEVDGQLFDLTLMKKGKGQVPIKSSDERLTDISKYGGYNKATGAYFTFVKSKKRGKVVRSFEYVPLHLSKQFENNNELLKEYIEKDRGLTDVEILIPKVLINSLFRYNGSLVRITGRGDTRLLLVHEQPLYVSNSFVQQLKSVSSYKLKKSENDNAKLTKTATEKLSNIDELYDGLLRKLDLPIYSYWFSSIKEYLVESRTKYIKLSIEEKALVIFEILHLFQSDAQVPNLKILGLSTKPSRIRIQKNLKDTDKMSIIHQSPSGIFEHEIELTSL

SEQ ID NO: 19 - protein, 1312 aa, unidentified
  source 1..1312: note = Anaerostipes hadrus
MQNGFLGITVSSEQVGWAVTNPKYELERASRKDLWGVRLFDKAETAEDRRMFRTNRRLNQRKKNRIHYLRDIFHEEVNQKDPNFFQQLDESNFCEDDRTVEFNFDTNLYKNQFPTVYHLRKYLMETKDKPDIRLVYLAFSKFMKNRGHFLYKGNLGEVMDFENSMKGFCESLEKFNIDFPTLSDEQVKEVRDILCDHKIAKTVKKKNIITITKVKSKTAKAWIGLFCGCSVPVKVLFQDIDEEIVTDPEKISFEDASYDDYIANIEKGVGIYYEAIVSAKMLFDWSILNEILGDHQLLSDAMIAEYNKHHDDLKRLQKIIKGTGSRELYQDIFINDVSGNYVCYVGHAKTMSSADQKQFYTFLKNRLKNVNGISSEDAEWIDTEIKNGTLLPKQTKRDNSVIPHQLQLREFELILDNMQEMYPFLKENREKLLKIFNFVIPYYVGPLKGVVRKGESTNWMVPKKDGVIHPWNFDEMVDKEASAECFISRMTGNCSYLFNEKVLPKNSLLYETFEVLNELNPLKINGEPISVELKQRIYEQLFLTGKKVTKKSLTKYLIKNGYDKDIELSGIDNEFHSNLKSHIDFEDYDNLSDEEVEQIILRITVFEDKQLLKDYLNREFVKLSEDERKQICSLSYKGWGNLSEMLLNGITVTDSNGVEVSVMDMLWNTNLNLMQILSKKYGYKAEIEHYNKEHEKTIYNREDLMDYLNIPPAQRRKVNQLITIVKSLKKTYGVPNKIFFKISREHQDDPKRTSSRKEQLKYLYKSLKSEDEKHLMKELDELNDHELSNDKVYLYFLQKGRCIYSGKKLNLSRLRKSNYQNDIDYIYPLSAVNDRSMNNKVLTGIQENRADKYTYFPVDSEIQKKMKGFWMELVLQGFMTKEKYFRLSRENDFSKSELVSFIEREISDNQQSGRMIASVLQYYFPESKIVFVKEKLISSFKRDFHLISSYGHNHLQAAKDAYITIVVGNVYHTKFTMDPAIYFKNHKRKDYDLNRLFLENISRDGQIAWESGPYGSIQTVRKEYAQNHIAVTKRVVEVKGGLFKQMPLKKGHGEYPLKTNDPRFGNIAQYGGYTNVTGSYFVLVESMEKGKKRISLEYVPVYLHERLEDDPGHKLLKEYLVDHRKLNHPKILLAKVRKNSLLKIDGFYYRLNGRSGNALILTNAVELIMDDWQTKTANKISGYMKRRAIDKKARVYQNEFHIQELEQLYDFYLDKLKNGVYKNRKNNQAELIHNEKEQFMELKTEDQCVLLTEIKKLFVCSPMQADLTLIGGSKHTGMIAMSSNVTKADFAVIAEDPLGLRNKVIYSHKGEK

SEQ ID NO: 20 - protein, 1344 aa, Kandleria vitulina
MSQNNNKIYNIGLDIGDASVGWAVVDEHYNLLKRHGKHMWGSRLFTQANTAVERRSSRSTRRRYNKRRERIRLLREIMEDMVLDVDPTFFIRLANVSFLDQEDKKDYLKENYHSNYNLFIDKDFNDKTYYDKYPTIYHLRKHLCESKEKEDPRLIYLALHHIVKYRGNFLYEGQKFSMDVSNIEDKMIDVLRQFNEINLFEYVEDRKKIDEVLNVLKEPLSKKHKAEKAFALFDTTKDNKAAYKELCAALAGNKFNVTKMLKEAELHDEDEKDISFKFSDATFDDAFVEKQPLLGDCVEFIDLLHDIYSWVELQNILGSAHTSEPSISAAMIQRYEDHKNDLKLLKDVIRKYLPKKYFEVFRDEKSKKNNYCNYINHPSKTPVDEFYKYIKKLIEKIDDPDVKTILNKIELESFMLKQNSRTNGAVPYQMQLDELNKILENQSVYYSDLKDNEDKIRSILTFRIPYYFGPLNITKDRQFDWIIKKEGKENERILPWNANEIVDVDKTADEFIKRMRNFCTYFPDEPVMAKNSLTVSKYEVLNEINKLRINDHLIKRDMKDKMLHTLFMDHKSISANAMKKWLVKNQYFSNTDDIKIEGFQKENACSTSLTPWIDFTKIFGKINESNYDFIEKIIYDVTVFEDKKILRRRLKKEYDLDEEKIKKILKLKYSGWSRLSKKLLSGIKTKYKDSTRTPETVLEVMERTNMNLMQVINDEKLGFKKTIDDANSTSVSGKFSYAEVQELAGSPAIKRGIWQALLIVDEIKKIMKHEPAHVYIEFARNEDEKERKDSFVNQMLKLYKDYDFEDETEKEANKHLKGEDAKSKIRSERLKLYYTQMGKCMYTGKSLDIDRLDTYQVDHIVPQSLLKDDSIDNKVLVLSSENQRKLDDLVIPSSIRNKMYGFWEKLFNNKIISPKKFYSLIKTEFNEKDQERFINRQIVETRQITKHVAQIIDNHYENTKVVTVRADLSHQFRERYHIYKNRDINDFHHAHDAYIATILGTYIGHRFESLDAKYIYGEYKRIFRNQKNKGKEMKKNNDGFILNSMRNIYADKDTGEIVWDPNYIDRIKKCFYYKDCFVTKKLEENNGTFFNVTVLPNDTNSDKDNTLATVPVNKYRSNVNKYGGFSGVNSFIVAIKGKKKKGKKVIEVNKLTGIPLMYKNADEEIKINYLKQAEDLEEVQIGKEILKNQLIEKDGGLYYIVAPTEIINAKQLILNESQTKLVCEIYKAMKYKNYDNLDSEKIIDLYRLLINKMELYYPEYRKQLVKKFEDRYEQLKVISIEEKCNIIKQILATLHCNSSIGKIMYSDFKISTTIGRLNGRTISLDDISFIAESPTGMYSKKYKL

SEQ ID NO: 21 - protein, 1288 aa, Enterococcus faecalis
MRLFEEGHTAEDRRLKRTARRRISRRRNRLRYLQAFFEEAMTDLDENFFARLQESFLVPEDKKWHRHPIFAKLEDEVAYHETYPTIYHLRKKLADSSEQADLRLIYLALAHIVKYRGHFLIEGKLSTENTSVKDQFQQFMVIYNQTFVNGESRLVSAPLPESVLIEEELTEKASRTKKSEKVLQQFPQEKANGLFGQFLKLMVGNKADFKKVFGLEEEAKITYASESYEEDLEGILAKVGDEYSDVFLAAKNVYDAVELSTILADSDKKSHAKLSSSMIVRFTEHQEDLKKFKRFIRENCPDEYDNLFKNEQKDGYAGYIAHAGKVSQLKFYQYVKKIIQDIAGAEYFLEKIAQENFLRKQRTFDNGVIPHQIHLAELQAIIHRQAAYYPFLKENQEKIEQLVTFRIPYYVGPLSKGDASTFAWLKRQSEEPIRPWNLQETVDLDQSATAFIERMTNFDTYLPSEKVLPKHSLLYEKFMVFNELTKISYTDDRGIKANFSGKEKEKIFDYLFKTRRKVKKKDIIQFYRNEYNTEIVTLSGLEEDQFNASFSTYQDLLKCGLTRAELDHPDNAEKLEDIIKILTIFEDRQRIRTQLSTFKGQFSAEVLKKLERKHYTGWGRLSKKLINGIYDKESGKTILDYLVKDDGVSKHYNRNFMQLINDSQLSFKNAIQKAQSSEHEETLSETVNELAGSPAIKKGIYQSLKIVDELVAIMGYAPKRIVVEMARENQTTSTGKRRSIQRLKIVEKAMAEIGSNLLKEQPTTNEQLRDTRLFLYYMQNGKDMYTGDELSLHRLSHYDIDHIIPQSFMKDDSLDNLVLVGSTENRGKSDDVPSKEVVKDMKAYWEKLYAAGLISQRKFQRLTKGEQGGLTLEDKAHFIQRQLVETRQITKNVAGILDQRYNAKSKEKKVQIITLKASLTSQFRSIFGLYKVREVNDYHHGQDAYLNCVVATTLLKVYPNLAPEFVYGEYPKFQTFKENKATAKAIIYTNLLRFFTEDEPRFTKDGEILWSNSYLKTIKKELNYHQMNIVKKVEVQKGGFSKESIKPKGPSNKLIPVKNGLDPQKYGGFDSPVVAYTVLFTHEKGKKPLIKQEILGITIMEKTRFEQNPILFLEEKGFLRPRVLMKLPKYTLYEFPEGRRRLLASAKEAQKGNQMVLPEHLLTLLYHAKQCLLPNQSESLAYVEQHQPEFQEILERVVDFAEVHTLAKSKVQQIVKLFEANQTADVKEIAASFIQLMQFNAMGAPSTFKFFQKDIERARYTSIKEIFDATIIYQSPTGLYETRRKVVD

SEQ ID NO: 22 - protein, 1052 aa, Staphylococcus aureus
KRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 23 - protein, 1082 aa, Geobacillus thermodenitrificans
MKYKIGLDIGITSIGWAVINLDIPRIEDLGVRIFDRAENPKTGESLALPRRLARSARRRLRRRKHRLERIRRLFVREGILTKEELNKLFEKKHEIDVWQLRVEALDRKLNNDELARILLHLAKRRGFRSNRKSERTNKENSTMLKHIEENQSILSSYRTVAEMVVKDPKFSLHKRNKEDNYTNTVARDDLEREIKLIFAKQREYGNIVCTEAFEHEYISIWASQRPFASKDDIEKKVGFCTFEPKEKRAPKATYTFQSFTVWEHINKLRLVSPGGIRALTDDERRLIYKQAFHKNKITFHDVRTLLNLPDDTRFKGLLYDRNTTLKENEKVRFLELGAYHKIRKAIDSVYGKGAAKSFRPIDFDTFGYALTMFKDDTDIRSYLRNEYEQNGKRMENLADKVYDEELIEELLNLSFSKFGHLSLKALRNILPYMEQGEVYSTACERAGYTFTGPKKKQKTVLLPNIPPIANPVVMRALTQARKVVNAIIKKYGSPVSIHIELARELSQSFDERRKMQKEQEGNRKKNETAIRQLVEYGLTLNPTGLDIVKFKLWSEQNGKCAYSLQPIEIERLLEPGYTEVDHVIPYSRSLDDSYTNKVLVLTKENREKGNRTPAEYLGLGSERWQQFETFVLTNKQFSKKKRDRLLRLHYDENEENEFKNRNLNDTRYISRFLANFIREHLKFADSDDKQKVYTVNGRITAHLRSRWNFNKNREESNLHHAVDAAIVACTTPSDIARVTAFYQRREQNKELSKKTDPQFPQPWPHFADELQARLSKNPKESIKALNLGNYDNEKLESLQPVFVSRMPKRSITGAAHQETLRRYIGIDERSGKIQTVVKKKLSEIQLDKTGHFPMYGKESDPRTYEAIRQRLLEHNNDPKKAFQEPLYKPKKNGELGPIIRTIKIIDTTNQVIPLNDGKTVAYNSNIVRVDVFEKDGKYYCVPIYTIDMMKGILPNKAIEPNKPYSEWKEMTEDYTFRFSLYPNDLIRIEFPREKTIKTAVGEEIKIKDLFAYYQTIDSSNGGLSLVSHDNNFSLRSIGSRTLKRFEKYQVDVLGNIYKVRGEKRVGVASSSHSKAGETIRPL

SEQ ID NO: 24 - protein, 1375 aa, Streptococcus canis
MEKKYSIGLDIGTNSVGWAVITDDYKVPSKKFKVLGNTNRKSIKKNLMGALLFDSGETAEATRLKRTARRRYTRRKNRIRYLQEIFANEMAKLDDSFFQRLEESFLVEEDKKNERHPIFGNLADEVAYHRNYPTIYHLRKKLADSPEKADLRLIYLALAHIIKFRGHFLIEGKLNAENSDVAKLFYQLIQTYNQLFEESPLDEIEVDAKGILSARLSKSKRLEKLIAVFPNEKKNGLFGNIIALALGLTPNFKSNFDLTEDAKLQLSKDTYDDDLDELLGQIGDQYADLFSAAKNLSDAILLSDILRSNSEVTKAPLSASMVKRYDEHHQDLALLKTLVRQQFPEKYAEIFKDDTKNGYAGYVGIGIKHRKRTTKLATQEEFYKFIKPILEKMDGAEELLAKLNRDDLLRKQRTFDNGSIPHQIHLKELHAILRRQEEFYPFLKENREKIEKILTFRIPYYVGPLARGNSRFAWLTRKSEEAITPWNFEEVVDKGASAQSFIERMTNFDEQLPNKKVLPKHSLLYEYFTVYNELTKVKYVTERMRKPEFLSGEQKKAIVDLLFKTNRKVTVKQLKEDYFKKIECFDSVEIIGVEDRFNASLGTYHDLLKIIKDKDFLDNEENEDILEDIVLTLTLFEDREMIEERLKTYAHLFDDKVMKQLKRRHYTGWGRLSRKMINGIRDKQSGKTILDFLKSDGFSNRNFMQLIHDDSLTFKEEIEKAQVSGQGDSLHEQIADLAGSPAIKKGILQTVKIVDELVKVMGHKPENIVIEMARENQTTTKGLQQSRERKKRIEEGIKELESQILKENPVENTQLQNEKLYLYYLQNGRDMYVDQELDINRLSDYDVDHIVPQSFIKDDSIDNKVLTRSVENRGKSDNVPSEEVVKKMKNYWRQLLNAKLITQRKFDNLTKAERGGLSEADKAGFIKRQLVETRQITKHVARILDSRMNTKRDKNDKPIREVKVITLKSKLVSDFRKDFQLYKVRDINNYHHAHDAYLNAVVGTALIKKYPKLESEFVYGDYKVYDVRKMIAKSEQEIGKATAKRFFYSNIMNFFKTEVKLANGEIRKRPLIETNGETGEVVWNKEKDFATVRKVLAMPQVNIVKKTEVQTGGFSKESILSKRESAKLIPRKKGWDTRKYGGFGSPTVAYSILVVAKVEKGKAKKLKSVKVLVGITIMEKGSYEKDPIGFLEAKGYKDIKKELIFKLPKYSLFELENGRRRMLASATELQKANELVLPQHLVRLLYYTQNISATTGSNNLGYIEQHREEFKEIFEKIIDFSEKYILKNKVNSNLKSSFDEQFAVSDSILLSNSFVSLLKYTSFGASGGFTFLDLDVKQGRLRYQTVTEVLDATLIYQSITGLYETRTDLSQLGGD

SEQ ID NO: 25 - DNA, 4104 bp, Streptococcus pyogenes
  source 1..4104: mol_type = genomic DNA
atggataagaaatactcaataggcttagatatcggcacaaatagcgtcggatgggcggtgatcactgatgattataaggttccgtctaaaaagttcaaggttctgggaaatacagaccgccacagtatcaaaaaaaatcttataggggctcttttatttggcagtggagagacagcggaagcgactcgtctcaaacggacagctcgtagaaggtatacacgtcggaagaatcgtatttgttatctacaggagattttttcaaatgagatggcgaaagtagatgatagtttctttcatcgacttgaagagtcttttttggtggaagaagacaagaagcatgaacgtcatcctatttttggaaatatagtagatgaagttgcttatcatgagaaatatccaactatctatcatctgcgaaaaaaattggcagattctactgataaagcggatttgcgcttaatctatttggccttagcgcatatgattaagtttcgtggtcattttttgattgagggagatttaaatcctgataatagtgatgtggacaaactatttatccagttggtacaaatctacaatcaattatttgaagaaaaccctattaacgcaagtagagtagatgctaaagcgattctttctgcacgattgagtaaatcaagacgattagaaaatctcattgctcagctccccggtgagaagagaaatggcttgtttgggaatctcattgctttgtcattgggattgacccctaattttaaatcaaattttgatttggcagaagatgctaaattacagctttcaaaagatacttacgatgatgatttagataatttattggcgcaaattggagatcaatatgctgatttgtttttggcagctaagaatttatcagatgctattttactttcagatatcctaagagtaaatagtgaaataactaaggctcccctatcagcttcaatgattaagcgctacgatgaacatcatcaagacttgactcttttaaaagctttagttcgacaacaacttccagaaaagtataaagaaatcttttttgatcaatcaaaaaacggatatgcaggttatattgatgggggagctagccaagaagaattttataaatttatcaaaccaattttagaaaaaatggatggtactgaggaattattggtgaaactaaatcgtgaagatttgctgcgcaagcaacggacctttgacaacggctctattccccatcaaattcacttgggtgagctgcatgctattttgagaagacaagaagacttttatccatttttaaaagacaatcgtgagaagattgaaaaaatcttgacttttcgaattccttattatgttggtccattggcgcgtggcaatagtcgttttgcatggatgactcggaagtctgaagaaacaattaccccatggaattttgaagaagttgtcgataaaggtgcttcagctcaatcatttattgaacgcatgacaaactttgataaaaatcttccaaatgaaaaagtactaccaaaacatagtttgctttatgagtattttacggtttataacgaattgacaaaggtcaaatatgttactgagggaatgcgaaaaccagcatttctttcaggtgaacagaagaaagccattgttgatttactcttcaaaacaaatcgaaaagtaaccgttaagcaattaaaagaagattatttcaaaaaaatagaatgttttgatagtgttgaaatttcaggagttgaagatagatttaatgcttcattaggcgcctaccatgatttgctaaaaattattaaagataaagattttttggataatgaagaaaatgaagatatcttagaggatattgttttaacattgaccttatttgaagatagggggatgattgaggaaagacttaaaacatatgctcacctctttgatgataaggtgatgaaacagcttaaacgtcgccgttatactggttggggacgtttgtctcgaaaattgattaatggtattagggataagcaatctggcaaaacaatattagattttttgaaatcagatggttttgccaatcgcaattttatgcagctgatccatgatgatagtttgacatttaaagaagatattcaaaaagcacaggtgtctggacaaggccatagtttacatgaacagattgctaacttagctggcagtcctgctattaaaaaaggtattttacagactgtaaaaattgttgatgaactggtcaaagtaatggggcataagccagaaaatatcgttattgaaatggcacgtgaaaatcagacaactcaaaagggccagaaaaattcgcgagagcgtatgaaacgaatcgaagaaggtatcaaagaattaggaagtcagattcttaaagagcatcctgttgaaaatactcaattgcaaaatgaaaagctctatctctattatctacaaaatggaagagacatgtatgtggaccaagaattagatattaatcgtttaagtgattatgatgtcgatcacattgttccacaaagtttcattaaagacgattcaatagacaataaggtactaacgcgttctgataaaaatcgtggtaaatcggataacgttccaagtgaagaagtagtcaaaaagatgaaaaactattggagacaacttctaaacgccaagttaatcactcaacgtaagtttgataatttaacgaaagctgaacgtggaggtttgagtgaacttgataaagctggttttatcaaacgccaattggttgaaactcgccaaatcactaagcatgtggcacaaattttggatagtcgcatgaatactaaatacgatgaaaatgataaacttattcgagaggttaaagtgattaccttaaaatctaaattagtttctgacttccgaaaagatttccaattctataaagtacgtgagattaacaattaccatcatgcccatgatgcgtatctaaatgccgtcgttggaactgctttgattaagaaatatccaaaacttgaatcggagtttgtctatggtgattataaagtttatgatgttcgtaaaatgattgctaagtctgagcaagaaataggcaaagcaaccgcaaaatatttcttttactctaatatcatgaacttcttcaaaacagaaattacacttgcaaatggagagattcgcaaacgccctctaatcgaaactaatggggaaactggagaaattgtctgggataaagggcgagattttgccacagtgcgcaaagtattgtccatgccccaagtcaatattgtcaagaaaacagaagtacagacaggcggattctccaaggagtcaattttaccaaaaagaaattcggacaagcttattgctcgtaaaaaagactgggatccaaaaaaatatggtggttttgatagtccaacggtagcttattcagtcctagtggttgctaaggtggaaaaagggaaatcgaagaagttaaaatccgttaaagagttactagggatcacaattatggaaagaagttcctttgaaaaaaatccgattgactttttagaagctaaaggatataaggaagttaaaaaagacttaatcattaaactacctaaatatagtctttttgagttagaaaacggtcgtaaacggatgctggctagtgccggagaattacaaaaaggaaatgagctggctctgccaagcaaatatgtgaattttttatatttagctagtcattatgaaaagttgaagggtagtccagaagataacgaacaaaaacaattgtttgtggagcagcataagcattatttagatgagattattgagcaaatcagtgaattttctaagcgtgttattttagcagatgccaatttagataaagttcttagtgcatataacaaacatagagacaaaccaatacgtgaacaagcagaaaatattattcatttatttacgttgacgaatcttggagctcccgctgcttttaaatattttgatacaacaattgatcgtaaacgatatacgtctacaaaagaagttttagatgccactcttatccatcaatccatcactggtctttatgaaacacgcattgatttgagtcagctaggaggtgactga

SEQ ID NO: 26 - DNA, 4212 bp, Streptococcus pyogenes
  source 1..4212: mol_type = genomic DNA
atggataaaaagtattctattggtttagacatcggcactaattccgttggatgggctgtcataaccgatgaatacaaagtaccttcaaagaaatttaaggtgttggggaacacagaccgtcattcgattaaaaagaatcttatcggtgccctcctattcgatagtggcgaaacggcagaggcgactcgcctgaaacgaaccgctcggagaaggtatacacgtcgcaagaaccgaatatgttacttacaagaaatttttagcaatgagatggccaaagttgacgattctttctttcaccgtttggaagagtccttccttgtcgaagaggacaagaaacatgaacggcaccccatctttggaaacatagtagatgaggtggcatatcatgaaaagtacccaacgatttatcacctcagaaaaaagctagttgactcaactgataaagcggacctgaggttaatctacttggctcttgcccatatgataaagttccgtgggcactttctcattgagggtgatctaaatccggacaactcggatgtcgacaaactgttcatccagttagtacaaacctataatcagttgtttgaagagaaccctataaatgcaagtggcgtggatgcgaaggctattcttagcgcccgcctctctaaatcccgacggctagaaaacctgatcgcacaattacccggagagaagaaaaatgggttgttcggtaaccttatagcgctctcactaggcctgacaccaaattttaagtcgaacttcgacttagctgaagatgccaaattgcagcttagtaaggacacgtacgatgacgatctcgacaatctactggcacaaattggagatcagtatgcggacttatttttggctgccaaaaaccttagcgatgcaatcctcctatctgacatactgagagttaatactgagattaccaaggcgccgttatccgcttcaatgatcaaaaggtacgatgaacatcaccaagacttgacacttctcaaggccctagtccgtcagcaactgcctgagaaatataaggaaatattctttgatcagtcgaaaaacgggtacgcaggttatattgacggcggagcgagtcaagaggaattctacaagtttatcaaacccatattagagaagatggatgggacggaagagttgcttgtaaaactcaatcgcgaagatctactgcgaaagcagcggactttcgacaacggtagcattccacatcaaatccacttaggcgaattgcatgctatacttagaaggcaggaggatttttatccgttcctcaaagacaatcgtgaaaagattgagaaaatcctaacctttcgcataccttactatgtgggacccctggcccgagggaactctcggttcgcatggatgacaagaaagtccgaagaaacgattactccatggaattttgaggaagttgtcgataaaggtgcgtcagctcaatcgttcatcgagaggatgaccaactttgacaagaatttaccgaacgaaaaagtattgcctaagcacagtttactttacgagtatttcacagtgtacaatgaactcacgaaagttaagtatgtcactgagggcatgcgtaaacccgcctttctaagcggagaacagaagaaagcaatagtagatctgttattcaagaccaaccgcaaagtgacagttaagcaattgaaagaggactactttaagaaaattgaatgcttcgattctgtcgagatctccggggtagaagatcgatttaatgcgtcacttggtacgtatcatgacctcctaaagataattaaagataaggacttcctggataacgaagagaatgaagatatcttagaagatatagtgttgactcttaccctctttgaagatcgggaaatgattgaggaaagactaaaaacatacgctcacctgttcgacgataaggttatgaaacagttaaagaggcgtcgctatacgggctggggacgattgtcgcggaaacttatcaacgggataagagacaagcaaagtggtaaaactattctcgattttctaaagagcgacggcttcgccaataggaactttatgcagctgatccatgatgactctttaaccttcaaagaggatatacaaaaggcacaggtttccggacaaggggactcattgcacgaacatattgcgaatcttgctggttcgccagccatcaaaaagggcatactccagacagtcaaagtagtggatgagctagttaaggtcatgggacgtcacaaaccggaaaacattgtaatcgagatggcacgcgaaaatcaaacgactcagaaggggcaaaaaaacagtcgagagcggatgaagagaatagaagagggtattaaagaactgggcagccagatcttaaaggagcatcctgtggaaaatacccaattgcagaacgagaaactttacctctattacctacaaaatggaagggacatgtatgttgatcaggaactggacataaaccgtttatctgattacgacgtcgatcacattgtaccccaatcctttttgaaggacgattcaatcgacaataaagtgcttacacgctcggataagaaccgagggaaaagtgacaatgttccaagcgaggaagtcgtaaagaaaatgaagaactattggcggcagctcctaaatgcgaaactgataacgcaaagaaagttcgataacttaactaaagctgagaggggtggcttgtctgaacttgacaaggccggatttattaaacgtcagctcgtggaaacccgccaaatcacaaagcatgttgcacagatactagattcccgaatgaatacgaaatacgacgagaacgataagctgattcgggaagtcaaagtaatcactttaaagtcaaaattggtgtcggacttcagaaaggattttcaattctataaagttagggagataaataactaccaccatgcgcacgacgcttatcttaatgccgtcgtagggaccgcactcattaagaaatacccgaagctagaaagtgagtttgtgtatggtgattacaaagtttatgacgtccgtaagatgatcgcgaaaagcgaacaggagataggcaaggctacagccaaatacttcttttattctaacattatgaatttctttaagacggaaatcactctggcaaacggagagatacgcaaacgacctttaattgaaaccaatggggagacaggtgaaatcgtatgggataagggccgggacttcgcgacggtgagaaaagttttgtccatgccccaagtcaacatagtaaagaaaactgaggtgcagaccggagggttttcaaaggaatcgattcttccaaaaaggaatagtgataagctcatcgctcgtaaaaaggactgggacccgaaaaagtacggtggcttcgatagccctacagttgcctattctgtcctagtagtggcaaaagttgagaagggaaaatccaagaaactgaagtcagtcaaagaattattggggataacgattatggagcgctcgtcttttgaaaagaaccccatcgacttccttgaggcgaaaggttacaaggaagtaaaaaaggatctcataattaaactaccaaagtatagtctgtttgagttagaaaatggccgaaaacggatgttggctagcgccggagagcttcaaaaggggaacgaactcgcactaccgtctaaatacgtgaatttcctgtatttagcgtcccattacgagaagttgaaaggttcacctgaagataacgaacagaagcaactttttgttgagcagcacaaacattatctcgacgaaatcatagagcaaatttcggaattcagtaagagagtcatcctagctgatgccaatctggacaaagtattaagcgcatacaacaagcacagggataaacccatacgtgagcaggcggaaaatattatccatttgtttactcttaccaacctcggcgctccagccgcattcaagtattttgacacaacgatagatcgcaaacgatacacttctaccaaggaggtgctagacgcgacactgattcaccaatccatcacgggattatatgaaactcggatagatttgtcacagcttgggggtgacggatcccccaagaagaagaggaaagtctcgagcgactacaaagaccatgacggtgattataaagatcatgacatcgattacaaggatgacgatgacaaggctgcagga

SEQ ID NO: 27 - DNA, 4107 bp, Streptococcus pyogenes
  source 1..4107: mol_type = genomic DNA
atggataagaaatactcaataggcttagatatcggcacaaatagcgtcggatgggcggtgatcactgatgaatataaggttccgtctaaaaagttcaaggttctgggaaatacagaccgccacagtatcaaaaaaaatcttataggggctcttttatttgacagtggagagacagcggaagcgactcgtctcaaacggacagctcgtagaaggtatacacgtcggaagaatcgtatttgttatctacaggagattttttcaaatgagatggcgaaagtagatgatagtttctttcatcgacttgaagagtcttttttggtggaagaagacaagaagcatgaacgtcatcctatttttggaaatatagtagatgaagttgcttatcatgagaaatatccaactatctatcatctgcgaaaaaaattggtagattctactgataaagcggatttgcgcttaatctatttggccttagcgcatatgattaagtttcgtggtcattttttgattgagggagatttaaatcctgataatagtgatgtggacaaactatttatccagttggtacaaacctacaatcaattatttgaagaaaaccctattaacgcaagtggagtagatgctaaagcgattctttctgcacgattgagtaaatcaagacgattagaaaatctcattgctcagctccccggtgagaagaaaaatggcttatttgggaatctcattgctttgtcattgggtttgacccctaattttaaatcaaattttgatttggcagaagatgctaaattacagctttcaaaagatacttacgatgatgatttagataatttattggcgcaaattggagatcaatatgctgatttgtttttggcagctaagaatttatcagatgctattttactttcagatatcctaagagtaaatactgaaataactaaggctcccctatcagcttcaatgattaaacgctacgatgaacatcatcaagacttgactcttttaaaagctttagttcgacaacaacttccagaaaagtataaagaaatcttttttgatcaatcaaaaaacggatatgcaggttatattgatgggggagctagccaagaagaattttataaatttatcaaaccaattttagaaaaaatggatggtactgaggaattattggtgaaactaaatcgtgaagatttgctgcgcaagcaacggacctttgacaacggctctattccccatcaaattcacttgggtgagctgcatgctattttgagaagacaagaagacttttatccatttttaaaagacaatcgtgagaagattgaaaaaatcttgacttttcgaattccttattatgttggtccattggcgcgtggcaatagtcgttttgcatggatgactcggaagtctgaagaaacaattaccccatggaattttgaagaagttgtcgataaaggtgcttcagctcaatcatttattgaacgcatgacaaactttgataaaaatcttccaaatgaaaaagtactaccaaaacatagtttgctttatgagtattttacggtttataacgaattgacaaaggtcaaatatgttactgaaggaatgcgaaaaccagcatttctttcaggtgaacagaagaaagccattgttgatttactcttcaaaacaaatcgaaaagtaaccgttaagcaattaaaagaagattatttcaaaaaaatagaatgttttgatagtgttgaaatttcaggagttgaagatagatttaatgcttcattaggtacctaccatgatttgctaaaaattattaaagataaagattttttggataatgaagaaaatgaagatatcttagaggatattgttttaacattgaccttatttgaagatagggagatgattgaggaaagacttaaaacatatgctcacctctttgatgataaggtgatgaaacagcttaaacgtcgccgttatactggttggggacgtttgtctcgaaaattgattaatggtattagggataagcaatctggcaaaacaatattagattttttgaaatcagatggttttgccaatcgcaattttatgcagctgatccatgatgatagtttgacatttaaagaagacattcaaaaagcacaagtgtctggacaaggcgatagtttacatgaacatattgcaaatttagctggtagccctgctattaaaaaaggtattttacagactgtaaaagttgttgatgaattggtcaaagtaatggggcggcataagccagaaaatatcgttattgaaatggcacgtgaaaatcagacaactcaaaagggccagaaaaattcgcgagagcgtatgaaacgaatcgaagaaggtatcaaagaattaggaagtcagattcttaaagagcatcctgttgaaaatactcaattgcaaaatgaaaagctctatctctattatctccaaaatggaagagacatgtatgtggaccaagaattagatattaatcgtttaagtgattatgatgtcgatcacattgttccacaaagtttccttaaagacgattcaatagacaataaggtcttaacgcgttctgataaaaatcgtggtaaatcggataacgttccaagtgaagaagtagtcaaaaagatgaaaaactattggagacaacttctaaacgccaagttaatcactcaacgtaagtttgataatttaacgaaagctgaacgtggaggtttgagtgaacttgataaagctggttttatcaaacgccaattggttgaaactcgccaaatcactaagcatgtggcacaaattttggatagtcgcatgaatactaaatacgatgaaaatgataaacttattcgagaggttaaagtgattaccttaaaatctaaattagtttctgacttccgaaaagatttccaattctataaagtacgtgagattaacaattaccatcatgcccatgatgcgtatctaaatgccgtcgttggaactgctttgattaagaaatatccaaaacttgaatcggagtttgtctatggtgattataaagtttatgatgttcgtaaaatgattgctaagtctgagcaagaaataggcaaagcaaccgcaaaatatttcttttactctaatatcatgaacttcttcaaaacagaaattacacttgcaaatggagagattcgcaaacgccctctaatcgaaactaatggggaaactggagaaattgtctgggataaagggcgagattttgccacagtgcgcaaagtattgtccatgccccaagtcaatattgtcaagaaaacagaagtacagacaggcggattctccaaggagtcaattttaccaaaaagaaattcggacaagcttattgctcgtaaaaaagactgggatccaaaaaaatatggtggttttgatagtccaacggtagcttattcagtcctagtggttgctaaggtggaaaaagggaaatcgaagaagttaaaatccgttaaagagttactagggatcacaattatggaaagaagttcctttgaaaaaaatccgattgactttttagaagctaaaggatataaggaagttaaaaaagacttaatcattaaactacctaaatatagtctttttgagttagaaaacggtcgtaaacggatgctggctagtgccggagaattacaaaaaggaaatgagctggctctgccaagcaaatatgtgaattttttatatttagctagtcattatgaaaagttgaagggtagtccagaagataacgaacaaaaacaattgtttgtggagcagcataagcattatttagatgagattattgagcaaatcagtgaattttctaagcgtgttattttagcagatgccaatttagataaagttcttagtgcatataacaaacatagagacaaaccaatacgtgaacaagcagaaaatattattcatttatttacgttgacgaatcttggagctcccgctgcttttaaatattttgatacaacaattgatcgtaaacgatatacgtctacaaaagaagttttagatgccactcttatccatcaatccatcactggtctttatgaaacacgcattgatttgagtcagctaggaggtgactga

SEQ ID NO: 28 - protein, 1368 aa, Staphylococcus aureus [sequence omitted]

SEQ ID NO: 29 - skipped

SEQ ID NO: 30 - protein, 677 aa, unidentified
  source 1..677: note = Moloney murine leukemia virus
TLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGLLTSEGKEIKNKDEILALLKALFLPKRLSIIHCPGHQKGHSAEARGNRMADQAARKAAITETPDTSTLLIENSSP

SEQ ID NO: 31 - protein, 677 aa, synthetic construct
TLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSP

SEQ ID NO: 32 - protein, 677 aa, synthetic construct
TLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGLLTSEGKEIKNKDEILALLKALFLPKRLSIIHCPGHQKGHSAEARGNRMADQAARKAAITETPDTSTLLIENSSP

SEQ ID NO: 33 - protein, 677 aa, synthetic construct
TLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGLLTSEGKEIKNKDEILALLKALFLPKRLSIIHCPGHQKGHSAEARGNRMADQAARKAAITETPDTSTLLIENSSP

SEQ ID NO: 34 - protein, 677 aa, synthetic construct
TLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSP

SEQ ID NO: 35 - protein, 677 aa, synthetic construct
TLNIEDEYRLHETSKEPDVSLGSTWLSDFPQAWAETGGMGLAVRQAPLIIPLKATSTPVSIKQYPMSQKARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSP

SEQ ID NO: 36 - protein, 677 aa, synthetic construct
TLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGTAGFCRLWIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSP

SEQ ID NO: 37 - protein, 677 aa, synthetic construct
TLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIHCPGHQKGHSAEARGNRMADQAARKAAITETPDTSTLLIENSSP

SEQ ID NO: 38 - protein, 677 aa, synthetic construct
TLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSP

SEQ ID NO: 39 - protein, 677 aa, synthetic construct
TLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSP

SEQ ID NO: 40 - protein, 677 aa, synthetic construct
TLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSKARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSP

SEQ ID NO: 41 - protein, 677 aa, synthetic construct
TLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLWIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSP

SEQ ID NO: 42 - protein, 677 aa, synthetic construct
TLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSP

SEQ ID NO: 43 - protein, 677 aa, synthetic construct
TLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTGGSSLLQEGQRKAGAAVTTETEVIWAKALPAGTSAQRAQLIALTQALKMAEGKKLNVYTNSRYAFATAHIHGEIYRRRGWLTSEGKEIKNKDEILALLKALFLPKRLSIIHCPGHQKGHSAEARGNRMADQAARKAAITETPDTSTLLIENSSP

SEQ ID NO: 44 - protein, 677 aa, synthetic construct
TLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGTAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSP

SEQ ID NO: 45 - protein, 677 aa, synthetic construct
TLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIHCPGHQKGHSAEARGNRMADQAARKAAITETPDTSTLLIENSSP

SEQ ID NO: 46 - protein, 677 aa, synthetic construct
TLNIEDEYRLHETSKEPDVSLGSTWLSDFPQAWAETGGMGLAVRQAPLIILLKATSTPVSIKQYPMKQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPALFDEALRRDLADFRIQHPDLILLQYVDDLLLAATSELDCQQGTRALLQTLGNLGYRASAKKAQICQKQVKYLGYLLKEGQRWLTEARKETVMGQPTPKTPRQLRKFLGTAGNCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPPDRWLSKARMTHYQALLLDTDRVQFGPVVALNPATLLPLPEEGLQHNCLDILAEAHGTRPDLTDQPLPDADHTWYTGGSSLLQEGQRKAGAAVTTETEVIWAKALPAGTSAQRAELIALTQALKMAEGKKLNVYTNSRYAFATAHIQGEIYRRRGLLTSEGKEIKNKDEILALLKALFLPKRLSIIHCPGHQKGHSAEARGNRMANQAARKAAITETPDTSTLLIENSSP

SEQ ID NO: 47 - protein, 677 aa, synthetic construct
TLNIEDEYRLHETSKEPDVSLGSTWLSDFPQAWAETGGMGLAVRQAPLIILLKATSTPVSIKQYPMKQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPALFNEALRRDLADFRIQHPDLILLQYVDDLLLAATSELDCQQGTRALLQTLGNLGYRASAKKAQICQKQVKYLGYLLKEGQRWLTEARKETVMGQPTPKTPRQLRKFLGTAGNCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPPDRWLSKARMTHYQALLLDTDRVQFGPVVALNPATLLPLPEEGLQHNCLDILAEAHGTRPDLTDQPLPDADHTWYTGGSSLLQEGQRKAGAAVTTETEVIWAKALPAGTSAQRAELIALTQALKMAEGKKLNVYTNSRYAFATAHIQGEIYRRRGLLTSEGKEIKNKDEILALLKALFLPKRLSIIHCPGHQKGHSAEARGNRMANQAARKAAITETPDTSTLLIENSSP

SEQ ID NO: 48 - DNA, 56 bp, synthetic construct
  source 1..56: mol_type = other DNA
gggtcaggagccccccccctgaacccaggataaccctcaaagtcggggggcaaccc

SEQ ID NO: 49 - DNA, 51 bp, synthetic construct
  source 1..51: mol_type = other DNA
gggtcaggagccccccccctgaacccaggataaccctcaaagtcggggggc

SEQ ID NO: 50 - DNA, 60 bp, synthetic construct
  source 1..60: mol_type = other DNA
gtcagggtcaggagccccccccctgaacccaggataaccctcaaagtcggggggcaaccc

SEQ ID NO: 51 - DNA, 56 bp, synthetic construct
  source 1..56: mol_type = other DNA
gggtcaggagccccccccctgaacccaggaaaaccctcaaagtcggggggcaaccc

SEQ ID NO: 52 - DNA, 56 bp, synthetic construct
  source 1..56: mol_type = other DNA
gggtcaggagccccccccctgcacccaggaaaaccctcaaagtcggggggcaaccc

SEQ ID NO: 53 - DNA, 56 bp, synthetic construct
  source 1..56: mol_type = other DNA
gggtcaggagccccccccctgcacccaggataaccctcaaagtcggggggcaaccc

SEQ ID NO: 54 - DNA, 60 bp, synthetic construct
  source 1..60: mol_type = other DNA
gtcagggtcaggagccccccccctgaacccaggaaaaccctcaaagtcggggggcaaccc

SEQ ID NO: 55 - DNA, 60 bp, synthetic construct
  source 1..60: mol_type = other DNA
gtcagggtcaggagccccccccctgcacccaggaaaaccctcaaagtcggggggcaaccc

SEQ ID NO: 56 - DNA, 60 bp, synthetic construct
  source 1..60: mol_type = other DNA
gtcagggtcaggagccccccccctgcacccaggataaccctcaaagtcggggggcaaccc

SEQ ID NO: 57 - DNA, 19 bp, synthetic construct
  source 1..19: mol_type = other DNA
gggctgggatgggaaaggg

SEQ ID NO: 58 - DNA, 20 bp, synthetic construct
  source 1..20: mol_type = other DNA
gggacagggcagggacaggg

SEQ ID NO: 59 - DNA, 21 bp, synthetic construct
  source 1..21: mol_type = other DNA
gggtccgggtctgggtctggg

SEQ ID NO: 60 - DNA, 19 bp, synthetic construct
  source 1..19: mol_type = other DNA
gggctctgggtgggccggg

SEQ ID NO: 61 - DNA, 17 bp, synthetic construct
  source 1..17: mol_type = other DNA
gggtgggctgggaaggg

SEQ ID NO: 62 - DNA, 17 bp, synthetic construct
  source 1..17: mol_type = other DNA
gggagggagggctaggg

SEQ ID NO: 63 - DNA, 18 bp, synthetic construct
  source 1..18: mol_type = other DNA
gggctgggctgggcaggg

SEQ ID NO: 64 - DNA, 17 bp, synthetic construct
  source 1..17: mol_type = other DNA
gggcagggctgggaggg

SEQ ID NO: 65 - DNA, 16 bp, synthetic construct
  source 1..16: mol_type = other DNA
gggtgggagggctggg

SEQ ID NO: 66 - DNA, 19 bp, synthetic construct
  source 1..19: mol_type = other DNA
gggcagggtctgggctggg

SEQ ID NO: 67 - DNA, 12 bp, synthetic construct
  source 1..12: mol_type = other DNA
tggtggtggtgg

SEQ ID NO: 68 - DNA, 42 bp, synthetic construct
  source 1..42: mol_type = other DNA
ttgacgcggttctatctagttacgcgttaaaccaactagaaa

SEQ ID NO: 69 - DNA, 44 bp, synthetic construct
  source 1..44: mol_type = other DNA
cgcgagtctaggggataacgcgttaaacttcctagaaggcggtt

SEQ ID NO: 70 - DNA, 44 bp, synthetic construct
  source 1..44: mol_type = other DNA
cgcggatctagattgtaacgcgttaaaccatctagaaggcggtt

SEQ ID NO: 71 - DNA, 44 bp, synthetic construct
  source 1..44: mol_type = other DNA
cgcgtcgctaccgcccggcgcgttaaacacactagaaggcggtt

SEQ ID NO: 72 - DNA, 37 bp, synthetic construct
  source 1..37: mol_type = other DNA
cgcggttctatctagttacgcgttaaaccaactagaa

SEQ ID NO: 73 - DNA, 42 bp, synthetic construct
  source 1..42: mol_type = other DNA
ttgacgcgcttctatctagttacgcgttaaaccaactagaaa

SEQ ID NO: 74 - DNA, 42 bp, synthetic construct
  source 1..42: mol_type = other DNA
ttgacgcggttctatctacttacgcgttaaaccaactagaaa

SEQ ID NO: 75 - DNA, 81 bp, synthetic construct
  source 1..81: mol_type = other DNA
ggcggggctcgttggtctaggggtatgattctcgcttcgggtgcgagaggtcccgggttcaaatcccggacgagccccgcc

SEQ ID NO: 76 - DNA, 43 bp, synthetic construct
  source 1..43: mol_type = other DNA
gcgtaacctccatccgagttgcaagagagggaaacgcagtctc

SEQ ID NO: 77 - DNA, 160 bp, synthetic construct
  source 1..160: mol_type = other DNA
ggaattgcgggaaaggggtcaacagccgttcagtaccaagtctcaggggaaactttgagatggccttgcaaagggtatggtaataagctgacggacatggtcctaaccacgcagccaagtcctaagtcaacagatcttctgttgatatggatgcagttca

SEQ ID NO: 78 - DNA, 27 bp, synthetic construct
  source 1..27: mol_type = other DNA
atggtgcacctgactcctgaagagaag

SEQ ID NO: 79 - protein, 34 aa, synthetic construct
SGGSSGGSKRTADGSEFESPKKKRKVSGGSSGGS

SEQ ID NO: 80 - protein, 33 aa, synthetic construct
SGGSSGGSSGSETPGTSESATPESSGGSSGGSS

SEQ ID NO: 81 - protein, 120 aa, synthetic construct
  VARIANT 5..120: note = 1 to 29 copies of SGGS may be absent
SGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGS

SEQ ID NO: 82 - protein, 4 aa, synthetic construct
SGGS

SEQ ID NO: 83 - protein, 60 aa, synthetic construct
SGGSSGGSSGSETPGTSESATPESAGSYPYDVPDYAGSAAPAAKKKKLDGSGSGGSSGGS

SEQ ID NO: 84 - protein, 150 aa, synthetic construct
  VARIANT 6..150: note = 1 to 29 copies of GGGGS may be absent
GGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGS

SEQ ID NO: 85 - protein, 30 aa, synthetic construct
  VARIANT 2..30: note = 1 to 29 copies of G may be absent
GGGGGGGGGGGGGGGGGGGGGGGGGGGGGG

SEQ ID NO: 86 - protein, 150 aa, synthetic construct
  VARIANT 6..150: note = 1 to 29 copies of EAAAK may be absent
EAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAK

SEQ ID NO: 87 - protein, 90 aa, synthetic construct
  VARIANT 4..90: note = 1 to 29 copies of GGS may be absent
GGSGGSGGSGGSGGSGGSGGSGGSGGSGGSGGSGGSGGSGGSGGSGGSGGSGGSGGSGGSGGSGGSGGSGGSGGSGGSGGSGGSGGSGGS

SEQ ID NO: 88 - protein, 60 aa, synthetic construct
  VARIANT 3..60: note = 1 to 29 copies of XP may be absent
XPXPXPXPXPXPXPXPXPXPXPXPXPXPXPXPXPXPXPXPXPXPXPXPXPXPXPXPXPXP

SEQ ID NO: 89 - protein, 16 aa, synthetic construct
SGSETPGTSESATPES

SEQ ID NO: 90 - protein, 32 aa, synthetic construct
SGGSSGGSSGSETPGTSESATPESSGGSSGGS

SEQ ID NO: 91 - protein, 10 aa, synthetic construct
SGGSGGSGGS

SEQ ID NO: 92 - protein, 6 aa, synthetic construct
GGSGGS

SEQ ID NO: 93 - protein, 9 aa, synthetic construct
GGSGGSGGS

SEQ ID NO: 94 - protein, 7 aa, synthetic construct
PKKKRKV

SEQ ID NO: 95 - protein, 19 aa, synthetic construct
MKRTADGSEFESPKKKRKV

SEQ ID NO: 96 - protein, 21 aa, synthetic construct
SGGSKRTADGSEFEPKKKRKV

SEQ ID NO: 97 - protein, 18 aa, synthetic construct
KRTADGSEFESPKKKRKV

SEQ ID NO: 98 - protein, 9 aa, synthetic construct
PAAKRVKLD

SEQ ID NO: 99 - protein, 30 aa, synthetic construct
MDSLLMNRRKFLYQFKNVRWAKGRRETYLC

SEQ ID NO: 100 - protein, 20 aa, synthetic construct
AVKRPAATKKAGQAKKKKLD

SEQ ID NO: 101 - protein, 25 aa, synthetic construct
MSRRRKANPTKLSENAKKLAKEVEN

SEQ ID NO: 102 - protein, 9 aa, synthetic construct
KLKIKRPVK

SEQ ID NO: 103 - protein, 8 aa, synthetic construct
VSRKRPRP

SEQ ID NO: 104 - protein, 10 aa, synthetic construct
EGAPPAKRAR

SEQ ID NO: 105 - protein, 12 aa, synthetic construct
PPQPKKKPLDGE

SEQ ID NO: 106 - protein, 17 aa, synthetic construct
KRTADGSEFEPKKKRKV

SEQ ID NO: 107 - protein, 19 aa, synthetic construct
NLSKRPAAIKKAGQAKKKK

SEQ ID NO: 108 - protein, 11 aa, synthetic construct
RQRRNELKRSF

SEQ ID NO: 109 - protein, 38 aa, synthetic construct
NQSSNFGPMKGGNFGGRSSGPYGGGGQYFAKPRNQGGY

SEQ ID NO: 110 - protein, 16 aa, unidentified
  source 1..16: note = Xenopus nucleoplasmin
KRXXXXXXXXXXKKKL

SEQ ID NO: 111 - DNA, 27 bp, synthetic construct
  source 1..27: mol_type = other DNA
agacttctcttcaggagtcaggtgcac

SEQ ID NO: 112 - DNA, 27 bp, synthetic construct
  source 1..27: mol_type = other DNA
agactcctcttcaggagtcaggtgcac

SEQ ID NO: 113 - DNA, 168 bp, synthetic construct
  source 1..168: mol_type = other DNA
catggtgcacctgactcctggttttagagctagaaatagcaagttaaaataaggctagtccgttatcaacttgaaaaagtggcaccgagtcggtgcagactcctcttcaggagtcaggtgcacagaataaacgcggttctatctagttacgcgttaaaccaactagaa

SEQ ID NO: 114 - DNA, 171 bp, synthetic construct
  source 1..171: mol_type = other DNA
catggtgcacctgactcctggttttagagctagaaatagcaagttaaaataaggctagtccgttatcaacttgaaaaagtggcaccgagtcggtgcagactcctcttcaggagtcaggtgcacagaataaacgcggttctatctagttacgcgttaaaccaactagaattt

SEQ ID NO: 115 - DNA, 168 bp, synthetic construct
  source 1..168: mol_type = other DNA
catggtgcacctgactcctggttttagagctagaaatagcaagttaaaataaggctagtccgttatcaacttgaaaaagtggcaccgagtcggtgcagacttctcttcaggagtcaggtgcacagaataaacgcggttctatctagttacgcgttaaaccaactagaa

SEQ ID NO: 116 - DNA, 171 bp, synthetic construct
  source 1..171: mol_type = other DNA
catggtgcacctgactcctggttttagagctagaaatagcaagttaaaataaggctagtccgttatcaacttgaaaaagtggcaccgagtcggtgcagacttctcttcaggagtcaggtgcacagaataaacgcggttctatctagttacgcgttaaaccaactagaattt

SEQ ID NO: 117 - DNA, 166 bp, synthetic construct
  source 1..166: mol_type = other DNA
gattcctggtgccagaaacagttttagagctagaaatagcaagttaaaataaggctagtccgttatcaacttgaaaaagtggcaccgagtcggtgcgtcaccactgtttctggcaccaggataaataaccgcggttctatctagttacgcgttaaaccaactagaa

SEQ ID NO: 118 - DNA, 171 bp, synthetic construct
  source 1..171: mol_type = other DNA
gattcctggtgccagaaacagttttagagctagaaatagcaagttaaaataaggctagtccgttatcaacttgaaaaagtggcaccgagtcggtgcgtcaccactgtttctggcaccaggacctcttctttgacgcggttctatctagttacgcgttaaaccaactagaaa

SEQ ID NO: 119 - DNA, 168 bp, synthetic construct
  source 1..168: mol_type = other DNA
ggcccagactgagcacgtgagttttagagctagaaatagcaagttaaaataaggctagtccgttatcaacttgaaaaagtggcaccgagtcggtgcttcctctgccatctcgtgctcagtctgttataaaccgcggttctatctagttacgcgttaaaccaactagaa

SEQ ID NO: 120 - DNA, 181 bp, synthetic construct
  source 1..181: mol_type = other DNA
gtcatcttagtcattacctggttttagagctagaaatagcaagttaaaataaggctagtccgttatcaacttgaaaaagtggcaccgagtcggtgcatgggaactcagtttatatgagttagtaatgactaagatgtaaaggaacgcggttctatctagttacgcgttaaaccaactagaa

SEQ ID NO: 121 - DNA, 171 bp, synthetic construct
  source 1..171: mol_type = other DNA
gcattttcaggaggaagcgagttttagagctagaaatagcaagttaaaataaggctagtccgttatcaacttgaaaaagtggcaccgagtcggtgctgtctgaagcaatcgcttcctcctgaaaattatacacgcgcggttctatctagttacgcgttaaaccaactagaa

SEQ ID NO: 122 - DNA, 21 bp, synthetic construct
  source 1..21: mol_type = other DNA
gcccttcagctaaaataaagg

SEQ ID NO: 123 - DNA, 20 bp, synthetic construct
  source 1..20: mol_type = other DNA
gtcaaccagtatcccggtgc

SEQ ID NO: 124 - DNA, 20 bp, synthetic construct
  source 1..20: mol_type = other DNA
tcaaccattaagcaaaacat

SEQ ID NO: 125 - DNA, 20 bp, synthetic construct
  source 1..20: mol_type = other DNA
atgaagcactgtgggtacga

SEQ ID NO: 126 - DNA, 76 bp, synthetic construct
  source 1..76: mol_type = other DNA
gttttagagctagaaatagcaagttaaaataaggctagtccgttatcaacttgaaaaagtggcaccgagtcggtgc

SEQ ID NO: 127 - DNA, 20 bp, synthetic construct
  source 1..20: mol_type = other DNA
catggtgcacctgactcctg

SEQ ID NO: 128 - DNA, 20 bp, synthetic construct
  source 1..20: mol_type = other DNA
ccttgataccaacctgccca

SEQ ID NO: 129 - DNA, 20 bp, synthetic construct
  source 1..20: mol_type = other DNA
gtaacggcagacttctcttc

SEQ ID NO: 130 - DNA, 20 bp, synthetic construct
  source 1..20: mol_type = other DNA
cacgttcaccttgccccaca

SEQ ID NO: 131 - DNA, 20 bp, synthetic construct
  source 1..20: mol_type = other DNA
tccacatgcccagtttctat

SEQ ID NO: 132 - protein, 147 aa, synthetic construct [sequence omitted]

SEQ ID NO: 133 - DNA, 1606 bp, Homo sapiens
  source 1..1606: mol_type = genomic DNA
acatttgcttctgacacaactgtgttcactagcaacctcaaacagacaccatggtgcacctgactcctgaggagaagtctgccgttactgccctgtggggcaaggtgaacgtggatgaagttggtggtgaggccctgggcaggttggtatcaaggttacaagacaggtttaaggagaccaatagaaactgggcatgtggagacagagaagactcttgggtttctgataggcactgactctctctgcctattggtctattttcccacccttaggctgctggtggtctacccttggacccagaggttctttgagtcctttggggatctgtccactcctgatgctgttatgggcaaccctaaggtgaaggctcatggcaagaaagtgctcggtgcctttagtgatggcctggctcacctggacaacctcaagggcacctttgccacactgagtgagctgcactgtgacaagctgcacgtggatcctgagaacttcagggtgagtctatgggacgcttgatgttttctttccccttcttttctatggttaagttcatgtcataggaaggggataagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatctctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattcaccccaccagtgcaggctgcctatcagaaagtggtggctggtgtggctaatgccctggcccacaagtatcactaagctcgctttcttgctgtccaatttctattaaaggttcctttgttccctaagtccaactactaaactgggggatattatgaagggccttgagcatctggattctgcctaataaaaaacatttattttcattgc

SEQ ID NO: 134 - DNA, 1606 bp, synthetic construct
  source 1..1606: mol_type = other DNA
acatttgcttctgacacaactgtgttcactagcaacctcaaacagacaccatggtgcacctgactcctgtggagaagtctgccgttactgccctgtggggcaaggtgaacgtggatgaagttggtggtgaggccctgggcaggttggtatcaaggttacaagacaggtttaaggagaccaatagaaactgggcatgtggagacagagaagactcttgggtttctgataggcactgactctctctgcctattggtctattttcccacccttaggctgctggtggtctacccttggacccagaggttctttgagtcctttggggatctgtccactcctgatgctgttatgggcaaccctaaggtgaaggctcatggcaagaaagtgctcggtgcctttagtgatggcctggctcacctggacaacctcaagggcacctttgccacactgagtgagctgcactgtgacaagctgcacgtggatcctgagaacttcagggtgagtctatgggacgcttgatgttttctttccccttcttttctatggttaagttcatgtcataggaaggggataagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatctctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattcaccccaccagtgcaggctgcctatcagaaagtggtggctggtgtggctaatgccctggcccacaagtatcactaagctcgctttcttgctgtccaatttctattaaaggttcctttgttccctaagtccaactactaaactgggggatattatgaagggccttgagcatctggattctgcctaataaaaaacatttattttcattgc

SEQ ID NO: 135 - DNA, 444 bp, synthetic construct
  source 1..444: mol_type = other DNA
atggtgcacctgactcctgtggagaagtctgccgttactgccctgtggggcaaggtgaacgtggatgaagttggtggtgaggccctgggcaggctgctggtggtctacccttggacccagaggttctttgagtcctttggggatctgtccactcctgatgctgttatgggcaaccctaaggtgaaggctcatggcaagaaagtgctcggtgcctttagtgatggcctggctcacctggacaacctcaagggcacctttgccacactgagtgagctgcactgtgacaagctgcacgtggatcctgagaacttcaggctcctgggcaacgtgctggtctgtgtgctggcccatcactttggcaaagaattcaccccaccagtgcaggctgcctatcagaaagtggtggctggtgtggctaatgccctggcccacaagtatcactaa

SEQ ID NO: 136 - DNA, 58 bp, synthetic construct
  source 1..58: mol_type = other DNA
acactctttccctacacgacgctcttccgatctnnnntgggtgttatggccatcatga

SEQ ID NO: 137 - DNA, 37 bp, synthetic construct
  source 1..37: mol_type = other DNA
acactctttccctacacgacgctcttccgatctnnnn

SEQ ID NO: 138 - DNA, 21 bp, synthetic construct
  source 1..21: mol_type = other DNA
cacaacagcttcatgtcagcc

SEQ ID NO: 139 - DNA, 17 bp, synthetic construct
  source 1..17: mol_type = other DNA
gccacaccctagggttg

SEQ ID NO: 140 - DNA, 23 bp, synthetic construct
  source 1..23: mol_type = other DNA
cagggaaacgcccatgcaattag

SEQ ID NO: 141 - DNA, 25 bp, synthetic construct
  source 1..25: mol_type = other DNA
  misc_feature 21: note = RNA
atgtttaatatcccaggcccctgct

SEQ ID NO: 142 - DNA, 24 bp, synthetic construct
  source 1..24: mol_type = other DNA
  misc_feature 20: note = RNA
agggttagagctaggacaagtggg

SEQ ID NO: 143 - DNA, 26 bp, synthetic construct
  source 1..26: mol_type = other DNA
  misc_feature 22: note = RNA
acattgactttggagagtctgatgac

SEQ ID NO: 144 - DNA, 28 bp, synthetic construct
  source 1..28: mol_type = other DNA
  misc_feature 24: note = RNA
tctaagtagctagattacaggcacctgc

SEQ ID NO: 145 - DNA, 28 bp, synthetic construct
  source 1..28: mol_type = other DNA
  misc_feature 24: note = RNA
gcaagtagaatcatcagagtgacggctt

SEQ ID NO: 146 - DNA, 26 bp, synthetic construct
  source 1..26: mol_type = other DNA
  misc_feature 22: note = RNA
gtatgcactgcaagagagtttgtaca

SEQ ID NO: 147 - DNA, 25 bp, synthetic construct
  source 1..25: mol_type = other DNA
  misc_feature 21: note = RNA
aagcagtactcccagaagaagtttg

SEQ ID NO: 148 - DNA, 28 bp, synthetic construct
  source 1..28: mol_type = other DNA
  misc_feature 24: note = RNA
acaaatgggagaaagatcatactagagg

SEQ ID NO: 149 - DNA, 28 bp, synthetic construct
  source 1..28: mol_type = other DNA
  misc_feature 24: note = RNA
cctgtacctcatcacttcaataccacag

SEQ ID NO: 150 - DNA, 25 bp, synthetic construct
  source 1..25: mol_type = other DNA
  misc_feature 21: note = RNA
caccatgttaagtgctagcggcttt

SEQ ID NO: 151 - DNA, 27 bp, synthetic construct
  source 1..27: mol_type = other DNA
  misc_feature 23: note = RNA
ctcttttgtgcacactctaagtcttcc

SEQ ID NO: 152 - DNA, 29 bp, synthetic construct
  source 1..29: mol_type = other DNA
  misc_feature 25: note = RNA
caggttgcacatgatttctattgacatac

SEQ ID NO: 153 - DNA, 24 bp, synthetic construct
  source 1..24: mol_type = other DNA
  misc_feature 20: note = RNA
ctactcaagacttgtggctgatgt

SEQ ID NO: 154 - DNA, 27 bp, synthetic construct
  source 1..27: mol_type = other DNA
  misc_feature 23: note = RNA
caggattacagttggaaaaggtctagc

SEQ ID NO: 155 - DNA, 22 bp, synthetic construct
  source 1..22: mol_type = other DNA
  misc_feature 18: note = RNA
caccaacagcttgtaccctcag

SEQ ID NO: 156 - DNA, 28 bp, synthetic construct
  source 1..28: mol_type = other DNA
  misc_feature 24: note = RNA
tgctatattgcccagactattctcgaac

SEQ ID NO: 157 - DNA, 27 bp, synthetic construct
  source 1..27: mol_type = other DNA
  misc_feature 23: note = RNA
ctcacaaggatacacagagttcccact

SEQ ID NO: 158 - DNA, 31 bp, synthetic construct
  source 1..31: mol_type = other DNA
  misc_feature 27: note = RNA
gtacattcaaaagttttgcattaagccagga

SEQ ID NO: 159 - DNA, 26 bp, synthetic construct
  source 1..26: mol_type = other DNA
  misc_feature 22: note = RNA
tgctgatgctactctacctttggatg

SEQ ID NO: 160 - DNA, 25 bp, synthetic construct
  source 1..25: mol_type = other DNA
  misc_feature 21: note = RNA
acatgcccagtttccatttgcctcc

SEQ ID NO: 161 - DNA, 28 bp, synthetic construct
  source 1..28: mol_type = other DNA
  misc_feature 24: note = RNA
gcaatacactaggtcatctacttcattt

SEQ ID NO: 162 - DNA, 24 bp, synthetic construct
  source 1..24: mol_type = other DNA
  misc_feature 20: note = RNA
cagaatccagaggcaagatgaagc

SEQ ID NO: 163 - DNA, 33 bp, synthetic construct
  source 1..33: mol_type = other DNA
  misc_feature 29: note = RNA
tgccaaaaatgaggtataagatttgattgcttt

SEQ ID NO: 164 - DNA, 28 bp, synthetic construct
  source 1..28: mol_type = other DNA
  misc_feature 24: note = RNA
agaatgtttctgtcattccagaaaagtc

SEQ ID NO: 165 - DNA, 26 bp, synthetic construct
  source 1..26: mol_type = other DNA
  misc_feature 22: note = RNA
ttgagaggtaaaattccaggccagag

SEQ ID NO: 166 - DNA, 26 bp, synthetic construct
  source 1..26: mol_type = other DNA
  misc_feature 22: note = RNA
ccttgtgtccttttgacatgtgctta

SEQ ID NO: 167 - DNA, 24 bp, synthetic construct
  source 1..24: mol_type = other DNA
  misc_feature 20: note = RNA
ctctcagtcacgtggaactgtatg

SEQ ID NO: 168 - DNA, 30 bp, synthetic construct
  source 1..30: mol_type = other DNA
  misc_feature 26: note = RNA
acttatcatcaggcatctggtatatgctca

SEQ ID NO: 169 - DNA, 26 bp, synthetic construct
  source 1..26: mol_type = other DNA
  misc_feature 22: note = RNA
gtttcagttagttgggtgacactacc

SEQ ID NO: 170 - DNA, 25 bp, synthetic construct
  source 1..25: mol_type = other DNA
  misc_feature 21: note = RNA
tagaaatgctctgtttgggcctgac

SEQ ID NO: 171 - DNA, 27 bp, synthetic construct
  source 1..27: mol_type = other DNA
  misc_feature 23: note = RNA
cactcacaggttgcagataatccagat

SEQ ID NO: 172 - DNA, 27 bp, synthetic construct
  source 1..27: mol_type = other DNA
  misc_feature 23: note = RNA
agcaaccctccaatattatcaccactc

SEQ ID NO: 173 - DNA, 22 bp, synthetic construct
  source 1..22: mol_type = other DNA
  misc_feature 18: note = RNA
tcagacctcgccttgtccaacc

SEQ ID NO: 174 - DNA, 24 bp, synthetic construct
  source 1..24: mol_type = other DNA
  misc_feature 20: note = RNA
gttcaacaaggccttcagagttcc

SEQ ID NO: 175 - DNA, 25 bp, synthetic construct
  source 1..25: mol_type = other DNA
  misc_feature 21: note = RNA
atttggagtggtcctctgtgctgtt

SEQ ID NO: 176 - DNA, 32 bp, synthetic construct
  source 1..32: mol_type = other DNA
  misc_feature 28: note = RNA
ctgttaacatactgtacatcctcataactgca

SEQ ID NO: 177 - DNA, 22 bp, synthetic construct
  source 1..22: mol_type = other DNA
  misc_feature 18: note = RNA
gcatagggagcctccttgagtt

SEQ ID NO: 178 - DNA, 25 bp, synthetic construct
  source 1..25: mol_type = other DNA
  misc_feature 21: note = RNA
tctgtgtacgtactgatccccaaca

SEQ ID NO: 179 - DNA, 26 bp, synthetic construct
  source 1..26: mol_type = other DNA
  misc_feature 22: note = RNA
tttcagtcccttgattgagtccacgg

SEQ ID NO: 180 - DNA, 24 bp, synthetic construct
  source 1..24: mol_type = other DNA
  misc_feature 20: note = RNA
tgaactcatagggcttgctcagtc

SEQ ID NO: 181 - DNA, 24 bp, synthetic construct
  source 1..24: mol_type = other DNA
  misc_feature 20: note = RNA
aagaaccagagttgggcttcacca

SEQ ID NO: 182 - DNA, 25 bp, synthetic construct
  source 1..25: mol_type = other DNA
  misc_feature 21: note = RNA
gatggctcactctcctgaatgtgtt

SEQ ID NO: 183 - DNA, 25 bp, synthetic construct
  source 1..25: mol_type = other DNA
  misc_feature 21: note = RNA
ttcaagcaattgtcttgcgacagcc

SEQ ID NO: 184 - DNA, 24 bp, synthetic construct
  source 1..24: mol_type = other DNA
  misc_feature 20: note = RNA
cagagtacactttgcccaagcaac

SEQ ID NO: 185 - DNA, 24 bp, synthetic construct
  source 1..24: mol_type = other DNA
  misc_feature 20: note = RNA
gctgtttggatgccttgaactgag

SEQ ID NO: 186 - DNA, 24 bp, synthetic construct
  source 1..24: mol_type = other DNA
  misc_feature 20: note = RNA
gctagccttccctcaacaacacat

SEQ ID NO: 187 - DNA, 26 bp, synthetic construct
  source 1..26: mol_type = other DNA
  misc_feature 22: note = RNA
tctagacaactgagctccatggtagg

SEQ ID NO: 188 - DNA, 25 bp, synthetic construct
  source 1..25: mol_type = other DNA
  misc_feature 21: note = RNA
actagtgggcctttctgttacctcc

SEQ ID NO: 189 - DNA, 27 bp, synthetic construct
  source 1..27: mol_type = other DNA
  misc_feature 23: note = RNA
gtaaacactaccaatatgctgcctccc

SEQ ID NO: 190 - DNA, 26 bp, synthetic construct
  source 1..26: mol_type = other DNA
  misc_feature 22: note = RNA
gtgccctgtttcaactacaatggaca

SEQ ID NO: 191 - DNA, 25 bp, synthetic construct
  source 1..25: mol_type = other DNA
  misc_feature 21: note = RNA
gtcacagtctctggaactacgctcc

SEQ ID NO: 192 - DNA, 25 bp, synthetic construct
  source 1..25: mol_type = other DNA
  misc_feature 21: note = RNA
ttcaggtgcatgatccatttccagt

SEQ ID NO: 193 - DNA, 23 bp, synthetic construct
  source 1..23: mol_type = other DNA
  misc_feature 19: note = RNA
gcattgtggtcgaggtctgaaca

SEQ ID NO: 194 - DNA, 27 bp, synthetic construct
  source 1..27: mol_type = other DNA
  misc_feature 23: note = RNA
aagtttcattcctcagtctcctctacc

SEQ ID NO: 195 - DNA, 26 bp, synthetic construct
  source 1..26: mol_type = other DNA
  misc_feature 22: note = RNA
cttcctccaagatctcagtctctcag

SEQ ID NO: 196 - DNA, 26 bp, synthetic construct
  source 1..26: mol_type = other DNA
  misc_feature 22: note = RNA
tctcatctcctaatgccatcacactg

SEQ ID NO: 197 - DNA, 28 bp, synthetic construct
  source 1..28: mol_type = other DNA
  misc_feature 24: note = RNA
accggtctgatcaaaatttatttggcag

SEQ ID NO: 198 - DNA, 24 bp, synthetic construct
  source 1..24: mol_type = other DNA
  misc_feature 20: note = RNA
tatgcatctgtgagctccggaaca

SEQ ID NO: 199 - DNA, 22 bp, synthetic construct
  source 1..22: mol_type = other DNA
  misc_feature 18: note = RNA
agatcccttctgcctctcccag

SEQ ID NO: 200 - DNA, 24 bp, synthetic construct
  source 1..24: mol_type = other DNA
  misc_feature 20: note = RNA
gcttgggtgagagtgagaccttgt

SEQ ID NO: 201 - DNA, 23 bp, synthetic construct
  source 1..23: mol_type = other DNA
  misc_feature 19: note = RNA
atggacgccgcatatacgcgtga

SEQ ID NO: 202 - DNA, 25 bp, synthetic construct
  source 1..25: mol_type = other DNA
  misc_feature 21: note = RNA
atttcctgcctgtggtttgactcac

SEQ ID NO: 203 - DNA, 22 bp, synthetic construct
  source 1..22: mol_type = other DNA
  misc_feature 18: note = RNA
cctgctaagccagacatcctgc

SEQ ID NO: 204 - DNA, 28 bp, synthetic construct
  source 1..28: mol_type = other DNA
  misc_feature 24: note = RNA
tcattttgtatagaccagtgctgcccat

SEQ ID NO: 205 - DNA, 25 bp, synthetic construct
  source 1..25: mol_type = other DNA
  misc_feature 21: note = RNA
tgctactctcattctcctccctgca

SEQ ID NO: 206 - DNA, 24 bp, synthetic construct
  source 1..24: mol_type = other DNA
  misc_feature 20: note = RNA
gagaacgtgactcatccagctcca

SEQ ID NO: 207 - DNA, 25 bp, synthetic construct
  source 1..25: mol_type = other DNA
  misc_feature 21: note = RNA
caaagttgaaggtgctcacaaaact

SEQ ID NO: 208 - DNA, 23 bp, synthetic construct
  source 1..23: mol_type = other DNA
  misc_feature 19: note = RNA
agagaggactgaagggtgcagtc

SEQ ID NO: 209 - DNA, 25 bp, synthetic construct
  source 1..25: mol_type = other DNA
  misc_feature 21: note = RNA
caaactgccctgcatgtaatcttca

SEQ ID NO: 210 - DNA, 33 bp, synthetic construct
  source 1..33: mol_type = other DNA
  misc_feature 29: note = RNA
gcaattgttaagaaataattcagagagccagaa

SEQ ID NO: 211 - DNA, 26 bp, synthetic construct
  source 1..26: mol_type = other DNA
  misc_feature 22: note = RNA
cccaagctagactcatcttctcactc

SEQ ID NO: 212 - DNA, 29 bp, synthetic construct
  source 1..29: mol_type = other DNA
  misc_feature 25: note = RNA
ataagcaaaaaggaatcatcgctaggaca

SEQ ID NO: 213 - DNA, 26 bp, synthetic construct
  source 1..26: mol_type = other DNA
  misc_feature 22: note = RNA
gcttaggttaccccaattagccagac

SEQ ID NO: 214 - DNA, 24 bp, synthetic construct
  source 1..24: mol_type = other DNA
  misc_feature 20: note = RNA
cagctcactggaactacaggcaca

SEQ ID NO: 215 - DNA, 28 bp, synthetic construct
  source 1..28: mol_type = other DNA
  misc_feature 24: note = RNA
cttgctttttcatgtttctgtgggttgg

SEQ ID NO: 216 - DNA, 28 bp, synthetic construct
  source 1..28: mol_type = other DNA
  misc_feature 24: note = RNA
gatgaagaagtacatagggtgaggtctg

SEQ ID NO: 217 - DNA, 21 bp, synthetic construct
  source 1..21: mol_type = other DNA
  misc_feature 17: note = RNA
aggagctgggaccaagggatg

SEQ ID NO: 218 - DNA, 24 bp, synthetic construct
  source 1..24: mol_type = other DNA
  misc_feature 20: note = RNA
cagaccagaaaggaagtggcacag

SEQ ID NO: 219 - DNA, 27 bp, synthetic construct
  source 1..27: mol_type = other DNA
  misc_feature 23: note = RNA
ataacgaggttgagaaggtgaactgag

SEQ ID NO: 220 - DNA, 27 bp, synthetic construct
  source 1..27: mol_type = other DNA
  misc_feature 23: note = RNA
gaccctggatcctctgataaatctctg

SEQ ID NO: 221 - DNA, 24 bp, synthetic construct
  source 1..24: mol_type = other DNA
  misc_feature 20: note = RNA
acagttggacatggtgctagagct

SEQ ID NO: 222 - DNA, 23 bp, synthetic construct
  source 1..23: mol_type = other DNA
  misc_feature 19: note = RNA
atgctgaggacccattctcacct

SEQ ID NO: 223 - DNA, 27 bp, synthetic construct
  source 1..27: mol_type = other DNA
  misc_feature 23: note = RNA
ccactgcccttacaaatgtatagcaat

SEQ ID NO: 224 - DNA, 25 bp, synthetic construct
  source 1..25: mol_type = other DNA
  misc_feature 21: note = RNA
gccggtctttgaatttctgacctca

SEQ ID NO: 225 - DNA, 22 bp, synthetic construct
  source 1..22: mol_type = other DNA
  misc_feature 18: note = RNA
tcacttgaacccggaggcgaag

SEQ ID NO: 226 - DNA, 26 bp, synthetic construct
  source 1..26: mol_type = other DNA
  misc_feature 22: note = RNA
ttggatttgtttctacagccagagac

SEQ ID NO: 227 - DNA, 24 bp, synthetic construct
  source 1..24: mol_type = other DNA
  misc_feature 20: note = RNA
tgaaagttcctgcagtcttcccac

SEQ ID NO: 228 - DNA, 28 bp, synthetic construct
  source 1..28: mol_type = other DNA
  misc_feature 24: note = RNA
tgagtatctggttagaggtctttcagac

SEQ ID NO: 229 - DNA, 23 bp, synthetic construct
  source 1..23: mol_type = other DNA
  misc_feature 19: note = RNA
gctgtctccccaaattggcaatc

SEQ ID NO: 230 - DNA, 25 bp, synthetic construct
  source 1..25: mol_type = other DNA
  misc_feature 21: note = RNA
agaagcaaaggccaaactctgtctt

SEQ ID NO: 231 - DNA, 24 bp, synthetic construct
  source 1..24: mol_type = other DNA
  misc_feature 20: note = RNA
ggataacatcgggcacatggtatg

SEQ ID NO: 232 - DNA, 24 bp, synthetic construct
  source 1..24: mol_type = other DNA
  misc_feature 20: note = RNA
tgaaggcaaaaggagtgggctctg

SEQ ID NO: 233 - DNA, 28 bp, synthetic construct
  source 1..28: mol_type = other DNA
  misc_feature 24: note = RNA
ataatggattttggagattcagaatggg

SEQ ID NO: 234 - DNA, 27 bp, synthetic construct
  source 1..27: mol_type = other DNA
  misc_feature 23: note = RNA
tccgacatagtatgaaactgcagctgc

SEQ ID NO: 235 - DNA, 26 bp, synthetic construct
  source 1..26: mol_type = other DNA
  misc_feature 22: note = RNA
aattgggcttgtttggtacttcctca

SEQ ID NO: 236 - DNA, 28 bp, synthetic construct
  source 1..28: mol_type = other DNA
  misc_feature 24: note = RNA
aggactgaaaaacttcctatcctgtaca

SEQ ID NO: 237 - DNA, 24 bp, synthetic construct
  source 1..24: mol_type = other DNA
  misc_feature 20: note = RNA
tctccatttccagcaccttctcct

SEQ ID NO: 238 - DNA, 22 bp, synthetic construct
  source 1..22: mol_type = other DNA
  misc_feature 18: note = RNA
aaagcttcctggtgctcctccc

SEQ ID NO: 239 - DNA, 20 bp, synthetic construct
  source 1..20: mol_type = other DNA
acgtctcatatgccccttgg

SEQ ID NO: 240 - DNA, 21 bp, synthetic construct
  source 1..21: mol_type = other DNA
acaaacaagacagggaactgg

SEQ ID NO: 241 - DNA, 24 bp, synthetic construct
  source 1..24: mol_type = other DNA
  misc_feature 20: note = RNA
tcttcactgacactcccagctctg

SEQ ID NO: 242 - DNA, 25 bp, synthetic construct
  source 1..25: mol_type = other DNA
  misc_feature 21: note = RNA
ctaattccctcccaagtccaggaaa

SEQ ID NO: 243 - DNA, 22 bp, synthetic construct
  source 1..22: mol_type = other DNA
  misc_feature 18: note = RNA
ggtctccaataccgggcctaag

SEQ ID NO: 244 - DNA, 25 bp, synthetic construct
  source 1..25: mol_type = other DNA
  misc_feature 21: note = RNA
ggtacctcgctatccactttgtaag

SEQ ID NO: 245 - DNA, 24 bp, synthetic construct
  source 1..24: mol_type = other DNA
  misc_feature 20: note = RNA
tagcaataggtggttggccccagt

SEQ ID NO: 246 - DNA, 25 bp, synthetic construct
  source 1..25: mol_type = other DNA
  misc_feature 21: note = RNA
ggcatcatgagggttcataactcag

SEQ ID NO: 247 - DNA, 22 bp, synthetic construct
  source 1..22: mol_type = other DNA
  misc_feature 18: note = RNA
aggtgaggagcagcagaccaca

SEQ ID NO: 248 - DNA, 27 bp, synthetic construct
  source 1..27: mol_type = other DNA
  misc_feature 23: note = RNA
tggctggctagattttctaaacccttc

SEQ ID NO: 249 - DNA, 22 bp, synthetic construct
  source 1..22: mol_type = other DNA
  misc_feature 18: note = RNA
agcctaagccgctctttgaaca

SEQ ID NO: 250 - DNA, 30 bp, synthetic construct
  source 1..30: mol_type = other DNA
  misc_feature 26: note = RNA
gaaaacttaagtgtaagcagcagatggaat

SEQ ID NO: 251 - DNA, 28 bp, synthetic construct
  source 1..28: mol_type = other DNA
  misc_feature 24: note = RNA
agaccttttgattctgtgtcttcctgtg

SEQ ID NO: 252 - DNA, 26 bp, synthetic construct
  source 1..26: mol_type = other DNA
  misc_feature 22: note = RNA
cattgctttcttcactgggaacacat

SEQ ID NO: 253 - DNA, 30 bp, synthetic construct
  source 1..30: mol_type = other DNA
  misc_feature 26: note = RNA
cagatggactatgctatgtttttgaggacc

SEQ ID NO: 254 - DNA, 50 bp, synthetic construct
  source 1..50: mol_type = other DNA
tggagttcagacgtgtgctcttccgatctcctcaacaaactgaggcatac

SEQ ID NO: 255 - DNA, 29 bp, synthetic construct
  source 1..29: mol_type = other DNA
tggagttcagacgtgtgctcttccgatct

SEQ ID NO: 256 - DNA, 23 bp, synthetic construct
  source 1..23: mol_type = other DNA
acgttaatgtttcctgatggtcc

SEQ ID NO: 257 - DNA, 22 bp, synthetic construct
  source 1..22: mol_type = other DNA
gggaaaatagaccaataggcag

SEQ ID NO: 258 - DNA, 22 bp, synthetic construct
  source 1..22: mol_type = other DNA
ctctgttgagctcgaccctgaa

SEQ ID NO: 259 - DNA, 24 bp, synthetic construct
  source 1..24: mol_type = other DNA
  misc_feature 20: note = RNA
cctgtgtcctcaaaatgcagtaga

SEQ ID NO: 260 - DNA, 25 bp, synthetic construct
  source 1..25: mol_type = other DNA
  misc_feature 21: note = RNA
aaattggcaggacaccttttcaggc

SEQ ID NO: 261 - DNA, 27 bp, synthetic construct
  source 1..27: mol_type = other DNA
  misc_feature 23: note = RNA
gtattgccaaaagagaaggagacaaac

SEQ ID NO: 262 - DNA, 27 bp, synthetic construct
  source 1..27: mol_type = other DNA
  misc_feature 23: note = RNA
ccacagtcatgtaacaaacctgcacat

SEQ ID NO: 263 - DNA, 27 bp, synthetic construct
  source 1..27: mol_type = other DNA
  misc_feature 23: note = RNA
cagcctttgggtctgtaaaattctctg

SEQ ID NO: 264 - DNA, 25 bp, synthetic construct
  source 1..25: mol_type = other DNA
  misc_feature 21: note = RNA
actgagagtgaagacaacgaagaag

SEQ ID NO: 265 - DNA, 25 bp, synthetic construct
  source 1..25: mol_type = other DNA
  misc_feature 21: note = RNA
tcatctttatgccacccagacagtt

SEQ ID NO: 266 - DNA, 26 bp, synthetic construct
  source 1..26: mol_type = other DNA
  misc_feature 22: note = RNA
gtaatcaccttcctcctccatgcctg

SEQ ID NO: 267 - DNA, 28 bp, synthetic construct
  source 1..28: mol_type = other DNA
  misc_feature 24: note = RNA
gatgatattaggtcctcttctgccttac

SEQ ID NO: 268 - DNA, 24 bp, synthetic construct
  source 1..24: mol_type = other DNA
  misc_feature 20: note = RNA
tcagtgagtgagacaggaacagtg

SEQ ID NO: 269 - DNA, 26 bp, synthetic construct
  source 1..26: mol_type = other DNA
  misc_feature 22: note = RNA
gactctgtgattctgcaagatgagct

SEQ ID NO: 270 - DNA, 28 bp, synthetic construct
  source 1..28: mol_type = other DNA
  misc_feature 24: note = RNA
agaaatcaaagaggatgggaatgggaca

SEQ ID NO: 271 - DNA, 26 bp, synthetic construct
  source 1..26: mol_type = other DNA
  misc_feature 22: note = RNA
gaagctttttcaagcactgttcaact

SEQ ID NO: 272 - DNA, 27 bp, synthetic construct
  source 1..27: mol_type = other DNA
  misc_feature 23: note = RNA
ttgggtgacagcataaatacatgcaag

SEQ ID NO: 273 - DNA, 22 bp, synthetic construct
  source 1..22: mol_type = other DNA
  misc_feature 18: note = RNA
gaaggcaaaagggatgggtagg

SEQ ID NO: 274 - DNA, 27 bp, synthetic construct
  source 1..27: mol_type = other DNA
  misc_feature 23: note = RNA
cagaactactctcgtcctaacacctgt

SEQ ID NO: 275 - DNA, 26 bp, synthetic construct
  source 1..26: mol_type = other DNA
  misc_feature 22: note = RNA
ctgatgatcatttgccagtctcagga

SEQ ID NO: 276 - DNA, 30 bp, synthetic construct
  source 1..30: mol_type = other DNA
  misc_feature 26: note = RNA
gtgacctgatatttgggttaattcaatttg

SEQ ID NO: 277 - DNA, 26 bp, synthetic construct
  source 1..26: mol_type = other DNA
  misc_feature 22: note = RNA
tgctctatgcttctttggagagagga

SEQ ID NO: 278 - DNA, 26 bp, synthetic construct
  source 1..26: mol_type = other DNA
  misc_feature 22: note = RNA
ctagcaacctcaaacagacaccatgg

SEQ ID NO: 279 - DNA, 28 bp, synthetic construct
  source 1..28: mol_type = other DNA
  misc_feature 24: note = RNA
aagtatatgggacaatgtgcataggtta

SEQ ID NO: 280 - DNA, 23 bp, synthetic construct
  source 1..23: mol_type = other DNA
  misc_feature 19: note = RNA
ggtgtcccacatttgtgacttcc

SEQ ID NO: 281 - DNA, 32 bp, synthetic construct
  source 1..32: mol_type = other DNA
  misc_feature 28: note = RNA
agacccttaggtaagagataaacataacaaac

SEQ ID NO: 282 - DNA, 28 bp, synthetic construct
  source 1..28: mol_type = other DNA
  misc_feature 24: note = RNA
tcatgagaataacataggtgactctcaa

SEQ ID NO: 283 - DNA, 25 bp, synthetic construct
  source 1..25: mol_type = other DNA
  misc_feature 21: note = RNA
gaagcaacgtgattctacgtgtctt

SEQ ID NO: 284 - DNA, 26 bp, synthetic construct
  source 1..26: mol_type = other DNA
  misc_feature 22: note = RNA
cagggttgacaacataagctacaaga

SEQ ID NO: 285 - DNA, 25 bp, synthetic construct
  source 1..25: mol_type = other DNA
  misc_feature 21: note = RNA
cccttgccatatcctgatcacatgt

SEQ ID NO: 286 - DNA, 30 bp, synthetic construct
  source 1..30: mol_type = other DNA
  misc_feature 26: note = RNA
ttccctgaaaatgtaaagtatcagagcaat

SEQ ID NO: 287 - DNA, 29 bp, synthetic construct
  source 1..29: mol_type = other DNA
  misc_feature 25: note = RNA
cattaaaaaccagcatttgattggatgtc

SEQ ID NO: 288 - DNA, 26 bp, synthetic construct
  source 1..26: mol_type = other DNA
  misc_feature 22: note = RNA
caccatgcccagctaattttgcattt

SEQ ID NO: 289 - DNA, 27 bp, synthetic construct
  source 1..27: mol_type = other DNA
  misc_feature 23: note = RNA
gctgcccaagattttagactttgtgtt

SEQ ID NO: 290 - DNA, 27 bp, synthetic construct
  source 1..27: mol_type = other DNA
  misc_feature 23: note = RNA
ggacatggaaagcctatacttgaaatg

SEQ ID NO: 291 - DNA, 23 bp, synthetic construct
  source 1..23: mol_type = other DNA
  misc_feature 19: note = RNA
caaactgtcctggcaactctgac

SEQ ID NO: 292 - DNA, 23 bp, synthetic construct
  source 1..23: mol_type = other DNA
  misc_feature 19: note = RNA
agatgccaggaacagcttgtgtg

SEQ ID NO: 293 - DNA, 25 bp, synthetic construct
  source 1..25: mol_type = other DNA
  misc_feature 21: note = RNA
tctccatcctttgcagacctgtact

SEQ ID NO: 294 - DNA, 34 bp, synthetic construct
  source 1..34: mol_type = other DNA
  misc_feature 30: note = RNA
gggaaattacaatcttagttctacttttccacct

SEQ ID NO: 295 - DNA, 22 bp, synthetic construct
  source 1..22: mol_type = other DNA
  misc_feature 18: note = RNA
aagccagcatccagagagacag

SEQ ID NO: 296 - DNA, 23 bp, synthetic construct
  source 1..23: mol_type = other DNA
  misc_feature 19: note = RNA
aaacaacgcagctgtgtggaagc

SEQ ID NO: 297 - DNA, 26 bp, synthetic construct
  source 1..26: mol_type = other DNA
  misc_feature 22: note = RNA
ggccactagtatattgcctaaaagcg

SEQ ID NO: 298 - DNA, 24 bp, synthetic construct
  source 1..24: mol_type = other DNA
  misc_feature 20: note = RNA
tggccaggactctttaagtcgtga

SEQ ID NO: 299 - DNA, 24 bp, synthetic construct
  source 1..24: mol_type = other DNA
  misc_feature 20: note = RNA
aggaactgtgtgccatctgctcca

SEQ ID NO: 300 - DNA, 24 bp, synthetic construct
  source 1..24: mol_type = other DNA
  misc_feature 20: note = RNA
gaatttggctgggcttgttccaaa

SEQ ID NO: 301 - DNA, 26 bp, synthetic construct
  source 1..26: mol_type = other DNA
  misc_feature 22: note = RNA
aagccaagtcacacctgtaatcccag

SEQ ID NO: 302 - DNA, 24 bp, synthetic construct
  source 1..24: mol_type = other DNA
  misc_feature 20: note = RNA
acactctgagagatgcaggccagg

SEQ ID NO: 303 - DNA, 23 bp, synthetic construct
  source 1..23: mol_type = other DNA
  misc_feature 19: note = RNA
cagagcagtggagggaaaagctt

SEQ ID NO: 304 - DNA, 26 bp, synthetic construct
  source 1..26: mol_type = other DNA
  misc_feature 22: note = RNA
gcaacaagagcgaaactccatctaaa

SEQ ID NO: 305 - DNA, 24 bp, synthetic construct
  source 1..24: mol_type = other DNA
  misc_feature 20: note = RNA
ttggtcaacagcaatgacagattc

SEQ ID NO: 306 - DNA, 25 bp, synthetic construct
  source 1..25: mol_type = other DNA
  misc_feature 21: note = RNA
tgattttgagcaacactccccaccc

SEQ ID NO: 307 - DNA, 27 bp, synthetic construct
  source 1..27: mol_type = other DNA
  misc_feature 23: note = RNA
gatcccaaatctgttacctctgcctct

SEQ ID NO: 308 - DNA, 28 bp, synthetic construct
  source 1..28: mol_type = other DNA
  misc_feature 24: note = RNA
tgagtaaaagctaccctaaatgcctggg

SEQ ID NO: 309 - DNA, 26 bp, synthetic construct
  source 1..26: mol_type = other DNA
  misc_feature 22: note = RNA
ctttcttcctgcctgaactaagccac

SEQ ID NO: 310 - DNA, 26 bp, synthetic construct
  source 1..26: mol_type = other DNA
  misc_feature 22: note = RNA
agtgtccaatcaggattaggagctgt

SEQ ID NO: 311 - DNA, 22 bp, synthetic construct
  source 1..22: mol_type = other DNA
  misc_feature 18: note = RNA
gtgcacctgaccttccggaact

SEQ ID NO: 312 - DNA, 28 bp, synthetic construct
  source 1..28: mol_type = other DNA
  misc_feature 24: note = RNA
aagccaaacatttacaaaagggacagga

SEQ ID NO: 313 - DNA, 26 bp, synthetic construct
  source 1..26: mol_type = other DNA
  misc_feature 22: note = RNA
tctgaggtctgtatgtgctaagccac

SEQ ID NO: 314 - DNA, 27 bp, synthetic construct
  source 1..27: mol_type = other DNA
  misc_feature 23: note = RNA
ttggtcaaattctctcttctgaggagg

SEQ ID NO: 315 - DNA, 28 bp, synthetic construct
  source 1..28: mol_type = other DNA
  misc_feature 24: note = RNA
gaattctttttctcagcatggaacatcc

SEQ ID NO: 316 - DNA, 25 bp, synthetic construct
  source 1..25: mol_type = other DNA
  misc_feature 21: note = RNA
ttactcctccaaccacccaacagaa

SEQ ID NO: 317 - DNA, 23 bp, synthetic construct
  source 1..23: mol_type = other DNA
  misc_feature 19: note = RNA
gtgggcaaagtcacaacacctgt

SEQ ID NO: 318 - DNA, 28 bp, synthetic construct
  source 1..28: mol_type = other DNA
  misc_feature 24: note = RNA
ctgcatgtatcaattttgagagaattca

SEQ ID NO: 319 - DNA, 23 bp, synthetic construct
  source 1..23: mol_type = other DNA
  misc_feature 19: note = RNA
accttgtgatcctcccgtctcag

SEQ ID NO: 320 - DNA, 26 bp, synthetic construct
  source 1..26: mol_type = other DNA
  misc_feature 22: note = RNA
gcgccttgggtaattaattcccaaag

SEQ ID NO: 321 - DNA, 22 bp, synthetic construct
  source 1..22: mol_type = other DNA
  misc_feature 18: note = RNA
ggtatggatgacccagtgggtg

SEQ ID NO: 322 - DNA, 28 bp, synthetic construct
  source 1..28: mol_type = other DNA
  misc_feature 24: note = RNA
attctcctttggaaacatacctcgcact

SEQ ID NO: 323 - DNA, 24 bp, synthetic construct
  source 1..24: mol_type = other DNA
  misc_feature 20: note = RNA
tgcttcctctgactgaacacaaag

SEQ ID NO: 324 - DNA, 25 bp, synthetic construct
  source 1..25: mol_type = other DNA
  misc_feature 21: note = RNA
tgttttatgctccccaagtgcctca

SEQ ID NO: 325 - DNA, 26 bp, synthetic construct
  source 1..26: mol_type = other DNA
  misc_feature 22: note = RNA
ataaacaaacacgtggcagacggtta

SEQ ID NO: 326 - DNA, 25 bp, synthetic construct
  source 1..25: mol_type = other DNA
  misc_feature 21: note = RNA
gtcttagtggatcttgagcagagct

SEQ ID NO: 327 - DNA, 24 bp, synthetic construct
  source 1..24: mol_type = other DNA
  misc_feature 20: note = RNA
agtactgggctcccttactgatgt

SEQ ID NO: 328 - DNA, 28 bp, synthetic construct
  source 1..28: mol_type = other DNA
  misc_feature 24: note = RNA
gaaagacctcctacgtaattgcacccaa

SEQ ID NO: 329 - DNA, 25 bp, synthetic construct
  source 1..25: mol_type = other DNA
  misc_feature 21: note = RNA
ggtttgagctgattcagggaggaaa

SEQ ID NO: 330 - DNA, 28 bp, synthetic construct
  source 1..28: mol_type = other DNA
  misc_feature 24: note = RNA
tctcttctacagatgaagagtctgggtc

SEQ ID NO: 331 - DNA, 27 bp, synthetic construct
  source 1..27: mol_type = other DNA
  misc_feature 23: note = RNA
gttgccctatatgacttccaaggcaga

SEQ ID NO: 332 - DNA, 26 bp, synthetic construct
  source 1..26: mol_type = other DNA
  misc_feature 22: note = RNA
aaagtttttataggcccggagcggtg

SEQ ID NO: 333 - DNA, 28 bp, synthetic construct
  source 1..28: mol_type = other DNA
  misc_feature 24: note = RNA
gcttgttctgagcccatagataaggatc

SEQ ID NO: 334 - DNA, 28 bp, synthetic construct
  source 1..28: mol_type = other DNA
  misc_feature 24: note = RNA
gatggtcagtaagttaatcatgcccatg

SEQ ID NO: 335 - DNA, 22 bp, synthetic construct
  source 1..22: mol_type = other DNA
  misc_feature 18: note = RNA
gtcagccttgctacctccagga

SEQ ID NO: 336 - DNA, 24 bp, synthetic construct
  source 1..24: mol_type = other DNA
  misc_feature 20: note = RNA
acccacttgagatgtcacagtgga

SEQ ID NO: 337 - DNA, 27 bp, synthetic construct
  source 1..27: mol_type = other DNA
  misc_feature 23: note = RNA
gatgaaacagtgcaacatgtgtgtgaa

SEQ ID NO: 338 - DNA, 26 bp, synthetic construct
  source 1..26: mol_type = other DNA
  misc_feature 22: note = RNA
cgatgctatttccatgaggatcttgg

SEQ ID NO: 339 - DNA, 25 bp, synthetic construct
  source 1..25: mol_type = other DNA
  misc_feature 21: note = RNA
caagtcaagctgatcgagaggtaca

SEQ ID NO: 340 - DNA, 24 bp, synthetic construct
  source 1..24: mol_type = other DNA
  misc_feature 20: note = RNA
ctgttcagccgtaccatgagatgt

SEQ ID NO: 341 - DNA, 27 bp, synthetic construct
  source 1..27: mol_type = other DNA
  misc_feature 23: note = RNA
gctgcagagattttgtttatggccagt

SEQ ID NO: 342 - DNA, 26 bp, synthetic construct
  source 1..26: mol_type = other DNA
  misc_feature 22: note = RNA
cactcaccagtctttgtgaaaacact

SEQ ID NO: 343 - DNA, 23 bp, synthetic construct
  source 1..23: mol_type = other DNA
  misc_feature 19: note = RNA
taatgatgctgggccacaggcat

SEQ ID NO: 344 - DNA, 25 bp, synthetic construct
  source 1..25: mol_type = other DNA
  misc_feature 21: note = RNA
acacttagattagtgcccaccagga

SEQ ID NO: 345 - DNA, 26 bp, synthetic construct
  source 1..26: mol_type = other DNA
  misc_feature 22: note = RNA
ggttggtatatgctgttccaaggaac

SEQ ID NO: 346 - DNA, 28 bp, synthetic construct
  source 1..28: mol_type = other DNA
  misc_feature 24: note = RNA
aaatctctgggatcatgatagggctttg

SEQ ID NO: 347 - DNA, 23 bp, synthetic construct
  source 1..23: mol_type = other DNA
  misc_feature 19: note = RNA
acttcccagcctttccaggagct

SEQ ID NO: 348 - DNA, 26 bp, synthetic construct
  source 1..26: mol_type = other DNA
  misc_feature 22: note = RNA
atgtctgggtatggagtttgagaaac

SEQ ID NO: 349 - DNA, 23 bp, synthetic construct
  source 1..23: mol_type = other DNA
  misc_feature 19: note = RNA
gattcaacgggcaggagagaaga

SEQ ID NO: 350 - DNA, 24 bp, synthetic construct
  source 1..24: mol_type = other DNA
  misc_feature 20: note = RNA
gccttttggttggtgaacaccaga

SEQ ID NO: 351 - DNA, 27 bp, synthetic construct
  source 1..27: mol_type = other DNA
  misc_feature 23: note = RNA
tttgggagtacaagtgattttgggtac

SEQ ID NO: 352 - DNA, 27 bp, synthetic construct
  source 1..27: mol_type = other DNA
  misc_feature 23: note = RNA
cgtgggattgtatgtttagcatgcctc

SEQ ID NO: 353 - DNA, 25 bp, synthetic construct
  source 1..25: mol_type = other DNA
  misc_feature 21: note = RNA
ggccaaggatctctgtaaaaccctt

SEQ ID NO: 354 - DNA, 29 bp, synthetic construct
  source 1..29: mol_type = other DNA
  misc_feature 25: note = RNA
tggaaagtttcctgtcactattttcttcc

SEQ ID NO: 355 - DNA, 25 bp, synthetic construct
  source 1..25: mol_type = other DNA
  misc_feature 21: note = RNA
gcagggatcacaaattccttcctct

SEQ ID NO: 356 - DNA, 22 bp, synthetic construct
  source 1..22: mol_type = other DNA
  misc_feature 18: note = RNA
cacgagcagggagaatggtgct

SEQ ID NO: 357 - DNA, 22 bp, synthetic construct
  source 1..22: mol_type = other DNA
acgtaggaattttggtgggaca

SEQ ID NO: 358 - DNA, 21 bp, synthetic construct
  source 1..21: mol_type = other DNA
ctagaggggtgaggctgaaac

SEQ ID NO: 359 - DNA, 25 bp, synthetic construct
  source 1..25: mol_type = other DNA
  misc_feature 21: note = RNA
ttaaggtctctctcccaccaccaga

SEQ ID NO: 360 - DNA, 25 bp, synthetic construct
  source 1..25: mol_type = other DNA
  misc_feature 21: note = RNA
aattagctaggtgaggcagtgggat

SEQ ID NO: 361 - DNA, 23 bp, synthetic construct
  source 1..23: mol_type = other DNA
  misc_feature 19: note = RNA
cagcattaaccactgggcaaatg

SEQ ID NO: 362 - DNA, 24 bp, synthetic construct
  source 1..24: mol_type = other DNA
  misc_feature 20: note = RNA
cactgtgaaacctgcaacacacaa

SEQ ID NO: 363 - DNA, 24 bp, synthetic construct
  source 1..24: mol_type = other DNA
  misc_feature 20: note = RNA
tagccaggtgctgaacattctggt

SEQ ID NO: 364 - DNA, 24 bp, synthetic construct
  source 1..24: mol_type = other DNA
  misc_feature 20: note = RNA
agaaggacatcagagaagccctgg

SEQ ID NO: 365 - DNA, 23 bp, synthetic construct
  source 1..23: mol_type = other DNA
  misc_feature 19: note = RNA
cgcccctccttaaacagtctctg

SEQ ID NO: 366 - DNA, 26 bp, synthetic construct
  source 1..26: mol_type = other DNA
  misc_feature 22: note = RNA
cagcagaacatgtgtctttgtcaaat

SEQ ID NO: 367 - DNA, 23 bp, synthetic construct
  source 1..23: mol_type = other DNA
  misc_feature 19: note = RNA
gaaggagggacagagtcaaacaa

SEQ ID NO: 368 - DNA, 31 bp, synthetic construct
  source 1..31: mol_type = other DNA
  misc_feature 27: note = RNA
taggacaaaggaatcttaagtagtcactctg

SEQ ID NO: 369 - DNA, 29 bp, synthetic construct
  source 1..29: mol_type = other DNA
  misc_feature 25: note = RNA
cattttggaattccatgagaaacccacaa

SEQ ID NO: 370 - DNA, 27 bp, synthetic construct
  source 1..27: mol_type = other DNA
  misc_feature 23: note = RNA
tgagtgcttccaaaatccttaagggaa

SEQ ID NO: 371 - DNA, 30 bp, synthetic construct
  source 1..30: mol_type = other DNA
  misc_feature 26: note = RNA
catacacatttgagtaccagactaacacac

SEQ ID NO: 372 - protein, 9 aa, synthetic construct
MVHLTPVEK

SEQ ID NO: 373 - DNA, 30 bp, synthetic construct
  source 1..30: mol_type = other DNA
accatggtgcacctgactcctgtggagagg

SEQ ID NO: 374 - DNA, 30 bp, synthetic construct
  source 1..30: mol_type = other DNA
accatggtgcacctgactcctgaagagaag

SEQ ID NO: 375 - protein, 9 aa, synthetic construct
MVHLTPEEK

SEQ ID NO: 376 - DNA, 14 bp, synthetic construct
  source 1..14: mol_type = other DNA
ctgaagagaagtct

SEQ ID NO: 377 - DNA, 40 bp, synthetic construct
  source 1..40: mol_type = other DNA
ggtaagggtgccctaactcctgtggggaccttctttcatg

SEQ ID NO: 378 - DNA, 40 bp, synthetic construct
  source 1..40: mol_type = other DNA
ggtaagggtgccctaactcctgtggggaccttctttggtg

SEQ ID NO: 379 - DNA, 40 bp, synthetic construct
  source 1..40: mol_type = other DNA
ggtaagggtgccctaactcctgtggggaccttctttcgtg

SEQ ID NO: 380 - DNA, 40 bp, synthetic construct
  source 1..40: mol_type = other DNA
ggtaaggctgccctaacgcctgtggggaccttctttggtg

SEQ ID NO: 381 - DNA, 40 bp, synthetic construct; identical to SEQ ID NO: 377
  source 1..40: mol_type = other DNA

SEQ ID NO: 382 - DNA, 40 bp, synthetic construct
  source 1..40: mol_type = other DNA
ggtaagggtgccctaagtcctgtggggaacttctttggtg

SEQ ID NO: 383 - DNA, 40 bp, synthetic construct
  source 1..40: mol_type = other DNA
ggtaagggtgccctaacgcctgtggggaccttctttggtg

SEQ ID NO: 384 - DNA, 40 bp, synthetic construct
  source 1..40: mol_type = other DNA
ggtaagcatcgcctaactcctgtggggaccttctttggtg

SEQ ID NO: 385 - DNA, 40 bp, synthetic construct
  source 1..40: mol_type = other DNA
ggtaagggtgccctaactcctctggggaccttctttggtg

SEQ ID NO: 386 - DNA, 40 bp, synthetic construct
  source 1..40: mol_type = other DNA
ggtaagggtgccctaactcctgtggggaccttctttcttg

SEQ ID NO: 387 - DNA, 40 bp, synthetic construct
  source 1..40: mol_type = other DNA
ggtaagggtaccctaactcctgtggggaccttctttggtg

SEQ ID NO: 388 - DNA, 40 bp, synthetic construct
  source 1..40: mol_type = other DNA
ggtaagggtgccctaacttctgtgggggccttctttcgtg

SEQ ID NO: 389 - DNA, 40 bp, synthetic construct
  source 1..40: mol_type = other DNA
ggtaagggtgccctaactcctgtggggaccttctttcgta

SEQ ID NO: 390 - DNA, 40 bp, synthetic construct
  source 1..40: mol_type = other DNA
ggtaagggtgccctaacttctgtggggaccttctttcgtg

SEQ ID NO: 391 - DNA, 40 bp, synthetic construct
  source 1..40: mol_type = other DNA
ggtaagggtgctctaacgcctgtggggaccttctttggtg

SEQ ID NO: 392 - DNA, 40 bp, synthetic construct
  source 1..40: mol_type = other DNA
ggtaagggtgccctaactcctgtggggatcttctttcgta

SEQ ID NO: 393 - DNA, 40 bp, synthetic construct
  source 1..40: mol_type = other DNA
ggtaagggtgccgtaactcctgtggggacctcctttggtg

SEQ ID NO: 394 - DNA, 40 bp, synthetic construct
  source 1..40: mol_type = other DNA
ggtaaggctgccctaacgcctgtggggaccttctttcgtg

SEQ ID NO: 395 - DNA, 40 bp, synthetic construct
  source 1..40: mol_type = other DNA
ggtaagggtgccctaagtcctgtggggaccttctttggtg

SEQ ID NO: 396 - DNA, 40 bp, synthetic construct
  source 1..40: mol_type = other DNA
ggtaagggtgcactaactcctgtggggaccatctttggtg

SEQ ID NO: 397 - DNA, 40 bp, synthetic construct
  source 1..40: mol_type = other DNA
ggtaagggtgccctaacttctgtggggaccttctttggtg